(19) United States
(12) Patent Application Publication
(10) Pub. No.: US 2022/0380798 A1
(43) Pub. Date: Dec. 1, 2022

(54) AAV Expression Cassette and AAV Vectors Comprising the Same

(71) Applicant: Exonics Therapeutics, Inc., Watertown, MA (US)

(72) Inventor: Nathan Jones, Watertown, MA (US)

(73) Assignee: Exonics Therapeutics, Inc., Watertown, MA (US)

(21) Appl. No.: 17/358,618

(22) Filed: Jun. 25, 2021

Related U.S. Application Data
(63) Continuation of application No. PCT/US2020/012223, filed on Jan. 3, 2020.
(60) Provisional application No. 62/788,444, filed on Jan. 4, 2019.
(60) Provisional application No. 62/852,206, filed on May 23, 2019.
(60) Provisional application No. 62/849,140, filed on May 16, 2019.

(51) Int. Cl. C12N 15/86; C12N 15/11; C12N 9/22; A61K 48/00; A61P 43/00

(52) U.S. Cl. CPC C12N 15/86; C12N 15/11; A61P 43/00; A61K 48/005; C12N 9/22

(57) ABSTRACT

The disclosure provides AAV expression cassettes for production of AAV viral vectors, wherein the expression cassettes comprise a first inverted terminal repeat (ITR); a first promoter; a first gRNA comprising a first gRNA targeting region; a second promoter; a second gRNA comprising a second gRNA targeting region; a third promoter; a third gRNA comprising a third gRNA targeting region; and a second ITR. The disclosure also provides AAV viral vectors, including self-complimentary AAVs, comprising the expression cassettes of the disclosure. The AAVs disclosed herein may be used to treat genetic diseases, such as Duchenne Muscular Dystrophy (DMD).

### CROSS REFERENCE TO RELATED APPLICATIONS

This application is a Continuation of International Application No. PCT/US2020/012223, filed Jan. 3, 2020, which claims priority to U.S. Provisional Application Ser. No. 62/852,206, filed May 23, 2019, U.S Provisional Application Ser. No. 62/849,140, filed May 16, 2019, and U.S. Provisional Application Ser. No., 62/788,444, filed Jan. 4, 2019, each of which is incorporated by reference herein in its entirety for all purposes.

### SEQUENCE LISTING

The contents of the text file submitted electronically herewith are incorporated by reference in their entirety: a computer readable format copy of the Sequence Listing (filename: 01245-0010-00US_SeqList_ST25.txt, date recorded Jan. 4, 2020, file size ˜6 MB).

### FIELD

The disclosure relates to reagents for gene delivery. More particularly, the disclosure relates to AAV expression cassettes and AAV vectors comprising the same.

### BACKGROUND

Gene editing holds great promise for treating/preventing many genetic diseases including, for example, Duchenne Muscular Dystrophy (DMD). However, safe and effective delivery of gene editing machinery into the desired cells is necessary to achieve therapeutic benefit. There remains a need in the art for compositions and methods for delivering gene editing machinery to cells in vitro and/or in vivo.

### SUMMARY

Provided herein are adeno-associated virus (AAV) expression cassettes and AAV vectors comprising the same that are useful for delivering gene editing machinery to cells in vitro and in vivo. The AAV expression cassettes and AAV vectors described herein may be used to treat and/or prevent genetic diseases, such as DMD.

In some embodiments, the disclosure provides an AAV expression cassette comprising a first inverted terminal repeat (ITR), a first promoter, a sequence encoding a first guide (gRNA) comprising a first gRNA targeting region and a scaffold region, a second promoter, a sequence encoding a second gRNA comprising a second gRNA targeting region and a scaffold region, a third promoter, a sequence encoding a third gRNA comprising a third gRNA targeting region and a scaffold region, and a second ITR, wherein the AAV expression cassette is self-complimentary. In some embodiments, the first gRNA, the second gRNA, and the third gRNA are the same.

In some embodiments, the disclosure provides an AAV expression cassette comprising a first ITR, a first promoter, a sequence encoding a first gRNA comprising a first gRNA targeting region and a scaffold region, a second promoter, a sequence encoding a second gRNA comprising a second gRNA targeting region and a scaffold sequence, a third promoter, a sequence encoding a third gRNA comprising a third gRNA targeting region and a scaffold sequence, a first stuffer sequence, and a second ITR, wherein the stuffer sequence is a 3′ UTR sequence, for example, a 3′ UTR sequence isolated or derived from a gene expressed in muscle.

The disclosure also provides vectors, including viral vectors (e.g., AAV vectors or baculovirus vectors) and non-viral vectors, comprising an AAV expression cassette.

The disclosure also provides AAV particles comprising an AAV expression cassette encapsidated by an AAV capsid protein.

The disclosure also provides methods for producing an AAV vector comprising contacting a vector comprising an AAV expression cassette with an AAV producer cell.

The disclosure also provides methods for correcting a gene defect in a cell, the method comprising contacting an AAV vector comprising an AAV expression cassette with the cell.

Also provided are methods for treating a subject in need thereof comprising administering to the subject an AAV vector comprising an AAV expression cassette. The methods may further comprise administering to the subject an AAV vector comprising an expression cassette for a nuclease (e.g., a Cas9 or Cpf1 nuclease).

These and other embodiments are addressed in more detail in the detailed description set forth below.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic drawing depicting an exemplary AAV9-H-sgRNA construct of the disclosure.

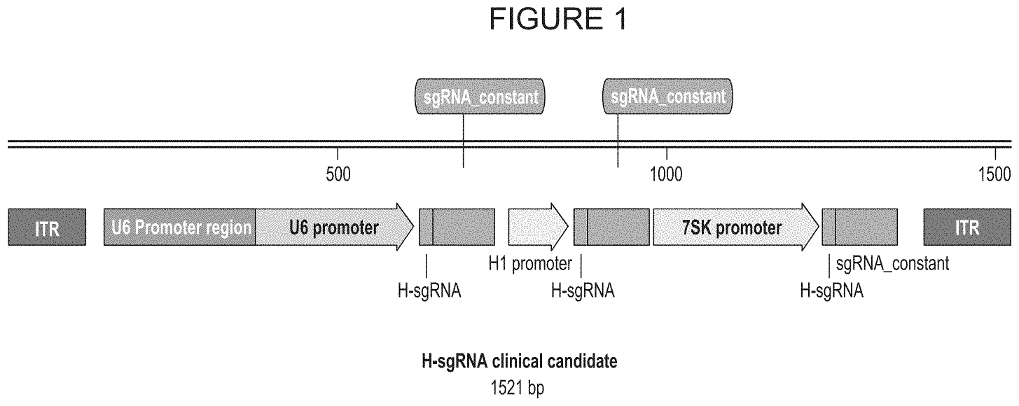

FIG. 2 is a schematic drawing depicting an exemplary AAV9-CK8e-SpCas9 construct of the disclosure.

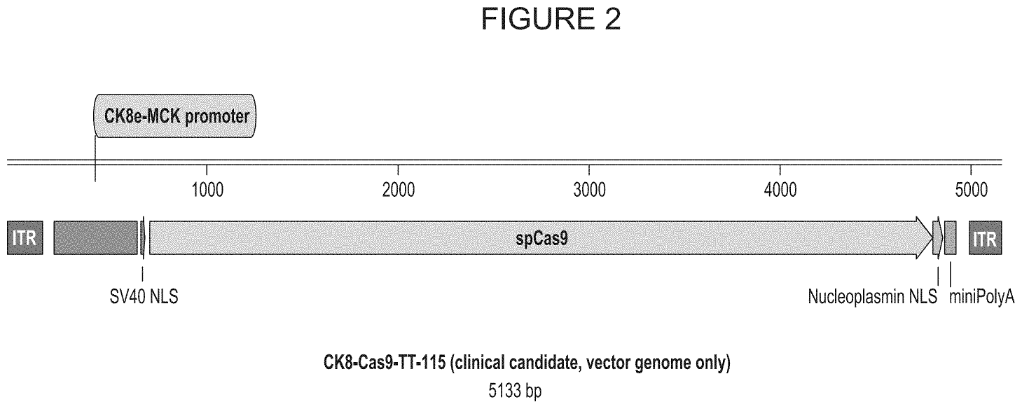

FIG. 3A-3F shows dystrophin restoration (percent of wildtype) in quadriceps (FIG. 3A), triceps (FIG. 3B), tibialis anterior (FIG. 3C), gastrocnemius (FIG. 3D), diaphragm (FIG. 3E), and heart (FIG. 3F) at various doses of AAV-Cas9 and AAV-sgRNA. Data are represented as mean±standard deviation.

FIG. 4A-4B show editing efficiency, as determined using TIDE analysis in samples form heart (FIG. 4A) and quadriceps (FIG. 4B) at various doses of AAV-Cas9 and AAV-sgRNA. Data are represented as mean±standard deviation.

FIG. 5A-5F show tissue Cas9 expression in quadriceps (FIG. 5A), triceps (FIG. 5B), tibialis anterior (FIG. 5C), gastrocnemius (FIG. 5D), diaphragm (FIG. 5E), heart (FIG. 5F) at various doses of AAV-Cas9 and AAV-sgRNA. Data are represented as mean±standard deviation.

### DETAILED DESCRIPTION

Gene editing is a promising treatment option for many diseases, including genetic diseases such as DMD. DMD is a rare genetic disease caused by loss-of-function mutations in the dystrophin gene. An X-linked condition, the disease mostly affects boys. It usually manifests in the form of muscle weakness in children between the ages of 3 and 5. There is no cure for DMD. Steroids can slow the progression of symptoms, however the disease eventually causes life-threatening damage to the heart muscles. Few patients live beyond their 30s.

However, there remains a need in the art for effective compositions and methods for delivering genes and other therapeutic sequences to patients in need thereof.

Unless otherwise defined, all technical and scientific terms used herein have the same meaning as commonly understood by one of ordinary skill in the art to which this disclosure belongs. The terminology used in the detailed description herein is for the purpose of describing particular embodiments only and is not intended to be limiting.

All publications, patent applications, patents, GenBank or other accession numbers and other references mentioned herein are each incorporated by reference herein in their entirety.

The singular forms “a,” “an” and “the” are intended to include the plural forms as well, unless the context clearly indicates otherwise.

Furthermore, the terms “about” and “approximately” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

Reference to a vector or other DNA sequences as “recombinant” merely acknowledges the operable linkage of DNA sequences which are not typically operably linked as isolated from or found in nature.

Unless the context indicates otherwise, it is specifically intended that the various features described herein can be used in any combination.

### AAV Expression Cassettes

The wildtype AAV genome comprises two open reading frames, Rep and Cap, flanked by two inverted terminal repeats (ITRs). Typically, when producing a recombinant AAV, the sequence between the two ITRs is replaced with one or more sequence of interest (e.g., a transgene), and the Rep and Cap sequences are provided in trans. The recombinant AAV genome construct, comprising two ITRs flanking a sequence of interest (such as a transgene), is referred to herein as an AAV expression cassette. The disclosure provides AAV expression cassettes for production of AAV viral vectors.

In some embodiments, an AAV expression cassette comprises a first ITR, a transgene sequence, and a second ITR. In some embodiments, an AAV expression cassette comprises a first ITR, an expression control sequence (such as a promoter or enhancer), a transgene sequence, and a second ITR. In some embodiments, an AAV expression cassette comprises a first ITR, an expression control sequence (such as a promoter or enhancer), a transgene sequence, a stuffer sequence, and a second ITR.

The transgene may comprise, for example, a gRNA sequence. In some embodiments, an AAV expression cassette comprises a first ITR, a gRNA sequence, and a second ITR. In some embodiments, an AAV expression cassette comprises a first ITR, an expression control sequence (such as a promoter or enhancer), a gRNA sequence, and a second ITR. In some embodiments, an AAV expression cassette comprises a first ITR, an expression control sequence (such as a promoter or enhancer), a gRNA sequence, a stuffer sequence, and a second ITR.

In some embodiments, the transgene comprises more than one guide RNA sequence, such as two, three, four, five, six, seven, or eight gRNA sequences. In some embodiments, the transgene comprises three, four or five gRNA sequences. In some embodiments, each gRNA sequence is operably linked to an expression control sequence (such as a promoter or enhancer). In some embodiments, an AAV expression cassette comprises a first ITR, a first expression control sequence (such as a promoter or enhancer), a first gRNA sequence, a second expression control sequence (such as a promoter or enhancer), a second gRNA sequence, and a second ITR. In some embodiments, an AAV expression cassette comprises a first ITR, a first expression control sequence (such as a promoter or enhancer), a first gRNA sequence, a second expression control sequence (such as a promoter or enhancer), a second gRNA sequence, a third expression control sequence (such as a promoter or enhancer), a third gRNA sequence, and a second ITR. In some embodiments, an AAV expression cassette comprises a first ITR, a first expression control sequence (such as a promoter or enhancer), a first gRNA sequence, a second expression control sequence (such as a promoter or enhancer), a second gRNA sequence, a third expression control sequence (such as a promoter or enhancer), a third gRNA sequence, a fourth expression control sequence (such as a promoter or enhancer), a fourth gRNA sequence, and a second ITR. In some embodiments, an AAV expression cassette comprises a first ITR, a first expression control sequence (such as a promoter or enhancer), a first gRNA sequence, a second expression control sequence (such as a promoter or enhancer), a second gRNA sequence, a third expression control sequence (such as a promoter or enhancer), a third gRNA sequence, a fourth expression control sequence (such as a promoter or enhancer), a fourth gRNA sequence, a fifth expression control sequence (such as a promoter or enhancer), a fifth gRNA sequence, and a second ITR. In some embodiments, all of the gRNA sequences are the same. In some embodiments, two or more of the gRNA sequences are different. In some embodiments, the AAV expression cassette further comprises a stuffer sequence.

In some embodiments, the AAV expression cassette may comprise a self-complimentary sequence, i.e. be capable of forming a self-complimentary AAV (scAAV). In some embodiments, the AAV expression cassette has been designed to form an intra-molecular double stranded DNA template. Self-complimentary AAVs are described, for example, in WO 2001/92551 and WO 2001/11034, which are incorporated by reference herein in their entireties.

In some embodiments, the AAV expression cassette is approximately one half the size of a wildtype AAV genome. In some embodiments, the AAV expression cassette forms a double-stranded DNA under appropriate conditions. In some embodiments, a sequence of the AAV expression cassette is substantially self-complimentary (i.e., at least 85%, at least 90%, at least 95%, at least 96%, at least 97%, at least 98%, at least 99% self-complimentary).

For a self-complementary expression cassette, not all bases in a single strand must be complementary to each and every base of the opposing complementary strand. There need only be a sufficient number of complementary nucleotide or nucleoside bases to enable the two polynucleotide or nucleic acid molecules to be able to specifically hybridize or bind (anneal) to each other. Hence, there may be short sequence segments or regions of non-complementary bases between the self-complementary polynucleotide or nucleic acid molecules. For example, 1-5, 5-10, 10-20, 20-30, 30-40, 40-50, 50-75, 75-100, or 100-150 or more contiguous or non-contiguous non-complementary bases may be present but there may be sufficient complementary bases over the lengths of the two sequences such that the two polynucleotide or nucleic acid molecules are able to specifically hybridize or bind (anneal) to each other and form a double-strand (or duplex) sequence. Accordingly, sequences of the two single stranded regions may be less than 100% complementary to each other and yet still be able to form a double-strand duplex molecule. In particular embodiments, two single strand sequences have at least 70%, at least 75%, at least 80%, at least 85%, at least 90%, at least 95%, at least 98%, at least 99% or more complementarity to each other.

In some embodiments, the self-complementary expression cassette is expressed faster (more rapid onset) than a single stranded transgene counterpart. Thus, such expression can be detected by measuring expression over time, such as at defined time points (e.g., 1, 2, 3, 4, 5, 6, 7, 8, 9, 10, 11, 12, 12-16, 16-20, 20-24 hours, for example). Furthermore, the amount of expression of the double stranded self-complementary expression cassette typically is greater than a single stranded reporter transgene counterpart. Thus, such expression can be detected by measuring at a point in time in which expression would be considered to be approaching or at a maximum.

In some embodiments, an AAV expression cassette comprises sequences encoding a first ITR, a first promoter, a first gRNA comprising a first gRNA targeting region, a second promoter, a second gRNA comprising a second gRNA targeting region, a third promoter, a third gRNA comprising a third gRNA targeting region; and a second ITR; wherein the AAV expression cassette is self-complimentary. At least one of the first, second, and third gRNA targeting sequences may target the dystrophin gene. In some embodiments, the first, second, and third gRNA targeting sequences are each individually selected from any one of the gRNA sequences in any one of Tables 3-14, or a sequence at least 95% identical thereto. In some embodiments, at least two of the first, second, and third gRNA targeting sequences are different. In some embodiments, the first, second, and third gRNA targeting sequences are the same. In some embodiments, the first, second, and/or third gRNA targeting sequences have a sequence that is at least 95% identical or 100% identical to the sequence of SEQ ID NO: 13.

In some embodiments, an AAV expression cassette comprises a first gRNA comprising a first gRNA targeting region, a second gRNA comprising a second gRNA targeting region, a third gRNA comprising a third gRNA targeting region, and a fourth gRNA comprising a fourth gRNA targeting region. In some embodiments, two, three, or four of the gRNAs are the same. In some embodiments, two, three, or four of the gRNAs are different. In some embodiments, an AAV expression cassette comprises a first promoter, a first gRNA comprising a first gRNA targeting region, a second promoter, a second gRNA comprising a second gRNA targeting region, a third promoter, a third gRNA comprising a third gRNA targeting region, a fourth promoter, and a fourth gRNA comprising a fourth gRNA targeting region. In some embodiments, an AAV expression cassette comprises a first ITR, a first promoter, a first gRNA comprising a first gRNA targeting region, a second promoter, a second gRNA comprising a second gRNA targeting region, a third promoter, a third gRNA comprising a third gRNA targeting region, a fourth promoter, a fourth gRNA comprising a fourth gRNA targeting region, and a second ITR. In some embodiments, the expression cassette further comprises a stuffer sequence. In some embodiments, the expression cassette is self-complimentary.

In some embodiments, an AAV expression cassette comprises a first gRNA comprising a first gRNA targeting region, a second gRNA comprising a second gRNA targeting region, a third gRNA comprising a third gRNA targeting region, a fourth gRNA comprising a fourth gRNA targeting region, and a fifth gRNA comprising a fifth gRNA targeting region. In some embodiments, two, three, four, or five of the gRNAs are the same. In some embodiments, two, three, four or five of the gRNAs are different. In some embodiments, an AAV expression cassette comprises a first promoter, a first gRNA comprising a first gRNA targeting region, a second promoter, a second gRNA comprising a second gRNA targeting region, a third promoter, a third gRNA comprising a third gRNA targeting region, a fourth promoter, a fourth gRNA comprising a fourth gRNA targeting region, a fifth promoter, and a fifth gRNA comprising a fifth gRNA targeting region. In some embodiments, an AAV expression cassette comprises a first ITR, a first promoter, a first gRNA comprising a first gRNA targeting region, a second promoter, a second gRNA comprising a second gRNA targeting region, a third promoter, a third gRNA comprising a third gRNA targeting region, a fourth promoter, a fourth gRNA comprising a fourth gRNA targeting region, a fifth promoter, a fifth gRNA comprising a fifth gRNA targeting region, and a second ITR. In some embodiments, the expression cassette further comprises a stuffer sequence. In some embodiments, the expression cassette is self-complimentary.

In some embodiments, an AAV expression cassette comprises sequences encoding a first inverted terminal repeat (ITR), a first promoter, a first gRNA comprising a first gRNA targeting region (e.g., a sequence at least 95% or 100% identical to SEQ ID NO: 13) and a scaffold region, a second promoter, a second gRNA comprising a second gRNA targeting region (e.g., a sequence at least 95% or 100% identical to SEQ ID NO: 13) and a scaffold region, a third promoter, a third gRNA comprising a third gRNA targeting region (e.g., a sequence at least 95% or 100% identical to SEQ ID NO: 13) and a scaffold region; and a second ITR; wherein the AAV expression cassette is self-complimentary.

In some embodiments, an AAV expression cassette comprises sequences encoding a first ITR, a first promoter (e.g., a sequence at least 95% or 100% identical to SEQ ID NO: 15), a first gRNA comprising a first gRNA targeting region (e.g., a sequence at least 95% or 100% identical to SEQ ID NO: 13) and a scaffold region, a second promoter (e.g., a sequence at least 95% or 100% identical to SEQ ID NO: 16), a second gRNA comprising a second gRNA targeting region (e.g., a sequence at least 95% or 100% identical to SEQ ID NO: 13) and a scaffold region, a third promoter (e.g., a sequence at least 95% or 100% identical to SEQ ID NO: 17), a third gRNA comprising a third gRNA targeting region (e.g., a sequence at least 95% or 100% identical to SEQ ID NO: 13) and a scaffold region; and a second ITR; wherein the AAV expression cassette is self-complimentary.

In some embodiments, an AAV expression cassette comprises sequences encoding a first ITR (e.g., a sequence at least 95% or 100% identical to SEQ ID NO: 1), a first promoter (e.g., a sequence at least 95% or 100% identical to SEQ ID NO: 15), a first gRNA comprising a first gRNA targeting region (e.g., a sequence at least 95% or 100% identical to SEQ ID NO: 13) and a scaffold region, a second promoter (e.g., a sequence at least 95% or 100% identical to SEQ ID NO: 16), a second gRNA comprising a second gRNA targeting region (e.g., a sequence at least 95% or 100% identical to SEQ ID NO: 13) and a scaffold region, a third promoter (e.g., a sequence at least 95% or 100% identical to SEQ ID NO: 17), a third gRNA comprising a third gRNA targeting region (e.g., a sequence at least 95% or 100% identical to SEQ ID NO: 13) and a scaffold region; and a second ITR (e.g., a sequence at least 95% or 100% identical to SEQ ID NO: 2); wherein the AAV expression cassette is self-complimentary.

In some embodiments, an AAV expression cassette comprising sequences encoding a first ITR, a first promoter, a first gRNA comprising a first gRNA targeting sequence, a second promoter, a second gRNA comprising a first gRNA targeting sequence, a third promoter, a third gRNA comprising a first gRNA targeting sequence, a first stuffer sequence, and a second ITR; wherein the stuffer sequence is a 3′ UTR sequence isolated or derived from a gene expressed in muscle (e.g., desmin).

In some embodiments, an AAV expression cassette comprises sequences encoding a first ITR, a first promoter, a first gRNA comprising a first gRNA targeting sequence, a second promoter, a second gRNA comprising a first gRNA targeting sequence, a third promoter, a third gRNA comprising a first gRNA targeting sequence, a first stuffer sequence, and a second ITR; wherein the stuffer sequence is a 3′ UTR sequence isolated or derived from a gene expressed in muscle. At least one of the first, second, and third gRNA targeting sequences may target the dystrophin gene. In some embodiments, the first, second, and third gRNA targeting sequences are each individually selected from any one of the gRNA sequences in any one of Tables 3-14 or a sequence at least 95% identical thereto. In some embodiments, at least two of the first, second, and third gRNA targeting sequences are different. In some embodiments, the first, second, and third gRNA targeting sequences are the same. In some embodiments, the first, second, and/or the third gRNA targeting sequences have a sequence that is at least 95% identical or 100% identical to the sequence of SEQ ID NO: 13.

In some embodiments, an AAV expression cassette comprises sequences encoding a first ITR, a first promoter, a first gRNA comprising a first gRNA targeting sequence (e.g., a sequence at least 95% or 100% identical to SEQ ID NO: 13), a second promoter, a second gRNA comprising a first gRNA targeting sequence (e.g., a sequence at least 95% or 100% identical to SEQ ID NO: 13), a third promoter, a third gRNA comprising a first gRNA targeting sequence (e.g., a sequence at least 95% or 100% identical to SEQ ID NO: 13), a first stuffer sequence, and a second ITR; wherein the stuffer sequence is a 3′ UTR sequence isolated or derived from a gene expressed in muscle (e.g., desmin).

In some embodiments, an AAV expression cassette comprises sequences encoding a first ITR, a first promoter (e.g., a sequence at least 95% or 100% identical to SEQ ID NO: 15), a first gRNA comprising a first gRNA targeting sequence (e.g., a sequence at least 95% or 100% identical to SEQ ID NO: 13), a second promoter (e.g., a sequence at least 95% or 100% identical to SEQ ID NO: 16), a second gRNA comprising a first gRNA targeting sequence (e.g., a sequence at least 95% or 100% identical to SEQ ID NO: 13), a third promoter (e.g., a sequence at least 95% or 100% identical to SEQ ID NO: 17), a third gRNA comprising a first gRNA targeting sequence (e.g., a sequence at least 95% or 100% identical to SEQ ID NO: 13), a first stuffer sequence, and a second ITR; wherein the stuffer sequence is a 3′ UTR sequence isolated or derived from a gene expressed in muscle (e.g., desmin).

In some embodiments, an AAV expression cassette comprises sequences encoding a first ITR (e.g., a sequence at least 95% or 100% identical to SEQ ID NO: 3), a first promoter (e.g., a sequence at least 95% or 100% identical to SEQ ID NO: 15), a first gRNA comprising a first gRNA targeting sequence (e.g., a sequence at least 95% or 100% identical to SEQ ID NO: 13), a second promoter (e.g., a sequence at least 95% or 100% identical to SEQ ID NO: 16), a second gRNA comprising a first gRNA targeting sequence (e.g., a sequence at least 95% or 100% identical to SEQ ID NO: 13), a third promoter (e.g., a sequence at least 95% or 100% identical to SEQ ID NO: 17), a third gRNA comprising a first gRNA targeting sequence (e.g., a sequence at least 95% or 100% identical to SEQ ID NO: 13), a first stuffer sequence, and a second ITR (e.g., a sequence at least 95% or 100% identical to SEQ ID NO: 12); wherein the stuffer sequence is a 3′ UTR sequence isolated or derived from a gene expressed in muscle (e.g., desmin).

In some embodiments, an AAV expression cassette comprises sequences encoding a first ITR (e.g., a sequence at least 95% or 100% identical to SEQ ID NO: 3), a first promoter (e.g., a sequence at least 95% or 100% identical to SEQ ID NO: 15), a first gRNA comprising a first gRNA targeting sequence (e.g., a sequence at least 95% or 100% identical to SEQ ID NO: 13), a second promoter (e.g., a sequence at least 95% or 100% identical to SEQ ID NO: 16), a second gRNA comprising a first gRNA targeting sequence (e.g., a sequence at least 95% or 100% identical to SEQ ID NO: 13), a third promoter (e.g., a sequence at least 95% or 100% identical to SEQ ID NO: 17), a third gRNA comprising a first gRNA targeting sequence (e.g., a sequence at least 95% or 100% identical to SEQ ID NO: 13), a first stuffer sequence (e.g., a sequence at least 95% or 100% identical to SEQ ID NO: 20), and a second ITR (e.g., a sequence at least 95% or 100% identical to SEQ ID NO: 12); wherein the stuffer sequence is a 3′ UTR sequence isolated or derived from a gene expressed in muscle (e.g., desmin).

In some embodiments, an AAV expression cassette comprises a sequence at least 95%, at least 96%, at least 97%, at least 98%, at least 99% or 100% identical to the sequence of SEQ ID NO: 25 or 26.

### ITRs

The AAV expression cassettes may comprise one or more ITR sequence. ITR sequences assist in replication, packaging into AAV particles, and efficient integration into cell chromosomes. Typically, ITRs are palindromic.

The AAV expression cassettes of the disclosure may comprise a first ITR 5′ to a sequence of interest (e.g., a gRNA) and a second ITR 3′ to the sequence of interest. The ITRs may each comprise or consist of 1 to 145 nucleotides, such as 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, or 145 nucleotides. In some embodiments, a first ITR and a second ITR each comprise or consist of 130 nucleotides. In some embodiments, a first ITR comprises or consists of 117 nucleotides, and a second ITR comprises or consists of 130 nucleotides. In some embodiments, a first ITR comprises or consists of 130 nucleotides, and a second ITR comprises or consists of 117 nucleotides. In some embodiments, a first ITR and a second ITR each comprise or consist of 117 nucleotides.

In some embodiments, the ITRs are isolated or derived from naturally occurring AAV sequences. For example, in some embodiments, the ITRs may each independently be isolated or derived from any one of AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV. In particular embodiments, the ITRs are isolated or derived from AAV2. In some embodiments, an ITR may have a sequence that is at least 90%, at least 95%, at least 96%, at least 97%, at least 98%, or at least 99% identical to the sequence of an ITR from any one of AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV. In some embodiments, an ITR may be synthetic.

Exemplary ITR sequences which may be used in the AAV expression cassettes of the disclosure are provided in Table 1 below:

TABLE 1
Exemplary ITR Sequences

| | | SEQ ID |
|---|---|---|
| 5′ or 3′ | Sequence (5′ to 3′) | NO: |
| 5′ | CCTGCAGGCAGCTGCGCGCTCGCTCGCTCACTGAGGCCGCCCGGGCAA | 1 |
| | AGCCCGGGCGTCGGGCGACCTTTGGTCGCCCGGCCTCAGTGAGCGAGC | |
| | GAGCGCGCAGAGAGGGAGTGG | |
| 5′ | CTGCGCGCTCGCTCGCTCACTGAGGCCGCCCGGGCAAAGCCCGGGCGT | 2 |
| | CGGGCGACCTTTGGTCGCCCGGCCTCAGTGAGCGAGCGAGCGCGCAGA | |
| | GAGGGAGTGGCCAACTCCATCACTAGGGGTTCCT | |
| 3′ | AGGAACCCCTAGTGATGGAGTTGGCCACTCCCTCTCTGCGCGCTCGCTC | 3 |
| | GCTCACTGAGGCCGGGCGACCAAAGGTCGCCCGACGCCCGGGCTTTGC | |
| | CCGGGCGGCCTCAGTGAGCGAGCGAGCGCGCAG | |
| 5′ | TTGGCCACTCCCTCTCTGCGCGCTCGCTCGCTCACTGAGGCCGGGCGACCAA | 27 |
| | AGGTCGCCCGACGCCCGGGCTTTGCCCGGGCGGCCTCAGTGAGCGAGCGA | |
| | GCGCGCAGAGAGGGAGTGGCCAACTCCATCACTAGGGGTTCCT | |
| 3′ | AGGAACCCCTAGTGATGGAGTTGGCCACTCCCTCTCTGCGCGCTCGCTCGCT | 28 |
| | CACTGAGGCCGCCCGGGCAAAGCCCGGGCGTCGGGCGACCTTTGGTCGCCC | |
| | GGCCTCAGTGAGCGAGCGAGCGCGCAGAGAGGGAGTGGCCAA | |

In some embodiments, an ITR has a sequence that is at least about 95%, about 96%, about 97%, about 98%, about 99%, or 100% identical to any one of SEQ ID NO: 1-3 or 27-28. In some embodiments, an ITR is a truncated form of any one of SEQ ID NO: 1-3 or 27-28. For example, an ITR may have a sequence identical to any one of SEQ ID NO: 1-3 or 27-28, except that 1 to 25 base pairs have been removed from either the 5′ end or the 3′ end of the sequence. In some embodiments, an ITR may have a sequence identical to any one of SEQ ID NO: 1-3 or 27-28, except that 1-25 base pairs have been removed from the sequence, either consecutively or at different points throughout the sequence.

In some embodiments, when packaging large coding regions into AAV vector particles, it short ITR sequences may be used in order to increase the packaging size of the AAV while maintaining efficient expression. In some embodiments, the ITR sequences enable production of a self-complimentary AAV (scAAV).

Transgenes (e.q., gRNAs)

In some embodiments, the transgene comprises a sequence encoding a guide RNA (gRNA). In some embodiments, the transgene comprises more than one gRNA sequence, such as two, three, four, five, six, seven, or eight gRNA sequences.

In some embodiments, the gRNA may be a crRNA-tracrRNA fusion transcript. In some embodiments, the gRNA may comprise a polyA tail.

The gRNAs may comprise a targeting region that is specific for a genomic sequence of interest, including coding and non-coding sequences. In some embodiments, the gRNA targeting region may be about 17-24 base pairs in length, such as about 20 base pairs in length. In some embodiments, the gRNA targeting sequence may be about 15, 16, 17, 18, 19, 20, 21, 22, 23, 24, 25, 26, 27, 28, 29, or 30 base pairs in length. In some embodiments, the gRNA targeting sequence has between about 40% to about 80% GC content.

In some embodiments, the gRNA targeting sequence targets a site that immediately precedes a 5′ protospacer adjacent motif (PAM). The PAM sequence may be selected based on the desired nuclease. For example, the PAM sequence may be any one of the PAM sequences shown in Table 2 below, wherein N refers to any nucleic acid, R refers to A or G, Y refers to C or T, W refers to A or T, and V refers to A or C or G.

TABLE 2
Nucleases and PAM sequences

| PAM | | | |
|---|---|---|---|
| sequence | | | |
| (5′ to 3′) | SEQ ID NO: | Nuclease | Isolated from |
| NGG | 4 | SpCas9 | Streptococcus pyogenes |
| NGRRT or | 5, 6 | SaCas9 | Staphylococcus aureus |
| NGRRN | | | |
| NNNNGATT | 7 | NmeCas9 | Neisseria meningitidis |
| NNNNRYAC | 8 | CjCas9 | Campylobacter jejuni |
| NNAGAAW | 9 | StCas9 | Streptococcus thermophilus |
| TTTV | 10 | LbCpf1 | Lachnospiraceae bacterium |
| TTTV | 11 | AsCpf1 | Acidaminococcus sp. |

In some embodiments, a gRNA targeting region may target a sequence of a mammalian gene, such as a human gene. In some embodiments, the gRNA targeting region may target an intronic sequence. In some embodiments, the gRNA targeting region may target an exonic sequence. In some embodiments, the gRNA may target an intron-exon junction. In some embodiments, the gRNA may target a splice site. In some embodiments, a gRNA may target a mutant gene. In some embodiments, a gRNA may target a wildtype gene.

In some embodiments, a gRNA may target the dystrophin gene. For example, the gRNA may target a wildtype or a mutant dystrophin gene. An exemplary wildtype dystrophin sequence includes the human sequence (see GenBank Accession No. NC_000023.11), located on the human X chromosome, which codes for the protein dystrophin (GenBank Accession No. AAA53189; SEQ ID NO: 12), the sequence of which is reproduced below:

| | |
|---|---|
| 1 | MLWWEEVEDC YEREDVQKKT FTKWVNAQFS KFGKQHIENL FSDLQDGRRL LDLLEGLTGQ |
| 61 | KLPKEKGSTR VHALNNVNKA LRVLQNNNVD LVNIGSTDIV DGNHKLTLGL IWNIILHWQV |
| 121 | KNVMKNIMAG LQQTNSEKIL LSWVRQSTRN YPQVNVINFT TSWSDGLALN ALIHSHRPDL |
| 181 | FDWNSVVCQQ SATQRLEHAF NIARYQLGIE KLLDPEDVDT TYPDKKSILM YITSLFQVLP |
| 241 | QQVSIEAIQE VEMLPRPPKV TKEEHFQLHH QMHYSQQITV SLAQGYERTS SPKPRFKSYA |
| 301 | YTQAAYVTTS DPTRSPFPSQ HLEAPEDKSF GSSLMESEVN LDRYQTALEE VLSWLLSAED |
| 361 | TLQAQGEISN DVEVVKDQFH THEGYMMDLT AHQGRVGNIL QLGSKLIGTG KLSEDEETEV |
| 421 | QEQMNLLNSR WECLRVASME KQSNLHRVLM DLQNQKLKEL NDWLTKTEER TRKMEEEPLG |
| 481 | PDLEDLKRQV QQHKVLQEDL EQEQVRVNSL THMVVVVDES SGDHATAALE EQLKVLGDRW |
| 541 | ANICRWTEDR WVLLQDILLK WQRLTEEQCL FSAWLSEKED AVNKIHTTGF KDQNEMLSSL |
| 601 | QKLAVLKADL EKKKQSMGKL YSLKQDLLST LKNKSVTQKT EAWLDNFARC WDNLVQKLEK |
| 661 | STAQISQAVT TTQPSLTQTT VMETVTTVTT REQILVKHAQ EELPPPPPQK KRQITVDSEI |
| 721 | RKRLDVDITE LHSWITRSEA VLQSPEFAIF RKEGNFSDLK EKVNAIEREK AEKFRKLQDA |
| 781 | SRSAQALVEQ MVNEGVNADS IKQASEQLNS RWIEFCQLLS ERLNWLEYQN NIIAFYNQLQ |
| 841 | QLEQMTTTAE NWLKIQPTTP SEPTAIKSQL KICKDEVNRL SGLQPQIERL KIQSIALKEK |
| 901 | GQGPMFLDAD FVAFTNHFKQ VFSDVQAREK ELQTIFDTLP PMRYQETMSA IRTWVQQSET |
| 961 | KLSIPQLSVT DYEIMEQRLG ELQALQSSLQ EQQSGLYYLS TTVKEMSKKA PSEISRKYQS |
| 1021 | EFEEIEGRWK KLSSQLVEHC QKLEEQMNKL RKIQNHIQTL KKWMAEVDVF LKEEWPALGD |
| 1081 | SEILKKQLKQ CRLLVSDIQT IQPSLNSVNE GGQKIKNEAE PEFASRLETE LKELNTQWDH |
| 1141 | MCQQVYARKE ALKGGLEKTV SLQKDLSEMH EWMTQAEEEY LERDFEYKTP DELQKAVEEM |
| 1201 | KRAKEEAQQK EAKVKLLTES VNSVIAQAPP VAQEALKKEL ETLTTNYQWL CTRLNGKCKT |
| 1261 | LEEVWACWHE LLSYLEKANK WLNEVEFKLK TTENIPGGAE EISEVLDSLE NLMRHSEDNP |
| 1321 | NQIRILAQTL TDGGVMDELI NEELETFNSR WRELHEEAVR RQKLLEQSIQ SAQETEKSLH |
| 1381 | LIQESLTFID KQLAAYIADK VDAAQMPQEA QKIQSDLTSH EISLEEMKKH NQGKEAAQRV |
| 1441 | LSQIDVAQKK LQDVSMKFRL FQKPANFELR LQESKMILDE VKMHLPALET KSVEQEVVQS |
| 1501 | QLNHCVNLYK SLSEVKSEVE MVIKTGRQIV QKKQTENPKE LDERVTALKL HYNELGAKVT |
| 1561 | ERKQQLEKCL KLSRKMRKEM NVLTEWLAAT DMELTKRSAV EGMPSNLDSE VAWGKATQKE |
| 1621 | IEKQKVHLKS ITEVGEALKT VLGKKETLVE DKLSLLNSNW IAVTSRAEEW LNLLLEYQKH |
| 1681 | METFDQNVDH ITKWIIQADT LLDESEKKKP QQKEDVLKRL KAELNDIRPK VDSTRDQAAN |
| 1741 | LMANRGDHCR KLVEPQISEL NHRFAAISHR IKTGKASIPL KELEQFNSDI QKLLEPLEAE |
| 1801 | IQQGVNLKEE DFNKDMNEDN EGTVKELLQR GDNLQQRITD ERKREEIKIK QQLLQTKHNA |
| 1861 | LKDLRSQRRK KALEISHQWY QYKRQADDLL KCLDDIEKKL ASLPEPRDER KIKEIDRELQ |
| 1921 | KKKEELNAVR RQAEGLSEDG AAMAVEPTQI QLSKRWREIE SKFAQFRRLN FAQIHTVREE |
| 1981 | TMMVMTEDMP LEISYVPSTY LTEITHVSQA LLEVEQLLNA PDLCAKDFED LFKQEESLKN |
| 2041 | IKDSLQQSSG RIDIIHSKKT AALQSATPVE RVKLQEALSQ LDFQWEKVNK MYKDRQGRFD |
| 2101 | RSVEKWRRFH YDIKIFNQWL TEAEQFLRKT QIPENWEHAK YKWYLKELQD GIGQRQTVVR |
| 2161 | TLNATGEEII QQSSKTDASI LQEKLGSLNL RWQEVCKQLS DRKKRLEEQK NILSEFQRDL |
| 2221 | NEFVLWLEEA DNIASIPLEP GKEQQLKEKL EQVKLLVEEL PLRQGILKQL NETGGPVLVS |
| 2281 | APISPEEQDK LENKLKQTNL QWIKVSRALP EKQGEIEAQI KDLGQLEKKL EDLEEQLNHL |
| 2341 | LLWLSPIRNQ LEIYNQPNQE GPFDVQETEI AVQAKQPDVE EILSKGQHLY KEKPATQPVK |
| 2401 | RKLEDLSSEW KAVNRLLQEL RAKQPDLAPG LTTIGASPTQ TVTLVTQPVV TKETAISKLE |
| 2461 | MPSSLMLEVP ALADFNRAWT ELTDWLSLLD QVIKSQRVMV GDLEDINEMI IKQKATMQDL |
| 2521 | EQRRPQLEEL ITAAQNLKNK TSNQEARTII TDRIERIQNQ WDEVQEHLQN RRQQLNEMLK |
| 2581 | DSTQWLEAKE EAEQVLGQAR AKLESWKEGP YTVDAIQKKI TETKQLAKDL RQWQTNVDVA |
| 2641 | NDLALKLLRD YSADDTRKVH MITENINASW RSIHKRVSER EAALEETHRL LQQFPLDLEK |
| 2701 | FLAWLTEAET TANVLQDATR KERLLEDSKG VKELMKQWQD LQGEIEAHTD VYHNLDENSQ |
| 2761 | KILRSLEGSD DAVLLQRRLD NMNFKWSELR KKSLNIRSHL EASSDQWKRL HLSLQELLVW |
| 2821 | LQLKDDELSR QAPIGGDFPA VQKQNDVHRA FKRELKTKEP VIMSTLETVR IFLTEQPLEG |
| 2881 | LEKLYQEPRE LPPEERAQNV TRLLRKQAEE VNTEWEKLNL HSADWQRKID ETLERLQELQ |
| 2941 | EATDELDLKL RQAEVIKGSW QPVGDLLIDS LQDHLEKVKA LRGEIAPLKE NVSHVNDLAR |
| 3001 | QLTTLGIQLS PYNLSTLEDL NTRWKLLQVA VEDRVRQLHE AHRDFGPASQ HFLSTSVQGP |
| 3061 | WERAISPNKV PYYINHETQT TCWDHPKMTE LYQSLADLNN VRFSAYRTAM KLRRLQKALC |
| 3121 | LDLLSLSAAC DALDQHNLKQ NDQPMDILQI INCLTTIYDR LEQEHNNLVN VPLCVDMCLN |
| 3181 | WLLNVYDTGR TGRIRVLSFK TGIISLCKAH LEDKYRYLFK QVASSTGFCD QRRLGLLLHD |
| 3241 | SIQIPRQLGE VASFGGSNIE PSVRSCFQFA NNKPEIEAAL FLDWMRLEPQ SMVWLPVLHR |
| 3301 | VAAAETAKHQ AKCNICKECP IIGFRYRSLK HFNYDICQSC FFSGRVAKGH KMHYPMVEYC |
| 3361 | TPTTSGEDVR DFAKVLKNKF RTKRYFAKHP RMGYLPVQTV LEGDNMETPV TLINFWPVDS |
| 3421 | APASSPQLSH DDTHSRIEHY ASRLAEMENS NGSYLNDSIS PNESIDDEHL LIQHYCQSLN |
| 3481 | QDSPLSQPRS PAQILISLES EERGELERIL ADLEEENRNL QAEYDRLKQQ HEHKGLSPLP |
| 3541 | SPPEMMPTSP QSPRDAELIA EAKLLRQHKG RLEARMQILE DHNKQLESQL HRLRQLLEQP |
| 3601 | QAEAKVNGTT VSSPSTSLQR SDSSQPMLLR VVGSQTSDSM GEEDLLSPPQ DTSTGLEEVM |
| 3661 | EQLNNSFPSS RGRNTPGKPM REDTM. |

In some embodiments, a gRNA targets a dystrophin intron or a dystrophin exon. In some embodiments, the gRNA targets a dystrophin splice site. In some embodiments, the gRNA targets a dystrophin splice donor site. In embodiments, the gRNA targets a dystrophin splice acceptor site.

In some embodiments, a gRNA targets DMD exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79. In some embodiments, a gRNA targets DMD exon 6, 7, 8, 43, 44, 45, 46, 50, 51, 52, 53, or 55. In some embodiments, a gRNA targets an intron immediately 5′ or immediately 3′ to DMD exon 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, or 79. In some embodiments, a gRNA targets an intron immediately 5′ or immediately 3′ to DMD exon 6, 7, 8, 43, 44, 45, 46, 50, 51, 52, 53, or 55.

In some embodiments, the gRNA targeting sequence may comprise or may encode any one of the sequences shown in Tables 3-14 below. In some embodiments, the gRNA targeting sequence comprises the sequence CACCAGAGTAACAGTCTGAG (SEQ ID NO: 13), or a sequence at least 95% or at least 99% similar thereto. In some embodiments, the gRNA targeting sequence and scaffold comprise the sequence CACCAGAGTAACAGTCTGAGGTTTAAGAGCTATGCTGGAAACAGCATAGCAAGTTT AAATAAG GCTAGTCCGTTATCAACTTGAAAAAGTGGCACCGAGTCGGTGCTTTTTT T (SEQ ID NO: 14), or a sequence at least 95% or at least 99% similar thereto. In some embodiments, the gRNA targeting sequence encodes a gRNA comprising the sequence CACCAGAGUAACAGUCUGAG (SEQ ID NO: 853).

### Spacer

A spacer sequence is a short nucleic acid sequence used to target a nuclease (e.g., a Cas9 nuclease) to a specific nucleotide region of interest (e.g., a genomic DNA sequence to be cleaved).

In some embodiments, the spacer may be about 17-24 base pairs in length, such as about 20 base pairs in length. In some embodiments, the spacer may be about 15, about 16, about 17, about 18, about 19, about 20, about 21, about 22, about 23, about 24, about 25, about 26, about 27, about 28, about 29, or about 30 base pairs in length. In some embodiments, the spacer may be at least 15, at least 16, at least 17, at least 18, at least 19, at least 20, at least 21, at least 22, at least 23, at least 24, at least 25, at least 26, at least 27, at least 28, at least 29, or at least 30 base pairs in length. In some embodiments, the spacer may be 15, 16, 17, 18, 19, 20, 21, 22, 23, 24, 25, 26, 27, 28, 29, or 30 base pairs in length. In some embodiments, the spacer sequence has between about 40% to about 80% GC content.

In some embodiments, the spacer targets a site that immediately precedes a 5′ protospacer adjacent motif (PAM). The PAM sequence may be selected based on the desired nuclease. For example, the PAM sequence may be any one of the PAM sequences shown in Table 2.

In some embodiments, a spacer may target a sequence of a mammalian gene, such as a human gene. In some embodiments, the spacer may target a non-coding sequence. In some embodiments, the spacer may target a coding sequence. In some embodiments, the spacer may target an intronic sequence. In some embodiments, the spacer may target an exonic sequence. In some embodiments, the spacer may target an intron-exon junction. In some embodiments, a spacer may target an intronic splicing silencer. In some embodiments, a spacer may target an intronic splicing enhancer. In some embodiments, a spacer may target an exonic splicing silencer. In some embodiments, a spacer may target an exonic splicing enhancer. In some embodiments, a spacer may target a 5′UTR. In some embodiments, a spacer may target a 3′UTR. In some embodiments, a spacer may target a mutant gene. In some embodiments, a spacer may target a wildtype gene.

In some embodiments, a spacer may have a sequence of any one of SEQ ID NOs: 13 or 29 to 2024. In some embodiments, a spacer may have a sequence at least 90%, at least 95%, at least 96%, at least 97%, or at least 99% identical to the sequence of any one of SEQ ID NOs: 13 or 29 to 2024. In some embodiments, a spacer may have a sequence of any one of the spacers shown in Tables 3-15, or a sequence that is at least 90%, at least 95%, at least 96%, at least 97%, or at least 99% identical thereto.

TABLE 3
Exemplary gRNA targeting sequences

| | | | | SEQ ID | |
|---|---|---|---|---|---|
| Targeted gRNA Exon | Guide # | Strand | DNA Sequence* | NO | PAM |
| Human-Exon 51 | 4 | 1 | tctttttcttcttttttccttttt | 29 | tttt |
| Human-Exon 51 | 5 | 1 | ctttttcttcttttttcctttttG | 30 | tttt |
| Human-Exon 51 | 6 | 1 | tttttcttcttttttcctttttGC | 31 | tttc |
| Human-Exon 51 | 7 | 1 | tcttcttttttcctttttGCAAAA | 32 | tttt |
| Human-Exon 51 | 8 | 1 | cttcttttttcctttttGCAAAAA | 33 | tttt |
| Human-Exon 51 | 9 | 1 | ttcttttttcctttttGCAAAAAC | 34 | tttc |
| Human-Exon 51 | 10 | 1 | ttcctttttGCAAAAACCCAAAAT | 35 | tttt |
| Human-Exon 51 | 11 | 1 | tcctttttGCAAAAACCCAAAATA | 36 | tttt |
| Human-Exon 51 | 12 | 1 | cctttttGCAAAAACCCAAAATAT | 37 | tttt |
| Human-Exon 51 | 13 | 1 | ctttttGCAAAAACCCAAAATATT | 38 | tttc |
| Human-Exon 51 | 14 | 1 | tGCAAAAACCCAAAATATTTTAGC | 39 | tttt |
| Human-Exon 51 | 15 | 1 | GCAAAAACCCAAAATATTTTAGCT | 40 | tttt |
| Human-Exon 51 | 16 | 1 | CAAAAACCCAAAATATTTTAGCTC | 41 | tttG |
| Human-Exon 51 | 17 | 1 | AGCTCCTACTCAGACTGTTACTCT | 42 | TTTT |
| Human-Exon 51 | 18 | 1 | GCTCCTACTCAGACTGTTACTCTG | 43 | TTTA |
| Human-Exon 51 | 19 | −1 | CTTAGTAACCACAGGTTGTGTCAC | 44 | TTTC |
| Human-Exon 51 | 20 | −1 | GAGATGGCAGTTTCCTTAGTAACC | 45 | TTTG |
| Human-Exon 51 | 21 | −1 | TAGTTTGGAGATGGCAGTTTCCTT | 46 | TTTC |
| Human-Exon 51 | 22 | −1 | TTCTCATACCTTCTGCTTGATGAT | 47 | TTTT |
| Human-Exon 51 | 23 | −1 | TCATTTTTTCTCATACCTTCTGCT | 48 | TTTA |
| Human-Exon 51 | 24 | −1 | ATCATTTTTTCTCATACCTTCTGC | 49 | TTTT |
| Human-Exon 51 | 25 | −1 | AAGAAAAACTTCTGCCAACTTTTA | 50 | TTTA |
| Human-Exon 51 | 26 | −1 | AAAGAAAAACTTCTGCCAACTTTT | 51 | TTTT |
| Human-Exon 51 | 27 | 1 | TCTTTAAAATGAAGATTTTCCACC | 52 | TTTT |
| Human-Exon 51 | 28 | 1 | CTTTAAAATGAAGATTTTCCACCA | 53 | TTTT |
| Human-Exon 51 | 29 | 1 | TTTAAAATGAAGATTTTCCACCAA | 54 | TTTC |
| Human-Exon 51 | 30 | 1 | AAATGAAGATTTTCCACCAATCAC | 55 | TTTA |
| Human-Exon 51 | 31 | 1 | CCACCAATCACTTTACTCTCCTAG | 56 | TTTT |
| Human-Exon 51 | 32 | 1 | CACCAATCACTTTACTCTCCTAGA | 57 | TTTC |
| Human-Exon 51 | 33 | 1 | CTCTCCTAGACCATTTCCCACCAG | 58 | TTTA |
| Human-Exon 45 | 1 | −1 | agaaaagattaaacagtgtgctac | 59 | tttg |
| Human-Exon 45 | 2 | −1 | tttgagaaaagattaaacagtgtg | 60 | TTTa |
| Human-Exon 45 | 3 | −1 | atttgagaaaagattaaacagtgt | 61 | TTTT |
| Human-Exon 45 | 4 | −1 | Tatttgagaaaagattaaacagtg | 62 | TTTT |
| Human-Exon 45 | 5 | 1 | atcttttctcaaatAAAAAGACAT | 63 | ttta |
| Human-Exon 45 | 6 | 1 | ctcaaatAAAAAGACATGGGGCTT | 64 | tttt |
| Human-Exon 45 | 7 | 1 | tcaaatAAAAAGACATGGGGCTTC | 65 | tttc |
| Human-Exon 45 | 8 | 1 | TGTTTTGCCTTTTTGGTATCTTAC | 66 | TTTT |
| Human-Exon 45 | 9 | 1 | GTTTTGCCTTTTTGGTATCTTACA | 67 | TTTT |
| Human-Exon 45 | 10 | 1 | TTTTGCCTTTTTGGTATCTTACAG | 68 | TTTG |
| Human-Exon 45 | 11 | 1 | GCCTTTTTGGTATCTTACAGGAAC | 69 | TTTT |
| Human-Exon 45 | 12 | 1 | CCTTTTTGGTATCTTACAGGAACT | 70 | TTTG |
| Human-Exon 45 | 13 | 1 | TGGTATCTTACAGGAACTCCAGGA | 71 | TTTT |
| Human-Exon 45 | 14 | 1 | GGTATCTTACAGGAACTCCAGGAT | 72 | TTTT |
| Human-Exon 45 | 15 | −1 | AGGATTGCTGAATTATTTCTTCCC | 73 | TTTG |
| Human-Exon 45 | 16 | −1 | GAGGATTGCTGAATTATTTCTTCC | 74 | TTTT |
| Human-Exon 45 | 17 | −1 | TGAGGATTGCTGAATTATTTCTTC | 75 | TTTT |
| Human-Exon 45 | 18 | −1 | CTGTAGAATACTGGCATCTGTTTT | 76 | TTTC |
| Human-Exon 45 | 19 | −1 | CCTGTAGAATACTGGCATCTGTTT | 77 | TTTT |
| Human-Exon 45 | 20 | −1 | TCCTGTAGAATACTGGCATCTGTT | 78 | TTTT |
| Human-Exon 45 | 21 | −1 | CAGACCTCCTGCCACCGCAGATTC | 79 | TTTG |
| Human-Exon 45 | 22 | −1 | TGTCTGACAGCTGTTTGCAGACCT | 80 | TTTC |
| Human-Exon 45 | 23 | −1 | CTGTCTGACAGCTGTTTGCAGACC | 81 | TTTT |
| Human-Exon 45 | 24 | −1 | TCTGTCTGACAGCTGTTTGCAGAC | 82 | TTTT |
| Human-Exon 45 | 25 | −1 | TTCTGTCTGACAGCTGTTTGCAGA | 83 | TTTT |
| Human-Exon 45 | 26 | −1 | ATTCCTATTAGATCTGTCGCCCTA | 84 | TTTC |
| Human-Exon 45 | 27 | −1 | CATTCCTATTAGATCTGTCGCCCT | 85 | TTTT |
| Human-Exon 45 | 28 | 1 | AGCAGACTTTTTAAGCTTTCTTTA | 86 | TTTT |
| Human-Exon 45 | 29 | 1 | GCAGACTTTTTAAGCTTTCTTTAG | 87 | TTTA |
| Human-Exon 45 | 30 | 1 | TAAGCTTTCTTTAGAAGAATATTT | 88 | TTTT |
| Human-Exon 45 | 31 | 1 | AAGCTTTCTTTAGAAGAATATTTC | 89 | TTTT |
| Human-Exon 45 | 32 | 1 | AGCTTTCTTTAGAAGAATATTTCA | 90 | TTTA |
| Human-Exon 45 | 33 | 1 | TTTAGAAGAATATTTCATGAGAGA | 91 | TTTC |
| Human-Exon 45 | 34 | 1 | GAAGAATATTTCATGAGAGATTAT | 92 | TTTA |
| Human-Exon 44 | 1 | 1 | TCAGTATAACCAAAAAATATACGC | 93 | TTTG |
| Human-Exon 44 | 2 | 1 | acataatccatctatttttcttga | 94 | tttt |
| Human-Exon 44 | 3 | 1 | cataatccatctatttttcttgat | 95 | ttta |
| Human-Exon 44 | 4 | 1 | tcttgatccatatgcttttACCTG | 96 | tttt |
| Human-Exon 44 | 5 | 1 | cttgatccatatgcttttACCTGC | 97 | tttt |
| Human-Exon 44 | 6 | 1 | ttgatccatatgcttttACCTGCA | 98 | tttc |
| Human-Exon 44 | 7 | −1 | TCAACAGATCTGTCAAATCGCCTG | 99 | TTTC |
| Human-Exon 44 | 8 | 1 | ACCTGCAGGCGATTTGACAGATCT | 100 | tttt |
| Human-Exon 44 | 9 | 1 | CCTGCAGGCGATTTGACAGATCTG | 101 | tttA |
| Human-Exon 44 | 10 | 1 | ACAGATCTGTTGAGAAATGGCGGC | 102 | TTTG |
| Human-Exon 44 | 11 | −1 | TATCATAATGAAAACGCCGCCATT | 103 | TTTA |
| Human-Exon 44 | 12 | 1 | CATTATGATATAAAGATATTTAAT | 104 | TTTT |
| Human-Exon 44 | 13 | −1 | TATTTAGCATGTTCCCAATTCTCA | 105 | TTTG |
| Human-Exon 44 | 14 | −1 | GAAAAAACAAATCAAAGACTTACC | 106 | TTTC |
| Human-Exon 44 | 15 | 1 | ATTTGTTTTTTCGAAATTGTATTT | 107 | TTTG |
| Human-Exon 44 | 16 | 1 | TTTTTTCGAAATTGTATTTATCTT | 108 | TTTG |
| Human-Exon 44 | 17 | 1 | TTCGAAATTGTATTTATCTTCAGC | 109 | TTTT |
| Human-Exon 44 | 18 | 1 | TCGAAATTGTATTTATCTTCAGCA | 110 | TTTT |
| Human-Exon 44 | 19 | 1 | CGAAATTGTATTTATCTTCAGCAC | 111 | TTTT |
| Human-Exon 44 | 20 | 1 | GAAATTGTATTTATCTTCAGCACA | 112 | TTTC |
| Human-Exon 44 | 21 | −1 | AGAAGTTAAAGAGTCCAGATGTGC | 113 | TTTA |
| Human-Exon 44 | 22 | 1 | TCTTCAGCACATCTGGACTCTTTA | 114 | TTTA |
| Human-Exon 44 | 23 | −1 | CATCACCCTTCAGAACCTGATCTT | 115 | TTTC |
| Human-Exon 44 | 24 | 1 | ACTTCTTAAAGATCAGGTTCTGAA | 116 | TTTA |
| Human-Exon 44 | 25 | 1 | GACTGTTGTTGTCATCATTATATT | 117 | TTTT |
| Human-Exon 44 | 26 | 1 | ACTGTTGTTGTCATCATTATATTA | 118 | TTTG |
| Human-Exon 53 | 1 | −1 | AACTAGAATAAAAGGAAAAATAAA | 119 | TTTC |
| Human-Exon 53 | 2 | 1 | CTACTATATATTTATTTTTCCTTT | 120 | TTTA |
| Human-Exon 53 | 3 | 1 | TTTTTCCTTTTATTCTAGTTGAAA | 121 | TTTA |
| Human-Exon 53 | 4 | 1 | TCCTTTTATTCTAGTTGAAAGAAT | 122 | TTTT |
| Human-Exon 53 | 5 | 1 | CCTTTTATTCTAGTTGAAAGAATT | 123 | TTTT |
| Human-Exon 53 | 6 | 1 | CTTTTATTCTAGTTGAAAGAATTC | 124 | TTTC |
| Human-Exon 53 | 7 | 1 | ATTCTAGTTGAAAGAATTCAGAAT | 125 | TTTT |
| Human-Exon 53 | 8 | 1 | TTCTAGTTGAAAGAATTCAGAATC | 126 | TTTA |
| Human-Exon 53 | 9 | −1 | ATTCAACTGTTGCCTCCGGTTCTG | 127 | TTTC |
| Human-Exon 53 | 10 | −1 | ACATTTCATTCAACTGTTGCCTCC | 128 | TTTA |
| Human-Exon 53 | 11 | −1 | CTTTTGGATTGCATCTACTGTATA | 129 | TTTT |
| Human-Exon 53 | 12 | −1 | TGTGATTTTCTTTTGGATTGCATC | 130 | TTTC |
| Human-Exon 53 | 13 | −1 | ATACTAACCTTGGTTTCTGTGATT | 131 | TTTG |
| Human-Exon 53 | 14 | −1 | AAAAGGTATCTTTGATACTAACCT | 132 | TTTA |
| Human-Exon 53 | 15 | −1 | AAAAAGGTATCTTTGATACTAACC | 133 | TTTT |
| Human-Exon 53 | 16 | −1 | TTTTAAAAAGGTATCTTTGATACT | 134 | TTTA |
| Human-Exon 53 | 17 | −1 | ATTTTAAAAAGGTATCTTTGATAC | 135 | TTTT |
| Human-Exon 46 | 1 | −1 | TTAATGCAAACTGGGACACAAACA | 136 | TTTG |
| Human-Exon 46 | 2 | 1 | TAAATTGCCATGTTTGTGTCCCAG | 137 | TTTT |
| Human-Exon 46 | 3 | 1 | AAATTGCCATGTTTGTGTCCCAGT | 138 | TTTT |
| Human-Exon 46 | 4 | 1 | AATTGCCATGTTTGTGTCCCAGTT | 139 | TTTA |
| Human-Exon 46 | 5 | 1 | TGTCCCAGTTTGCATTAACAAATA | 140 | TTTG |
| Human-Exon 46 | 6 | −1 | CAACATAGTTCTCAAACTATTTGT | 141 | tttC |
| Human-Exon 46 | 7 | −1 | CCAACATAGTTCTCAAACTATTTG | 142 | tttt |
| Human-Exon 46 | 8 | −1 | tCCAACATAGTTCTCAAACTATTT | 143 | tttt |
| Human-Exon 46 | 9 | −1 | tttCCAACATAGTTCTCAAACTAT | 144 | tttt |
| Human-Exon 46 | 10 | −1 | ttttCCAACATAGTTCTCAAACTA | 145 | tttt |
| Human-Exon 46 | 11 | −1 | tttttCCAACATAGTTCTCAAACT | 146 | tttt |
| Human-Exon 46 | 12 | 1 | CATTAACAAATAGTTTGAGAACTA | 147 | TTTG |
| Human-Exon 46 | 13 | 1 | AGAACTATGTTGGaaaaaaaaaTA | 148 | TTTG |
| Human-Exon 46 | 14 | −1 | GTTCTTCTAGCCTGGAGAAAGAAG | 149 | TTTT |
| Human-Exon 46 | 15 | 1 | ATTCTTCTTTCTCCAGGCTAGAAG | 150 | TTTT |
| Human-Exon 46 | 16 | 1 | TTCTTCTTTCTCCAGGCTAGAAGA | 151 | TTTA |
| Human-Exon 46 | 17 | 1 | TCCAGGCTAGAAGAACAAAAGAAT | 152 | TTTC |
| Human-Exon 46 | 18 | −1 | AAATTCTGACAAGATATTCTTTTG | 153 | TTTG |
| Human-Exon 46 | 19 | −1 | CTTTTAGTTGCTGCTCTTTTCCAG | 154 | TTTT |
| Human-Exon 46 | 20 | −1 | AGAAAATAAAATTACCTTGACTTG | 155 | TTTG |
| Human-Exon 46 | 21 | −1 | TGCAAGCAGGCCCTGGGGGATTTG | 156 | TTTA |
| Human-Exon 46 | 22 | 1 | ATTTTCTCAAATCCCCCAGGGCCT | 157 | TTTT |
| Human-Exon 46 | 23 | 1 | TTTTCTCAAATCCCCCAGGGCCTG | 158 | TTTA |
| Human-Exon 46 | 24 | 1 | CTCAAATCCCCCAGGGCCTGCTTG | 159 | TTTT |
| Human-Exon 46 | 25 | 1 | TCAAATCCCCCAGGGCCTGCTTGC | 160 | TTTC |
| Human-Exon 46 | 26 | 1 | TTAATTCAATCATTGGTTTTCTGC | 161 | TTTT |
| Human-Exon 46 | 27 | 1 | TAATTCAATCATTGGTTTTCTGCC | 162 | TTTT |
| Human-Exon 46 | 28 | 1 | AATTCAATCATTGGTTTTCTGCCC | 163 | TTTT |
| Human-Exon 46 | 29 | 1 | ATTCAATCATTGGTTTTCTGCCCA | 164 | TTTA |
| Human-Exon 46 | 30 | −1 | GCAAGGAACTATGAATAACCTAAT | 165 | TTTA |
| Human-Exon 46 | 31 | 1 | CTGCCCATTAGGTTATTCATAGTT | 166 | TTTT |
| Human-Exon 46 | 32 | 1 | TGCCCATTAGGTTATTCATAGTTC | 167 | TTTC |
| Human-Exon 52 | 1 | −1 | TAGAAAACAATTTAACAGGAAATA | 168 | TTTA |
| Human-Exon 52 | 2 | 1 | CTGTTAAATTGTTTTCTATAAACC | 169 | TTTC |
| Human-Exon 52 | 3 | −1 | GAAATAAAAAAGATGTTACTGTAT | 170 | TTTA |
| Human-Exon 52 | 4 | −1 | AGAAATAAAAAAGATGTTACTGTA | 171 | TTTT |
| Human-Exon 52 | 5 | 1 | CTATAAACCCTTATACAGTAACAT | 172 | TTTT |
| Human-Exon 52 | 6 | 1 | TATAAACCCTTATACAGTAACATC | 173 | TTTC |
| Human-Exon 52 | 7 | 1 | TTATTTCTAAAAGTGTTTTGGCTG | 174 | TTTT |
| Human-Exon 52 | 8 | 1 | TATTTCTAAAAGTGTTTTGGCTGG | 175 | TTTT |
| Human-Exon 52 | 9 | 1 | ATTTCTAAAAGTGTTTTGGCTGGT | 176 | TTTT |
| Human-Exon 52 | 10 | 1 | TTTCTAAAAGTGTTTTGGCTGGTC | 177 | TTTA |
| Human-Exon 52 | 11 | 1 | TAAAAGTGTTTTGGCTGGTCTCAC | 178 | TTTC |
| Human-Exon 52 | 12 | −1 | CATAATACAAAGTAAAGTACAATT | 179 | TTTA |
| Human-Exon 52 | 13 | −1 | ACATAATACAAAGTAAAGTACAAT | 180 | TTTT |
| Human-Exon 52 | 14 | 1 | GGCTGGTCTCACAATTGTACTTTA | 181 | TTTT |
| Human-Exon 52 | 15 | 1 | GCTGGTCTCACAATTGTACTTTAC | 182 | TTTG |
| Human-Exon 52 | 16 | 1 | CTTTGTATTATGTAAAAGGAATAC | 183 | TTTA |
| Human-Exon 52 | 17 | 1 | TATTATGTAAAAGGAATACACAAC | 184 | TTTG |
| Human-Exon 52 | 18 | 1 | TTCTTACAGGCAACAATGCAGGAT | 185 | TTTG |
| Human-Exon 52 | 19 | 1 | GAACAGAGGCGTCCCCAGTTGGAA | 186 | TTTG |
| Human-Exon 52 | 20 | −1 | GGCAGCGGTAATGAGTTCTTCCAA | 187 | TTTG |
| Human-Exon 52 | 21 | −1 | TCAAATTTTGGGCAGCGGTAATGA | 188 | TTTT |
| Human-Exon 52 | 22 | 1 | AAAAACAAGACCAGCAATCAAGAG | 189 | TTTG |
| Human-Exon 52 | 23 | −1 | TGTGTCCCATGCTTGTTAAAAAAC | 190 | TTTG |
| Human-Exon 52 | 24 | 1 | TTAACAAGCATGGGACACACAAAG | 191 | TTTT |
| Human-Exon 52 | 25 | 1 | TAACAAGCATGGGACACACAAAGC | 192 | TTTT |
| Human-Exon 52 | 26 | 1 | AACAAGCATGGGACACACAAAGCA | 193 | TTTT |
| Human-Exon 52 | 27 | 1 | ACAAGCATGGGACACACAAAGCAA | 194 | TTTA |
| Human-Exon 52 | 28 | −1 | TTGAAACTTGTCATGCATCTTGCT | 195 | TTTA |
| Human-Exon 52 | 29 | −1 | ATTGAAACTTGTCATGCATCTTGC | 196 | TTTT |
| Human-Exon 52 | 30 | −1 | TATTGAAACTTGTCATGCATCTTG | 197 | TTTT |
| Human-Exon 52 | 31 | 1 | AATAAAAACTTAAGTTCATATATC | 198 | TTTC |
| Human-Exon 50 | 1 | −1 | GTGAATATATTATTGGATTTCTAT | 199 | TTTG |
| Human-Exon 50 | 2 | −1 | AAGATAATTCATGAACATCTTAAT | 200 | TTTG |
| Human-Exon 50 | 3 | −1 | ACAGAAAAGCATACACATTACTTA | 201 | TTTA |
| Human-Exon 50 | 4 | 1 | CTGTTAAAGAGGAAGTTAGAAGAT | 202 | TTTT |
| Human-Exon 50 | 5 | 1 | TGTTAAAGAGGAAGTTAGAAGATC | 203 | TTTC |
| Human-Exon 50 | 6 | −1 | CCGCCTTCCACTCAGAGCTCAGAT | 204 | TTTA |
| Human-Exon 50 | 7 | −1 | CCCTCAGCTCTTGAAGTAAACGGT | 205 | TTTG |
| Human-Exon 50 | 8 | 1 | CTTCAAGAGCTGAGGGCAAAGCAG | 206 | TTTA |
| Human-Exon 50 | 9 | −1 | AACAAATAGCTAGAGCCAAAGAGA | 207 | TTTG |
| Human-Exon 50 | 10 | −1 | GAACAAATAGCTAGAGCCAAAGAG | 208 | TTTT |
| Human-Exon 50 | 11 | 1 | GCTCTAGCTATTTGTTCAAAAGTG | 209 | TTTG |
| Human-Exon 50 | 12 | 1 | TTCAAAAGTGCAACTATGAAGTGA | 210 | TTTG |
| Human-Exon 50 | 13 | −1 | TCTCTCACCCAGTCATCACTTCAT | 211 | TTTC |
| Human-Exon 50 | 14 | −1 | CTCTCTCACCCAGTCATCACTTCA | 212 | TTTT |
| Human-Exon 43 | 1 | 1 | tatatatatatatatTTTTCTCTT | 213 | TTTG |
| Human-Exon 43 | 2 | 1 | TCTCTTTCTATAGACAGCTAATTC | 214 | tTTT |
| Human-Exon 43 | 3 | 1 | CTCTTTCTATAGACAGCTAATTCA | 215 | TTTT |
| Human-Exon 43 | 4 | −1 | AAACAGTAAAAAAATGAATTAGCT | 216 | TTTA |
| Human-Exon 43 | 5 | 1 | TCTTTCTATAGACAGCTAATTCAT | 217 | TTTC |
| Human-Exon 43 | 6 | −1 | AAAACAGTAAAAAAATGAATTAGC | 218 | TTTT |
| Human-Exon 43 | 7 | 1 | TATAGACAGCTAATTCATTTTTTT | 219 | TTTC |
| Human-Exon 43 | 8 | −1 | TATTCTGTAATATAAAAATTTTAA | 220 | TTTA |
| Human-Exon 43 | 9 | −1 | ATATTCTGTAATATAAAAATTTTA | 221 | TTTT |
| Human-Exon 43 | 10 | 1 | TTTACTGTTTTAAAATTTTTATAT | 222 | TTTT |
| Human-Exon 43 | 11 | 1 | TTACTGTTTTAAAATTTTTATATT | 223 | TTTT |
| Human-Exon 43 | 12 | 1 | TACTGTTTTAAAATTTTTATATTA | 224 | TTTT |
| Human-Exon 43 | 13 | 1 | ACTGTTTTAAAATTTTTATATTAC | 225 | TTTT |
| Human-Exon 43 | 14 | 1 | CTGTTTTAAAATTTTTATATTACA | 226 | TTTA |
| Human-Exon 43 | 15 | 1 | AAAATTTTTATATTACAGAATATA | 227 | TTTT |
| Human-Exon 43 | 16 | 1 | AAATTTTTATATTACAGAATATAA | 228 | TTTA |
| Human-Exon 43 | 17 | −1 | TTGTAGACTATCTTTTATATTCTG | 229 | TTTG |
| Human-Exon 43 | 18 | 1 | TATATTACAGAATATAAAAGATAG | 230 | TTTT |
| Human-Exon 43 | 19 | 1 | ATATTACAGAATATAAAAGATAGT | 231 | TTTT |
| Human-Exon 43 | 20 | 1 | TATTACAGAATATAAAAGATAGTC | 232 | TTTA |
| Human-Exon 43 | 21 | −1 | CAATGCTGCTGTCTTCTTGCTATG | 233 | TTTG |
| Human-Exon 43 | 22 | 1 | CAATGGGAAAAAGTTAACAAAATG | 234 | TTTC |
| Human-Exon 43 | 23 | −1 | TGCAAGTATCAAGAAAAATATATG | 235 | TTTC |
| Human-Exon 43 | 24 | 1 | TCTTGATACTTGCAGAAATGATTT | 236 | TTTT |
| Human-Exon 43 | 25 | 1 | CTTGATACTTGCAGAAATGATTTG | 237 | TTTT |
| Human-Exon 43 | 26 | 1 | TTGATACTTGCAGAAATGATTTGT | 238 | TTTC |
| Human-Exon 43 | 27 | 1 | TTTTCAGGGAACTGTAGAATTTAT | 239 | TTTG |
| Human-Exon 43 | 28 | −1 | CATGGAGGGTACTGAAATAAATTC | 240 | TTTC |
| Human-Exon 43 | 29 | −1 | CCATGGAGGGTACTGAAATAAATT | 241 | TTTT |
| Human-Exon 43 | 30 | 1 | CAGGGAACTGTAGAATTTATTTCA | 242 | TTTT |
| Human-Exon 43 | 31 | −1 | TCCATGGAGGGTACTGAAATAAAT | 243 | TTTT |
| Human-Exon 43 | 32 | 1 | AGGGAACTGTAGAATTTATTTCAG | 244 | TTTC |
| Human-Exon 43 | 33 | −1 | TTCCATGGAGGGTACTGAAATAAA | 245 | TTTT |
| Human-Exon 43 | 34 | −1 | CCTGTCTTTTTTCCATGGAGGGTA | 246 | TTTC |
| Human-Exon 43 | 35 | −1 | CCCTGTCTTTTTTCCATGGAGGGT | 247 | TTTT |
| Human-Exon 43 | 36 | −1 | TCCCTGTCTTTTTTCCATGGAGGG | 248 | TTTT |
| Human-Exon 43 | 37 | 1 | TTTCAGTACCCTCCATGGAAAAAA | 249 | TTTA |
| Human-Exon 43 | 38 | 1 | AGTACCCTCCATGGAAAAAAGACA | 250 | TTTC |
| Human-Exon 6 | 1 | 1 | AGTTTGCATGGTTCTTGCTCAAGG | 251 | TTTA |
| Human-Exon 6 | 2 | −1 | ATAAGAAAATGCATTCCTTGAGCA | 252 | TTTC |
| Human-Exon 6 | 3 | −1 | CATAAGAAAATGCATTCCTTGAGC | 253 | TTTT |
| Human-Exon 6 | 4 | 1 | CATGGTTCTTGCTCAAGGAATGCA | 254 | TTTG |
| Human-Exon 6 | 5 | −1 | ACCTACATGTGGAAATAAATTTTC | 255 | TTTG |
| Human-Exon 6 | 6 | −1 | GACCTACATGTGGAAATAAATTTT | 256 | TTTT |
| Human-Exon 6 | 7 | −1 | TGACCTACATGTGGAAATAAATTT | 257 | TTTT |
| Human-Exon 6 | 8 | 1 | CTTATGAAAATTTATTTCCACATG | 258 | TTTT |
| Human-Exon 6 | 9 | 1 | TTATGAAAATTTATTTCCACATGT | 259 | TTTC |
| Human-Exon 6 | 10 | −1 | ATTACATTTTTGACCTACATGTGG | 260 | TTTC |
| Human-Exon 6 | 11 | −1 | CATTACATTTTTGACCTACATGTG | 261 | TTTT |
| Human-Exon 6 | 12 | −1 | TCATTACATTTTTGACCTACATGT | 262 | TTTT |
| Human-Exon 6 | 13 | 1 | TTTCCACATGTAGGTCAAAAATGT | 263 | TTTA |
| Human-Exon 6 | 14 | 1 | CACATGTAGGTCAAAAATGTAATG | 264 | TTTC |
| Human-Exon 6 | 15 | −1 | TTGCAATCCAGCCATGATATTTTT | 265 | TTTG |
| Human-Exon 6 | 16 | −1 | ACTGTTGGTTTGTTGCAATCCAGC | 266 | TTTC |
| Human-Exon 6 | 17 | −1 | CACTGTTGGTTTGTTGCAATCCAG | 267 | TTTT |
| Human-Exon 6 | 18 | 1 | AATGCTCTCATCCATAGTCATAGG | 268 | TTTG |
| Human-Exon 6 | 19 | −1 | ATGTCTCAGTAATCTTCTTACCTA | 269 | TTTA |
| Human-Exon 6 | 20 | −1 | CAAGTTATTTAATGTCTCAGTAAT | 270 | TTTA |
| Human-Exon 6 | 21 | −1 | ACAAGTTATTTAATGTCTCAGTAA | 271 | TTTT |
| Human-Exon 6 | 22 | 1 | GACTCTGATGACATATTTTTCCCC | 272 | TTTA |
| Human-Exon 6 | 23 | 1 | TCCCCAGTATGGTTCCAGATCATG | 273 | TTTT |
| Human-Exon 6 | 24 | 1 | CCCCAGTATGGTTCCAGATCATGT | 274 | TTTT |
| Human-Exon 6 | 25 | 1 | CCCAGTATGGTTCCAGATCATGTC | 275 | TTTC |
| Human-Exon 7 | 1 | 1 | TATTTGTCTTtgtgtatgtgtgta | 276 | TTTA |
| Human-Exon 7 | 2 | 1 | TCTTtgtgtatgtgtgtatgtgta | 277 | TTTG |
| Human-Exon 7 | 3 | 1 | tgtatgtgtgtatgtgtatgtgtt | 278 | TTtg |
| Human-Exon 7 | 4 | 1 | AGGCCAGACCTATTTGACTGGAAT | 279 | ttTT |
| Human-Exon 7 | 5 | 1 | GGCCAGACCTATTTGACTGGAATA | 280 | tTTA |
| Human-Exon 7 | 6 | 1 | ACTGGAATAGTGTGGTTTGCCAGC | 281 | TTTG |
| Human-Exon 7 | 7 | 1 | CCAGCAGTCAGCCACACAACGACT | 282 | TTTG |
| Human-Exon 7 | 8 | −1 | TCTATGCCTAATTGATATCTGGCG | 283 | TTTC |
| Human-Exon 7 | 9 | −1 | CCAACCTTCAGGATCGAGTAGTTT | 284 | TTTA |
| Human-Exon 7 | 10 | 1 | TGGACTACCACTGCTTTTAGTATG | 285 | TTTC |
| Human-Exon 7 | 11 | 1 | AGTATGGTAGAGTTTAATGTTTTC | 286 | TTTT |
| Human-Exon 7 | 12 | 1 | GTATGGTAGAGTTTAATGTTTTCA | 287 | TTTA |
| Human-Exon 8 | 1 | −1 | AGACTCTAAAAGGATAATGAACAA | 288 | TTTG |
| Human-Exon 8 | 2 | 1 | ACTTTGATTTGTTCATTATCCTTT | 289 | TTTA |
| Human-Exon 8 | 3 | −1 | TATATTTGAGACTCTAAAAGGATA | 290 | TTTC |
| Human-Exon 8 | 4 | 1 | ATTTGTTCATTATCCTTTTAGAGT | 291 | TTTG |
| Human-Exon 8 | 5 | −1 | GTTTCTATATTTGAGACTCTAAAA | 292 | TTTG |
| Human-Exon 8 | 6 | −1 | GGTTTCTATATTTGAGACTCTAAA | 293 | TTTT |
| Human-Exon 8 | 7 | −1 | TGGTTTCTATATTTGAGACTCTAA | 294 | TTTT |
| Human-Exon 8 | 8 | 1 | TTCATTATCCTTTTAGAGTCTCAA | 295 | TTTG |
| Human-Exon 8 | 9 | 1 | AGAGTCTCAAATATAGAAACCAAA | 296 | TTTT |
| Human-Exon 8 | 10 | 1 | GAGTCTCAAATATAGAAACCAAAA | 297 | TTTA |
| Human-Exon 8 | 11 | −1 | CACTTCCTGGATGGCTTCAATGCT | 298 | TTTC |
| Human-Exon 8 | 12 | 1 | GCCTCAACAAGTGAGCATTGAAGC | 299 | TTTT |
| Human-Exon 8 | 13 | 1 | CCTCAACAAGTGAGCATTGAAGCC | 300 | TTTG |
| Human-Exon 8 | 14 | −1 | GGTGGCCTTGGCAACATTTCCACT | 301 | TTTA |
| Human-Exon 8 | 15 | −1 | GTCACTTTAGGTGGCCTTGGCAAC | 302 | TTTA |
| Human-Exon 8 | 16 | −1 | ATGATGTAACTGAAAATGTTCTTC | 303 | TTTG |
| Human-Exon 8 | 17 | −1 | CCTGTTGAGAATAGTGCATTTGAT | 304 | TTTA |
| Human-Exon 8 | 18 | 1 | CAGTTACATCATCAAATGCACTAT | 305 | TTTT |
| Human-Exon 8 | 19 | 1 | AGTTACATCATCAAATGCACTATT | 306 | TTTC |
| Human-Exon 8 | 20 | −1 | CACACTTTACCTGTTGAGAATAGT | 307 | TTTA |
| Human-Exon 8 | 21 | 1 | CTGTTTTATATGCATTTTTAGGTA | 308 | TTTT |
| Human-Exon 8 | 22 | 1 | TGTTTTATATGCATTTTTAGGTAT | 309 | TTTC |
| Human-Exon 8 | 23 | 1 | ATATGCATTTTTAGGTATTACGTG | 310 | TTTT |
| Human-Exon 8 | 24 | 1 | TATGCATTTTTAGGTATTACGTGC | 311 | TTTA |
| Human-Exon 8 | 25 | 1 | TAGGTATTACGTGCACatatatat | 312 | TTTT |
| Human-Exon 8 | 26 | 1 | AGGTATTACGTGCACatatatata | 313 | TTTT |
| Human-Exon 8 | 27 | 1 | GGTATTACGTGCACatatatatat | 314 | TTTA |
| Human-Exon 55 | 1 | −1 | AGCAACAACTATAATATTGTGCAG | 315 | TTTA |
| Human-Exon 55 | 2 | 1 | GTTCCTCCATCTTTCTCTTTTTAT | 316 | TTTA |
| Human-Exon 55 | 3 | 1 | TCTTTTTATGGAGTTCACTAGGTG | 317 | TTTC |
| Human-Exon 55 | 4 | 1 | TATGGAGTTCACTAGGTGCACCAT | 318 | TTTT |
| Human-Exon 55 | 5 | 1 | ATGGAGTTCACTAGGTGCACCATT | 319 | TTTT |
| Human-Exon 55 | 6 | 1 | TGGAGTTCACTAGGTGCACCATTC | 320 | TTTA |
| Human-Exon 55 | 7 | 1 | ATAATTGCATCTGAACATTTGGTC | 321 | TTTA |
| Human-Exon 55 | 8 | 1 | GTCCTTTGCAGGGTGAGTGAGCGA | 322 | TTTG |
| Human-Exon 55 | 9 | −1 | TTCCAAAGCAGCCTCTCGCTCACT | 323 | TTTC |
| Human-Exon 55 | 10 | 1 | CAGGGTGAGTGAGCGAGAGGCTGC | 324 | TTTG |
| Human-Exon 55 | 11 | 1 | GAAGAAACTCATAGATTACTGCAA | 325 | TTTG |
| Human-Exon 55 | 12 | −1 | CAGGTCCAGGGGGAACTGTTGCAG | 326 | TTTC |
| Human-Exon 55 | 13 | −1 | CCAGGTCCAGGGGGAACTGTTGCA | 327 | TTTT |
| Human-Exon 55 | 14 | −1 | AGCTTCTGTAAGCCAGGCAAGAAA | 328 | TTTC |
| Human-Exon 55 | 15 | 1 | TTGCCTGGCTTACAGAAGCTGAAA | 329 | TTTC |
| Human-Exon 55 | 16 | −1 | CTTACGGGTAGCATCCTGTAGGAC | 330 | TTTC |
| Human-Exon 55 | 17 | −1 | CTCCCTTGGAGTCTTCTAGGAGCC | 331 | TTTA |
| Human-Exon 55 | 18 | −1 | ACTCCCTTGGAGTCTTCTAGGAGC | 332 | TTTT |
| Human-Exon 55 | 19 | −1 | ATCAGCTCTTTTACTCCCTTGGAG | 333 | TTTC |
| Human-Exon 55 | 20 | 1 | CGCTTTAGCACTCTTGTGGATCCA | 334 | TTTC |
| Human-Exon 55 | 21 | 1 | GCACTCTTGTGGATCCAATTGAAC | 335 | TTTA |
| Human-Exon 55 | 22 | −1 | TCCCTGGCTTGTCAGTTACAAGTA | 336 | TTTG |
| Human-Exon 55 | 23 | −1 | GTCCCTGGCTTGTCAGTTACAAGT | 337 | TTTT |
| Human-Exon 55 | 24 | −1 | TTTTGTCCCTGGCTTGTCAGTTAC | 338 | TTTG |
| Human-Exon 55 | 25 | −1 | GTTTTGTCCCTGGCTTGTCAGTTA | 339 | TTTT |
| Human-Exon 55 | 26 | 1 | TACTTGTAACTGACAAGCCAGGGA | 340 | TTTG |
| Human-G1-exon51 | | 1 | gCTCCTACTCAGACTGTTACTCTG | 341 | TTTA |
| Human-G2-exon51 | | 1 | taccatgtattgctaaacaaagta | 342 | TTTC |
| Human-G3-exon51 | | −1 | attgaagagtaacaatttgagcca | 343 | TTTA |
| mouse-Exon23-G1 | | 1 | aggctctgcaaagttctTTGAAAG | 344 | TTTG |
| mouse-Exon23-G2 | | 1 | AAAGAGCAACAAAATGGCttcaac | 345 | TTTG |
| mouse-Exon23-G3 | | 1 | AAAGAGCAATAAAATGGCttcaac | 346 | TTTG |
| mouse-Exon23-G4 | | −1 | AAAGAACTTTGCAGAGCctcaaaa | 347 | TTTC |
| mouse-Exon23-G5 | | −1 | ctgaatatctatgcattaataact | 348 | TTTA |
| mouse-Exon23-G6 | | −1 | tattatattacagggcatattata | 349 | TTTC |
| mouse-Exon23-G7 | | 1 | Aggtaagccgaggtttggccttta | 350 | TTTC |
| mouse-Exon23-G8 | | 1 | cccagagtccttcaaagatattga | 351 | TTTA |

*In this table, upper case letters represent nucleotides that align to the exon sequence of the gene. Lower case letters represent nucleotides that align to the intron sequence of the gene.

TABLE 4
Exemplary gRNA targeting sequences

| Targeted gRNA Exon | Guide # | Strand | gRNA sequence* | SEQ ID NO. | PAM |
|---|---|---|---|---|---|
| Human-Exon 51 | 4 | −1 | aaaaaggaaaaaagaagaaaaaga | 352 | tttt |
| Human-Exon 51 | 5 | −1 | Caaaaaggaaaaaagaagaaaaag | 353 | tttt |
| Human-Exon 51 | 6 | −1 | GCaaaaaggaaaaaagaagaaaaa | 354 | tttc |
| Human-Exon 51 | 7 | −1 | UUUUGCaaaaaggaaaaaagaaga | 355 | tttt |
| Human-Exon 51 | 8 | −1 | UUUUUGCaaaaaggaaaaaagaag | 356 | tttt |
| Human-Exon 51 | 9 | −1 | GUUUUUGCaaaaaggaaaaaagaa | 357 | tttc |
| Human-Exon 51 | 10 | −1 | AUUUUGGGUUUUUGCaaaaaggaa | 358 | tttt |
| Human-Exon 51 | 11 | −1 | UAUUUUGGGUUUUUGCaaaaagga | 359 | tttt |
| Human-Exon 51 | 12 | −1 | AUAUUUUGGGUUUUUGCaaaaagg | 360 | tttt |
| Human-Exon 51 | 13 | −1 | AAUAUUUUGGGUUUUUGCaaaaag | 361 | tttc |
| Human-Exon 51 | 14 | −1 | GCUAAAAUAUUUUGGGUUUUUGCa | 362 | tttt |
| Human-Exon 51 | 15 | −1 | AGCUAAAAUAUUUUGGGUUUUUGC | 363 | tttt |
| Human-Exon 51 | 16 | −1 | GAGCUAAAAUAUUUUGGGUUUUUG | 364 | tttG |
| Human-Exon 51 | 17 | −1 | AGAGUAACAGUCUGAGUAGGAGCU | 365 | TTTT |
| Human-Exon 51 | 18 | −1 | CAGAGUAACAGUCUGAGUAGGAGC | 366 | TTTA |
| Human-Exon 51 | 19 | 1 | GUGACACAACCUGUGGUUACUAAG | 367 | TTTC |
| Human-Exon 51 | 20 | 1 | GGUUACUAAGGAAACUGCCAUCU | 368 | TTTG |
| Human-Exon 51 | 21 | 1 | AAGGAAACUGCCAUCUCCAAACUA | 369 | TTTC |
| Human-Exon 51 | 22 | 1 | AUCAUCAAGCAGAAGGUAUGAGAA | 370 | TTTT |
| Human-Exon 51 | 23 | 1 | AGCAGAAGGUAUGAGAAAAAAUGA | 371 | TTTA |
| Human-Exon 51 | 24 | 1 | GCAGAAGGUAUGAGAAAAAAUGAU | 372 | TTTT |
| Human-Exon 51 | 25 | 1 | UAAAAGUUGGCAGAAGUUUUUCUU | 373 | TTTA |
| Human-Exon 51 | 26 | 1 | AAAAGUUGGCAGAAGUUUUUCUUU | 374 | TTTT |
| Human-Exon 51 | 27 | −1 | GGUGGAAAAUCUUCAUUUUAAAGA | 375 | TTTT |
| Human-Exon 51 | 28 | −1 | UGGUGGAAAAUCUUCAUUUUAAAG | 376 | TTTT |
| Human-Exon 51 | 29 | −1 | UUGGUGGAAAAUCUUCAUUUUAAA | 377 | TTTC |
| Human-Exon 51 | 30 | −1 | GUGAUUGGUGGAAAAUCUUCAUUU | 378 | TTTA |
| Human-Exon 51 | 31 | −1 | CUAGGAGAGUAAAGUGAUUGGUGG | 379 | TTTT |
| Human-Exon 51 | 32 | −1 | UCUAGGAGAGUAAAGUGAUUGGUG | 380 | TTTC |
| Human-Exon 51 | 33 | −1 | CUGGUGGGAAAUGGUCUAGGAGA | 381 | TTTA |
| Human-Exon 45 | 1 | 1 | guagcacacuguuuaaucuuuucu | 382 | tttg |
| Human-Exon 45 | 2 | 1 | cacacuguuuaaucuuuucucaaa | 383 | TTTa |
| Human-Exon 45 | 3 | 1 | acacuguuuaaucuuuucucaaau | 384 | TTTT |
| Human-Exon 45 | 4 | 1 | cacuguuuaaucuuuucucaaauA | 385 | TTTT |
| Human-Exon 45 | 5 | −1 | AUGUCUUUUUauuugagaaaagau | 386 | ttta |
| Human-Exon 45 | 6 | −1 | AAGCCCCAUGUCUUUUUauuugag | 387 | tttt |
| Human-Exon 45 | 7 | −1 | GAAGCCCCAUGUCUUUUUauuuga | 388 | tttc |
| Human-Exon 45 | 8 | −1 | GUAAGAUACCAAAAAGGCAAAACA | 389 | TTTT |
| Human-Exon 45 | 9 | −1 | UGUAAGAUACCAAAAAGGCAAAAC | 390 | TTTT |
| Human-Exon 45 | 10 | −1 | CUGUAAGAUACCAAAAAGGCAAAA | 391 | TTTG |
| Human-Exon 45 | 11 | −1 | GUUCCUGUAAGAUACCAAAAAGGC | 392 | TTTT |
| Human-Exon 45 | 12 | −1 | AGUUCCUGUAAGAUACCAAAAAGG | 393 | TTTG |
| Human-Exon 45 | 13 | −1 | UCCUGGAGUUCCUGUAAGAUACCA | 394 | TTTT |
| Human-Exon 45 | 14 | −1 | AUCCUGGAGUUCCUGUAAGAUACC | 395 | TTTT |
| Human-Exon 45 | 15 | 1 | GGGAAGAAAUAAUUCAGCAAUCCU | 396 | TTTG |
| Human-Exon 45 | 16 | 1 | GGAAGAAAUAAUUCAGCAAUCCUC | 397 | TTTT |
| Human-Exon 45 | 17 | 1 | GAAGAAAUAAUUCAGCAAUCCUCA | 398 | TTTT |
| Human-Exon 45 | 18 | 1 | AAAACAGAUGCCAGUAUUCUACAG | 399 | TTTC |
| Human-Exon 45 | 19 | 1 | AAACAGAUGCCAGUAUUCUACAGG | 400 | TTTT |
| Human-Exon 45 | 20 | 1 | AACAGAUGCCAGUAUUCUACAGGA | 401 | TTTT |
| Human-Exon 45 | 21 | 1 | GAAUCUGCGGUGGCAGGAGGUCUG | 402 | TTTG |
| Human-Exon 45 | 22 | 1 | AGGUCUGCAAACAGCUGUCAGACA | 403 | TTTC |
| Human-Exon 45 | 23 | 1 | GGUCUGCAAACAGCUGUCAGACAG | 404 | TTTT |
| Human-Exon 45 | 24 | 1 | GUCUGCAAACAGCUGUCAGACAGA | 405 | TTTT |
| Human-Exon 45 | 25 | 1 | UCUGCAAACAGCUGUCAGACAGAA | 406 | TTTT |
| Human-Exon 45 | 26 | 1 | UAGGGCGACAGAUCUAAUAGGAAU | 407 | TTTC |
| Human-Exon 45 | 27 | 1 | AGGGCGACAGAUCUAAUAGGAAUG | 408 | TTTT |
| Human-Exon 45 | 28 | −1 | UAAAGAAAGCUUAAAAAGUCUGCU | 409 | TTTT |
| Human-Exon 45 | 29 | −1 | CUAAAGAAAGCUUAAAAAGUCUGC | 410 | TTTA |
| Human-Exon 45 | 30 | −1 | AAAUAUUCUUCUAAAGAAAGCUUA | 411 | TTTT |
| Human-Exon 45 | 31 | −1 | GAAAUAUUCUUCUAAAGAAAGCUU | 412 | TTTT |
| Human-Exon 45 | 32 | −1 | UGAAAUAUUCUUCUAAAGAAAGCU | 413 | TTTA |
| Human-Exon 45 | 33 | −1 | UCUCUCAUGAAAUAUUCUUCUAAA | 414 | TTTC |
| Human-Exon 45 | 34 | −1 | AUAAUCUCUCAUGAAAUAUUCUUC | 415 | TTTA |
| Human-Exon 44 | 1 | −1 | GCGUAUAUUUUUUGGUUAUACUGA | 416 | TTTG |
| Human-Exon 44 | 2 | −1 | ucaagaaaaauagauggauuaugu | 417 | tttt |
| Human-Exon 44 | 3 | −1 | aucaagaaaaauagauggauuaug | 418 | ttta |
| Human-Exon 44 | 4 | −1 | CAGGUaaaagcauauggaucaaga | 419 | tttt |
| Human-Exon 44 | 5 | −1 | GCAGGUaaaagcauauggaucaag | 420 | tttt |
| Human-Exon 44 | 6 | −1 | UGCAGGUaaaagcauauggaucaa | 421 | tttc |
| Human-Exon 44 | 7 | 1 | CAGGCGAUUUGACAGAUCUGUUGA | 422 | TTTC |
| Human-Exon 44 | 8 | −1 | AGAUCUGUCAAAUCGCCUGCAGGU | 423 | tttt |
| Human-Exon 44 | 9 | −1 | CAGAUCUGUCAAAUCGCCUGCAGG | 424 | tttA |
| Human-Exon 44 | 10 | −1 | GCCGCCAUUUCUCAACAGAUCUGU | 425 | TTTG |
| Human-Exon 44 | 11 | 1 | AAUGGCGGCGUUUUCAUUAUGAUA | 426 | TTTA |
| Human-Exon 44 | 12 | −1 | AUUAAAUAUCUUUAUAUCAUAAUG | 427 | TTTT |
| Human-Exon 44 | 13 | 1 | UGAGAAUUGGGAACAUGCUAAAUA | 428 | TTTG |
| Human-Exon 44 | 14 | 1 | GGUAAGUCUUUGAUUUGUUUUUUC | 429 | TTTC |
| Human-Exon 44 | 15 | −1 | AAAUACAAUUUCGAAAAAACAAAU | 430 | TTTG |
| Human-Exon 44 | 16 | −1 | AAGAUAAAUACAAUUUCGAAAAAA | 431 | TTTG |
| Human-Exon 44 | 17 | −1 | GCUGAAGAUAAAUACAAUUUCGAA | 432 | TTTT |
| Human-Exon 44 | 18 | −1 | UGCUGAAGAUAAAUACAAUUUCGA | 433 | TTTT |
| Human-Exon 44 | 19 | −1 | GUGCUGAAGAUAAAUACAAUUUCG | 434 | TTTT |
| Human-Exon 44 | 20 | −1 | UGUGCUGAAGAUAAAUACAAUUUC | 435 | TTTC |
| Human-Exon 44 | 21 | 1 | GCACAUCUGGACUCUUUAACUUCU | 436 | TTTA |
| Human-Exon 44 | 22 | −1 | UAAAGAGUCCAGAUGUGCUGAAGA | 437 | TTTA |
| Human-Exon 44 | 23 | −1 | AAGAUCAGGUUCUGAAGGGUGAUG | 438 | TTTC |
| Human-Exon 44 | 24 | −1 | UUCAGAACCUGAUCUUUAAGAAGU | 439 | TTTA |
| Human-Exon 44 | 25 | −1 | AAUAUAAUGAUGACAACAACAGUC | 440 | TTTT |
| Human-Exon 44 | 26 | −1 | UAAUAUAAUGAUGACAACAACAGU | 441 | TTTG |
| Human-Exon 53 | 1 | 1 | UUUAUUUUUCCUUUUAUUCUAGUU | 442 | TTTC |
| Human-Exon 53 | 2 | −1 | AAAGGAAAAAUAAAUAUAUAGUAG | 443 | TTTA |
| Human-Exon 53 | 3 | −1 | UUUCAACUAGAAUAAAAGGAAAAA | 444 | TTTA |
| Human-Exon 53 | 4 | −1 | AUUCUUUCAACUAGAAUAAAAGGA | 445 | TTTT |
| Human-Exon 53 | 5 | −1 | AAUUCUUUCAACUAGAAUAAAAGG | 446 | TTTT |
| Human-Exon 53 | 6 | −1 | GAAUUCUUUCAACUAGAAUAAAAG | 447 | TTTC |
| Human-Exon 53 | 7 | −1 | AUUCUGAAUUCUUUCAACUAGAAU | 448 | TTTT |
| Human-Exon 53 | 8 | −1 | GAUUCUGAAUUCUUUCAACUAGAA | 449 | TTTA |
| Human-Exon 53 | 9 | 1 | CAGAACCGGAGGCAACAGUUGAAU | 450 | TTTC |
| Human-Exon 53 | 10 | 1 | GGAGGCAACAGUUGAAUGAAAUGU | 451 | TTTA |
| Human-Exon 53 | 11 | 1 | UAUACAGUAGAUGCAAUCCAAAAG | 452 | TTTT |
| Human-Exon 53 | 12 | 1 | GAUGCAAUCCAAAAGAAAAUCACA | 453 | TTTC |
| Human-Exon 53 | 13 | 1 | AAUCACAGAAACCAAGGUUAGUAU | 454 | TTTG |
| Human-Exon 53 | 14 | 1 | AGGUUAGUAUCAAAGAUACCUUU | 455 | TTTA |
| Human-Exon 53 | 15 | 1 | GGUUAGUAUCAAAGAUACCUUUUU | 456 | TTTT |
| Human-Exon 53 | 16 | 1 | AGUAUCAAAGAUACCUUUUUAAAA | 457 | TTTA |
| Human-Exon 53 | 17 | 1 | GUAUCAAAGAUACCUUUUUAAAAU | 458 | TTTT |
| Human-Exon 46 | 1 | 1 | UGUUUGUGUCCCAGUUUGCAUUAA | 459 | TTTG |
| Human-Exon 46 | 2 | −1 | CUGGGACACAAACAUGGCAAUUUA | 460 | TTTT |
| Human-Exon 46 | 3 | −1 | ACUGGGACACAAACAUGGCAAUUU | 461 | TTTT |
| Human-Exon 46 | 4 | −1 | AACUGGGACACAAACAUGGCAAUU | 462 | TTTA |
| Human-Exon 46 | 5 | −1 | UAUUUGUUAAUGCAAACUGGGACA | 463 | TTTG |
| Human-Exon 46 | 6 | 1 | ACAAAUAGUUUGAGAACUAUGUUG | 464 | tttC |
| Human-Exon 46 | 7 | 1 | CAAAUAGUUUGAGAACUAUGUUGG | 465 | tttt |
| Human-Exon 46 | 8 | 1 | AAAUAGUUUGAGAACUAUGUUGGa | 466 | tttt |
| Human-Exon 46 | 9 | 1 | AUAGUUUGAGAACUAUGUUGGaaa | 467 | tttt |
| Human-Exon 46 | 10 | 1 | UAGUUUGAGAACUAUGUUGGaaaa | 468 | tttt |
| Human-Exon 46 | 11 | 1 | AGUUUGAGAACUAUGUUGGaaaaa | 469 | tttt |
| Human-Exon 46 | 12 | −1 | UAGUUCUCAAACUAUUUGUUAAUG | 470 | TTTG |
| Human-Exon 46 | 13 | −1 | UAuuuuuuuuuCCAACAUAGUUCU | 471 | TTTG |
| Human-Exon 46 | 14 | 1 | CUUCUUUCUCCAGGCUAGAAGAAC | 472 | TTTT |
| Human-Exon 46 | 15 | −1 | CUUCUAGCCUGGAGAAAGAAGAAU | 473 | TTTT |
| Human-Exon 46 | 16 | −1 | UCUUCUAGCCUGGAGAAAGAAGAA | 474 | TTTA |
| Human-Exon 46 | 17 | −1 | AUUCUUUUGUUCUUCUAGCCUGGA | 475 | TTTC |
| Human-Exon 46 | 18 | 1 | CAAAAGAAUAUCUUGUCAGAAUUU | 476 | TTTG |
| Human-Exon 46 | 19 | 1 | CUGGAAAAGAGCAGCAACUAAAAG | 477 | TTTT |
| Human-Exon 46 | 20 | 1 | CAAGUCAAGGUAAUUUUAUUUUCU | 478 | TTTG |
| Human-Exon 46 | 21 | 1 | CAAAUCCCCCAGGGCCUGCUUGCA | 479 | TTTA |
| Human-Exon 46 | 22 | −1 | AGGCCCUGGGGGAUUUGAGAAAAU | 480 | TTTT |
| Human-Exon 46 | 23 | −1 | CAGGCCCUGGGGGAUUUGAGAAAA | 481 | TTTA |
| Human-Exon 46 | 24 | −1 | CAAGCAGGCCCUGGGGGAUUUGAG | 482 | TTTT |
| Human-Exon 46 | 25 | −1 | GCAAGCAGGCCCUGGGGGAUUUGA | 483 | TTTC |
| Human-Exon 46 | 26 | −1 | GCAGAAAACCAAUGAUUGAAUUAA | 484 | TTTT |
| Human-Exon 46 | 27 | −1 | GGCAGAAAACCAAUGAUUGAAUUA | 485 | TTTT |
| Human-Exon 46 | 28 | −1 | GGGCAGAAAACCAAUGAUUGAAUU | 486 | TTTT |
| Human-Exon 46 | 29 | −1 | UGGGCAGAAAACCAAUGAUUGAAU | 487 | TTTA |
| Human-Exon 46 | 30 | 1 | AUUAGGUUAUUCAUAGUUCCUUGC | 488 | TTTA |
| Human-Exon 46 | 31 | −1 | AACUAUGAAUAACCUAAUGGGCAG | 489 | TTTT |
| Human-Exon 46 | 32 | −1 | GAACUAUGAAUAACCUAAUGGGCA | 490 | TTTC |
| Human-Exon 52 | 1 | 1 | UAUUUCCUGUUAAAUUGUUUUCUA | 491 | TTTA |
| Human-Exon 52 | 2 | −1 | GGUUUAUAGAAAACAAUUUAACAG | 492 | TTTC |
| Human-Exon 52 | 3 | 1 | AUACAGUAACAUCUUUUUUAUUUC | 493 | TTTA |
| Human-Exon 52 | 4 | 1 | UACAGUAACAUCUUUUUUAUUUCU | 494 | TTTT |
| Human-Exon 52 | 5 | −1 | AUGUUACUGUAUAAGGGUUUAUAG | 495 | TTTT |
| Human-Exon 52 | 6 | −1 | GAUGUUACUGUAUAAGGGUUUAUA | 496 | TTTC |
| Human-Exon 52 | 7 | −1 | CAGCCAAAACACUUUUAGAAAUAA | 497 | TTTT |
| Human-Exon 52 | 8 | −1 | CCAGCCAAAACACUUUUAGAAAUA | 498 | TTTT |
| Human-Exon 52 | 9 | −1 | ACCAGCCAAAACACUUUUAGAAAU | 499 | TTTT |
| Human-Exon 52 | 10 | −1 | GACCAGCCAAAACACUUUUAGAAA | 500 | TTTA |
| Human-Exon 52 | 11 | −1 | GUGAGACCAGCCAAAACACUUUUA | 501 | TTTC |
| Human-Exon 52 | 12 | 1 | AAUUGUACUUUACUUUGUAUUAUG | 502 | TTTA |
| Human-Exon 52 | 13 | 1 | AUUGUACUUUACUUUGUAUUAUGU | 503 | TTTT |
| Human-Exon 52 | 14 | −1 | UAAAGUACAAUUGUGAGACCAGCC | 504 | TTTT |
| Human-Exon 52 | 15 | −1 | GUAAAGUACAAUUGUGAGACCAGC | 505 | TTTG |
| Human-Exon 52 | 16 | −1 | GUAUUCCUUUUACAUAAUACAAAG | 506 | TTTA |
| Human-Exon 52 | 17 | −1 | GUUGUGUAUUCCUUUUACAUAAUA | 507 | TTTG |
| Human-Exon 52 | 18 | −1 | AUCCUGCAUUGUUGCCUGUAAGAA | 508 | TTTG |
| Human-Exon 52 | 19 | −1 | UUCCAACUGGGGACGCCUCUGUUC | 509 | TTTG |
| Human-Exon 52 | 20 | 1 | UUGGAAGAACUCAUUACCGCUGCC | 510 | TTTG |
| Human-Exon 52 | 21 | 1 | UCAUUACCGCUGCCCAAAAUUUGA | 511 | TTTT |
| Human-Exon 52 | 22 | −1 | CUCUUGAUUGCUGGUCUUGUUUUU | 512 | TTTG |
| Human-Exon 52 | 23 | 1 | GUUUUUUAACAAGCAUGGGACACA | 513 | TTTG |
| Human-Exon 52 | 24 | −1 | CUUUGUGUGUCCCAUGCUUGUUAA | 514 | TTTT |
| Human-Exon 52 | 25 | −1 | GCUUUGUGUGUCCCAUGCUUGUUA | 515 | TTTT |
| Human-Exon 52 | 26 | −1 | UGCUUUGUGUGUCCCAUGCUUGUU | 516 | TTTT |
| Human-Exon 52 | 27 | −1 | UUGCUUUGUGUGUCCCAUGCUUGU | 517 | TTTA |
| Human-Exon 52 | 28 | 1 | AGCAAGAUGCAUGACAAGUUUCAA | 518 | TTTA |
| Human-Exon 52 | 29 | 1 | GCAAGAUGCAUGACAAGUUUCAAU | 519 | TTTT |
| Human-Exon 52 | 30 | 1 | CAAGAUGCAUGACAAGUUUCAAUA | 520 | TTTT |
| Human-Exon 52 | 31 | −1 | GAUAUAUGAACUUAAGUUUUUAUU | 521 | TTTC |
| Human-Exon 50 | 1 | 1 | AUAGAAAUCCAAUAAUAUAUUCAC | 522 | TTTG |
| Human-Exon 50 | 2 | 1 | AUUAAGAUGUUCAUGAAUUAUCUU | 523 | TTTG |
| Human-Exon 50 | 3 | 1 | UAAGUAAUGUGUAUGCUUUUCUGU | 524 | TTTA |
| Human-Exon 50 | 4 | −1 | AUCUUCUAACUUCCUCUUUAACAG | 525 | TTTT |
| Human-Exon 50 | 5 | −1 | GAUCUUCUAACUUCCUCUUUAACA | 526 | TTTC |
| Human-Exon 50 | 6 | 1 | AUCUGAGCUCUGAGUGGAAGGCGG | 527 | TTTA |
| Human-Exon 50 | 7 | 1 | ACCGUUUACUUCAAGAGCUGAGGG | 528 | TTTG |
| Human-Exon 50 | 8 | −1 | CUGCUUUGCCCUCAGCUCUUGAAG | 529 | TTTA |
| Human-Exon 50 | 9 | 1 | UCUCUUUGGCUCUAGCUAUUUGUU | 530 | TTTG |
| Human-Exon 50 | 10 | 1 | CUCUUUGGCUCUAGCUAUUUGUUC | 531 | TTTT |
| Human-Exon 50 | 11 | −1 | CACUUUUGAACAAAUAGCUAGAGC | 532 | TTTG |
| Human-Exon 50 | 12 | −1 | UCACUUCAUAGUUGCACUUUUGAA | 533 | TTTG |
| Human-Exon 50 | 13 | 1 | AUGAAGUGAUGACUGGGUGAGAGA | 534 | TTTC |
| Human-Exon 50 | 14 | 1 | UGAAGUGAUGACUGGGUGAGAGAG | 535 | TTTT |
| Human-Exon 43 | 1 | −1 | AAGAGAAAAauauauauauauaua | 536 | TTTG |
| Human-Exon 43 | 2 | −1 | GAAUUAGCUGUCUAUAGAAAGAGA | 537 | tTTT |
| Human-Exon 43 | 3 | −1 | UGAAUUAGCUGUCUAUAGAAAGAG | 538 | TTTT |
| Human-Exon 43 | 4 | 1 | AGCUAAUUCAUUUUUUUACUGUUU | 539 | TTTA |
| Human-Exon 43 | 5 | −1 | AUGAAUUAGCUGUCUAUAGAAAGA | 540 | TTTC |
| Human-Exon 43 | 6 | 1 | GCUAAUUCAUUUUUUUACUGUUUU | 541 | TTTT |
| Human-Exon 43 | 7 | −1 | AAAAAAAUGAAUUAGCUGUCUAUA | 542 | TTTC |
| Human-Exon 43 | 8 | 1 | UUAAAAUUUUUAUAUUACAGAAUA | 543 | TTTA |
| Human-Exon 43 | 9 | 1 | UAAAAUUUUUAUAUUACAGAAUAU | 544 | TTTT |
| Human-Exon 43 | 10 | −1 | AUAUAAAAAUUUUAAAACAGUAAA | 545 | TTTT |
| Human-Exon 43 | 11 | −1 | AAUAUAAAAAUUUUAAAACAGUAA | 546 | TTTT |
| Human-Exon 43 | 12 | −1 | UAAUAUAAAAAUUUUAAAACAGUA | 547 | TTTT |
| Human-Exon 43 | 13 | −1 | GUAAUAUAAAAAUUUUAAAACAGU | 548 | TTTT |
| Human-Exon 43 | 14 | −1 | UGUAAUAUAAAAAUUUUAAAACAG | 549 | TTTA |
| Human-Exon 43 | 15 | −1 | UAUAUUCUGUAAUAUAAAAAUUUU | 550 | TTTT |
| Human-Exon 43 | 16 | −1 | UUAUAUUCUGUAAUAUAAAAAUUU | 551 | TTTA |
| Human-Exon 43 | 17 | 1 | CAGAAUAUAAAAGAUAGUCUACAA | 552 | TTTG |
| Human-Exon 43 | 18 | −1 | CUAUCUUUUAUAUUCUGUAAUAUA | 553 | TTTT |
| Human-Exon 43 | 19 | −1 | ACUAUCUUUUAUAUUCUGUAAUAU | 554 | TTTT |
| Human-Exon 43 | 20 | −1 | GACUAUCUUUUAUAUUCUGUAAUA | 555 | TTTA |
| Human-Exon 43 | 21 | 1 | CAUAGCAAGAAGACAGCAGCAUUG | 556 | TTTG |
| Human-Exon 43 | 22 | −1 | CAUUUUGUUAACUUUUUCCCAUUG | 557 | TTTC |
| Human-Exon 43 | 23 | 1 | CAUAUAUUUUUCUUGAUACUUGCA | 558 | TTTC |
| Human-Exon 43 | 24 | −1 | AAAUCAUUUCUGCAAGUAUCAAGA | 559 | TTTT |
| Human-Exon 43 | 25 | −1 | CAAAUCAUUUCUGCAAGUAUCAAG | 560 | TTTT |
| Human-Exon 43 | 26 | −1 | ACAAAUCAUUUCUGCAAGUAUCAA | 561 | TTTC |
| Human-Exon 43 | 27 | −1 | AUAAAUUCUACAGUUCCCUGAAAA | 562 | TTTG |
| Human-Exon 43 | 28 | 1 | GAAUUUAUUUCAGUACCCUCCAUG | 563 | TTTC |
| Human-Exon 43 | 29 | 1 | AAUUUAUUUCAGUACCCUCCAUGG | 564 | TTTT |
| Human-Exon 43 | 30 | −1 | UGAAAUAAAUUCUACAGUUCCCUG | 565 | TTTT |
| Human-Exon 43 | 31 | 1 | AUUUAUUUCAGUACCCUCCAUGGA | 566 | TTTT |
| Human-Exon 43 | 32 | −1 | CUGAAAUAAAUUCUACAGUUCCCU | 567 | TTTC |
| Human-Exon 43 | 33 | 1 | UUUAUUUCAGUACCCUCCAUGGAA | 568 | TTTT |
| Human-Exon 43 | 34 | 1 | UACCCUCCAUGGAAAAAAGACAGG | 569 | TTTC |
| Human-Exon 43 | 35 | 1 | ACCCUCCAUGGAAAAAAGACAGGG | 570 | TTTT |
| Human-Exon 43 | 36 | 1 | CCCUCCAUGGAAAAAAGACAGGGA | 571 | TTTT |
| Human-Exon 43 | 37 | −1 | UUUUUUCCAUGGAGGGUACUGAAA | 572 | TTTA |
| Human-Exon 43 | 38 | −1 | UGUCUUUUUUCCAUGGAGGGUACU | 573 | TTTC |
| Human-Exon 6 | 1 | −1 | CCUUGAGCAAGAACCAUGCAAACU | 574 | TTTA |
| Human-Exon 6 | 2 | 1 | UGCUCAAGGAAUGCAUUUUCUUAU | 575 | TTTC |
| Human-Exon 6 | 3 | 1 | GCUCAAGGAAUGCAUUUUCUUAUG | 576 | TTTT |
| Human-Exon 6 | 4 | −1 | UGCAUUCCUUGAGCAAGAACCAUG | 577 | TTTG |
| Human-Exon 6 | 5 | 1 | GAAAAUUUAUUUCCACAUGUAGGU | 578 | TTTG |
| Human-Exon 6 | 6 | 1 | AAAAUUUAUUUCCACAUGUAGGUC | 579 | TTTT |
| Human-Exon 6 | 7 | 1 | AAAUUUAUUUCCACAUGUAGGUCA | 580 | TTTT |
| Human-Exon 6 | 8 | −1 | CAUGUGGAAAUAAAUUUUCAUAAG | 581 | TTTT |
| Human-Exon 6 | 9 | −1 | ACAUGUGGAAAUAAAUUUUCAUAA | 58 | TTTC |
| Human-Exon 6 | 10 | 1 | CCACAUGUAGGUCAAAAAUGUAAU | 583 | TTTC |
| Human-Exon 6 | 11 | 1 | CACAUGUAGGUCAAAAAUGUAAUG | 584 | TTTT |
| Human-Exon 6 | 12 | 1 | ACAUGUAGGUCAAAAAUGUAAUGA | 585 | TTTT |
| Human-Exon 6 | 13 | −1 | ACAUUUUUGACCUACAUGUGGAAA | 586 | TTTA |
| Human-Exon 6 | 14 | −1 | CAUUACAUUUUUGACCUACAUGUG | 587 | TTTC |
| Human-Exon 6 | 15 | 1 | AAAAAUAUCAUGGCUGGAUUGCAA | 588 | TTTG |
| Human-Exon 6 | 16 | 1 | GCUGGAUUGCAACAAACCAACAGU | 589 | TTTC |
| Human-Exon 6 | 17 | 1 | CUGGAUUGCAACAAACCAACAGUG | 590 | TTTT |
| Human-Exon 6 | 18 | −1 | CCUAUGACUAUGGAUGAGAGCAUU | 591 | TTTG |
| Human-Exon 6 | 19 | 1 | UAGGUAAGAAGAUUACUGAGACAU | 592 | TTTA |
| Human-Exon 6 | 20 | 1 | AUUACUGAGACAUUAAAUAACUUG | 593 | TTTA |
| Human-Exon 6 | 21 | 1 | UUACUGAGACAUUAAAUAACUUGU | 594 | TTTT |
| Human-Exon 6 | 22 | −1 | GGGGAAAAAUAUGUCAUCAGAGUC | 595 | TTTA |
| Human-Exon 6 | 23 | −1 | CAUGAUCUGGAACCAUACUGGGGA | 596 | TTTT |
| Human-Exon 6 | 24 | −1 | ACAUGAUCUGGAACCAUACUGGGG | 597 | TTTT |
| Human-Exon 6 | 25 | −1 | GACAUGAUCUGGAACCAUACUGGG | 598 | TTTC |
| Human-Exon 7 | 1 | −1 | uacacacauacacaAAGACAAAUA | 599 | TTTA |
| Human-Exon 7 | 2 | −1 | uacacauacacacauacacaAAGA | 600 | TTTG |
| Human-Exon 7 | 3 | −1 | aacacauacacauacacacauaca | 601 | TTtg |
| Human-Exon 7 | 4 | −1 | AUUCCAGUCAAAUAGGUCUGGCCU | 602 | ttTT |
| Human-Exon 7 | 5 | −1 | UAUUCCAGUCAAAUAGGUCUGGCC | 603 | tTTA |
| Human-Exon 7 | 6 | −1 | GCUGGCAAACCACACUAUUCCAGU | 604 | TTTG |
| Human-Exon 7 | 7 | −1 | AGUCGUUGUGUGGCUGACUGCUGG | 605 | TTTG |
| Human-Exon 7 | 8 | 1 | CGCCAGAUAUCAAUUAGGCAUAGA | 606 | TTTC |
| Human-Exon 7 | 9 | 1 | AAACUACUCGAUCCUGAAGGUUGG | 607 | TTTA |
| Human-Exon 7 | 10 | −1 | CAUACUAAAAGCAGUGGUAGUCCA | 608 | TTTC |
| Human-Exon 7 | 11 | −1 | GAAAACAUUAAACUCUACCAUACU | 609 | TTTT |
| Human-Exon 7 | 12 | −1 | UGAAAACAUUAAACUCUACCAUAC | 610 | TTTA |
| Human-Exon 8 | 1 | 1 | UUGUUCAUUAUCCUUUUAGAGUCU | 611 | TTTG |
| Human-Exon 8 | 2 | −1 | AAAGGAUAAUGAACAAAUCAAAGU | 612 | TTTA |
| Human-Exon 8 | 3 | 1 | UAUCCUUUUAGAGUCUCAAAUAUA | 613 | TTTC |
| Human-Exon 8 | 4 | −1 | ACUCUAAAAGGAUAAUGAACAAAU | 614 | TTTG |
| Human-Exon 8 | 5 | 1 | UUUUAGAGUCUCAAAUAUAGAAAC | 615 | TTTG |
| Human-Exon 8 | 6 | 1 | UUUAGAGUCUCAAAUAUAGAAACC | 616 | TTTT |
| Human-Exon 8 | 7 | 1 | UUAGAGUCUCAAAUAUAGAAACCA | 617 | TTTT |
| Human-Exon 8 | 8 | −1 | UUGAGACUCUAAAAGGAUAAUGAA | 618 | TTTG |
| Human-Exon 8 | 9 | −1 | UUUGGUUUCUAUAUUUGAGACUCU | 619 | TTTT |
| Human-Exon 8 | 10 | −1 | UUUUGGUUUCUAUAUUUGAGACUC | 620 | TTTA |
| Human-Exon 8 | 11 | 1 | AGCAUUGAAGCCAUCCAGGAAGUG | 621 | TTTC |
| Human-Exon 8 | 12 | −1 | GCUUCAAUGCUCACUUGUUGAGGC | 622 | TTTT |
| Human-Exon 8 | 13 | −1 | GGCUUCAAUGCUCACUUGUUGAGG | 623 | TTTG |
| Human-Exon 8 | 14 | 1 | AGUGGAAAUGUUGCCAAGGCCACC | 624 | TTTA |
| Human-Exon 8 | 15 | 1 | GUUGCCAAGGCCACCUAAAGUGAC | 625 | TTTA |
| Human-Exon 8 | 16 | 1 | GAAGAACAUUUUCAGUUACAUCAU | 626 | TTTG |
| Human-Exon 8 | 17 | 1 | AUCAAAUGCACUAUUCUCAACAGG | 627 | TTTA |
| Human-Exon 8 | 18 | −1 | AUAGUGCAUUUGAUGAUGUAACUG | 628 | TTTT |
| Human-Exon 8 | 19 | −1 | AAUAGUGCAUUUGAUGAUGUAACU | 629 | TTTC |
| Human-Exon 8 | 20 | 1 | ACUAUUCUCAACAGGUAAAGUGUG | 630 | TTTA |
| Human-Exon 8 | 21 | −1 | UACCUAAAAAUGCAUAUAAAACAG | 631 | TTTT |
| Human-Exon 8 | 22 | −1 | AUACCUAAAAAUGCAUAUAAAACA | 632 | TTTC |
| Human-Exon 8 | 23 | −1 | CACGUAAUACCUAAAAAUGCAUAU | 633 | TTTT |
| Human-Exon 8 | 24 | −1 | GCACGUAAUACCUAAAAAUGCAUA | 634 | TTTA |
| Human-Exon 8 | 25 | −1 | auauauauGUGCACGUAAUACCUA | 635 | TTTT |
| Human-Exon 8 | 26 | −1 | uauauauauGUGCACGUAAUACCU | 636 | TTTT |
| Human-Exon 8 | 27 | −1 | auauauauauGUGCACGUAAUACC | 637 | TTTA |
| Human-Exon 55 | 1 | 1 | CUGCACAAUAUUAUAGUUGUUGCU | 638 | TTTA |
| Human-Exon 55 | 2 | −1 | AUAAAAAGAGAAAGAUGGAGGAAC | 638 | TTTA |
| Human-Exon 55 | 3 | −1 | CACCUAGUGAACUCCAUAAAAAGA | 640 | TTTC |
| Human-Exon 55 | 4 | −1 | AUGGUGCACCUAGUGAACUCCAUA | 641 | TTTT |
| Human-Exon 55 | 5 | −1 | AAUGGUGCACCUAGUGAACUCCAU | 642 | TTTT |
| Human-Exon 55 | 6 | −1 | GAAUGGUGCACCUAGUGAACUCCA | 643 | TTTA |
| Human-Exon 55 | 7 | −1 | GACCAAAUGUUCAGAUGCAAUUAU | 644 | TTTA |
| Human-Exon 55 | 8 | −1 | UCGCUCACUCACCCUGCAAAGGAC | 645 | TTTG |
| Human-Exon 55 | 9 | 1 | AGUGAGCGAGAGGCUGCUUUGGAA | 646 | TTTC |
| Human-Exon 55 | 10 | −1 | GCAGCCUCUCGCUCACUCACCCUG | 647 | TTTG |
| Human-Exon 55 | 11 | −1 | UUGCAGUAAUCUAUGAGUUUCUUC | 648 | TTTG |
| Human-Exon 55 | 12 | 1 | CUGCAACAGUUCCCCCUGGACCUG | 649 | TTTC |
| Human-Exon 55 | 13 | 1 | UGCAACAGUUCCCCCUGGACCUGG | 650 | TTTT |
| Human-Exon 55 | 14 | 1 | UUUCUUGCCUGGCUUACAGAAGCU | 651 | TTTC |
| Human-Exon 55 | 15 | −1 | UUUCAGCUUCUGUAAGCCAGGCAA | 652 | TTTC |
| Human-Exon 55 | 16 | 1 | GUCCUACAGGAUGCUACCCGUAAG | 653 | TTTC |
| Human-Exon 55 | 17 | 1 | GGCUCCUAGAAGACUCCAAGGGAG | 654 | TTTA |
| Human-Exon 55 | 18 | 1 | GCUCCUAGAAGACUCCAAGGGAGU | 655 | TTTT |
| Human-Exon 55 | 19 | 1 | CUCCAAGGGAGUAAAAGAGCUGAU | 656 | TTTC |
| Human-Exon 55 | 20 | −1 | UGGAUCCACAAGAGUGCUAAAGCG | 657 | TTTC |
| Human-Exon 55 | 21 | −1 | GUUCAAUUGGAUCCACAAGAGUGC | 658 | TTTA |
| Human-Exon 55 | 22 | 1 | UACUUGUAACUGACAAGCCAGGGA | 659 | TTTG |
| Human-Exon 55 | 23 | 1 | ACUUGUAACUGACAAGCCAGGGAC | 660 | TTTT |
| Human-Exon 55 | 24 | 1 | GUAACUGACAAGCCAGGGACAAAA | 661 | TTTG |
| Human-Exon 55 | 25 | 1 | UAACUGACAAGCCAGGGACAAAAC | 662 | TTTT |
| Human-Exon 55 | 26 | −1 | UCCCUGGCUUGUCAGUUACAAGUA | 663 | TTTG |
| Human-G1-exon51 | | −1 | CAGAGUAACAGUCUGAGUAGGAGc | 664 | TTTA |
| Human-G2-exon51 | | −1 | uacuuuguuuagcaauacauggua | 665 | TTTC |
| Human-G3-exon51 | | 1 | uggcucaaauuguuacucuucaau | 666 | TTTA |
| mouse-Exon23-G1 | | −1 | CUUUCAAagaacuuugcagagccu | 667 | TTTG |
| mouse-Exon23-G2 | | −1 | guugaaGCCAUUUUGUUGCUCUUU | 668 | TTTG |
| mouse-Exon23-G3 | | −1 | guugaaGCCAUUUUAUUGCUCUUU | 669 | TTTG |
| mouse-Exon23-G4 | | 1 | uuuugagGCUCUGCAAAGUUCUUU | 670 | TTTC |
| mouse-Exon23-G5 | | 1 | aguuauuaaugcauagauauucag | 671 | TTTA |
| mouse-Exon23-G6 | | 1 | uauaauaugcccuguaauauaaua | 672 | TTTC |
| mouse-Exon23-G7 | | −1 | uaaaggccaaaccucggcuuaccU | 673 | TTTC |
| mouse-Exon23-G8 | | −1 | ucaauaucuuugaaggacucuggg | 674 | TTTA |

*In this table, upper case letters represent sgRNA nucleotides that align to the exon sequence of the gene. Lower case letters represent sgRNA nucleotides that align to the intron sequence of the gene.

TABLE 5
Exemplary gRNA targeting sequences for
targeting mouse Dmd Exon 51

| | | | SEQ | |
|---|---|---|---|---|
| | | | ID | |
| ID gRNA | Strand | sgRNA | NO: | PAM |
| Ex51-SA1 | 3′ | AGAGTAACAGTCTGACTGG | 675 | CAG |
| Ex51-SD | 5′ | GAAATGATCATCAAACAGA | 676 | AGG |
| Ex51-SA-2 | 3′ | CACTAGAGTAACAGTCTGAC | 677 | TGG |

TABLE 6
Exemplary gRNAs targeting mouse Dmd Exon 51

| | | | SEQ | |
|---|---|---|---|---|
| | | | ID | |
| ID gRNA | Strand | sgRNA | NO: | PAM |
| Ex51-SA1 | 3′ | CCAGUCAGACUGUUACUCU | 678 | CAG |
| Ex51-SD | 5′ | UCUGUUUGAUGAUCAUUUC | 679 | AGG |
| Ex51-SA-2 | 3′ | GUCAGACUGUUACUCUAGUG | 680 | TGG |
| Ex51-SA1′ | 3′ | AGAGUAACAGUCUGACUGG | 681 | CAG |
| Ex51-SD′ | 5′ | GAAAUGAUCAUCAAACAGA | 682 | AGG |
| Ex51-SA-2′ | 3′ | CACUAGAGUAACAGUCUGAC | 683 | TGG |

TABLE 7
Exemplary gRNAs targeting human Dmd Exon 51

| | | | SEQ ID | |
|---|---|---|---|---|
| ID gRNA | Strand | sgRNA | NO: | PAM |
| Ex51-SA | 3′ | AGAGTAACAGTCTGAGTAG | 684 | GAG |
| Ex51-SD | 5′ | GAGATGATCATCAAGCAGA | 685 | AGG |
| Ex51-SA-2 | 3′ | CACCAGAGTAACAGTCTGAG | 686 | TAG |

TABLE 8
gRNA sequences for targeting human Dmd Exon 51

| | | | SEQ ID | |
|---|---|---|---|---|
| ID gRNA | Strand | sgRNA | NO: | PAM |
| Ex51-SA | 3′ | CUACUCAGACUGUUACUCU | 687 | GAG |
| Ex51-SD | 5′ | UCUGCUUGAUGAUCAUCUC | 688 | AGG |
| Ex51-SA-2 | 3′ | CUCAGACUGUUACUCUGGUG | 689 | TAG |
| Ex51-SA′ | 3′ | AGAGUAACAGUCUGAGUAG | 690 | GAG |
| Ex51-SD′ | 5′ | GAGAUGAUCAUCAAGCAGA | 691 | AGG |
| Ex51-SA-2′ | 3′ | CACCAGAGUAACAGUCUGAG | 692 | TAG |

TABLE 9
gRNAs Targeting sites in various human Dmd Exons

| | | | SEQ | |
|---|---|---|---|---|
| | | | ID | |
| ID gRNA | Strand | sgRNA | NO: | PAM |
| Exon50-#1 | 3′ | TAGTGGTCAGTCCAGGAGCT | 693 | AGG |
| Exon50-#2 | 3′ | GCTCCAATAGTGGTCAGTCC | 694 | AGG |
| Exon50-#3 | 5′ | GCTCCTGGACTGACCACTAT | 695 | TGG |
| Exon50-#4 | 3′ | ATACTTACAGGCTCCAATAG | 696 | TGG |
| Exon50-#5 | 3′ | ATGGGATCCAGTATACTTAC | 697 | AGG |
| Exon50-#6 | 5′ | ATTGGAGCCTGTAAGTATAC | 698 | TGG |
| Exon51-#1 | 3′ | CAGAGTAACAGTCTGAGTAG | 699 | GAG |
| Exon51-#2 | 3′ | CACCAGAGTAACAGTCTGAG | 700 | TAG |
| Exon51-#3 | 3′ | TATTTTGGGTTTTTGCAAAA | 701 | AGG |
| Exon51-#4 | 3′ | AGTAGGAGCTAAAATATTTT | 702 | GGG |
| Exon51-#5 | 3′ | GAGTAGGAGCTAAAATATTT | 703 | TGG |
| Exon51-#6 | 3′ | ACCAGAGTAACAGTCTGAGT | 704 | AGG |
| Exon51-#7 | 5′ | TCCTACTCAGACTGTTACTC | 705 | TGG |
| Exon51-#8 | 5′ | TACTCTGGTGACACAACCTG | 706 | TGG |
| Exon51-#9 | 3′ | GCAGTTTCCTTAGTAACCAC | 707 | AGG |
| Exon51-#10 | 5′ | GACACAACCTGTGGTTACTA | 708 | AGG |
| Exon51-#11 | 3′ | TGTCACCAGAGTAACAGTCT | 709 | GAG |
| Exon51-#12 | 3′ | AGGTTGTGTCACCAGAGTAA | 710 | CAG |
| Exon51-#13 | 3′ | AACCACAGGTTGTGTCACCA | 711 | GAG |
| Exon51-#14 | 3′ | GTAACCACAGGTTGTGTCAC | 712 | CAG |
| Exon53-#1 | 5′ | ATTTATTTTTCCTTTTATTC | 713 | TAG |
| Exon53-#2 | 5′ | TTTCCTTTTATTCTAGTTGA | 714 | AAG |
| Exon53-#3 | 3′ | TGATTCTGAATTCTTTCAAC | 715 | TAG |
| Exon53-#4 | 3′ | AATTCTTTCAACTAGAATAA | 716 | AAG |
| Exon53-#6 | 5′ | TTATTCTAGTTGAAAGAATT | 717 | CAG |
| Exon53-#7 | 5′ | TAGTTGAAAGAATTCAGAAT | 718 | CAG |
| Exon53-#8 | 5′ | AATTCAGAATCAGTGGGATG | 719 | AAG |
| Exon53-#9 | 3′ | ATTCTTTCAACTAGAATAAA | 720 | AGG |
| Exon53-#10 | 5′ | TTGAAAGAATTCAGAATCAG | 721 | TGG |
| Exon53-#11 | 5′ | TGAAAGAATTCAGAATCAGT | 722 | GGG |
| Exon53-#12 | 3′ | ACTGTTGCCTCCGGTTCTGA | 723 | AGG |
| Exon44-#1 | 3′ | CAGATCTGTCAAATCGCCTG | 724 | CAG |
| Exon44-#2 | 3′ | AAAACGCCGCCATTTCTCAA | 725 | CAG |
| Exon44-#3 | 3′ | AGATCTGTCAAATCGCCTGC | 726 | AGG |
| Exon44-#4 | 3′ | TATGGATCAAGAAAAATAGA | 727 | TGG |
| Exon44-#5 | 3′ | CGCCTGCAGGTAAAAGCATA | 728 | TGG |
| Exon44-#6 | 5′ | ATCCATATGCTTTTACCTGC | 729 | AGG |
| Exon44-#8 | 5′ | TTGACAGATCTGTTGAGAAA | 730 | TGG |
| Exon44-#9 | 5′ | ACAGATCTGTTGAGAAATGG | 731 | CGG |
| Exon44-#11 | 5′ | GGCGATTTGACAGATCTGTT | 732 | GAG |
| Exon44-#13 | 5′ | GGCGTTTTCATTATGATATA | 733 | AAG |
| Exon44-#14 | 5′ | ATGATATAAAGATATTTAAT | 734 | CAG |
| Exon44-#15 | 5′ | GATATTTAATCAGTGGCTAA | 735 | CAG |
| Exon44-#16 | 5′ | ATTTAATCAGTGGCTAACAG | 736 | AAG |
| Exon44-#17 | 3′ | AGAAACTGTTCAGCTTCTGT | 737 | TAG |
| Exon44-#1 | 5′ | GGGAACATGCTAAATACAAA | 738 | TGG |
| Exon44-#2 | 5′ | TAAATACAAATGGTATCTTA | 739 | AGG |
| Exon43-#1 | 5′ | GTTTTAAAATTTTTATATTA | 740 | CAG |
| Exon43-#2 | 5′ | TTTTATATTACAGAATATAA | 741 | AAG |
| Exon43-#3 | 5′ | ATATTACAGAATATAAAAGA | 742 | TAG |
| Exon45-#1 | 3′ | GTTCCTGTAAGATACCAAAA | 743 | AGG |
| Exon45-#2 | 5′ | TTGCCTTTTTGGTATCTTAC | 744 | AGG |
| Exon45-#3 | 5′ | TGGTATCTTACAGGAACTCC | 745 | AGG |
| Exon45-#4 | 5′ | ATCTTACAGGAACTCCAGGA | 746 | TGG |
| Exon45-#4.1 | 5′ | CTTACAGGAACTCCAGGA | 2356 | TGG |
| Exon45-#5 | 3′ | GCCGCTGCCCAATGCCATCC | 747 | TGG |
| Exon45-#6 | 5′ | CAGGAACTCCAGGATGGCAT | 748 | TGG |
| Exon45-#7 | 5′ | AGGAACTCCAGGATGGCATT | 749 | GGG |
| Exon45-#8 | 5′ | TCCAGGATGGCATTGGGCAG | 750 | CGG |
| Exon45-#9 | 5′ | GTCAGAACATTGAATGCAAC | 751 | TGG |
| Exon45-#10 | 3′ | AGTTCCTGTAAGATACCAAA | 752 | AAG |
| Exon45-#11 | 3′ | TGCCATCCTGGAGTTCCTGT | 753 | AAG |
| Exon45-#12 | 5′ | TTGGTATCTTACAGGAACTC | 754 | CAG |
| Exon45-#13 | 3′ | CGCTGCCCAATGCCATCCTG | 755 | GAG |
| Exon45-#14 | 5′ | AACTCCAGGATGGCATTGGG | 756 | CAG |
| Exon45-#15 | 5′ | GGGCAGCGGCAAACTGTTGT | 757 | CAG |
| Exon46-#1 | 3′ | ATTCTTTTGTTCTTCTAGCC | 758 | TGG |
| Exon46-#2 | 3′ | TGCTCTTTTCCAGGTTCAAG | 759 | GGG |
| Exon46-#3 | 5′ | GCTAGTATCCCACTTGAACC | 760 | TGG |
| Exon46-#4 | 3′ | TTTAGTTGCTGCTCTTTTCC | 761 | TGG |
| Exon46-#5 | 5′ | AGAAAAGCTTGAGCAAGTCA | 762 | AGG |
| Exon52-#1 | 3′ | AGATCTGTCAAATCGCCTGC | 763 | AGG |
| Exon52-#2 | 3′ | AATCCTGCATTGTTGCCTGT | 764 | AAG |
| Exon52-#3 | 5′ | CGCTGAAGAACCCTGATACT | 765 | AAG |
| Exon52-#4 | 3′ | GAACAAATATCCCTTAGTAT | 766 | CAG |
| Exon52-#5 | 3′ | CTGTAAGAACAAATATCCCT | 767 | TAG |
| Exon52-#6 | 5′ | CTAAGGGATATTTGTTCTTA | 768 | CAG |
| Exon52-#8 | 5′ | TGTTCTTACAGGCAACAATG | 769 | CAG |
| Exon52-#9 | 5′ | CAACAATGCAGGATTTGGAA | 770 | CAG |
| Exon52-#10 | 5′ | ACAATGCAGGATTTGGAACA | 771 | GAG |
| Exon52-#11 | 5′ | ATTTGGAACAGAGGCGTCCC | 772 | CAG |
| Exon52-#12 | 5′ | ACAGAGGCGTCCCCAGTTGG | 773 | AAG |
| Exon2-#1 | 5′ | TATTTTTTTATTTTGCATTT | 774 | TAG |
| Exon2-#2 | 5′ | TTATTTTGCATTTTAGATGA | 775 | AAG |
| Exon2-#3 | 5′ | ATTTTGCATTTTAGATGAAA | 776 | GAG |
| Exon2-#4 | 5′ | TTGCATTTTAGATGAAAGAG | 777 | AAG |
| Exon2-#5 | 5′ | ATGAAAGAGAAGATGTTCAA | 778 | AAG |

TABLE 10
gRNA sequences for targeting sites in
various human DMD Exons

| | | | SEQ | |
|---|---|---|---|---|
| | | | ID | |
| ID sgRNA | Strand | sgRNA | NO: | PAM |
| Exon51-#1 | 3′ | CUACUCAGACUGUUACUCUG | 779 | GAG |
| Exon51-#2 | 3′ | CUCAGACUGUUACUCUGGUG | 780 | TAG |
| Exon51-#3 | 3′ | UUUUGCAAAAACCCAAAAUA | 781 | AGG |
| Exon51-#4 | 3′ | AAAAUAUUUUAGCUCCUACU | 782 | GGG |
| Exon51-#5 | 3′ | AAAUAUUUUAGCUCCUACUC | 783 | TGG |
| Exon51-#6 | 3′ | ACUCAGACUGUUACUCUGGU | 784 | AGG |
| Exon51-#7 | 5′ | GAGUAACAGUCUGAGUAGGA | 785 | TGG |
| Exon51-#8 | 5′ | CAGGUUGUGUCACCAGAGUA | 786 | TGG |
| Exon51-#9 | 3′ | GUGGUUACUAAGGAAACUGC | 787 | AGG |
| Exon51-#10 | 5′ | UAGUAACCACAGGUUGUGUC | 788 | AGG |
| Exon51-#11 | 3′ | AGACUGUUACUCUGGUGACA | 789 | GAG |
| Exon51-#12 | 3′ | UUACUCUGGUGACACAACCU | 790 | CAG |
| Exon51-#13 | 3′ | UGGUGACACAACCUGUGGUU | 791 | GAG |
| Exon51-#14 | 3′ | GUGACACAACCUGUGGUUAC | 792 | CAG |
| Exon53-#1 | 5′ | GAAUAAAAGGAAAAAUAAAU | 793 | TAG |
| Exon53-#2 | 5′ | UCAACUAGAAUAAAAGGAAA | 794 | AAG |
| Exon53-#3 | 3′ | GUUGAAAGAAUUCAGAAUCA | 795 | TAG |
| Exon53-#4 | 3′ | UUAUUCUAGUUGAAAGAAUU | 796 | AAG |
| Exon53-#6 | 5′ | AAUUCUUUCAACUAGAAUAA | 797 | CAG |
| Exon53-#7 | 5′ | AUUCUGAAUUCUUUCAACUA | 798 | CAG |
| Exon53-#8 | 5′ | CAUCCCACUGAUUCUGAAUU | 799 | AAG |
| Exon53-#9 | 3′ | UUUAUUCUAGUUGAAAGAAU | 800 | AGG |
| Exon53-#10 | 5′ | CUGAUUCUGAAUUCUUUCAA | 801 | TGG |
| Exon53-#11 | 5′ | ACUGAUUCUGAAUUCUUUCA | 802 | GGG |
| Exon53-#12 | 3′ | UCAGAACCGGAGGCAACAGU | 803 | AGG |
| Exon44-#1 | 3′ | CAGGCGAUUUGACAGAUCUG | 804 | CAG |
| Exon44-#2 | 3′ | UUGAGAAAUGGCGGCGUUUU | 805 | CAG |
| Exon44-#3 | 3′ | GCAGGCGAUUUGACAGAUCU | 806 | AGG |
| Exon44-#4 | 3′ | UCUAUUUUUCUUGAUCCAUA | 807 | TGG |
| Exon44-#5 | 3′ | UAUGCUUUUACCUGCAGGCG | 808 | TGG |
| Exon44-#6 | 5′ | GCAGGUAAAAGCAUAUGGAU | 809 | AGG |
| Exon44-#8 | 5′ | UUUCUCAACAGAUCUGUCAA | 810 | TGG |
| Exon44-#9 | 5′ | CCAUUUCUCAACAGAUCUGU | 811 | CGG |
| Exon44-#11 | 5′ | AACAGAUCUGUCAAAUCGCC | 812 | GAG |
| Exon44-#13 | 5′ | UAUAUCAUAAUGAAAACGCC | 813 | AAG |
| Exon44-#14 | 5′ | AUUAAAUAUCUUUAUAUCAU | 814 | CAG |
| Exon44-#15 | 5′ | UUAGCCACUGAUUAAAUAUC | 815 | CAG |
| Exon44-#16 | 5′ | CUGUUAGCCACUGAUUAAAU | 816 | AAG |
| Exon44-#17 | 3′ | ACAGAAGCUGAACAGUUUCU | 817 | TAG |
| Exon43-#1 | 5′ | UAAUAUAAAAAUUUUAAAAC | 818 | CAG |
| Exon43-#2 | 5′ | UUAUAUUCUGUAAUAUAAAA | 819 | AAG |
| Exon43-#3 | 5′ | UCUUUUAUAUUCUGUAAUAU | 820 | TAG |
| Exon45-#1 | 3′ | UUUUGGUAUCUUACAGGAAC | 821 | AGG |
| Exon45-#2 | 5′ | GUAAGAUACCAAAAAGGCAA | 822 | AGG |
| Exon45-#3 | 5′ | GGAGUUCCUGUAAGAUACCA | 823 | AGG |
| Exon45-#4 | 5′ | UCCUGGAGUUCCUGUAAGAU | 824 | TGG |
| Exon45-#5 | 3′ | GGAUGGCAUUGGGCAGCGGC | 825 | TGG |
| Exon45-#6 | 5′ | AUGCCAUCCUGGAGUUCCUG | 826 | TGG |
| Exon45-#7 | 5′ | AAUGCCAUCCUGGAGUUCCU | 827 | GGG |
| Exon45-#8 | 5′ | CUGCCCAAUGCCAUCCUGGA | 828 | CGG |
| Exon45-#9 | 5′ | GUUGCAUUCAAUGUUCUGAC | 829 | TGG |
| Exon45-#10 | 3′ | UUUGGUAUCUUACAGGAACU | 830 | AAG |
| Exon45-#11 | 3′ | ACAGGAACUCCAGGAUGGCA | 831 | AAG |
| Exon45-#12 | 5′ | GAGUUCCUGUAAGAUACCAA | 832 | CAG |
| Exon45-#13 | 3′ | CAGGAUGGCAUUGGGCAGCG | 833 | GAG |
| Exon45-#14 | 5′ | CCCAAUGCCAUCCUGGAGUU | 834 | CAG |
| Exon45-#15 | 5′ | ACAACAGUUUGCCGCUGCCC | 835 | CAG |
| Exon52-#1 | 3′ | GCAGGCGAUUUGACAGAUCU | 836 | AGG |
| Exon52-#2 | 3′ | ACAGGCAACAAUGCAGGAUU | 837 | AAG |
| Exon52-#3 | 5′ | AGUAUCAGGGUUCUUCAGCG | 838 | AAG |
| Exon52-#4 | 3′ | AUACUAAGGGAUAUUUGUUC | 839 | CAG |
| Exon52-#5 | 3′ | AGGGAUAUUUGUUCUUACAG | 840 | TAG |
| Exon52-#6 | 5′ | UAAGAACAAAUAUCCCUUAG | 841 | CAG |
| Exon52-#8 | 5′ | CAUUGUUGCCUGUAAGAACA | 842 | CAG |
| Exon52-#9 | 5′ | UUCCAAAUCCUGCAUUGUUG | 843 | CAG |
| Exon52-#10 | 5′ | UGUUCCAAAUCCUGCAUUGU | 844 | GAG |
| Exon52-#11 | 5′ | GGGACGCCUCUGUUCCAAAU | 845 | CAG |
| Exon52-#12 | 5′ | CCAACUGGGGACGCCUCUGU | 846 | AAG |
| Exon2-#1 | 5′ | ACAGAGGCGUCCCCAGUUGG | 847 | TAG |
| Exon2-#2 | 5′ | UCAUCUAAAAUGCAAAAUAA | 848 | AAG |
| Exon2-#3 | 5′ | UUUCAUCUAAAAUGCAAAAU | 849 | GAG |
| Exon2-#4 | 5′ | CUCUUUCAUCUAAAAUGCAA | 850 | AAG |
| Exon2-#5 | 5′ | UUGAACAUCUUCUCUUUCAU | 851 | AAG |
| Exon51-#1′ | 3′ | CAGAGUAACAGUCUGAGUAG | 852 | GAG |
| Exon51-#2′ | 3′ | CACCAGAGUAACAGUCUGAG | 853 | TAG |
| Exon51-#3′ | 3′ | UAUUUUGGGUUUUUGCAAAA | 854 | AGG |
| Exon51-#4′ | 3′ | AGUAGGAGCUAAAAUAUUUU | 855 | GGG |
| Exon51-#5′ | 3′ | GAGUAGGAGCUAAAAUAUUU | 856 | TGG |
| Exon51-#6′ | 3′ | ACCAGAGUAACAGUCUGAGU | 857 | AGG |
| Exon51-#7′ | 5′ | UCCUACUCAGACUGUUACUC | 858 | TGG |
| Exon51-#8′ | 5′ | UACUCUGGUGACACAACCUG | 859 | TGG |
| Exon51-#9′ | 3′ | GCAGUUUCCUUAGUAACCAC | 860 | AGG |
| Exon51-#10′ | 5′ | GACACAACCUGUGGUUACUA | 861 | AGG |
| Exon51-#11′ | 3′ | UGUCACCAGAGUAACAGUCU | 862 | GAG |
| Exon51-#12′ | 3′ | AGGUUGUGUCACCAGAGUAA | 863 | CAG |
| Exon51-#13′ | 3′ | AACCACAGGUUGUGUCACCA | 864 | GAG |
| Exon51-#14′ | 3′ | GUAACCACAGGUUGUGUCAC | 865 | CAG |
| Exon53-#1′ | 5′ | AUUUAUUUUUCCUUUUAUUC | 866 | TAG |
| Exon53-#2′ | 5′ | UUUCCUUUUAUUCUAGUUGA | 867 | AAG |
| Exon53-#3′ | 3′ | UGAUUCUGAAUUCUUUCAAC | 868 | TAG |
| Exon53-#4′ | 3′ | AAUUCUUUCAACUAGAAUAA | 869 | AAG |
| Exon53-#6′ | 5′ | UUAUUCUAGUUGAAAGAAUU | 870 | CAG |
| Exon53-#7′ | 5′ | UAGUUGAAAGAAUUCAGAAU | 871 | CAG |
| Exon53-#8′ | 5′ | AAUUCAGAAUCAGUGGGAUG | 872 | AAG |
| Exon53-#9′ | 3′ | AUUCUUUCAACUAGAAUAAA | 873 | AGG |
| Exon53-#10′ | 5′ | UUGAAAGAAUUCAGAAUCAG | 874 | TGG |
| Exon53-#11′ | 5′ | UGAAAGAAUUCAGAAUCAGU | 875 | GGG |
| Exon53-#12′ | 3′ | ACUGUUGCCUCCGGUUCUGA | 876 | AGG |
| Exon44-#1′ | 3′ | CAGAUCUGUCAAAUCGCCUG | 877 | CAG |
| Exon44-#2′ | 3′ | AAAACGCCGCCAUUUCUCAA | 878 | CAG |
| Exon44-#3′ | 3′ | AGAUCUGUCAAAUCGCCUGC | 879 | AGG |
| Exon44-#4′ | 3′ | UAUGGAUCAAGAAAAAUAGA | 880 | TGG |
| Exon44-#5′ | 3′ | CGCCUGCAGGUAAAAGCAUA | 881 | TGG |
| Exon44-#6′ | 5′ | AUCCAUAUGCUUUUACCUGC | 882 | AGG |
| Exon44-#8′ | 5′ | UUGACAGAUCUGUUGAGAAA | 883 | TGG |
| Exon44-#9′ | 5′ | ACAGAUCUGUUGAGAAAUGG | 884 | CGG |
| Exon44-#11′ | 5′ | GGCGAUUUGACAGAUCUGUU | 885 | GAG |
| Exon44-#13′ | 5′ | GGCGUUUUCAUUAUGAUAUA | 886 | AAG |
| Exon44-#14′ | 5′ | AUGAUAUAAAGAUAUUUAAU | 887 | CAG |
| Exon44-#15′ | 5′ | GAUAUUUAAUCAGUGGCUAA | 888 | CAG |
| Exon44-#16′ | 5′ | AUUUAAUCAGUGGCUAACAG | 889 | AAG |
| Exon44-#17′ | 3′ | AGAAACUGUUCAGCUUCUGU | 890 | TAG |
| Exon43-#1′ | 5′ | GUUUUAAAAUUUUUAUAUUA | 891 | CAG |
| Exon43-#2′ | 5′ | UUUUAUAUUACAGAAUAUAA | 892 | AAG |
| Exon43-#3′ | 5′ | AUAUUACAGAAUAUAAAAGA | 893 | TAG |
| Exon45-#1′ | 3′ | GUUCCUGUAAGAUACCAAAA | 894 | AGG |
| Exon45-#2′ | 5′ | UUGCCUUUUUGGUAUCUUAC | 895 | AGG |
| Exon45-#3′ | 5′ | UGGUAUCUUACAGGAACUCC | 896 | AGG |
| Exon45-#4′ | 5′ | AUCUUACAGGAACUCCAGGA | 897 | TGG |
| Exon45-#5′ | 3′ | GCCGCUGCCCAAUGCCAUCC | 898 | TGG |
| Exon45-#6′ | 5′ | CAGGAACUCCAGGAUGGCAU | 899 | TGG |
| Exon45-#7 | 5′ | AGGAACUCCAGGAUGGCAUU | 900 | GGG |
| Exon45-#8′ | 5′ | UCCAGGAUGGCAUUGGGCAG | 901 | CGG |
| Exon45-#9′ | 5′ | GUCAGAACAUUGAAUGCAAC | 902 | TGG |
| Exon45-#10′ | 3′ | AGUUCCUGUAAGAUACCAAA | 903 | AAG |
| Exon45-#11′ | 3′ | UGCCAUCCUGGAGUUCCUGU | 904 | AAG |
| Exon45-#12′ | 5′ | UUGGUAUCUUACAGGAACUC | 905 | CAG |
| Exon45-#13′ | 3′ | CGCUGCCCAAUGCCAUCCUG | 906 | GAG |
| Exon45-#14′ | 5′ | AACUCCAGGAUGGCAUUGGG | 907 | CAG |
| Exon45-#15′ | 5′ | GGGCAGCGGCAAACUGUUGU | 908 | CAG |
| Exon52-#1′ | 3′ | AGAUCUGUCAAAUCGCCUGC | 909 | AGG |
| Exon52-#2′ | 3′ | AAUCCUGCAUUGUUGCCUGU | 910 | AAG |
| Exon52-#3′ | 5′ | CGCUGAAGAACCCUGAUACU | 911 | AAG |
| Exon52-#4′ | 3′ | GAACAAAUAUCCCUUAGUAU | 912 | CAG |
| Exon52-#5′ | 3′ | CUGUAAGAACAAAUAUCCCU | 913 | TAG |
| Exon52-#6′ | 5′ | CUAAGGGAUAUUUGUUCUUA | 914 | CAG |
| Exon52-#8′ | 5′ | UGUUCUUACAGGCAACAAUG | 915 | CAG |
| Exon52-#9′ | 5′ | CAACAAUGCAGGAUUUGGAA | 916 | CAG |
| Exon52-#10′ | 5′ | ACAAUGCAGGAUUUGGAACA | 917 | GAG |
| Exon52-#11′ | 5′ | AUUUGGAACAGAGGCGUCCC | 918 | CAG |
| Exon52-#12′ | 5′ | ACAGAGGCGUCCCCAGUUGG | 919 | AAG |
| Exon2-#1′ | 5′ | UAUUUUUUUAUUUUGCAUUU | 920 | TAG |
| Exon2-#2′ | 5′ | UUAUUUUGCAUUUUAGAUGA | 921 | AAG |
| Exon2-#3′ | 5′ | AUUUUGCAUUUUAGAUGAAA | 922 | GAG |
| Exon2-#4′ | 5′ | UUGCAUUUUAGAUGAAAGAG | 923 | AAG |
| Exon2-#5′ | 5′ | AUGAAAGAGAAGAUGUUCAA | 924 | AAG |

TABLE 11
gRNAs targeting dog DMD Exon 51

| | | | SEQ | |
|---|---|---|---|---|
| | | | ID | |
| ID sgRNA | Strand | sgRNAs | NO: | PAM |
| Ex51-SA-2 | 3′ | CACCAGAGTAACAGTCTGAC | 925 | TGG |

TABLE 12
gRNA sequence for targeting dog DMD Exon 51

| | | | SEQ | |
|---|---|---|---|---|
| | | | ID | |
| ID sgRNA | Strand | sgRNAs | NO: | PAM |
| Ex51-SA-2 | 3′ | GUCAGACUGUUACUCUGGUG | 926 | TGG |
| Ex51-SA-2′ | 3′ | CACCAGAGUAACAGUCUGAC | 927 | TGG |

TABLE 13
gRNA sequences for targeting DMD Exon 43 & 45

| sgRNA ID | Sequence (5′-3′) | SEQ ID NO. |
|---|---|---|
| Ex45-gRNA#3 | CGCTGCCCAATGCCATCCTG | 928 |
| Ex45-gRNA#4 | ATCTTACAGGAACTCCAGGA | 929 |
| Ex45-gRNA#5 | AGGAACTCCAGGATGGCATT | 930 |
| Ex45-gRNA#6 | CGCTGCCCAATGCCATCC | 931 |
| Ex43-gRNA#1 | GTTTTAAAATTTTTATATTA | 932 |
| Ex43-gRNA#2 | TTTTATATTACAGAATATAA | 933 |
| Ex43-gRNA#4 | TATGTGTTACCTACCCTTGT | 934 |
| Ex43-gRNA#6 | GTACAAGGACCGACAAGGGT | 935 |
| Ex45-gRNA#3′ | CAGGAUGGCAUUGGGCAGCG | 936 |
| Ex45-gRNA#4′ | UCCUGGAGUUCCUGUAAGAU | 937 |
| Ex45-gRNA#5′ | AAUGCCAUCCUGGAGUUCCU | 938 |
| Ex45-gRNA#6′ | GGAUGGCAUUGGGCAGCG | 939 |
| Ex43-gRNA#1′ | UAAUAUAAAAAUUUUAAAAC | 940 |
| Ex43-gRNA#2′ | UUAUAUUCUGUAAUAUAAAA | 941 |
| Ex43-gRNA#4′ | ACAAGGGUAGGUAACACAUA | 942 |
| Ex43-gRNA#6′ | ACCCUUGUCGGUCCUUGUAC | 943 |
| Ex45-gRNA#3′′ | CGCUGCCCAAUGCCAUCCUG | 944 |
| Ex45-gRNA#4′′ | AUCUUACAGGAACUCCAGGA | 945 |
| Ex45-gRNA#5′′ | AGGAACUCCAGGAUGGCAUU | 946 |
| Ex45-gRNA#6′′ | CGCUGCCCAAUGCCAUCC | 947 |
| Ex43-gRNA#1′′ | GUUUUAAAAUUUUUAUAUUA | 948 |
| Ex43-gRNA#2′′ | UUUUAUAUUACAGAAUAUAA | 949 |
| Ex43-gRNA#4′′ | UAUGUGUUACCUACCCUUGU | 950 |
| Ex43-gRNA#6′′ | GUACAAGGACCGACAAGGGU | 951 |

TABLE 14
Additional exemplary gRNA sequences for targeting DMD

| Targeted gRNA | | | | SEQ | | SEQ ID |
|---|---|---|---|---|---|---|
| Exon | Strand | PAM | DNA sequence* | ID NO. | RNA sequence* | NO. |
| Human-Exon 51 | 1 | tttt | tctttttcttcttttttccttttt | 2025 | ucuuuuucuucuuuuuuccuuuuu | 952 |
| Human-Exon 51 | 1 | tttt | ctttttcttcttttttcctttttG | 2026 | cuuuuucuucuuuuuuccuuuuuG | 953 |
| Human-Exon 51 | 1 | tttc | tttttcttcttttttcctttttGC | 2027 | uuuuucuucuuuuuuccuuuuuGC | 954 |
| Human-Exon 51 | 1 | tttt | tcttcttttttcctttttGCAAAA | 2028 | ucuucuuuuuuccuuuuuGCAAAA | 955 |
| Human-Exon 51 | 1 | tttt | cttcttttttcctttttGCAAAAA | 2029 | cuucuuuuuuccuuuuuGCAAAAA | 956 |
| Human-Exon 51 | 1 | tttc | ttcttttttcctttttGCAAAAAC | 2030 | uucuuuuuuccuuuuuGCAAAAAC | 957 |
| Human-Exon 51 | 1 | tttt | ttcctttttGCAAAAACCCAAAAT | 2031 | uuccuuuuuGCAAAAACCCAAAAU | 958 |
| Human-Exon 51 | 1 | tttt | tcctttttGCAAAAACCCAAAATA | 2032 | uccuuuuuGCAAAAACCCAAAAUA | 959 |
| Human-Exon 51 | 1 | tttt | cctttttGCAAAAACCCAAAATAT | 2033 | ccuuuuuGCAAAAACCCAAAAUAU | 960 |
| Human-Exon 51 | 1 | tttc | ctttttGCAAAAACCCAAAATATT | 2034 | cuuuuuGCAAAAACCCAAAAUAUU | 961 |
| Human-Exon 51 | 1 | tttt | tGCAAAAACCCAAAATATTTTAGC | 2035 | uGCAAAAACCCAAAAUAUUUUAGC | 962 |
| Human-Exon 51 | 1 | tttt | GCAAAAACCCAAAATATTTTAGCT | 2036 | GCAAAAACCCAAAAUAUUUUAGCU | 963 |
| Human-Exon 51 | 1 | tttG | CAAAAACCCAAAATATTTTAGCTC | 2037 | CAAAAACCCAAAAUAUUUUAGCUC | 964 |
| Human-Exon 51 | 1 | TTTT | AGCTCCTACTCAGACTGTTACTCT | 2038 | AGCUCCUACUCAGACUGUUACUCU | 965 |
| Human-Exon 51 | 1 | TTTA | GCTCCTACTCAGACTGTTACTCTG | 2039 | GCUCCUACUCAGACUGUUACUCUG | 966 |
| Human-Exon 51 | −1 | TTTC | CTTAGTAACCACAGGTTGTGTCAC | 2040 | CUUAGUAACCACAGGUUGUGUCAC | 967 |
| Human-Exon 51 | −1 | TTTG | GAGATGGCAGTTTCCTTAGTAACC | 2041 | GAGAUGGCAGUUUCCUUAGUAACC | 968 |
| Human-Exon 51 | −1 | TTTC | TAGTTTGGAGATGGCAGTTTCCTT | 2042 | UAGUUUGGAGAUGGCAGUUUCCUU | 969 |
| Human-Exon 51 | −1 | TTTT | TTCTCATACCTTCTGCTTGATGAT | 2043 | UUCUCAUACCUUCUGCUUGAUGAU | 970 |
| Human-Exon 51 | −1 | TTTA | TCATTTTTTCTCATACCTTCTGCT | 2044 | UCAUUUUUUCUCAUACCUUCUGCU | 971 |
| Human-Exon 51 | −1 | TTTT | ATCATTTTTTCTCATACCTTCTGC | 2045 | AUCAUUUUUUCUCAUACCUUCUGC | 972 |
| Human-Exon 51 | −1 | TTTA | AAGAAAAACTTCTGCCAACTTTTA | 2046 | AAGAAAAACUUCUGCCAACUUUUA | 973 |
| Human-Exon 51 | −1 | TTTT | AAAGAAAAACTTCTGCCAACTTTT | 2047 | AAAGAAAAACUUCUGCCAACUUUU | 974 |
| Human-Exon 51 | 1 | TTTT | TCTTTAAAATGAAGATTTTCCACC | 2048 | UCUUUAAAAUGAAGAUUUUCCACC | 975 |
| Human-Exon 51 | 1 | TTTT | CTTTAAAATGAAGATTTTCCACCA | 2049 | CUUUAAAAUGAAGAUUUUCCACCA | 976 |
| Human-Exon 51 | 1 | TTTC | TTTAAAATGAAGATTTTCCACCAA | 2050 | UUUAAAAUGAAGAUUUUCCACCAA | 977 |
| Human-Exon 51 | 1 | TTTA | AAATGAAGATTTTCCACCAATCAC | 2051 | AAAUGAAGAUUUUCCACCAAUCAC | 978 |
| Human-Exon 51 | 1 | TTTT | CCACCAATCACTTTACTCTCCTAG | 2052 | CCACCAAUCACUUUACUCUCCUAG | 979 |
| Human-Exon 51 | 1 | TTTC | CACCAATCACTTTACTCTCCTAGA | 2053 | CACCAAUCACUUUACUCUCCUAGA | 980 |
| Human-Exon 51 | 1 | TTTA | CTCTCCTAGACCATTTCCCACCAG | 2054 | CUCUCCUAGACCAUUUCCCACCAG | 981 |
| Human-Exon 45 | −1 | tttg | agaaaagattaaacagtgtgctac | 2055 | agaaaagauuaaacagugugcuac | 982 |
| Human-Exon 45 | −1 | TTTa | tttgagaaaagattaaacagtgtg | 2056 | uuugagaaaagauuaaacagugug | 983 |
| Human-Exon 45 | −1 | TTTT | atttgagaaaagattaaacagtgt | 2057 | auuugagaaaagauuaaacagugu | 984 |
| Human-Exon 45 | −1 | TTTT | Tatttgagaaaagattaaacagtg | 2058 | Uauuugagaaaagauuaaacagug | 985 |
| Human-Exon 45 | 1 | ttta | atottttctcaaatAAAAAGACAT | 2059 | aucuuuucucaaauAAAAAGACAU | 986 |
| Human-Exon 45 | 1 | tttt | ctcaaatAAAAAGACATGGGGCTT | 2060 | cucaaauAAAAAGACAUGGGGCUU | 987 |
| Human-Exon 45 | 1 | tttc | tcaaatAAAAAGACATGGGGCTTC | 2061 | ucaaauAAAAAGACAUGGGGCUUC | 988 |
| Human-Exon 45 | 1 | TTTT | TGTTTTGCCTTTTTGGTATCTTAC | 2062 | UGUUUUGCCUUUUUGGUAUCUUAC | 989 |
| Human-Exon 45 | 1 | TTTT | GTTTTGCCTTTTTGGTATCTTACA | 2063 | GUUUUGCCUUUUUGGUAUCUUACA | 990 |
| Human-Exon 45 | 1 | TTTG | TTTTGCCTTTTTGGTATCTTACAG | 2064 | UUUUGCCUUUUUGGUAUCUUACAG | 991 |
| Human-Exon 45 | 1 | TTTT | GCCTTTTTGGTATCTTACAGGAAC | 2065 | GCCUUUUUGGUAUCUUACAGGAAC | 992 |
| Human-Exon 45 | 1 | TTTG | CCTTTTTGGTATCTTACAGGAACT | 2066 | CCUUUUUGGUAUCUUACAGGAACU | 993 |
| Human-Exon 45 | 1 | TTTT | TGGTATCTTACAGGAACTCCAGGA | 2067 | UGGUAUCUUACAGGAACUCCAGGA | 994 |
| Human-Exon 45 | 1 | TTTT | GGTATCTTACAGGAACTCCAGGAT | 2068 | GGUAUCUUACAGGAACUCCAGGAU | 995 |
| Human-Exon 45 | −1 | TTTG | AGGATTGCTGAATTATTTCTTCCC | 2069 | AGGAUUGCUGAAUUAUUUCUUCCC | 996 |
| Human-Exon 45 | −1 | TTTT | GAGGATTGCTGAATTATTTCTTCC | 2070 | GAGGAUUGCUGAAUUAUUUCUUCC | 997 |
| Human-Exon 45 | −1 | TTTT | TGAGGATTGCTGAATTATTTCTTC | 2071 | UGAGGAUUGCUGAAUUAUUUCUUC | 998 |
| Human-Exon 45 | −1 | TTTC | CTGTAGAATACTGGCATCTGTTTT | 2072 | CUGUAGAAUACUGGCAUCUGUUUU | 999 |
| Human-Exon 45 | −1 | TTTT | CCTGTAGAATACTGGCATCTGTTT | 2073 | CCUGUAGAAUACUGGCAUCUGUUU | 1000 |
| Human-Exon 45 | −1 | TTTT | TCCTGTAGAATACTGGCATCTGTT | 2074 | UCCUGUAGAAUACUGGCAUCUGUU | 1001 |
| Human-Exon 45 | −1 | TTTG | CAGACCTCCTGCCACCGCAGATTC | 2075 | CAGACCUCCUGCCACCGCAGAUUC | 1002 |
| Human-Exon 45 | −1 | TTTC | TGTCTGACAGCTGTTTGCAGACCT | 2076 | UGUCUGACAGCUGUUUGCAGACCU | 1003 |
| Human-Exon 45 | −1 | TTTT | CTGTCTGACAGCTGTTTGCAGACC | 2077 | CUGUCUGACAGCUGUUUGCAGACC | 1004 |
| Human-Exon 45 | −1 | TTTT | TCTGTCTGACAGCTGTTTGCAGAC | 2078 | UCUGUCUGACAGCUGUUUGCAGAC | 1005 |
| Human-Exon 45 | −1 | TTTT | TTCTGTCTGACAGCTGTTTGCAGA | 2079 | UUCUGUCUGACAGCUGUUUGCAGA | 1006 |
| Human-Exon 45 | −1 | TTTC | ATTCCTATTAGATCTGTCGCCCTA | 2080 | AUUCCUAUUAGAUCUGUCGCCCUA | 1007 |
| Human-Exon 45 | −1 | TTTT | CATTCCTATTAGATCTGTCGCCCT | 2081 | CAUUCCUAUUAGAUCUGUCGCCCU | 1008 |
| Human-Exon 45 | 1 | TTTT | AGCAGACTTTTTAAGCTTTCTTTA | 2082 | AGCAGACUUUUUAAGCUUUCUUUA | 1009 |
| Human-Exon 45 | 1 | TTTA | GCAGACTTTTTAAGCTTTCTTTAG | 2083 | GCAGACUUUUUAAGCUUUCUUUAG | 1010 |
| Human-Exon 45 | 1 | TTTT | TAAGCTTTCTTTAGAAGAATATTT | 2084 | UAAGCUUUCUUUAGAAGAAUAUUU | 1011 |
| Human-Exon 45 | 1 | TTTT | AAGCTTTCTTTAGAAGAATATTTC | 2085 | AAGCUUUCUUUAGAAGAAUAUUUC | 1012 |
| Human-Exon 45 | 1 | TTTA | AGCTTTCTTTAGAAGAATATTTCA | 2086 | AGCUUUCUUUAGAAGAAUAUUUCA | 1013 |
| Human-Exon 45 | 1 | TTTC | TTTAGAAGAATATTTCATGAGAGA | 2087 | UUUAGAAGAAUAUUUCAUGAGAGA | 1014 |
| Human-Exon 45 | 1 | TTTA | GAAGAATATTTCATGAGAGATTAT | 2088 | GAAGAAUAUUUCAUGAGAGAUUAU | 1015 |
| Human-Exon 44 | 1 | TTTG | TCAGTATAACCAAAAAATATACGC | 2089 | UCAGUAUAACCAAAAAAUAUACGC | 1016 |
| Human-Exon 44 | 1 | tttt | acataatccatctatttttcttga | 2090 | acauaauccaucuauuuuucuuga | 1017 |
| Human-Exon 44 | 1 | ttta | cataatccatctatttttcttgat | 2091 | cauaauccaucuauuuuucuugau | 1018 |
| Human-Exon 44 | 1 | tttt | tcttgatccatatgcttttACCTG | 2092 | ucuugauccauaugcuuuuACCUG | 1019 |
| Human-Exon 44 | 1 | tttt | cttgatccatatgcttttACCTGC | 2093 | cuugauccauaugcuuuuACCUGC | 1020 |
| Human-Exon 44 | 1 | tttc | ttgatccatatgcttttACCTGCA | 2094 | uugauccauaugcuuuuACCUGCA | 1021 |
| Human-Exon 44 | −1 | TTTC | TCAACAGATCTGTCAAATCGCCTG | 2095 | UCAACAGAUCUGUCAAAUCGCCUG | 1022 |
| Human-Exon 44 | 1 | tttt | ACCTGCAGGCGATTTGACAGATCT | 2096 | ACCUGCAGGCGAUUUGACAGAUCU | 1023 |
| Human-Exon 44 | 1 | tttA | CCTGCAGGCGATTTGACAGATCTG | 2097 | CCUGCAGGCGAUUUGACAGAUCUG | 1024 |
| Human-Exon 44 | 1 | TTTG | ACAGATCTGTTGAGAAATGGCGGC | 2098 | ACAGAUCUGUUGAGAAAUGGCGGC | 1025 |
| Human-Exon 44 | −1 | TTTA | TATCATAATGAAAACGCCGCCATT | 2099 | UAUCAUAAUGAAAACGCCGCCAUU | 1026 |
| Human-Exon 44 | 1 | TTTT | CATTATGATATAAAGATATTTAAT | 2100 | CAUUAUGAUAUAAAGAUAUUUAAU | 1027 |
| Human-Exon 44 | −1 | TTTG | TATTTAGCATGTTCCCAATTCTCA | 2101 | UAUUUAGCAUGUUCCCAAUUCUCA | 1028 |
| Human-Exon 44 | −1 | TTTC | GAAAAAACAAATCAAAGACTTACC | 2102 | GAAAAAACAAAUCAAAGACUUACC | 1029 |
| Human-Exon 44 | 1 | TTTG | ATTTGTTTTTTCGAAATTGTATTT | 2103 | AUUUGUUUUUUCGAAAUUGUAUUU | 1030 |
| Human-Exon 44 | 1 | TTTG | TTTTTTCGAAATTGTATTTATCTT | 2104 | UUUUUUCGAAAUUGUAUUUAUCUU | 1031 |
| Human-Exon 44 | 1 | TTTT | TTCGAAATTGTATTTATCTTCAGC | 2105 | UUCGAAAUUGUAUUUAUCUUCAGC | 1032 |
| Human-Exon 44 | 1 | TTTT | TCGAAATTGTATTTATCTTCAGCA | 2106 | UCGAAAUUGUAUUUAUCUUCAGCA | 1033 |
| Human-Exon 44 | 1 | TTTT | CGAAATTGTATTTATCTTCAGCAC | 2107 | CGAAAUUGUAUUUAUCUUCAGCAC | 1034 |
| Human-Exon 44 | 1 | TTTC | GAAATTGTATTTATCTTCAGCACA | 2108 | GAAAUUGUAUUUAUCUUCAGCACA | 1035 |
| Human-Exon 44 | −1 | TTTA | AGAAGTTAAAGAGTCCAGATGTGC | 2109 | AGAAGUUAAAGAGUCCAGAUGUGC | 1036 |
| Human-Exon 44 | 1 | TTTA | TCTTCAGCACATCTGGACTCTTTA | 2110 | UCUUCAGCACAUCUGGACUCUUUA | 1037 |
| Human-Exon 44 | −1 | TTTC | CATCACCCTTCAGAACCTGATCTT | 2111 | CAUCACCCUUCAGAACCUGAUCUU | 1038 |
| Human-Exon 44 | 1 | TTTA | ACTTCTTAAAGATCAGGTTCTGAA | 2112 | ACUUCUUAAAGAUCAGGUUCUGAA | 1039 |
| Human-Exon 44 | 1 | TTTT | GACTGTTGTTGTCATCATTATATT | 2113 | GACUGUUGUUGUCAUCAUUAUAUU | 1040 |
| Human-Exon 44 | 1 | TTTG | ACTGTTGTTGTCATCATTATATTA | 2114 | ACUGUUGUUGUCAUCAUUAUAUUA | 1041 |
| Human-Exon 53 | −1 | TTTC | AACTAGAATAAAAGGAAAAATAAA | 2115 | AACUAGAAUAAAAGGAAAAAUAAA | 1042 |
| Human-Exon 53 | 1 | TTTA | CTACTATATATTTATTTTTCCTTT | 2116 | CUACUAUAUAUUUAUUUUUCCUUU | 1043 |
| Human-Exon 53 | 1 | TTTA | TTTTTCCTTTTATTCTAGTTGAAA | 2117 | UUUUUCCUUUUAUUCUAGUUGAAA | 1044 |
| Human-Exon 53 | 1 | TTTT | TCCTTTTATTCTAGTTGAAAGAAT | 2118 | UCCUUUUAUUCUAGUUGAAAGAAU | 1045 |
| Human-Exon 53 | 1 | TTTT | CCTTTTATTCTAGTTGAAAGAATT | 2119 | CCUUUUAUUCUAGUUGAAAGAAUU | 1046 |
| Human-Exon 53 | 1 | TTTC | CTTTTATTCTAGTTGAAAGAATTC | 2120 | CUUUUAUUCUAGUUGAAAGAAUUC | 1047 |
| Human-Exon 53 | 1 | TTTT | ATTCTAGTTGAAAGAATTCAGAAT | 2121 | AUUCUAGUUGAAAGAAUUCAGAAU | 1048 |
| Human-Exon 53 | 1 | TTTA | TTCTAGTTGAAAGAATTCAGAATC | 2122 | UUCUAGUUGAAAGAAUUCAGAAUC | 1049 |
| Human-Exon 53 | −1 | TTTC | ATTCAACTGTTGCCTCCGGTTCTG | 2123 | AUUCAACUGUUGCCUCCGGUUCUG | 1050 |
| Human-Exon 53 | −1 | TTTA | ACATTTCATTCAACTGTTGCCTCC | 2124 | ACAUUUCAUUCAACUGUUGCCUCC | 1051 |
| Human-Exon 53 | −1 | TTTT | CTTTTGGATTGCATCTACTGTATA | 2125 | CUUUUGGAUUGCAUCUACUGUAUA | 1052 |
| Human-Exon 53 | −1 | TTTC | TGTGATTTTCTTTTGGATTGCATC | 2126 | UGUGAUUUUCUUUUGGAUUGCAUC | 1053 |
| Human-Exon 53 | −1 | TTTG | ATACTAACCTTGGTTTCTGTGATT | 2127 | AUACUAACCUUGGUUUCUGUGAUU | 1054 |
| Human-Exon 53 | −1 | TTTA | AAAAGGTATCTTTGATACTAACCT | 2128 | AAAAGGUAUCUUUGAUACUAACCU | 1055 |
| Human-Exon 53 | −1 | TTTT | AAAAAGGTATCTTTGATACTAACC | 2129 | AAAAAGGUAUCUUUGAUACUAACC | 1056 |
| Human-Exon 53 | −1 | TTTA | TTTTAAAAAGGTATCTTTGATACT | 2130 | UUUUAAAAAGGUAUCUUUGAUACU | 1057 |
| Human-Exon 53 | −1 | TTTT | ATTTTAAAAAGGTATCTTTGATAC | 2131 | AUUUUAAAAAGGUAUCUUUGAUAC | 1058 |
| Human-Exon 46 | −1 | TTTG | TTAATGCAAACTGGGACACAAACA | 2132 | UUAAUGCAAACUGGGACACAAACA | 1059 |
| Human-Exon 46 | 1 | TTTT | TAAATTGCCATGTTTGTGTCCCAG | 2133 | UAAAUUGCCAUGUUUGUGUCCCAG | 1060 |
| Human-Exon 46 | 1 | TTTT | AAATTGCCATGTTTGTGTCCCAGT | 2134 | AAAUUGCCAUGUUUGUGUCCCAGU | 1061 |
| Human-Exon 46 | 1 | TTTA | AATTGCCATGTTTGTGTCCCAGTT | 2135 | AAUUGCCAUGUUUGUGUCCCAGUU | 1062 |
| Human-Exon 46 | 1 | TTTG | TGTCCCAGTTTGCATTAACAAATA | 2136 | UGUCCCAGUUUGCAUUAACAAAUA | 1063 |
| Human-Exon 46 | −1 | tttC | CAACATAGTTCTCAAACTATTTGT | 2137 | CAACAUAGUUCUCAAACUAUUUGU | 1064 |
| Human-Exon 46 | −1 | tttt | CCAACATAGTTCTCAAACTATTTG | 2138 | CCAACAUAGUUCUCAAACUAUUUG | 1065 |
| Human-Exon 46 | −1 | tttt | tCCAACATAGTTCTCAAACTATTT | 2139 | uCCAACAUAGUUCUCAAACUAUUU | 1066 |
| Human-Exon 46 | −1 | tttt | tttCCAACATAGTTCTCAAACTAT | 2140 | uuuCCAACAUAGUUCUCAAACUAU | 1067 |
| Human-Exon 46 | −1 | tttt | ttttCCAACATAGTTCTCAAACTA | 2141 | uuuuCCAACAUAGUUCUCAAACUA | 1068 |
| Human-Exon 46 | −1 | tttt | tttttCCAACATAGTTCTCAAACT | 2142 | uuuuuCCAACAUAGUUCUCAAACU | 1069 |
| Human-Exon 46 | 1 | TTTG | CATTAACAAATAGTTTGAGAACTA | 2143 | CAUUAACAAAUAGUUUGAGAACUA | 1070 |
| Human-Exon 46 | 1 | TTTG | AGAACTATGTTGGaaaaaaaaaTA | 2144 | AGAACUAUGUUGGaaaaaaaaaUA | 1071 |
| Human-Exon 46 | −1 | TTTT | GTTCTTCTAGCCTGGAGAAAGAAG | 2145 | GUUCUUCUAGCCUGGAGAAAGAAG | 1072 |
| Human-Exon 46 | 1 | TTTT | ATTCTTCTTTCTCCAGGCTAGAAG | 2146 | AUUCUUCUUUCUCCAGGCUAGAAG | 1073 |
| Human-Exon 46 | 1 | TTTA | TTCTTCTTTCTCCAGGCTAGAAGA | 2147 | UUCUUCUUUCUCCAGGCUAGAAGA | 1074 |
| Human-Exon 46 | 1 | TTTC | TCCAGGCTAGAAGAACAAAAGAAT | 2148 | UCCAGGCUAGAAGAACAAAAGAAU | 1075 |
| Human-Exon 46 | −1 | TTTG | AAATTCTGACAAGATATTCTTTTG | 2149 | AAAUUCUGACAAGAUAUUCUUUUG | 1076 |
| Human-Exon 46 | −1 | TTTT | CTTTTAGTTGCTGCTCTTTTCCAG | 2150 | CUUUUAGUUGCUGCUCUUUUCCAG | 1077 |
| Human-Exon 46 | −1 | TTTG | AGAAAATAAAATTACCTTGACTTG | 2151 | AGAAAAUAAAAUUACCUUGACUUG | 1078 |
| Human-Exon 46 | −1 | TTTA | TGCAAGCAGGCCCTGGGGGATTTG | 2152 | UGCAAGCAGGCCCUGGGGGAUUUG | 1079 |
| Human-Exon 46 | 1 | TTTT | ATTTTCTCAAATCCCCCAGGGCCT | 2153 | AUUUUCUCAAAUCCCCCAGGGCCU | 1080 |
| Human-Exon 46 | 1 | TTTA | TTTTCTCAAATCCCCCAGGGCCTG | 2154 | UUUUCUCAAAUCCCCCAGGGCCUG | 1081 |
| Human-Exon 46 | 1 | TTTT | CTCAAATCCCCCAGGGCCTGCTTG | 2155 | CUCAAAUCCCCCAGGGCCUGCUUG | 1082 |
| Human-Exon 46 | 1 | TTTC | TCAAATCCCCCAGGGCCTGCTTGC | 2156 | UCAAAUCCCCCAGGGCCUGCUUGC | 1083 |
| Human-Exon 46 | 1 | TTTT | TTAATTCAATCATTGGTTTTCTGC | 2157 | UUAAUUCAAUCAUUGGUUUUCUGC | 1084 |
| Human-Exon 46 | 1 | TTTT | TAATTCAATCATTGGTTTTCTGCC | 2158 | UAAUUCAAUCAUUGGUUUUCUGCC | 1085 |
| Human-Exon 46 | 1 | TTTT | AATTCAATCATTGGTTTTCTGCCC | 2159 | AAUUCAAUCAUUGGUUUUCUGCCC | 1086 |
| Human-Exon 46 | 1 | TTTA | ATTCAATCATTGGTTTTCTGCCCA | 2160 | AUUCAAUCAUUGGUUUUCUGCCCA | 1087 |
| Human-Exon 46 | −1 | TTTA | GCAAGGAACTATGAATAACCTAAT | 2161 | GCAAGGAACUAUGAAUAACCUAAU | 1088 |
| Human-Exon 46 | 1 | TTTT | CTGCCCATTAGGTTATTCATAGTT | 2162 | CUGCCCAUUAGGUUAUUCAUAGUU | 1089 |
| Human-Exon 46 | 1 | TTTC | TGCCCATTAGGTTATTCATAGTTC | 2163 | UGCCCAUUAGGUUAUUCAUAGUUC | 1090 |
| Human-Exon 52 | −1 | TTTA | TAGAAAACAATTTAACAGGAAATA | 2164 | UAGAAAACAAUUUAACAGGAAAUA | 1091 |
| Human-Exon 52 | 1 | TTTC | CTGTTAAATTGTTTTCTATAAACC | 2165 | CUGUUAAAUUGUUUUCUAUAAACC | 1092 |
| Human-Exon 52 | −1 | TTTA | GAAATAAAAAAGATGTTACTGTAT | 2166 | GAAAUAAAAAAGAUGUUACUGUAU | 1093 |
| Human-Exon 52 | −1 | TTTT | AGAAATAAAAAAGATGTTACTGTA | 2167 | AGAAAUAAAAAAGAUGUUACUGUA | 1094 |
| Human-Exon 52 | 1 | TTTT | CTATAAACCCTTATACAGTAACAT | 2168 | CUAUAAACCCUUAUACAGUAACAU | 1095 |
| Human-Exon 52 | 1 | TTTC | TATAAACCCTTATACAGTAACATC | 2169 | UAUAAACCCUUAUACAGUAACAUC | 1096 |
| Human-Exon 52 | 1 | TTTT | TTATTTCTAAAAGTGTTTTGGCTG | 2170 | UUAUUUCUAAAAGUGUUUUGGCUG | 1097 |
| Human-Exon 52 | 1 | TTTT | TATTTCTAAAAGTGTTTTGGCTGG | 2171 | UAUUUCUAAAAGUGUUUUGGCUGG | 1098 |
| Human-Exon 52 | 1 | TTTT | ATTTCTAAAAGTGTTTTGGCTGGT | 2172 | AUUUCUAAAAGUGUUUUGGCUGGU | 1099 |
| Human-Exon 52 | 1 | TTTA | TTTCTAAAAGTGTTTTGGCTGGTC | 2173 | UUUCUAAAAGUGUUUUGGCUGGUC | 1100 |
| Human-Exon 52 | 1 | TTTC | TAAAAGTGTTTTGGCTGGTCTCAC | 2174 | UAAAAGUGUUUUGGCUGGUCUCAC | 1101 |
| Human-Exon 52 | −1 | TTTA | CATAATACAAAGTAAAGTACAATT | 2175 | CAUAAUACAAAGUAAAGUACAAUU | 1102 |
| Human-Exon 52 | −1 | TTTT | ACATAATACAAAGTAAAGTACAAT | 2176 | ACAUAAUACAAAGUAAAGUACAAU | 1103 |
| Human-Exon 52 | 1 | TTTT | GGCTGGTCTCACAATTGTACTTTA | 2177 | GGCUGGUCUCACAAUUGUACUUUA | 1104 |
| Human-Exon 52 | 1 | TTTG | GCTGGTCTCACAATTGTACTTTAC | 2178 | GCUGGUCUCACAAUUGUACUUUAC | 1105 |
| Human-Exon 52 | 1 | TTTA | CTTTGTATTATGTAAAAGGAATAC | 2179 | CUUUGUAUUAUGUAAAAGGAAUAC | 1106 |
| Human-Exon 52 | 1 | TTTG | TATTATGTAAAAGGAATACACAAC | 2180 | UAUUAUGUAAAAGGAAUACACAAC | 1107 |
| Human-Exon 52 | 1 | TTTG | TTCTTACAGGCAACAATGCAGGAT | 2181 | UUCUUACAGGCAACAAUGCAGGAU | 1108 |
| Human-Exon 52 | 1 | TTTG | GAACAGAGGCGTCCCCAGTTGGAA | 2182 | GAACAGAGGCGUCCCCAGUUGGAA | 1109 |
| Human-Exon 52 | −1 | TTTG | GGCAGCGGTAATGAGTTCTTCCAA | 2183 | GGCAGCGGUAAUGAGUUCUUCCAA | 1110 |
| Human-Exon 52 | −1 | TTTT | TCAAATTTTGGGCAGCGGTAATGA | 2184 | UCAAAUUUUGGGCAGCGGUAAUGA | 1111 |
| Human-Exon 52 | 1 | TTTG | AAAAACAAGACCAGCAATCAAGAG | 2185 | AAAAACAAGACCAGCAAUCAAGAG | 1112 |
| Human-Exon 52 | −1 | TTTG | TGTGTCCCATGCTTGTTAAAAAAC | 2186 | UGUGUCCCAUGCUUGUUAAAAAAC | 1113 |
| Human-Exon 52 | 1 | TTTT | TTAACAAGCATGGGACACACAAAG | 2187 | UUAACAAGCAUGGGACACACAAAG | 1114 |
| Human-Exon 52 | 1 | TTTT | TAACAAGCATGGGACACACAAAGC | 2188 | UAACAAGCAUGGGACACACAAAGC | 1115 |
| Human-Exon 52 | 1 | TTTT | AACAAGCATGGGACACACAAAGCA | 2189 | AACAAGCAUGGGACACACAAAGCA | 1116 |
| Human-Exon 52 | 1 | TTTA | ACAAGCATGGGACACACAAAGCAA | 2190 | ACAAGCAUGGGACACACAAAGCAA | 1117 |
| Human-Exon 52 | −1 | TTTA | TTGAAACTTGTCATGCATCTTGCT | 2191 | UUGAAACUUGUCAUGCAUCUUGCU | 1118 |
| Human-Exon 52 | −1 | TTTT | ATTGAAACTTGTCATGCATCTTGC | 2192 | AUUGAAACUUGUCAUGCAUCUUGC | 1119 |
| Human-Exon 52 | −1 | TTTT | TATTGAAACTTGTCATGCATCTTG | 2193 | UAUUGAAACUUGUCAUGCAUCUUG | 1120 |
| Human-Exon 52 | 1 | TTTC | AATAAAAACTTAAGTTCATATATC | 2194 | AAUAAAAACUUAAGUUCAUAUAUC | 1121 |
| Human-Exon 50 | −1 | TTTG | GTGAATATATTATTGGATTTCTAT | 2195 | GUGAAUAUAUUAUUGGAUUUCUAU | 1122 |
| Human-Exon 50 | −1 | TTTG | AAGATAATTCATGAACATCTTAAT | 2196 | AAGAUAAUUCAUGAACAUCUUAAU | 1123 |
| Human-Exon 50 | −1 | TTTA | ACAGAAAAGCATACACATTACTTA | 2197 | ACAGAAAAGCAUACACAUUACUUA | 1124 |
| Human-Exon 50 | 1 | TTTT | CTGTTAAAGAGGAAGTTAGAAGAT | 2198 | CUGUUAAAGAGGAAGUUAGAAGAU | 1125 |
| Human-Exon 50 | 1 | TTTC | TGTTAAAGAGGAAGTTAGAAGATC | 2199 | UGUUAAAGAGGAAGUUAGAAGAUC | 1126 |
| Human-Exon 50 | −1 | TTTA | CCGCCTTCCACTCAGAGCTCAGAT | 2200 | CCGCCUUCCACUCAGAGCUCAGAU | 1127 |
| Human-Exon 50 | −1 | TTTG | CCCTCAGCTCTTGAAGTAAACGGT | 2201 | CCCUCAGCUCUUGAAGUAAACGGU | 1128 |
| Human-Exon 50 | 1 | TTTA | CTTCAAGAGCTGAGGGCAAAGCAG | 2202 | CUUCAAGAGCUGAGGGCAAAGCAG | 1129 |
| Human-Exon 50 | −1 | TTTG | AACAAATAGCTAGAGCCAAAGAGA | 2203 | AACAAAUAGCUAGAGCCAAAGAGA | 1130 |
| Human-Exon 50 | −1 | TTTT | GAACAAATAGCTAGAGCCAAAGAG | 2204 | GAACAAAUAGCUAGAGCCAAAGAG | 1131 |
| Human-Exon 50 | 1 | TTTG | GCTCTAGCTATTTGTTCAAAAGTG | 2205 | GCUCUAGCUAUUUGUUCAAAAGUG | 1132 |
| Human-Exon 50 | 1 | TTTG | TTCAAAAGTGCAACTATGAAGTGA | 2206 | UUCAAAAGUGCAACUAUGAAGUGA | 1133 |
| Human-Exon 50 | −1 | TTTC | TCTCTCACCCAGTCATCACTTCAT | 2207 | UCUCUCACCCAGUCAUCACUUCAU | 1134 |
| Human-Exon 50 | −1 | TTTT | CTCTCTCACCCAGTCATCACTTCA | 2208 | CUCUCUCACCCAGUCAUCACUUCA | 1135 |
| Human-Exon 43 | 1 | TTTG | tatatatatatatatTTTTCTCTT | 2209 | uauauauauauauauUUUUCUCUU | 1136 |
| Human-Exon 43 | 1 | tTTT | TCTCTTTCTATAGACAGCTAATTC | 2210 | UCUCUUUCUAUAGACAGCUAAUUC | 1137 |
| Human-Exon 43 | 1 | TTTT | CTCTTTCTATAGACAGCTAATTCA | 2211 | CUCUUUCUAUAGACAGCUAAUUCA | 1138 |
| Human-Exon 43 | −1 | TTTA | AAACAGTAAAAAAATGAATTAGCT | 2212 | AAACAGUAAAAAAAUGAAUUAGCU | 1139 |
| Human-Exon 43 | 1 | TTTC | TCTTTCTATAGACAGCTAATTCAT | 2213 | UCUUUCUAUAGACAGCUAAUUCAU | 1140 |
| Human-Exon 43 | −1 | TTTT | AAAACAGTAAAAAAATGAATTAGC | 2214 | AAAACAGUAAAAAAAUGAAUUAGC | 1141 |
| Human-Exon 43 | 1 | TTTC | TATAGACAGCTAATTCATTTTTTT | 2215 | UAUAGACAGCUAAUUCAUUUUUUU | 1142 |
| Human-Exon 43 | −1 | TTTA | TATTCTGTAATATAAAAATTTTAA | 2216 | UAUUCUGUAAUAUAAAAAUUUUAA | 1143 |
| Human-Exon 43 | −1 | TTTT | ATATTCTGTAATATAAAAATTTTA | 2217 | AUAUUCUGUAAUAUAAAAAUUUUA | 1144 |
| Human-Exon 43 | 1 | TTTT | TTTACTGTTTTAAAATTTTTATAT | 2218 | UUUACUGUUUUAAAAUUUUUAUAU | 1145 |
| Human-Exon 43 | 1 | TTTT | TTACTGTTTTAAAATTTTTATATT | 2219 | UUACUGUUUUAAAAUUUUUAUAUU | 1146 |
| Human-Exon 43 | 1 | TTTT | TACTGTTTTAAAATTTTTATATTA | 2220 | UACUGUUUUAAAAUUUUUAUAUUA | 1147 |
| Human-Exon 43 | 1 | TTTT | ACTGTTTTAAAATTTTTATATTAC | 2221 | ACUGUUUUAAAAUUUUUAUAUUAC | 1148 |
| Human-Exon 43 | 1 | TTTA | CTGTTTTAAAATTTTTATATTACA | 2222 | CUGUUUUAAAAUUUUUAUAUUACA | 1149 |
| Human-Exon 43 | 1 | TTTT | AAAATTTTTATATTACAGAATATA | 2223 | AAAAUUUUUAUAUUACAGAAUAUA | 1150 |
| Human-Exon 43 | 1 | TTTA | AAATTTTTATATTACAGAATATAA | 2224 | AAAUUUUUAUAUUACAGAAUAUAA | 1151 |
| Human-Exon 43 | −1 | TTTG | TTGTAGACTATCTTTTATATTCTG | 2225 | UUGUAGACUAUCUUUUAUAUUCUG | 1152 |
| Human-Exon 43 | 1 | TTTT | TATATTACAGAATATAAAAGATAG | 2226 | UAUAUUACAGAAUAUAAAAGAUAG | 1153 |
| Human-Exon 43 | 1 | TTTT | ATATTACAGAATATAAAAGATAGT | 2227 | AUAUUACAGAAUAUAAAAGAUAGU | 1154 |
| Human-Exon 43 | 1 | TTTA | TATTACAGAATATAAAAGATAGTC | 2228 | UAUUACAGAAUAUAAAAGAUAGUC | 1155 |
| Human-Exon 43 | −1 | TTTG | CAATGCTGCTGTCTTCTTGCTATG | 2229 | CAAUGCUGCUGUCUUCUUGCUAUG | 1156 |
| Human-Exon 43 | 1 | TTTC | CAATGGGAAAAAGTTAACAAAATG | 2230 | CAAUGGGAAAAAGUUAACAAAAUG | 1157 |
| Human-Exon 43 | −1 | TTTC | TGCAAGTATCAAGAAAAATATATG | 2231 | UGCAAGUAUCAAGAAAAAUAUAUG | 1158 |
| Human-Exon 43 | 1 | TTTT | TCTTGATACTTGCAGAAATGATTT | 2232 | UCUUGAUACUUGCAGAAAUGAUUU | 1159 |
| Human-Exon 43 | 1 | TTTT | CTTGATACTTGCAGAAATGATTTG | 2233 | CUUGAUACUUGCAGAAAUGAUUUG | 1160 |
| Human-Exon 43 | 1 | TTTC | TTGATACTTGCAGAAATGATTTGT | 2234 | UUGAUACUUGCAGAAAUGAUUUGU | 1161 |
| Human-Exon 43 | 1 | TTTG | TTTTCAGGGAACTGTAGAATTTAT | 2235 | UUUUCAGGGAACUGUAGAAUUUAU | 1162 |
| Human-Exon 43 | −1 | TTTC | CATGGAGGGTACTGAAATAAATTC | 2236 | CAUGGAGGGUACUGAAAUAAAUUC | 1163 |
| Human-Exon 43 | −1 | TTTT | CCATGGAGGGTACTGAAATAAATT | 2237 | CCAUGGAGGGUACUGAAAUAAAUU | 1164 |
| Human-Exon 43 | 1 | TTTT | CAGGGAACTGTAGAATTTATTTCA | 2238 | CAGGGAACUGUAGAAUUUAUUUCA | 1165 |
| Human-Exon 43 | −1 | TTTT | TCCATGGAGGGTACTGAAATAAAT | 2239 | UCCAUGGAGGGUACUGAAAUAAAU | 1166 |
| Human-Exon 43 | 1 | TTTC | AGGGAACTGTAGAATTTATTTCAG | 2240 | AGGGAACUGUAGAAUUUAUUUCAG | 1167 |
| Human-Exon 43 | −1 | TTTT | TTCCATGGAGGGTACTGAAATAAA | 2241 | UUCCAUGGAGGGUACUGAAAUAAA | 1168 |
| Human-Exon 43 | −1 | TTTC | CCTGTCTTTTTTCCATGGAGGGTA | 2242 | CCUGUCUUUUUUCCAUGGAGGGUA | 1169 |
| Human-Exon 43 | −1 | TTTT | CCCTGTCTTTTTTCCATGGAGGGT | 2243 | CCCUGUCUUUUUUCCAUGGAGGGU | 1170 |
| Human-Exon 43 | −1 | TTTT | TCCCTGTCTTTTTTCCATGGAGGG | 2244 | UCCCUGUCUUUUUUCCAUGGAGGG | 1171 |
| Human-Exon 43 | 1 | TTTA | TTTCAGTACCCTCCATGGAAAAAA | 2245 | UUUCAGUACCCUCCAUGGAAAAAA | 1172 |
| Human-Exon 43 | 1 | TTTC | AGTACCCTCCATGGAAAAAAGACA | 2246 | AGUACCCUCCAUGGAAAAAAGACA | 1173 |
| Human-Exon 6 | 1 | TTTA | AGTTTGCATGGTTCTTGCTCAAGG | 2247 | AGUUUGCAUGGUUCUUGCUCAAGG | 1174 |
| Human-Exon 6 | −1 | TTTC | ATAAGAAAATGCATTCCTTGAGCA | 2248 | AUAAGAAAAUGCAUUCCUUGAGCA | 1175 |
| Human-Exon 6 | −1 | TTTT | CATAAGAAAATGCATTCCTTGAGC | 2249 | CAUAAGAAAAUGCAUUCCUUGAGC | 1176 |
| Human-Exon 6 | 1 | TTTG | CATGGTTCTTGCTCAAGGAATGCA | 2250 | CAUGGUUCUUGCUCAAGGAAUGCA | 1177 |
| Human-Exon 6 | −1 | TTTG | ACCTACATGTGGAAATAAATTTTC | 2251 | ACCUACAUGUGGAAAUAAAUUUUC | 1178 |
| Human-Exon 6 | −1 | TTTT | GACCTACATGTGGAAATAAATTTT | 2252 | GACCUACAUGUGGAAAUAAAUUUU | 1179 |
| Human-Exon 6 | −1 | TTTT | TGACCTACATGTGGAAATAAATTT | 2253 | UGACCUACAUGUGGAAAUAAAUUU | 1180 |
| Human-Exon 6 | 1 | TTTT | CTTATGAAAATTTATTTCCACATG | 2254 | CUUAUGAAAAUUUAUUUCCACAUG | 1181 |
| Human-Exon 6 | 1 | TTTC | TTATGAAAATTTATTTCCACATGT | 2255 | UUAUGAAAAUUUAUUUCCACAUGU | 1182 |
| Human-Exon 6 | −1 | TTTC | ATTACATTTTTGACCTACATGTGG | 2256 | AUUACAUUUUUGACCUACAUGUGG | 1183 |
| Human-Exon 6 | −1 | TTTT | CATTACATTTTTGACCTACATGTG | 2257 | CAUUACAUUUUUGACCUACAUGUG | 1184 |
| Human-Exon 6 | −1 | TTTT | TCATTACATTTTTGACCTACATGT | 2258 | UCAUUACAUUUUUGACCUACAUGU | 1185 |
| Human-Exon 6 | 1 | TTTA | TTTCCACATGTAGGTCAAAAATGT | 2259 | UUUCCACAUGUAGGUCAAAAAUGU | 1186 |
| Human-Exon 6 | 1 | TTTC | CACATGTAGGTCAAAAATGTAATG | 2260 | CACAUGUAGGUCAAAAAUGUAAUG | 1187 |
| Human-Exon 6 | −1 | TTTG | TTGCAATCCAGCCATGATATTTTT | 2261 | UUGCAAUCCAGCCAUGAUAUUUUU | 1188 |
| Human-Exon 6 | −1 | TTTC | ACTGTTGGTTTGTTGCAATCCAGC | 2262 | ACUGUUGGUUUGUUGCAAUCCAGC | 1189 |
| Human-Exon 6 | −1 | TTTT | CACTGTTGGTTTGTTGCAATCCAG | 2263 | CACUGUUGGUUUGUUGCAAUCCAG | 1190 |
| Human-Exon 6 | 1 | TTTG | AATGCTCTCATCCATAGTCATAGG | 2264 | AAUGCUCUCAUCCAUAGUCAUAGG | 1191 |
| Human-Exon 6 | −1 | TTTA | ATGTCTCAGTAATCTTCTTACCTA | 2265 | AUGUCUCAGUAAUCUUCUUACCUA | 1192 |
| Human-Exon 6 | −1 | TTTA | CAAGTTATTTAATGTCTCAGTAAT | 2266 | CAAGUUAUUUAAUGUCUCAGUAAU | 1193 |
| Human-Exon 6 | −1 | TTTT | ACAAGTTATTTAATGTCTCAGTAA | 2267 | ACAAGUUAUUUAAUGUCUCAGUAA | 1194 |
| Human-Exon 6 | 1 | TTTA | GACTCTGATGACATATTTTTCCCC | 2268 | GACUCUGAUGACAUAUUUUUCCCC | 1195 |
| Human-Exon 6 | 1 | TTTT | TCCCCAGTATGGTTCCAGATCATG | 2269 | UCCCCAGUAUGGUUCCAGAUCAUG | 1196 |
| Human-Exon 6 | 1 | TTTT | CCCCAGTATGGTTCCAGATCATGT | 2270 | CCCCAGUAUGGUUCCAGAUCAUGU | 1197 |
| Human-Exon 6 | 1 | TTTC | CCCAGTATGGTTCCAGATCATGTC | 2271 | CCCAGUAUGGUUCCAGAUCAUGUC | 1198 |
| Human-Exon 7 | 1 | TTTA | TATTTGTCTTtgtgtatgtgtgta | 2272 | UAUUUGUCUUuguguaugugugua | 1199 |
| Human-Exon 7 | 1 | TTTG | TCTTtgtgtatgtgtgtatgtgta | 2273 | UCUUuguguauguguguaugugua | 1200 |
| Human-Exon 7 | 1 | TTtg | tgtatgtgtgtatgtgtatgtgtt | 2274 | uguauguguguauguguauguguu | 1201 |
| Human-Exon 7 | 1 | ttTT | AGGCCAGACCTATTTGACTGGAAT | 2275 | AGGCCAGACCUAUUUGACUGGAAU | 1202 |
| Human-Exon 7 | 1 | tTTA | GGCCAGACCTATTTGACTGGAATA | 2276 | GGCCAGACCUAUUUGACUGGAAUA | 1203 |
| Human-Exon 7 | 1 | TTTG | ACTGGAATAGTGTGGTTTGCCAGC | 2277 | ACUGGAAUAGUGUGGUUUGCCAGC | 1204 |
| Human-Exon 7 | 1 | TTTG | CCAGCAGTCAGCCACACAACGACT | 2278 | CCAGCAGUCAGCCACACAACGACU | 1205 |
| Human-Exon 7 | −1 | TTTC | TCTATGCCTAATTGATATCTGGCG | 2279 | UCUAUGCCUAAUUGAUAUCUGGCG | 1206 |
| Human-Exon 7 | −1 | TTTA | CCAACCTTCAGGATCGAGTAGTTT | 2280 | CCAACCUUCAGGAUCGAGUAGUUU | 1207 |
| Human-Exon 7 | 1 | TTTC | TGGACTACCACTGCTTTTAGTATG | 2281 | UGGACUACCACUGCUUUUAGUAUG | 1208 |
| Human-Exon 7 | 1 | TTTT | AGTATGGTAGAGTTTAATGTTTTC | 2282 | AGUAUGGUAGAGUUUAAUGUUUUC | 1209 |
| Human-Exon 7 | 1 | TTTA | GTATGGTAGAGTTTAATGTTTTCA | 2283 | GUAUGGUAGAGUUUAAUGUUUUCA | 1210 |
| Human-Exon 8 | −1 | TTTG | AGACTCTAAAAGGATAATGAACAA | 2284 | AGACUCUAAAAGGAUAAUGAACAA | 1211 |
| Human-Exon 8 | 1 | TTTA | ACTTTGATTTGTTCATTATCCTTT | 2285 | ACUUUGAUUUGUUCAUUAUCCUUU | 1212 |
| Human-Exon 8 | −1 | TTTC | TATATTTGAGACTCTAAAAGGATA | 2286 | UAUAUUUGAGACUCUAAAAGGAUA | 1213 |
| Human-Exon 8 | 1 | TTTG | ATTTGTTCATTATCCTTTTAGAGT | 2287 | AUUUGUUCAUUAUCCUUUUAGAGU | 1214 |
| Human-Exon 8 | −1 | TTTG | GTTTCTATATTTGAGACTCTAAAA | 2288 | GUUUCUAUAUUUGAGACUCUAAAA | 1215 |
| Human-Exon 8 | −1 | TTTT | GGTTTCTATATTTGAGACTCTAAA | 2289 | GGUUUCUAUAUUUGAGACUCUAAA | 1216 |
| Human-Exon 8 | −1 | TTTT | TGGTTTCTATATTTGAGACTCTAA | 2290 | UGGUUUCUAUAUUUGAGACUCUAA | 1217 |
| Human-Exon 8 | 1 | TTTG | TTCATTATCCTTTTAGAGTCTCAA | 2291 | UUCAUUAUCCUUUUAGAGUCUCAA | 1218 |
| Human-Exon 8 | 1 | TTTT | AGAGTCTCAAATATAGAAACCAAA | 2292 | AGAGUCUCAAAUAUAGAAACCAAA | 1219 |
| Human-Exon 8 | 1 | TTTA | GAGTCTCAAATATAGAAACCAAAA | 2293 | GAGUCUCAAAUAUAGAAACCAAAA | 1220 |
| Human-Exon 8 | −1 | TTTC | CACTTCCTGGATGGCTTCAATGCT | 2294 | CACUUCCUGGAUGGCUUCAAUGCU | 1221 |
| Human-Exon 8 | 1 | TTTT | GCCTCAACAAGTGAGCATTGAAGC | 2295 | GCCUCAACAAGUGAGCAUUGAAGC | 1222 |
| Human-Exon 8 | 1 | TTTG | CCTCAACAAGTGAGCATTGAAGCC | 2296 | CCUCAACAAGUGAGCAUUGAAGCC | 1223 |
| Human-Exon 8 | −1 | TTTA | GGTGGCCTTGGCAACATTTCCACT | 2297 | GGUGGCCUUGGCAACAUUUCCACU | 1224 |
| Human-Exon 8 | −1 | TTTA | GTCACTTTAGGTGGCCTTGGCAAC | 2298 | GUCACUUUAGGUGGCCUUGGCAAC | 1225 |
| Human-Exon 8 | −1 | TTTG | ATGATGTAACTGAAAATGTTCTTC | 2299 | AUGAUGUAACUGAAAAUGUUCUUC | 1226 |
| Human-Exon 8 | −1 | TTTA | CCTGTTGAGAATAGTGCATTTGAT | 2300 | CCUGUUGAGAAUAGUGCAUUUGAU | 1227 |
| Human-Exon 8 | 1 | TTTT | CAGTTACATCATCAAATGCACTAT | 2301 | CAGUUACAUCAUCAAAUGCACUAU | 1228 |
| Human-Exon 8 | 1 | TTTC | AGTTACATCATCAAATGCACTATT | 2302 | AGUUACAUCAUCAAAUGCACUAUU | 1229 |
| Human-Exon 8 | −1 | TTTA | CACACTTTACCTGTTGAGAATAGT | 2303 | CACACUUUACCUGUUGAGAAUAGU | 1230 |
| Human-Exon 8 | 1 | TTTT | CTGTTTTATATGCATTTTTAGGTA | 2304 | CUGUUUUAUAUGCAUUUUUAGGUA | 1231 |
| Human-Exon 8 | 1 | TTTC | TGTTTTATATGCATTTTTAGGTAT | 2305 | UGUUUUAUAUGCAUUUUUAGGUAU | 1232 |
| Human-Exon 8 | 1 | TTTT | ATATGCATTTTTAGGTATTACGTG | 2306 | AUAUGCAUUUUUAGGUAUUACGUG | 1233 |
| Human-Exon 8 | 1 | TTTA | TATGCATTTTTAGGTATTACGTGC | 2307 | UAUGCAUUUUUAGGUAUUACGUGC | 1234 |
| Human-Exon 8 | 1 | TTTT | TAGGTATTACGTGCACatatatat | 2308 | UAGGUAUUACGUGCACauauauau | 1235 |
| Human-Exon 8 | 1 | TTTT | AGGTATTACGTGCACatatatata | 2309 | AGGUAUUACGUGCACauauauaua | 1236 |
| Human-Exon 8 | 1 | TTTA | GGTATTACGTGCACatatatatat | 2310 | GGUAUUACGUGCACauauauauau | 1237 |
| Human-Exon 55 | −1 | TTTA | AGCAACAACTATAATATTGTGCAG | 2311 | AGCAACAACUAUAAUAUUGUGCAG | 1238 |
| Human-Exon 55 | 1 | TTTA | GTTCCTCCATCTTTCTCTTTTTAT | 2312 | GUUCCUCCAUCUUUCUCUUUUUAU | 1239 |
| Human-Exon 55 | 1 | TTTC | TCTTTTTATGGAGTTCACTAGGTG | 2313 | UCUUUUUAUGGAGUUCACUAGGUG | 1240 |
| Human-Exon 55 | 1 | TTTT | TATGGAGTTCACTAGGTGCACCAT | 2314 | UAUGGAGUUCACUAGGUGCACCAU | 1241 |
| Human-Exon 55 | 1 | TTTT | ATGGAGTTCACTAGGTGCACCATT | 2315 | AUGGAGUUCACUAGGUGCACCAUU | 1242 |
| Human-Exon 55 | 1 | TTTA | TGGAGTTCACTAGGTGCACCATTC | 2316 | UGGAGUUCACUAGGUGCACCAUUC | 1243 |
| Human-Exon 55 | 1 | TTTA | ATAATTGCATCTGAACATTTGGTC | 2317 | AUAAUUGCAUCUGAACAUUUGGUC | 1244 |
| Human-Exon 55 | 1 | TTTG | GTCCTTTGCAGGGTGAGTGAGCGA | 2318 | GUCCUUUGCAGGGUGAGUGAGCGA | 1245 |
| Human-Exon 55 | −1 | TTTC | TTCCAAAGCAGCCTCTCGCTCACT | 2319 | UUCCAAAGCAGCCUCUCGCUCACU | 1246 |
| Human-Exon 55 | 1 | TTTG | CAGGGTGAGTGAGCGAGAGGCTGC | 2320 | CAGGGUGAGUGAGCGAGAGGCUGC | 1247 |
| Human-Exon 55 | 1 | TTTG | GAAGAAACTCATAGATTACTGCAA | 2321 | GAAGAAACUCAUAGAUUACUGCAA | 1248 |
| Human-Exon 55 | −1 | TTTC | CAGGTCCAGGGGGAACTGTTGCAG | 2322 | CAGGUCCAGGGGGAACUGUUGCAG | 1249 |
| Human-Exon 55 | −1 | TTTT | CCAGGTCCAGGGGGAACTGTTGCA | 2323 | CCAGGUCCAGGGGGAACUGUUGCA | 1250 |
| Human-Exon 55 | −1 | TTTC | AGCTTCTGTAAGCCAGGCAAGAAA | 2324 | AGCUUCUGUAAGCCAGGCAAGAAA | 1251 |
| Human-Exon 55 | 1 | TTTC | TTGCCTGGCTTACAGAAGCTGAAA | 2325 | UUGCCUGGCUUACAGAAGCUGAAA | 1252 |
| Human-Exon 55 | −1 | TTTC | CTTACGGGTAGCATCCTGTAGGAC | 2326 | CUUACGGGUAGCAUCCUGUAGGAC | 1253 |
| Human-Exon 55 | −1 | TTTA | CTCCCTTGGAGTCTTCTAGGAGCC | 2327 | CUCCCUUGGAGUCUUCUAGGAGCC | 1254 |
| Human-Exon 55 | −1 | TTTT | ACTCCCTTGGAGTCTTCTAGGAGC | 2328 | ACUCCCUUGGAGUCUUCUAGGAGC | 1255 |
| Human-Exon 55 | −1 | TTTC | ATCAGCTCTTTTACTCCCTTGGAG | 2329 | AUCAGCUCUUUUACUCCCUUGGAG | 1256 |
| Human-Exon 55 | 1 | TTTC | CGCTTTAGCACTCTTGTGGATCCA | 2330 | CGCUUUAGCACUCUUGUGGAUCCA | 1257 |
| Human-Exon 55 | 1 | TTTA | GCACTCTTGTGGATCCAATTGAAC | 2331 | GCACUCUUGUGGAUCCAAUUGAAC | 1258 |
| Human-Exon 55 | −1 | TTTG | TCCCTGGCTTGTCAGTTACAAGTA | 2332 | UCCCUGGCUUGUCAGUUACAAGUA | 1259 |
| Human-Exon 55 | −1 | TTTT | GTCCCTGGCTTGTCAGTTACAAGT | 2333 | GUCCCUGGCUUGUCAGUUACAAGU | 1260 |
| Human-Exon 55 | −1 | TTTG | TTTTGTCCCTGGCTTGTCAGTTAC | 2334 | UUUUGUCCCUGGCUUGUCAGUUAC | 1261 |
| Human-Exon 55 | −1 | TTTT | GTTTTGTCCCTGGCTTGTCAGTTA | 2335 | GUUUUGUCCCUGGCUUGUCAGUUA | 1262 |
| Human-Exon 55 | 1 | TTTG | TACTTGTAACTGACAAGCCAGGGA | 2336 | UACUUGUAACUGACAAGCCAGGGA | 1263 |
| Human-G1- | 1 | TTTA | gCTCCTACTCAGACTGTTACTCTG | 2337 | gCUCCUACUCAGACUGUUACUCUG | 1264 |
| exon51 | | | | | | |
| Human-G2- | 1 | TTTC | taccatgtattgctaaacaaagta | 2338 | uaccauguauugcuaaacaaagua | 1265 |
| exon51 | | | | | | |
| Human-G3- | −1 | TTTA | attgaagagtaacaatttgagcca | 2339 | auugaagaguaacaauuugagcca | 1266 |
| exon51 | | | | | | |
| mouse-Exon23- | 1 | TTTG | aggctctgcaaagttctTTGAAAG | 2340 | aggcucugcaaaguucuUUGAAAG | 1267 |
| G1 | | | | | | |
| mouse-Exon23- | 1 | TTTG | AAAGAGCAACAAAATGGCttcaac | 2341 | AAAGAGCAACAAAAUGGCuucaac | 1268 |
| G2 | | | | | | |
| mouse-Exon23- | 1 | TTTG | AAAGAGCAATAAAATGGCttcaac | 2342 | AAAGAGCAAUAAAAUGGCuucaac | 1269 |
| G3 | | | | | | |
| mouse-Exon23- | −1 | TTTC | AAAGAACTTTGCAGAGCctcaaaa | 2343 | AAAGAACUUUGCAGAGCcucaaaa | 1270 |
| G4 | | | | | | |
| mouse-Exon23- | −1 | TTTA | ctgaatatctatgcattaataact | 2344 | cugaauaucuaugcauuaauaacu | 1271 |
| G5 | | | | | | |
| mouse-Exon23- | −1 | TTTC | tattatattacagggcatattata | 2345 | uauuauauuacagggcauauuaua | 1272 |
| G6 | | | | | | |
| mouse-Exon23- | 1 | TTTC | Aggtaagccgaggtttggccttta | 2346 | Agguaagccgagguuuggccuuua | 1273 |
| G7 | | | | | | |
| mouse-Exon23- | 1 | TTTA | cccagagtccttcaaagatattga | 2347 | cccagaguccuucaaagauauuga | 1274 |
| G8 | | | | | | |

*In this table, upper case letters represent sgRNA nucleotides that align to the exon sequence of the gene. Lower case letters represent sgRNA nucleotides that align to the intron sequence of the gene

TABLE 15
Additional gRNA targeting sequences

| | | | | | | SEQ | |
|---|---|---|---|---|---|---|---|
| Name | Species | Gene | Target | Strand | Sequence | ID NO | PAM |
| DCR | Human | DMD | Intron | + | attggctttgatttcccta | 1275 | GGG |
| 1 | 50 | | | | | | |
| DCR | Human | DMD | Intron | − | tgtagagtaagtcagccta | 1276 | TGG |
| 2 | 50 | | | | | | |
| DCR | Human | DMD | Exon | + | cctactcagactgttactc | 1277 | TGG |
| 3 | 51-55′ | | | | | | |
| DCR | Human | DMD | Exon | + | ttggacagaacttaccgac | 1278 | TGG |
| 4 | 51-53′ | | | | | | |
| DCR | Human | DMD | Intron | − | cagttgcctaagaactggt | 1279 | GGG |
| 5 | 51 | | | | | | |
| DCR | Human | DMD | Intron | − | GGGCTCCACCCTCACGAGT | 1280 | GGG |
| 6 | 44 | | | | | | |
| DCR | Human | DMD | Intron | + | TTTGCTTCGCTATAAAACG | 1281 | AGG |
| 7 | 55 | | | | | | |
| DCR | Human | DMD | Exon 41 | + | TCTGAGGATGGGCCGCAA | 1282 | TGG |
| 8 | | | | | | | |
| DCR | Human | DMD | Exon 44 | − | GATCTGTCAAATCGCCTGG | 1283 | AGG |
| 9 | | | | | | | |
| DCR | Human | DMD | Exon 45 | + | CCAGGATGGCATTGGGCAG | 1284 | CGG |
| 10 | | | | | | | |
| DCR | Human | DMD | Exon 45 | + | CTGAATCTGCGGTGGCAGG | 1285 | AGG |
| 11 | | | | | | | |
| DCR | Human | DMD | Exon 46 | − | TTCTTTTGTTCTTCTAGCc | 1286 | TGG |
| 12 | | | | | | | |
| DCR | Human | DMD | Exon 46 | + | GAAAAGCTTGAGCAAGTCA | 1287 | AGG |
| 13 | | | | | | | |
| DCR | Human | DMD | Exon 47 | + | GAAGAGTTGCCCCTGCGCC | 1288 | AGG |
| 14 | | | | | | | |
| DCR | Human | DMD | Exon 47 | + | ACAAATCTCCAGTGGATAA | 1289 | AGG |
| 15 | | | | | | | |
| DCR | Human | DMD | Exon 48 | − | TGTTTCTCAGGTAAAGCTC | 1290 | TGG |
| 16 | | | | | | | |
| DCR | Human | DMD | Exon 48 | + | GAAGGACCATTTGACGTTa | 1291 | AGG |
| 17 | | | | | | | |
| DCR | Human | DMD | Exon 49 | − | AACTGCTATTTCAGTTTCc | 1292 | TGG |
| 18 | | | | | | | |
| DCR | Human | DMD | Exon 49 | + | CCAGCCACTCAGCCAGTGA | 1293 | AGG |
| 19 | | | | | | | |
| DCR | Human | DMD | Exon 50 | + | gtatgcttttctgttaaag | 1294 | AGG |
| 20 | | | | | | | |
| DCR | Human | DMD | Exon 50 | + | CTCCTGGACTGACCACTAT | 1295 | TGG |
| 21 | | | | | | | |
| DCR | Human | DMD | Exon 52 | + | GAACAGAGGCGTCCCCAGT | 1296 | TGG |
| 22 | | | | | | | |
| DCR | Human | DMD | Exon 52 | + | GAGGCTAGAACAATCATTA | 1297 | CGG |
| 23 | | | | | | | |
| DCR | Human | DMD | Exon 53 | + | ACAAGAACACCTTCAGAAC | 1298 | CGG |
| 24 | | | | | | | |
| DCR | Human | DMD | Exon 53 | − | GGTTTCTGTGATTTTCTTT | 1299 | TGG |
| 25 | | | | | | | |
| DCR | Human | DMD | Exon 54 | + | GGCCAAAGACCTCCGCCAG | 1300 | TGG |
| 26 | | | | | | | |
| DCR | Human | DMD | Exon 54 | + | TTGGAGAAGCATTCATAAA | 1301 | AGG |
| 27 | | | | | | | |
| DCR | Human | DMD | Exon 55 | − | TCGCTCACTCACCctgcaa | 1302 | AGG |
| 28 | | | | | | | |
| DCR | Human | DMD | Exon 55 | + | AAAAGAGCTGATGAAACAA | 1303 | TGG |
| 29 | | | | | | | |
| DCR | Human | DMD | 5′UTR/ | + | TAcACTTTTCaAAATGCTT | 1304 | TGG |
| 30 | | | Exon 1 | | | | |
| DCR | Human | DMD | Exon 51 | + | gagatgatcatcaagcaga | 1305 | AGG |
| 31 | | | | | | | |
| DCR | Mouse | DMD | mdx | + | ctttgaaagagcaaTaaaa | 1306 | TGG |
| 32 | | | | | | | |
| DCR | Human | DMD | Intron | − | CACAAAAGTCAAATCGGAA | 1307 | TGG |
| 33 | | | 44 | | | | |
| DCR | Human | DMD | Intron | − | ATTTCAATATAAGATTCGG | 1308 | AGG |
| 34 | | | 44 | | | | |
| DCR | Human | DMD | Intron | − | CTTAAGCAATCCCGAACTC | 1309 | TGG |
| 35 | | | 55 | | | | |
| DCR | Human | DMD | Intron | − | CCTTCTTTATCCCCTATCG | 1310 | AGG |
| 36 | | | 55 | | | | |
| DCR | Mouse | DMD | Exon 23 | − | aggccaaacctcggcttac | 1311 | NNGRR |
| 40 | | | | | | | |
| DCR | Mouse | DMD | Exon 23 | + | TTCGAAAATTTCAGgtaag | 1312 | NNGRR |
| 41 | | | | | | | |
| DCR | Mouse | DMD | Exon 23 | + | gcagaacaggagataacag | 1313 | NNGRRT |
| 42 | | | | | | | |
| DCR | Mouse | ACVR | Exon 1 | + | gcggccctcgcccttctct | 1314 | ggggat |
| 43 | | 2B | | | | | |
| DCR | Human | DMD | Intron | − | TAGTGATCGTGGATACGAG | 1315 | AGG |
| 48 | | | 45 | | | | |
| DCR | Human | DMD | Intron | − | TACAGCCCTCGGTGTATAT | 1316 | TGG |
| 49 | | | 45 | | | | |
| DCR | Human | DMD | Intron | − | GGAAGGAATTAAGCCCGAA | 1317 | TGG |
| 50 | | | 52 | | | | |
| DCR | Human | DMD | Intron | − | GGAACAGCTTTCGTAGTTG | 1318 | AGG |
| 51 | | | 53 | | | | |
| DCR | Human | DMD | Intron | + | ATAAAGTCCAGTGTCGATC | 1319 | AGG |
| 52 | | | 54 | | | | |
| DCR | | | Intron | + | AAAACCAGAGCTTCGGTCA | 1320 | AGG |
| 53 | | | 54 | | | | |
| DCR | Mouse | Rosa | ZFN | + | GAGTCTTCTGGGCAGGCTTAA | 1321 | TGG |
| 54 | | 26 | region | | | | |
| DCR | Mouse | Rosa | mRNA | − | TCGGGTGAGCATGTCTTTAAT | 1322 | TGG |
| 55 | | 26 | | | | | |
| DCR | Human | DMD | Ex 51 | − | gtgtcaccagagtaacagt | 1323 | ctgagt |
| 49 | | | | | | | |
| DCR | Human | DMD | Ex 51 | − | tgatcatcaagcagaaggt | 1324 | atgag |
| 50 | | | | | | | |
| DCR | Mouse | DMD | Exon 23 | + | AACTTCGAAAATTTCAGgta | 1325 | agccga |
| 60 | | | | | | | |
| DCR | Mouse | DMD | Intron | + | gaaactcatcaaatatgcgt | 1326 | gttagt |
| 61 | | | 22 | | | | gt |
| DCR | Mouse | DMD | Intron | − | tcatttacactaacacgcat | 1327 | atttqa |
| 62 | | | 22 | | | | tg |
| DCR | Mouse | DMD | Intron | + | gaatgaaactcatcaaatat | 1328 | gcgtgt |
| 63 | | | 22 | | | | ta |
| DCR | Mouse | DMD | Intron | − | tcatcaatatctttgaagga | 1329 | ctctgg |
| 64 | | | 23 | | | | gt |
| DCR | Mouse | DMD | Intron | − | tgttttcataggaaaaatag | 1330 | gcaagt |
| 65 | | | 23 | | | | tg |
| DCR | Mouse | DMD | Intron | + | aattggaaaatgtgatggga | 1331 | aacaga |
| 66 | | | 23 | | | | ta |
| DCR | Human | DMD | Exon 51 | + | atgatcatcaagcagaaggt | 1332 | atgaga |
| 67 | | | | | | | aa |
| DCR | Human | DMD | Exon 51 | + | agatgatcatcaagcagaag | 1333 | gtatga |
| 68 | | | | | | | ga |
| DCR | Human | DMD | Exon 51 | − | cattttttatcatacattct | 1334 | gcttga |
| 69 | | | | | | | tg |
| DCR | Human | DMD | Exon 51 | + | tcctactcagactgttactc | 1335 | tggtga |
| 70 | | | | | | | ca |
| DCR | Human | DMD | Exon 51 | − | acaggttgtgtcaccagagt | 1336 | aacagt |
| 71 | | | | | | | ct |
| DCR | Human | DMD | Exon 51 | − | ttatcattttttctcatacc | 1337 | ttctgc |
| 72 | | | | | | | tt |
| DCR | Human | DMD | Intron | − | ttgcctaagaactggtggga | 1338 | aatggt |
| 73 | | | 51 | | | | ct |
| DCR | Human | DMD | Intron | − | aaacagttgcctaagaactg | 1339 | gtggga |
| 74 | | | 51 | | | | aa |
| DCR | Human | DMD | Intron | + | tttcccaccagttcttaggc | 1340 | aactgt |
| 75 | | | 51 | | | | tt |
| DCR | Human | DMD | Intron | + | tggctttgatttccctaggg | 1341 | tccagc |
| 76 | | | 50 | | | | tt |
| DCR | Human | DMD | Intron | − | tagggaaatcaaagccaatg | 1342 | aaacqt |
| 77 | | | 50 | | | | tc |
| DCR | Human | DMD | Intron | − | gaccctagggaaatcaaagc | 1343 | caatga |
| 78 | | | 50 | | | | aa |
| DCR | Human | DMD | Intron | − | TGAGGGCTCCACCCTCACGA | 1344 | GTGGGT |
| 79 | | | 44 | | | | TT |
| DCR | Human | DMD | Intron | − | AAGGATTGAGGGCTCCACCC | 1345 | TCACGA |
| 80 | | | 44 | | | | GT |
| DCR | Human | DMD | Intron | − | GCTCCACCCTCACGAGTGGG | 1346 | TTTGGT |
| 81 | | | 44 | | | | TC |
| DCR | Human | DMD | Intron | − | TATCCCCTATCGAGGAAACC | 1347 | ACGAGT |
| 82 | | | 55 | | | | TT |
| DCR | Human | DMD | Intron | + | GATAAAGAAGGCCTATTTCA | 1348 | TAGAGT |
| 83 | | | 55 | | | | TG |
| DCR | Human | DMD | Intron | − | AGGCCTTCTTTATCCCCTAT | 1349 | CGAGGA |
| 84 | | | 55 | | | | AA |
| DCR | Human | DMD | Intron | − | TGAGGGCTCCACCCTCACGA | 1350 | GTGGGT |
| 85 | | | 44 | | | | |
| DCR | Human | DMD | Intron | + | GATAAAGAAGGCCTATTTCA | 1351 | TAGAGT |
| 86 | | | 55 | | | | |
| DCR | Human | DMD | Intron | + | attggctttgatttcccta | 1352 | GGG |
| 1 | | | 50 | | | | |
| DCR | Human | DMD | Intron | − | tgtagagtaagtcagccta | 1353 | TGG |
| 2 | | | 50 | | | | |
| DCR | Human | DMD | Exon | + | cctactcagactgttactc | 1354 | TGG |
| 3 | | | 51-5′ | | | | |
| DCR | Human | DMD | Exon | + | ttggacagaacttaccgac | 1355 | TGG |
| 4 | | | 51-3′ | | | | |
| DCR | Human | DMD | Intron | − | cagttgcctaagaactggt | 1356 | GGG |
| 5 | | | 51 | | | | |
| DCR | Human | DMD | Intron | − | GGGCTCCACCCTCACGAGT | 1357 | GGG |
| 6 | | | 44 | | | | |
| DCR | Human | DMD | Intron | + | TTTGCTTCGCTATAAAACG | 1358 | AGG |
| 7 | | | 55 | | | | |
| DCR | Human | DMD | Exon 41 | + | TCTGAGGATGGGGCCGCAA | 1359 | TGG |
| 8 | | | | | | | |
| DCR | Human | DMD | Exon 44 | − | GATCTGTCAAATCGCCTGC | 1360 | AGG |
| 9 | | | | | | | |
| DCR | Human | DMD | Exon 45 | + | CCAGGATGGCATTGGGCAG | 1361 | CGG |
| 10 | | | | | | | |
| DCR | Human | DMD | Exon 45 | + | CTGAATCTGCGGTGGCAGG | 1362 | AGG |
| 11 | | | | | | | |
| DCR | Human | DMD | Exon 46 | − | TTCTTTTGTTCTTCTAGCc | 1363 | TGG |
| 12 | | | | | | | |
| DCR | Human | DMD | Exon 46 | + | GAAAAGCTTGAGCAAGTCA | 1364 | AGG |
| 13 | | | | | | | |
| DCR | Human | DMD | Exon 47 | + | GAAGAGTTGCCCCTGCGCC | 1365 | AGE |
| 14 | | | | | | | |
| DCR | Human | DMD | Exon 47 | + | ACAAATCTCCAGTGGATAA | 1366 | AGE |
| 15 | | | | | | | |
| DCR | Human | DMD | Exon 48 | − | TGTTTCTCAGGTAAAGCTC | 1367 | TGG |
| 16 | | | | | | | |
| DCR | Human | DMD | Exon 48 | + | GAAGGACCATTTGACGTTa | 1368 | AGG |
| 17 | | | | | | | |
| DCR | Human | DMD | Exon 49 | − | AACTGCTATTTCAGTTTCc | 1369 | TGG |
| 18 | | | | | | | |
| DCR | Human | DMD | Exon 49 | + | CCAGCCACTCAGCCAGTGA | 1370 | AGG |
| 19 | | | | | | | |
| DCR | Human | DMD | Exon 50 | + | gtatgcttttctgttaaag | 1371 | AGG |
| 20 | | | | | | | |
| DCR | Human | DMD | Exon 50 | + | CTCCTGGACTGACCACTAT | 1372 | TGG |
| 21 | | | | | | | |
| DCR | Human | DMD | Exon 52 | + | GAACAGAGGCGTCCCCAGT | 1373 | TGG |
| 22 | | | | | | | |
| DCR | Human | DMD | Exon 52 | + | GAGGCTAGAACAATCATTA | 1374 | CGG |
| 23 | | | | | | | |
| DCR | Human | DMD | Exon 53 | + | ACAAGAACACCTTCAGAAC | 1375 | CGG |
| 24 | | | | | | | |
| DCR | Human | DMD | Exon 53 | − | GGTTTCTGTGATTTTCTTT | 1376 | TGG |
| 25 | | | | | | | |
| DCR | Human | DMD | Exon 54 | + | GGCCAAAGACCTCCGCCAG | 1377 | TGG |
| 26 | | | | | | | |
| DCR | Human | DMD | Exon 54 | + | TTGGAGAAGCATTCATAAA | 1378 | AGG |
| 27 | | | | | | | |
| DCR | Human | DMD | Exon 55 | − | TCGCTCACTCACCctgcaa | 1379 | AGG |
| 28 | | | | | | | |
| DCR | Human | DMD | Exon 55 | + | AAAAGAGCTGATGAAACAA | 1380 | TGG |
| 29 | | | | | | | |
| DCR | Human | DMD | 5′UTR/ | + | TAcACTTTTCaAAATGCTT | 1381 | TGG |
| 30 | | | Exon 1 | | | | |
| DCR | Human | DMD | Exon 51 | + | gagatgatcatcaagcaga | 1382 | AGG |
| 31 | | | | | | | |
| DCR | Mouse | DMD | mdx | + | ctttgaaagagcaaTaaaa | 1383 | TGG |
| 32 | | | | | | | |
| DCR | Human | DMD | Intron | − | CACAAAAGTCAAATCGGAA | 1384 | TGG |
| 33 | | | 44 | | | | |
| DCR | Human | DMD | Intron | − | ATTTCAATATAAGATTCGG | 1385 | AGG |
| 34 | | | 44 | | | | |
| DCR | Human | DMD | Intron | − | CTTAAGCAATCCCGAACTC | 1386 | TGG |
| 35 | | | 55 | | | | |
| DCR | Human | DMD | Intron | − | CCTTCTTTATCCCCTATCG | 1387 | AGG |
| 36 | | | 55 | | | | |
| DCR | Mouse | DMD | Exon 23 | − | aggccaaacctcggcttac | 1388 | NNGRR |
| 40 | | | | | | | |
| DCR | Mouse | DMD | Exon 23 | + | TTCGAAAATTTCAGgtaag | 1389 | NNGRR |
| 41 | | | | | | | |
| DCR | Mouse | DMD | Exon 23 | + | gcagaacaggagataacag | 1390 | NNGRRT |
| 42 | | | | | | | |
| DCR | Mouse | ACVR | Exon 1 | + | gcggccctcgccdctctct | 1391 | ggggat |
| 43 | | 2B | | | | | |
| DCR | Human | DMD | Intron | − | TAGTGATCGTGGATACGAG | 1392 | AGG |
| 48 | | | 45 | | | | |
| DCR | Human | DMD | Intron | − | TACAGCCCTCGGTGTATAT | 1393 | TGG |
| 49 | | | 45 | | | | |
| DCR | Human | DMD | Intron | − | GGAAGGAATTAAGCCCGAA | 1394 | TGG |
| 50 | | | 52 | | | | |
| DCR | Human | DMD | Intron | − | GGAACAGCTTTCGTAGTTG | 1395 | AGG |
| 51 | | | 53 | | | | |
| DCR | Human | DMD | Intron | + | ATAAAGTCCAGTGTCGATC | 1396 | AGG |
| 52 | | | 54 | | | | |
| DCR | Human | DMD | Intron | + | AAAACCAGAGCTTCGGTCA | 1397 | AGG |
| 53 | | | 54 | | | | |
| DCR | Mouse | Rosa | ZFN | + | GAGTCTTCTGGGCAGGCTTAA | 1398 | TGG |
| 54 | | 26 | region | | | | |
| DCR | Mouse | Rosa | mRNA | − | TCGGGTGAGCATGTCTTTAAT | 1299 | TGG |
| 55 | | 26 | | | | | |
| DCR | Human | DMD | Ex 51 | − | gtgtcaccagagtaacagt | 1400 | ctgagt |
| 49 | | | | | | | |
| DCR | Human | DMD | Ex 51 | + | tgatcatcaagcagaaggt | 1401 | atgag |
| 50 | | | | | | | |
| DCR | Mouse | DMD | Exon 23 | + | AACTTCGAAAATTTCAGgta | 1402 | agccga |
| 60 | | | | | | | gg |
| DCR | Mouse | DMD | Intron | + | gaaactcatcaaatatgcgt | 1403 | gttagt |
| 61 | | | 22 | | | | gt |
| DCR | Mouse | DMD | Intron | − | tcatttacactaacacgcat | 1404 | atttga |
| 62 | | | 22 | | | | tg |
| DCR | Mouse | DMD | Intron | + | gaatgaaactcatcaaatat | 1405 | gcgtgt |
| 63 | | | 22 | | | | ta |
| DCR | Mouse | DMD | Intron | − | tcatcaatatctttgaagga | 1406 | ctctgg |
| 64 | | | 23 | | | | gt |
| DCR | Mouse | DMD | Intron | − | tgttttcataggaaaaatag | 1407 | gcaagt |
| 65 | | | 23 | | | | tg |
| DCR | Mouse | DMD | Intron | + | aattggaaaatgtgatggga | 1408 | aacaga |
| 66 | | | 23 | | | | ta |
| DCR | Human | DMD | Exon 51 | + | atgatcatcaagcagaaggt | 1409 | atgaga |
| 67 | | | | | | | aa |
| DCR | Human | DMD | Exon 51 | + | agatgatcatcaagcagaag | 1410 | gtatga |
| 68 | | | | | | | ga |
| DCR | Human | DMD | Exon 51 | − | cattttttctcataccttct | 1411 | gcttga |
| 69 | | | | | | | tg |
| DCR | Human | DMD | Exon 51 | + | tcctactcagactgttactc | 1412 | tggtga |
| 70 | | | | | | | ca |
| DCR | Human | DMD | Exon 51 | − | acaggttgtgtcaccagagt | 1413 | aacagt |
| 71 | | | | | | | ct |
| DCR | Human | DMD | Exon 51 | − | ttatcattttttctcatacc | 1414 | ttctgc |
| 72 | | | | | | | tt |
| DCR | Human | DMD | Intron | − | ttgcctaagaactggtggga | 1415 | aatggt |
| 73 | | | 51 | | | | ct |
| DCR | Human | DMD | Intron | − | aaacagttgcctaagaactg | 1416 | gtggga |
| 74 | | | 51 | | | | aa |
| DCR | Human | DMD | Intron | + | tttcccaccagttcttaggc | 1417 | aactgt |
| 75 | | | 51 | | | | tt |
| DCR | Human | DMD | Intron | + | tggctttgatttccctaggg | 1418 | tccagc |
| 76 | | | 50 | | | | tt |
| DCR | Human | DMD | Intron | − | tagggaaatcaaagccaatg | 1419 | aaacgt |
| 77 | | | 50 | | | | tc |
| DCR | Human | DMD | Intron | − | gaccctagggaaatcaaagc | 1420 | caatga |
| 78 | | | 50 | | | | aa |
| DCR | Human | DMD | Intron | − | TGAGGGCTCCACCCTCACGA | 1421 | GTGGGT |
| 79 | | | 44 | | | | TT |
| DCR | Human | DMD | Intron | − | AAGGATTGAGGGCTCCACCC | 1422 | TCACGA |
| 80 | | | 44 | | | | GT |
| DCR | Human | DMD | Intron | − | GCTCCACCCTCACGAGTGGG | 1423 | TTTGGT |
| 81 | | | 44 | | | | TC |
| DCR | Human | DMD | Intron | − | TATCCCCTATCGAGGAAACC | 1424 | ACGAGT |
| 82 | | | 55 | | | | TT |
| DCR | Human | DMD | Intron | + | GATAAAGAAGGCCTATTTCA | 1425 | TAGAGT |
| 83 | | | 55 | | | | TG |
| DCR | Human | DMD | Intron | − | AGGCCTTCTTTATCCCCTAT | 1426 | CGAGGA |
| 84 | | | 55 | | | | AA |
| DCR | Human | DMD | Intron | − | TGAGGGCTCCACCCTCACGA | 1427 | GTGGGT |
| 85 | | | 44 | | | | |
| DCR | Human | DMD | Intron | + | GATAAAGAAGGCCTATTTCA | 1428 | TAGAGT |
| 86 | | | 55 | | | | |
| | | DMD | | | UAGAAGAUCUGAGCUCUGAG | 1429 | |
| | | DMD | | | AGAUCUGAGCUCUGAGUGGA | 1430 | |
| | | DMD | | | UCUGAGCUCUGAGUGGAAGG | 1431 | |
| | | DMD | | | CCGUUUACUUCAAGAGCUGA | 1432 | |
| | | DMD | | | AAGCAGCCUGACCUAGCUCC | 1433 | |
| | | DMD | | | GCUCCUGGACUGACCACUAU | 1434 | |
| | | DMD | | | CCCUCAGCUCUUGAAGUAAA | 1435 | |
| | | DMD | | | GUCAGUCCAGGAGCUAGGUC | 1436 | |
| | | DMD | | | UAGUGGUCAGUCCAGGAGCU | 1437 | |
| | | DMD | | | GCUCCAAUAGUGGUCAGUCC | 1438 | |
| | | DMD | | | UGGCCAAAGACCUCCGCCAG | 1439 | |
| | | DMD | | | GUGGCAGACAAAUGUAGAUG | 1440 | |
| | | DMD | | | UGUAGAUGUGGCAAAUGACU | 1441 | |
| | | DMD | | | CUUGGCCCUGAAACUUCUCC | 1442 | |
| | | DMD | | | CAGAGAAUAUCAAUGCCUCU | 1443 | |
| | | DMD | | | CAGAGAAUAUCAAUGCCUCU | 1444 | |
| | | DMD | | | CAUUUGUCUGCCACUGGCGG | 1445 | |
| | | DMD | | | CUACAUUUGUCUGCCACUGG | 1446 | |
| | | DMD | | | CAUCUACAUUUGUCUGCCAC | 1447 | |
| | | DMD | | | AUAAUCCCGGAGAAGUUUCA | 1448 | |
| | | DMD | | | UAUCAUCUGCAGAAUAAUCC | 1449 | |
| | | DMD | | | UGUUAUCAUGUGGACUUUUC | 1450 | |
| | | DMD | | | UGAUAUAUCAUUUCUCUGUG | 1451 | |
| | | DMD | | | UUUAUGAAUGCUUCUCCAAG | 1452 | |
| | | DMD | | | UUCUCCAGGCUAGAAGAACAA | 1453 | |
| | | DMD | | | CUGCUCUUUUCCAGGUUCAAG | 1454 | |
| | | DMD | | | GUCUGUUUCAGUUACUGGUGG | 1455 | |
| | | DMD | | | UCCAGUUUCAUUUAAUUGUUU | 1456 | |
| | | DMD | | | CUUAUGGGAGCACUUACAAGC | 1457 | |
| | | DMD | | | UUGCUUCAUUACCUUCACUGG | 1458 | |
| | | DMD | | | UUGUGUCACCAGAGUAACAGU | 1459 | |
| | | DMD | | | AGUAACCACAGGUUGUGUCAC | 1460 | |
| | | DMD | | | UUCAAAUUUUGGGCAGCGGUA | 1461 | |
| | | DMD | | | CAAGAGGCUAGAACAAUCAUU | 1462 | |
| | | DMD | | | UUGUACUUCAUCCCACUGAUU | 1463 | |
| | | DMD | | | CUUCAGAACCGGAGGCAACAG | 1464 | |
| | | DMD | | | CAACAGUUGAAUGAAAUGUUA | 1465 | |
| | | DMD | | | GCCAAGCUUGAGUCAUGGAAG | 1466 | |
| | | DMD | | | CUUGGUUUCUGUGAUUUUCUU | 1467 | |
| | | DMD | | | UCAUUUCACAGGCCUUCAAGA | 1468 | |
| | | DMD | | | CAGAAAUAUUCGUACAGUCUC | 1469 | |
| | | DMD | | | CAAUUACCUCUGGGCUCCUGG | 1470 | |
| | | DMD | | | GATACTAGGGTGGCAAATAG | 1471 | |
| | | DMD | | | GTGTTCTTAAAAGAATGGTG | 1472 | |
| | | DMD | | | GTCAAGAACAGCTGCAGAAC | 1473 | |
| | | DMD | | | GCAGTTGAATGAAATGTTAA | 1474 | |
| | | DMD | | | GATACTAGTGTGGCTCATAG | 1475 | |
| | | DMD | | | GATACGATGGTGGCAAATCG | 1476 | |
| | | DMD | | | GATACTAGGGTGGGGAATAA | 1477 | |
| | | DMD | | | TTTTTCTTAAAAGAATGGTA | 1478 | |
| | | DMD | | | TTGATCTTAGAAGAATGGTG | 1479 | |
| | | DMD | | | GTTTTCTTGAAAAAATGGTG | 1480 | |
| | | DMD | | | CTGTTCTTAAAAGGTTGGTG | 1481 | |
| | | DMD | | | GAGTTCTTCAAAGAATAGTG | 1482 | |
| | | DMD | | | TCTAGGGCAGCTGCAGAAC | 1483 | |
| | | DMD | | | TCATTCACAGCTGCAGAAC | 1484 | |
| | | DMD | | | CAAAGAATAGCTGCAGAAC | 1485 | |
| | | DMD | | | TCAAGAACAGCTGCAGCAG | 1486 | |
| | | DMD | | | TCAAGAACAGCTGCATCAC | 1487 | |
| | | DMD | | | CAGTTACATGAAATGTTAA | 1488 | |
| | | DMD | | | CATTTTAATGAAATGTTAA | 1489 | |
| | | DMD | | | AAGTTGAATGAAATTTTAA | 1490 | |
| | | DMD | | | CAGTGGAATAAAATGTTAA | 1491 | |
| | | DMD | | | AAAGATATATAATGTCATGAAT | 1492 | |
| | | DMD | | | GCAGAATCAAATATAATAGTCT | 1493 | |
| | | DMD | | | AACAAATATCCCTTAGTATC | 1494 | |
| | | DMD | | | AATGTATTTCTTCTATTCAA | 1495 | |
| | | DMD | | | AACAATAAGTCAAATTTAATTG | 1496 | |
| | | DMD | | | GAACTGGTGGGAAATGGTCTAG | 1497 | |
| | | DMD | | | TCCTTTGGTAAATAAAAGTCCT | 1498 | |
| | | DMD | | | TAGGAATCAAATGGACTTGGAT | 1499 | |
| | | DMD | | | TAATTCTTTCTAGAAAGAGCCT | 1500 | |
| | | DMD | | | CTCTTGCATCTTGCACATGTCC | 1501 | |
| | | DMD | | | ACTTAGAGGTCTTCTACATACA | 1502 | |
| | | DMD | | | TCAGAGGTGAGTGGTGAGGGGA | 1503 | |
| | | DMD | | | ACACACAGCTGGGTTATCAGAG | 1504 | |
| | | DMD | | | CACAGCTGGGTTATCAGAG | 1505 | |
| | | DMD | | | ACACAGCTGGGTTATCAGAG | 1506 | |
| | | DMD | | | CACACAGCTGGGTTATCAGAG | 1507 | |
| | | DMD | | | AACACACAGCTGGGTTATCAGAG | 1508 | |
| | | DMD | | | CTGSTGGGARATGGTCTAG | 1509 | |
| | | DMD | | | ACTGGTGGGAAATGGTCTAG | 1510 | |
| | | DMD | | | AACTGGTGGGAAATGGTCTAG | 1511 | |
| | | DMD | | | AGAACTGGTGGGAAATGGTCTAG | 1512 | |
| | | DMD | | | ATATCTTCTTAAATACCCGA | 1513 | |
| | | DMD | | | AGTCTCACAAAACTGCAGAG | 1514 | |
| | | DMD | | | TACTTATGTATTTTAAAAAC | 1515 | |
| | | DMD | | | GAATAATTTCTATTATATTACA | 1516 | |
| | | DMD | | | TTCGAAAATTTCAGGTAAGCCG | 1517 | |
| | | DMD | | | TCATTTCTAAAAGTCTTTTGCC | 1518 | |
| | | DMD | | | TTTGAGACACAGTATAGGTTAT | 1519 | |
| | | DMD | | | ATATAATAGAAATTATTCAT | 1520 | |
| | | DMD | | | TAATATGCCCTGTAATATAA | 1521 | |
| | | DMD | | | TGATATCATCAATATCTTTG | 1522 | |
| | | DMD | | | GCAATTAATTGGAAAATGTG | 1523 | |
| | | DMD | | | CTTTAAGCTTAGGTAAAATCA | 1524 | |
| | | DMD | | | CAGTAATGTGTCATACCTTC | 1525 | |
| | | DMD | | | CAGGGCATATTATATTTAGA | 1526 | |
| | | DMD | | | CAAAAGCCAAATCTATTTCA | 1527 | |
| | | DMD | | | ATGCTTTGGTGGGAAGAAGTAGAGGA | 1528 | |
| | | DMD | | | ATGCTTTGGTGGGAAGAATAGAGGAC | 1529 | |
| | | DMD | | | TTGTGACAAGCTCACTAATTAGG | 1530 | |
| | | DMD | | | AAGTTTGAAGAACTTTTACCAGG | 1531 | |
| | | DMD | | | AGGCAGCGATAAAAAAAACCTGG | 1532 | |
| | | DMD | | | GCTTTGGTGGGAAGAAGTAGAGG | 1533 | |
| | | DMD | | | GCTGGGTGTCCCATTGAAA | 1534 | |
| | | DMD | | | CAGCCGCTCGCTGCAGCAG | 1535 | |
| | | DMD | | | TGGAGAGTTTGCAAGGAGC | 1536 | |
| | | DMD | | | GTTTATTCAGCCGGGAGTC | 1537 | |
| | | DMD | | | CGCCAGGAGGGGTGGGTCTA | 1538 | |
| | | DMD | | | CCTTGGTGAGACTGGTAGA | 1539 | |
| | | DMD | | | GTCTTCAGGTTCTGTTGCT | 1540 | |
| | | DMD | | | ATATTCCTGATTTAAAAGT | 1541 | |
| | | DMD | | | TTAAAAGTCGGCTGGTAGC | 1542 | |
| | | DMD | | | CGGGCCGGGGGCGGGGTCC | 1543 | |
| | | DMD | | | GCCCGAGCCGCGTGTGGAA | 1544 | |
| | | DMD | | | CCTTCATTGCGGCGGGCTG | 1545 | |
| | | DMD | | | CCGACCCCTCCCGGGTCCC | 1546 | |
| | | DMD | | | CAGGACCGCGCTTCCCACG | 1547 | |
| | | DMD | | | TGCACCCTGGGAGCGCGAG | 1548 | |
| | | DMD | | | CCGCACGCACCTGTTCCCA | 1549 | |
| | | DMD | | | AAAACAGCGAGGGAGAAAC | 1550 | |
| | | DMD | | | TTAACTTGATTGTGAAATC | 1551 | |
| | | DMD | | | AAAACAATGCATATTTGCA | 1552 | |
| | | DMD | | | AAAATCCAGTATTTTAATG | 1553 | |
| | | DMD | | | ACCCAGCACTGCAGCCTGG | 1554 | |
| | | DMD | | | AACTTATGCGGCGTTTCCT | 1555 | |
| | | DMD | | | TCACTTTAAAACCACCTCT | 1556 | |
| | | DMD | | | GCATCTTTTTCTCTTTAAT | 1557 | |
| | | DMD | | | TGTACTCTCTGAGGTGCTC | 1558 | |
| | | DMD | | | ACGCAGATAAGAACCAGTT | 1559 | |
| | | DMD | | | CATCAAGTCAGCCATCAGC | 1560 | |
| | | DMD | | | GAGTCACCCTCCTGGAAAC | 1561 | |
| | | DMD | | | GCTAGGGATGAAGAATAAA | 1562 | |
| | | DMD | | | TTGACCAATAGCCTTGACA | 1563 | |
| | | DMD | | | TGCAAATATCTGTCTGAAA | 1564 | |
| | | DMD | | | AAATTAGCAGTATCCTCTT | 1565 | |
| | | DMD | | | CCTGGGCTCCGGGGCGTTT | 1566 | |
| | | DMD | | | GGCCCCTGCGGCCACCCCG | 1567 | |
| | | DMD | | | CTCCCTCCCTGCCCGGTAG | 1568 | |
| | | DMD | | | AGGTTTGGAAAGGGCGTGC | 1569 | |
| | | DMD | | | GATTGGCTTTGATTTCCCTA | 1570 | |
| | | DMD | | | GTGTAGAGTAAGTCAGCCTATGG | 1571 | |
| | | DMD | | | GCCTACTCAGACTGTTACTC | 1572 | |
| | | DMD | | | GTTGGACAGAACTTACCGACTGG | 1573 | |
| | | DMD | | | GCAGTTGCCTAAGAACTGGT | 1574 | |
| | | DMD | | | GGGGCTCCACCCTCACGAGT | 1575 | |
| | | DMD | | | GTTTGCTTCGCTATAAAACGAGG | 1576 | |
| | | DMD | | | GTCTGAGGATGGGGCCGCAATGG | 1577 | |
| | | DMD | | | GGATCTGTCAAATCGCCTGCAGG | 1578 | |
| | | DMD | | | GCCAGGATGGCATTGGGCAGCGG | 1579 | |
| | | DMD | | | GCTGAATCTGCGGTGGCAGGAGG | 1580 | |
| | | DMD | | | GTTCTTTTGTTCTTCTAGCCTGG | 1581 | |
| | | DMD | | | GGAAAAGCTTGAGCAAGTCAAGG | 1582 | |
| | | DMD | | | GGAAGAGTTGCCCCTGCGCCAGG | 1583 | |
| | | DMD | | | GACAAATCTCCAGTGGATAAAGG | 1584 | |
| | | DMD | | | GTGTTTCTCAGGTAAAGCTCTGG | 1585 | |
| | | DMD | | | GGAAGGACCATTTGACGTTAAGG | 1586 | |
| | | DMD | | | GAACTGCTATTTCAGTTTCCTGG | 1587 | |
| | | DMD | | | GCCAGCCACTCAGCCAGTGAAGG | 1588 | |
| | | DMD | | | GGTATGCTTTTCTGTTAAAGAGG | 1589 | |
| | | DMD | | | GCTCCTGGACTGACCACTATTGG | 1590 | |
| | | DMD | | | GGAACAGAGGCGTCCCCAGTTGG | 1591 | |
| | | DMD | | | GGAGGCTAGAACAATCATTACGG | 1592 | |
| | | DMD | | | GACAAGAACACCTTCAGAACCGG | 1593 | |
| | | DMD | | | GGGTTTCTGTGATTTTCTTTTGG | 1594 | |
| | | DMD | | | GGGCCAAAGACCTCCGCCAGTGG | 1595 | |
| | | DMD | | | GTTGGAGAAGCATTCATAAAAGG | 1596 | |
| | | DMD | | | GTCGCTCACTCACCCTGCAAAGG | 1597 | |
| | | DMD | | | GAAAAGAGCTGATGAAACAATGG | 1598 | |
| | | DMD | | | GTACACTTTTCAAAATGCTTTGG | 1599 | |
| | | DMD | | | GGAGATGATCATCAAGCAGAAGG | 1600 | |
| | | DMD | | | GCTTTGAAAGAGCAATAAAATGG | 1601 | |
| | | DMD | | | GCACAAAAGTCAAATCGGAATGG | 1602 | |
| | | DMD | | | GATTTCAATATAAGATTCGGAGG | 1603 | |
| | | DMD | | | GCTTAAGCAATCCCGAACTCTGG | 1604 | |
| | | DMD | | | GCCTTCTTTATCCCCTATCG | 1605 | |
| | | DMD | | | GAGGCCAAACCTCGGCTTACNNGRR | 1606 | |
| | | DMD | | | GTTCGAAAATTTCAGGTAAGNNGRR | 1607 | |
| | | DMD | | | GGCAGAACAGGAGATAACAGNNGRRT | 1608 | |
| | | DMD | | | GGCGGCCCTCGCCCTTCTCTGGGGAT | 1609 | |
| | | DMD | | | GTAGTGATCGTGGATACGAGAGG | 1610 | |
| | | DMD | | | GTACAGCCCTCGGTGTATATTGG | 1611 | |
| | | DMD | | | GGGAAGGAATTAAGCCCGAATGG | 1612 | |
| | | DMD | | | GGGAACAGCTTTCGTAGTTGAGG | 1613 | |
| | | DMD | | | GATAAAGTCCAGTGTCGATCAGG | 1614 | |
| | | DMD | | | GAAAACCAGAGCTTCGGTCAAGG | 1615 | |
| | | DMD | | | GGAGTCTTCTGGGCAGGCTTAAAGGCT | 1616 | |
| | | | | | AACCTGG | | |
| | | DMD | | | GTCGGGTGAGCATGTCTTTAATCTACC | 1617 | |
| | | | | | TCGATGG | | |
| | | DMD | | | GGTGTCACCAGAGTAACAGTCTGAGT | 1618 | |
| | | DMD | | | GTGATCATCAAGCAGAAGGTATGAG | 1619 | |
| | | DMD | | | GAACTTCGAAAATTTCAGGTAAGCCGA | 1620 | |
| | | | | | GG | | |
| | | DMD | | | GGAAACTCATCAAATATGCGTGTTAGT | 1621 | |
| | | | | | GT | | |
| | | DMD | | | GTCATTTACACTAACACGCATATTTGA | 1622 | |
| | | | | | TG | | |
| | | DMD | | | GGAATGAAACTCATCAAATATGCGTGT | 1623 | |
| | | | | | TA | | |
| | | DMD | | | GTCATCAATATCTTTGAAGGACTCTGG | 1624 | |
| | | | | | GT | | |
| | | DMD | | | GTGTTTTCATAGGAAAAATAGGCAAGT | 1625 | |
| | | | | | TG | | |
| | | DMD | | | GAATTGGAAAATGTGATGGGAAACAGA | 1626 | |
| | | | | | TA | | |
| | | DMD | | | GATGATCATCAAGCAGAAGGTATGAGA | 1627 | |
| | | | | | AA | | |
| | | DMD | | | GAGATGATCATCAAGCAGAAGGTATGA | 1628 | |
| | | | | | GA | | |
| | | DMD | | | GCATTTTTTCTCATACCTTCTGCTTGA | 1629 | |
| | | | | | TG | | |
| | | DMD | | | GTCCTACTCAGACTGTTACTCTGGTGA | 1630 | |
| | | | | | CA | | |
| | | DMD | | | GACAGGTTGTGTCACCAGAGTAACAGT | 1631 | |
| | | | | | CT | | |
| | | DMD | | | GTTATCATTTTTTCTCATACCTTCTGC | 1632 | |
| | | | | | TT | | |
| | | DMD | | | GTTGCCTAAGAACTGGTGGGAAATGGT | 1633 | |
| | | | | | CT | | |
| | | DMD | | | GAAACAGTTGCCTAAGAACTGGTGGGA | 1634 | |
| | | | | | AA | | |
| | | DMD | | | GTTTCCCACCAGTTCTTAGGCAACTGT | 1635 | |
| | | | | | TT | | |
| | | DMD | | | GTGGCTTTGATTTCCCTAGGGTCCAGC | 1636 | |
| | | | | | TT | | |
| | | DMD | | | GTAGGGAAATCAAAGCCAATGAAACGT | 1637 | |
| | | | | | TC | | |
| | | DMD | | | GGACCCTAGGGAAATCAAAGCCAATGA | 1638 | |
| | | | | | AA | | |
| | | DMD | | | GTGAGGGCTCCACCCTCACGAGTGGGT | 1639 | |
| | | | | | TT | | |
| | | DMD | | | GAAGGATTGAGGGCTCCACCCTCACGA | 1640 | |
| | | | | | GT | | |
| | | DMD | | | GGCTCCACCCTCACGAGTGGGTTTGGT | 1641 | |
| | | | | | TC | | |
| | | DMD | | | GTATCCCCTATCGAGGAAACCACGAGT | 1642 | |
| | | | | | TT | | |
| | | DMD | | | GGATAAAGAAGGCCTATTTCATAGAGT | 1643 | |
| | | | | | TG | | |
| | | DMD | | | GAGGCCTTCTTTATCCCCTATCGAGGA | 1644 | |
| | | | | | AA | | |
| | | DMD | | | GTGAGGGCTCCACCCTCACGAGTGGGT | 1645 | |
| | | DMD | | | GGATAAAGAAGGCCTATTTCATAGAGT | 1646 | |
| | | DMD | | | CACCGCAGCCGCTCGCTGCAGCAG | 1647 | |
| | | DMD | | | AAACCTGCTGCAGCGAGCGGCTGC | 1648 | |
| | | DMD | | | CACCGGCTGGGTGTCCCATTGAAA | 1649 | |
| | | DMD | | | AAACTTTCAATGGGACACCCAGCC | 1650 | |
| | | DMD | | | CACCGGTTTATTCAGCCGGGAGTC | 1651 | |
| | | DMD | | | AAACGACTCCCGGCTGAATAAACC | 1652 | |
| | | DMD | | | CACCGTGGAGAGTTTGCAAGGAGC | 1653 | |
| | | DMD | | | AAACGCTCCTTGCAAACTCTCCAC | 1654 | |
| | | DMD | | | CACCGCCCTCCAGACTTTCCACCT | 1655 | |
| | | DMD | | | AAACAGGTGGAAAGTCTGGAGGGC | 1656 | |
| | | DMD | | | CACCGAATTTTCTTCCAAGTTCTC | 1657 | |
| | | DMD | | | AAACGAGAACTTGGAAGAAAATTC | 1658 | |
| | | DMD | | | CACCGCTGCGGAGAGAAGAAAGGG | 1659 | |
| | | DMD | | | AAACCCCTTTCTTCTCTCCGCAGC | 1660 | |
| | | DMD | | | CACCGAGAGCCACCCCCTGGCTCC | 1661 | |
| | | DMD | | | AAACGGAGCCAGGGGGTGGCTCTC | 1662 | |
| | | DMD | | | CACCGCGAAGCCAACCGCGGCGGG | 1663 | |
| | | DMD | | | AAACCCCGCCGCGGTTGGCTTCGC | 1664 | |
| | | DMD | | | CACCGAGAGGGAAGACGATCGCCC | 1665 | |
| | | DMD | | | AAACGGGCGATCGTCTTCCCTCTC | 1666 | |
| | | DMD | | | CACCGCCCCTTTAACTTTCCTCCG | 1667 | |
| | | DMD | | | AAACCGGAGGAAAGTTAAAGGGGC | 1668 | |
| | | DMD | | | CACCGGCAGCCCCGCTTCCTTCAA | 1669 | |
| | | DMD | | | AAACTTGAAGGAAGCGGGGCTGCC | 1670 | |
| | | DMD | | | CACCGCGAGAGCGAGAGGAGGGAG | 1671 | |
| | | DMD | | | AAACCTCCCTCCTCTCGCTCTCGC | 1672 | |
| | | DMD | | | CACCGGAGAGAGCTTGAGAGCGCG | 1673 | |
| | | DMD | | | AAACCGCGCTCTCAAGCTCTCTCC | 1674 | |
| | | DMD | | | CACCGGGTGGAGGGGGCGGGGCCC | 1675 | |
| | | DMD | | | AAACGGGCCCCGCCCCCTCCACCC | 1676 | |
| | | DMD | | | CACCGGGTATCCACGTAAATCAAA | 1677 | |
| | | DMD | | | AAACTTTGATTTACGTGGATACCC | 1678 | |
| | | DMD | | | CACCGCCAATCACTGGCTCCGGTC | 1679 | |
| | | DMD | | | AAACGACCGGAGCCAGTGATTGGC | 1680 | |
| | | DMD | | | CACCGGGCGCCCGAGGGAAGAAGA | 1681 | |
| | | DMD | | | AAACTCTTCTTCCCTCGGGCGCCC | 1682 | |
| | | DMD | | | CACCGGGGTGGGGGTACCAGAGGA | 1683 | |
| | | DMD | | | AAACTCCTCTGGTACCCCCACCCC | 1684 | |
| | | DMD | | | CACCGCCGGGGACAGAAGAGAGGG | 1685 | |
| | | DMD | | | AAACCCCTCTCTTCTGTCCCCGGC | 1686 | |
| | | DMD | | | CACCGGAGAGAGAGTGGGAGAAGC | 1687 | |
| | | DMD | | | AAACGCTTCTCCCACTCTCTCTCC | 1688 | |
| | | DMD | | | CACCGAAAGTAACTGTCAAATGCG | 1689 | |
| | | DMD | | | AAACCGCATTTGACAGTTACTTTC | 1690 | |
| | | DMD | | | CACCGTTAACCAGAGCGCCCAGTC | 1691 | |
| | | DMD | | | AAACGACTGGGCGCTCTGGTTAAC | 1692 | |
| | | DMD | | | CACCGCGTCGGAGCTGCCCGCTAG | 1693 | |
| | | DMD | | | AAACCTAGCGGGCAGCTCCGACGC | 1694 | |
| | | DMD | | | TGTACTCTCTGAGGTGCTC | 1695 | |
| | | DMD | | | ACGCAGATAAGAACCAGTT | 1696 | |
| | | DMD | | | CATCAAGTCAGCCATCAGC | 1697 | |
| | | DMD | | | GAGTCACCCTCCTGGAAAC | 1698 | |
| | | DMD | | | CCTGGGCTCCGGGGCGTTT | 1699 | |
| | | DMD | | | GGCCCCTGCGGCCACCCCG | 1700 | |
| | | DMD | | | CTCCCTCCCTGCCCGGTAG | 1701 | |
| | | DMD | | | AGGTTTGGAAAGGGCGTGC | 1702 | |
| | | DMD | | | ACTCCACTGCACTCCAGTCT | 1703 | |
| | | DMD | | | TCTGTGGGGGACCTGCACTG | 1704 | |
| | | DMD | | | GGGGCGCCAGTTGTGTCTCC | 1705 | |
| | | DMD | | | ACACCATTGCCACCACCATT | 1706 | |
| | | DMD | | | CAATGACCCCTTCATTGACC | 1707 | |
| | | DMD | | | TTGATTTTGGAGGGATCTCG | 1708 | |
| | | DMD | | | GGAATCCATGGAGGGAAGAT | 1709 | |
| | | DMD | | | TGTTCTCGCTCAGGTCAGTG | 1710 | |
| | | DMD | | | CTCTCTGCTCCTTTGCCACA | 1711 | |
| | | DMD | | | GTGCTCTTCGGGTTTCAGGA | 1712 | |
| | | DMD | | | CGAAAGAGAAAGCGAACCAGTATCGAG | 1713 | |
| | | | | | AAC | | |
| | | DMD | | | CGTTGTGCATAGTCGCTGCTTGATCGC | 1714 | |
| | | DMD | | | UAGAAGAUCUGAGCUCUGAG | 1715 | |
| | | DMD | | | AGAUCUGAGCUCUGAGUGGA | 1716 | |
| | | DMD | | | UCUGAGCUCUGAGUGGAAGG | 1717 | |
| | | DMD | | | CCGUUUACUUCAAGAGCUGA | 1718 | |
| | | DMD | | | AAGCAGCCUGACCUAGCUCC | 1719 | |
| | | DMD | | | GCUCCUGGACUGACCACUAU | 1720 | |
| | | DMD | | | CCCUCAGCUCUUGAAGUAAA | 1721 | |
| | | DMD | | | GUCAGUCCAGGAGCUAGGUC | 1722 | |
| | | DMD | | | UAGUGGUCAGUCCAGGAGCU | 1723 | |
| | | DMD | | | GCUCCAAUAGUGGUCAGUCC | 1724 | |
| | | DMD | | | UGGCCAAAGACCUCCGCCAG | 1725 | |
| | | DMD | | | GUGGCAGACAAAUGUAGAUG | 1726 | |
| | | DMD | | | UGUAGAUGUGGCAAAUGACU | 1727 | |
| | | DMD | | | CUUGGCCCUCAAACUUCUCC | 1728 | |
| | | DMD | | | CAGAGAAUAUCAAUGCCUCU | 1729 | |
| | | DMD | | | CAGAGAAUAUCAAUGCCUCU | 1730 | |
| | | DMD | | | CAUUUGUCUGCCACUGGCGG | 1731 | |
| | | DMD | | | CUACAUUUGUCUGCCACUGG | 1732 | |
| | | DMD | | | CAUCUACAUUUGUCUGCCAC | 1733 | |
| | | DMD | | | AUAAUCCCGGAGAAGUUUCA | 1734 | |
| | | DMD | | | UAUCAUCUGCAGAAUAAUCC | 1735 | |
| | | DMD | | | UGUUAUCAUGUGGACUUUUC | 1736 | |
| | | DMD | | | UGAUAUAUCAUUUCUCUGUG | 1737 | |
| | | DMD | | | UUUAUGAAUGCUUCUCCAAG | 1738 | |
| | | DMD | | | UUCUCCAGGCUAGAAGAACAA | 1739 | |
| | | DMD | | | CUGCUCUUUUCCAGGUUCAAG | 1740 | |
| | | DMD | | | GUCUGUUUCAGUUACUGGUGG | 1741 | |
| | | DMD | | | UCCAGUUUCAUUUAAUUGUUU | 1742 | |
| | | DMD | | | CUUAUGGGAGCACUUACAAGC | 1743 | |
| | | DMD | | | UUGCUUCAUUACCUUCACUGG | 1744 | |
| | | DMD | | | UUGUGUCACCAGAGUAACAGU | 1745 | |
| | | DMD | | | AGUAACCACAGGUUGUGUCAC | 1746 | |
| | | DMD | | | UUCAAAUUUUGGGCAGCGGUA | 1747 | |
| | | DMD | | | CAAGAGGCUAGAACAAUCAUU | 1748 | |
| | | DMD | | | UUGUACUUCAUCCCACUGAUU | 1749 | |
| | | DMD | | | CUUCAGAACCGGAGGCAACAG | 1750 | |
| | | DMD | | | CAACAGUUGAAUGAAAUGUUA | 1751 | |
| | | DMD | | | GCCAAGCUUGAGUCAUGGAAG | 1752 | |
| | | DMD | | | CUUGGUUUCUGUGAUUUUCUU | 1753 | |
| | | DMD | | | UCAUUUCACAGGCCUUCAAGA | 1754 | |
| | | DMD | | | CAGAAAUAUUCGUACAGUCUC | 1755 | |
| | | DMD | | | CAAUUACCUCUGGGCUCCUGG | 1756 | |
| | | DMD | | | GAACUUCUAUUUAAUUUUG | 1757 | |
| | | DMD | | | AUUUCAGGUAAGCCGAGGUU | 1758 | |
| | | DMD | | | UCUUAAUAAUGUUUCACUGU | 1759 | |
| | | DMD | | | AUAAUUUCUAUUAUAUUACA | 1760 | |
| | | DMD | | | UUUCAUUCAUAUCAAGAAGA | 1761 | |
| | | DMD | | | AUAGUUUAAAGGCCAAACCU | 1762 | |
| | | DMD | | | UGUGAAAAAAUAUAGUUUAA | 1763 | |
| | | DMD | | | CGAAAAUUUCAGGUAAGCCG | 1764 | |
| | | DMD | | | CAAAAACCCAAAATATTTTAGCT | 1765 | |
| | | DMD | | | CCTTTTTGGTATCTTACAGGAAC | 1766 | |
| | | DMD | | | CCGCTGCCCAATGCCATCCTGGA | 1767 | |
| | | DMD | | | TTTTTCCTTTTATTCTAGTTGAA | 1768 | |
| | | DMD | | | TTGATCCATATGCTTTTACCTGC | 1769 | |
| | | DMD | | | TCAACAGATCTGTCAAATCGCCT | 1770 | |
| | | DMD | | | TTCTTCTTTCTCCAGGCTAGAAG | 1771 | |
| | | DMD | | | GTTCTTCTAGCCTGGAGAAAGAA | 1772 | |
| | | DMD | | | CAAATCCTGCATTGTTGCCTGTA | 1773 | |
| | | DMD | | | CTGTTAAAGAGGAAGTTAGAAGA | 1774 | |
| | | DMD | | | AAAATTTTTATATTACAGAATAT | 1775 | |
| | | DMD | | | TTGTAGACTATCTTTTATATTCT | 1776 | |
| | | DMD | | | TTTTGCATTTTAGATGAAAGAGA | 1777 | |
| | | DMD | | | AACATCTTCTCTTTCATCTAAAA | 1778 | |
| | | DMD | | | TTTTGAACATCTTCTCTTTCATC | 1779 | |
| | | DMD | | | CAAAAACCCAAAATATTTTAGCT | 1780 | |
| | | DMD | | | GCTTGTGTTTCTAATTTTTCTTT | 1781 | |
| | | DMD | | | ACTTATTGTTATTGAAATTGGCT | 1782 | |
| | | DMD | | | TACCATGTATTGCTAAACAAAGT | 1783 | |
| | | DMD | | | GTATCAATTCACACCAGCAAGTT | 1784 | |
| | | DMD | | | CTCCTCTGTAAAGTGGCGATTAT | 1785 | |
| | | DMD | | | TTTAAAATGAAGATTTTCCACCA | 1786 | |
| | | DMD | | | AAATGAAGATTTTCCACCAATCA | 1787 | |
| | | DMD | | | CCACCAATCACTTTACTCTCCTA | 1788 | |
| | | DMD | | | CCACCAGTTCTTAGGCAACTGTT | 1789 | |
| | | DMD | | | CATTAATTTATATCCTTGATTAT | 1790 | |
| | | DMD | | | GTTGTTGTTGTTAAGGTCAAAGT | 1791 | |
| | | DMD | | | AAATTACCCTAGATCTTAAAGTT | 1792 | |
| | | DMD | | | GCCTCTGATTAGGGTGGGGGCGTG | 1793 | |
| | | DMD | | | TCACAGGCTCCAGGAAGGGTTTGG | 1794 | |
| | | DMD | | | CCCAGGGGGGCCTCTTTCGGAAGG | 1795 | |
| | | DMD | | | GGAAGGCTCTCTTGGTGATGGAGA | 1796 | |
| | | DMD | | | AAGCTAGTCTAGTGCAAGCTAACA | 1797 | |
| | | DMD | | | CTGGCCTATGTTATTACCTGTATG | 1798 | |
| | | DMD | | | TGGCCTATGTTATTACCTGTATGG | 1799 | |
| | | DMD | | | TTCCATTCTAATGGGTGGCTGTT | 1800 | |
| | | DMD | | | CTCCTCTGTAAAGTGGCGAT | 1801 | |
| | | DMD | | | TTCCATTCTAATGGGTGGCT | 1802 | |
| | | DMD | | | GTATCAATTCACACCAGCAA | 1803 | |
| | | DMD | | | TACCATGTATTGCTAAACAA | 1804 | |
| | | DMD | | | ACTTATTGTTATTGAAATTG | 1805 | |
| | | DMD | | | GCTTGTGTTTCTAATTTTTC | 1806 | |
| | | DMD | | | CAAAAACCCAAAATATTTTA | 1807 | |
| | | DMD | | | TTTAAAATGAAGATTTTCCA | 1808 | |
| | | DMD | | | AAATGAAGATTTTCCACCAA | 1809 | |
| | | DMD | | | CCACCAATCACTTTACTCTC | 1810 | |
| | | DMD | | | CCACCAGTTCTTAGGCAACT | 1811 | |
| | | DMD | | | CATTAATTTATATCCTTGAT | 1812 | |
| | | DMD | | | AGTTATAGCTCTCTTTCAAT | 1813 | |
| | | DMD | | | ATGTATAACAATTCCAACAT | 1814 | |
| | | DMD | | | AAATTACCCTAGATCTTAAA | 1815 | |
| | | DMD | | | GTTGTTGTTGTTAAGGTCAA | 1816 | |
| | | DMD | | | GCTTGTGTTTCTAATTTTTC | 1817 | |
| | | DMD | | | TAATTTTTCTTTTTCTTCTT | 1818 | |
| | | DMD | | | GCAAAAAGGAAAAAAGAAGA | 1819 | |
| | | DMD | | | GGGTTTTTGCAAAAAGGAAA | 1820 | |
| | | DMD | | | AGCTCCTACTCAGACTGTTA | 1821 | |
| | | DMD | | | TGCAAAAACCCAAAATATTT | 1822 | |
| | | DMD | | | TGTCACCAGAGTAACAGTCT | 1823 | |
| | | DMD | | | CTTAGTAACCACAGGTTGTG | 1824 | |
| | | DMD | | | TAGTTTGGAGATGGCAGTTT | 1825 | |
| | | DMD | | | GAGATGGCAGTTTCCTTAGT | 1826 | |
| | | DMD | | | CTTGATGTTGGAGGTACCTG | 1827 | |
| | | DMD | | | ATGTTGGAGGTACCTGCTCT | 1828 | |
| | | DMD | | | TAACTTGATCAAGCAGAGAA | 1829 | |
| | | DMD | | | TCTGCTTGATCAAGTTATAA | 1830 | |
| | | DMD | | | TAAAATCACAGAGGGTGATG | 1831 | |
| | | DMD | | | ATATCCTCAAGGTCACCCAC | 1832 | |
| | | DMD | | | ATGATCATCTCGTTGATATC | 1833 | |
| | | DMD | | | TCATACCTTCTGCTTGATGA | 1834 | |
| | | DMD | | | TCATTTTTTCTCATACCTTC | 1835 | |
| | | DMD | | | TGCCAACTTTTATCATTTTT | 1836 | |
| | | DMD | | | AATCAGAAAGAAGATCTTAT | 1837 | |
| | | DMD | | | ATTTCCCTAGGGTCCAGCTT | 1838 | |
| | | DMD | | | GCTCAAATTGTTACTCTTCA | 1839 | |
| | | DMD | | | AGCTCCTACTCAGACTGTTA | 1840 | |
| | | DMD | | | ATTCTAGTACTATGCATCTT | 1841 | |
| | | DMD | | | ACTTAAGTTACTTGTCCAGG | 1842 | |
| | | DMD | | | CCAAGGTCCCAGAGTTCCTA | 1843 | |
| | | DMD | | | TTTCCCTGGCAAGGTCTGAA | 1844 | |
| | | DMD | | | GCTCATTCTCATGCCTGGAC | 1845 | |
| | | DMD | | | TTTAGCAATACATGGTAGAA | 1846 | |
| | | DMD | | | AGCCAAACTCTTATTCATGA | 1847 | |
| | | DMD | | | TAACAATGTGGATACTTTGT | 1848 | |
| | | DMD | | | GUGUUAUUACUUGCUACUGCA | 1849 | |
| | | DMD | | | GUGUAUUGCUUGUACUACUCA | 1850 | |
| | | DMD | | | GUUUAAAUGUAAAUAGCUCAG | 1851 | |
| | | DMD | | | GAAUUUUCAAUGAUGUUCUGGG | 1852 | |
| | | DMD | | | GAACUGGUGGGAAAUGGUCUAG | 1853 | |
| | | DMD | | | GUUUCAUUGGCUUUGAUUUCCC | 1854 | |
| | | DMD | | | GGCAAUUCUCCUGAAUAGAAA | 1855 | |
| | | DMD | | | GAUUAUACUUAGGCUGAAUAGU | 1856 | |
| | | DMD | | | GACUUCCAGAAUUAUGUGUUC | 1857 | |
| | | DMD | | | GUGAGGGCCUGACACAUGGUA | 1858 | |
| | | DMD | | | GUGAAGAUCAUUUCUUGGUAG | 1859 | |
| | | DMD | | | GCACAGUCAGAACUAGUGUGC | 1860 | |
| | | DMD | | | GAGUAAGCCCGAUCAUUAUUG | 1861 | |
| | | DMD | | | GGAAGGGACAUAUUCUAUGGG | 1862 | |
| | | DMD | | | GACCACAAGCUGACUUGGGGG | 1863 | |
| | | DMD | | | GGAUUUGUAUCCAUUAUCUGG | 1864 | |
| | | DMD | | | CUCUGCAUUGUUUUGGCCUC | 1865 | |
| | | DMD | | | UCCUCCAAAGAGUAGAAUGG | 1866 | |
| | | DMD | | | GCCCUAAACUUACACUGUUC | 1867 | |
| | | DMD | | | AAAGAUAGAUUAGAUUGUCC | 1868 | |
| | | DMD | | | GUUGCUAAAUUACAUAGUUU | 1869 | |
| | | DMD | | | UGUUGCAAUAGUCAAUCAAG | 1870 | |
| | | DMD | | | AUACUGAUUAAGACAGAUGA | 1871 | |
| | | DMD | | | AAUACUGAUUAAGACAGAUG | 1872 | |
| | | DMD | | | CUCUAUACAAAUGCCAACGC | 1873 | |
| | | DMD | | | ACUUGCAUGCACACCAGCGU | 1874 | |
| | | DMD | | | UUGGGCUAAUGUAGCAUAAU | 1875 | |
| | | DMD | | | GCGUUGGCAUUUGUAUAGAG | 1876 | |
| | | DMD | | | UGGGCUAAGUAGCAUAAUG | 1877 | |
| | | DMD | | | UUUGGGCUAAUGUAGCAUAA | 1878 | |
| | | DMD | | | GCUUAACUCCUUAAUAUUAA | 1879 | |
| | | DMD | | | UCUUCUAUAUUAAAGCAGAU | 1880 | |
| | | DMD | | | CUUCUAUAUUAAAGCAGAUU | 1881 | |
| | | DMD | | | AAUAUAUAACUACCUUGGGU | 1882 | |
| | | DMD | | | ACCUCCAUUCUACUCUUUGG | 1883 | |
| | | DMD | | | UUUCAAUGAUAUCCAACCCA | 1884 | |
| | | DMD | | | AGUACCUCCAUUCUACUCUU | 1885 | |
| | | DMD | | | CUAUCCUCCATAGAGUAGAA | 1886 | |
| | | DMD | | | UUUUGCUACAUAUUUCAGGC | 1887 | |
| | | DMD | | | UUUGCUACAUAUUUCAGGCU | 1888 | |
| | | DMD | | | GGGUUGGAUAUCAUUGAAAA | 1889 | |
| | | DMD | | | AUAUUUCAGGCUGGGUUCU | 1890 | |
| | | DMD | | | UUGAAAUAUAUAACUACCUU | 1891 | |
| | | DMD | | | AUUGAAAUAUAUAACUACCU | 1892 | |
| | | DMD | | | GUGAGUAGUGGGGCACUUUA | 1893 | |
| | | DMD | | | UGUAUGUAGAAGGUUAACUA | 1894 | |
| | | DMD | | | GAGCCUAAUAAAUGUACAAU | 1895 | |
| | | DMD | | | UUGUAUGUAGAAGGUUAACU | 1896 | |
| | | DMD | | | CAAUUUGUUUUGAGUAACU | 1897 | |
| | | DMD | | | UGCCUUCUGAAAUAGUCCAG | 1898 | |
| | | DMD | | | GUUAAUAGGGAAACAGCAUA | 1899 | |
| | | DMD | | | AACAAUGCAGAGUUAAUUGU | 1900 | |
| | | DMD | | | GAACAUGUUGAGUAGACACA | 1901 | |
| | | DMD | | | UUUAUCAUCUGUGUCUAUUC | 1902 | |
| | | DMD | | | UCUUUACUUUCUUGACUAUA | 1903 | |
| | | DMD | | | AAUAUUCUCAAACCUCGUUC | 1904 | |
| | | DMD | | | AUUAACUGUGUUCCAGAACG | 1905 | |
| | | DMD | | | UAACUGCUUCUUUGGAUGAC | 1906 | |
| | | DMD | | | GACCAGAACAGUGUAAGUUU | 1907 | |
| | | DMD | | | ACCAGAACAGUGUAAGUUUA | 1908 | |
| | | DMD | | | CUACUUUUUCCCCACUACUG | 1909 | |
| | | DMD | | | UGGAACACAGUUAAUUCACU | 1910 | |
| | | DMD | | | GUGUUGUUUAACUGCUUCUU | 1911 | |
| | | DMD | | | AACUGUCAGUUGCAUAUUCC | 1912 | |
| | | DMD | | | CAGAAAGGAAUGCUGGUACC | 1913 | |
| | | DMD | | | UCUGCCUACACAAUGAAUGG | 1914 | |
| | | DMD | | | CACAGAUCAAUCCAAUUGUU | 1915 | |
| | | DMD | | | UUGACAGGUGGAAAGUACAU | 1916 | |
| | | DMD | | | ACAUUUUUAGGCUUGACAGG | 1917 | |
| | | DMD | | | CUCUCCCAUGACAGACUCCC | 1918 | |
| | | DMD | | | UUGGUAAGAGUUAUGAUAAG | 1919 | |
| | | DMD | | | AACACAAAUUAAGUUCACCU | 1920 | |
| | | DMD | | | AGGAUCAGUGCUGUAGUGCC | 1921 | |
| | | DMD | | | GGCCGUUUAUUAUUAUUGAC | 1922 | |
| | | DMD | | | UCUCAGGAUUGCUAUGCAAC | 1923 | |
| | | DMD | | | CAGGAAGACAUACCAUGUAA | 1924 | |
| | | DMD | | | AGCAGGGCUCUUUCAGUUUC | 1925 | |
| | | DMD | | | UAACAUUUUCAGCUUGAACC | 1926 | |
| | | DMD | | | UCAAGCUGAAAAUGUUACAC | 1927 | |
| | | DMD | | | GUAACAUUUUCAGCUUGAAC | 1928 | |
| | | DMD | | | CAGAAUGAAUUUUGGAGCAC | 1929 | |
| | | DMD | | | UUUAUUAUUAUUGACUGGUG | 1930 | |
| | | DMD | | | AGAAGAAUCUGACCUUUACA | 1931 | |
| | | DMD | | | GCAGGGCUCUUUCAGUUUCU | 1932 | |
| | | DMD | | | CUAAACAGUAGCCAGGCGUG | 1933 | |
| | | DMD | | | CGCCUGGCUACUGUUUAGUG | 1934 | |
| | | DMD | | | CUCCGCACUAAACAGUAGCC | 1935 | |
| | | DMD | | | GUAGCCAGGCGUGUGGAUGU | 1936 | |
| | | DMD | | | CUUGGCUUUGACUAUUCUGC | 1937 | |
| | | DMD | | | AGUAGCCAGGCGUGUGGAUG | 1938 | |
| | | DMD | | | UCCUCCCACAUCCACACGCC | 1939 | |
| | | DMD | | | UUGGCUUUGACUAUUCUGCU | 1940 | |
| | | DMD | | | AUAAUGUCUCUGGCUUGUAA | 1941 | |
| | | DMD | | | UGGUACCCGGCAGCUCUCUG | 1942 | |
| | | DMD | | | GUGGGAGGAACCUCAAAGAG | 1943 | |
| | | DMD | | | UGACUAUUCUGCUGGGAACA | 1944 | |
| | | DMD | | | CUCUCUGAGGAAUGUUCCCU | 1945 | |
| | | DMD | | | AACAUUCCUCAGAGAGCUGC | 1946 | |
| | | DMD | | | AUUCUGAAGCUCCAAACAAU | 1947 | |
| | | DMD | | | UAAAUUACUCUGCUAAAGUA | 1948 | |
| | | DMD | | | AGUACAAACCAGGUUUGUAC | 1949 | |
| | | DMD | | | AUAUCCUUCCAGUACAAACC | 1950 | |
| | | DMD | | | CAAACCAGGUUUGUACUGGA | 1951 | |
| | | DMD | | | GGCAGCUAAAGCAUCACUGA | 1952 | |
| | | DMD | | | AUGUCUGAGUAGUACAAACC | 1953 | |
| | | DMD | | | GUGUCCCAUUCUCUUUGACU | 1954 | |
| | | DMD | | | UGUGUCCCAUUCUCUUUGAC | 1955 | |
| | | DMD | | | UUCUGAAUGUUGAACAAGUA | 1956 | |
| | | DMD | | | GUCUCCCAGUCAAAGAGAAU | 1957 | |
| | | DMD | | | AUUCUCUUUGACUGGGAGAC | 1958 | |
| | | DMD | | | UCUUUGACUGGGAGACAGGC | 1959 | |
| | | DMD | | | GUGGUGUCCUUUGAAUAUGC | 1960 | |
| | | DMD | | | AGAUUGUCCAGGAUAUAAUU | 1961 | |
| | | DMD | | | UUAGCAACCAAAUUAUAUCC | 1962 | |
| | | DMD | | | GUUGAAAUUAAACUACACAC | 1963 | |
| | | DMD | | | AUCUUUACCUGCAUAUUCAA | 1964 | |
| | | DMD | | | GUGUCCUUUGAAUAUGC | 1965 | |
| | | DMD | | | UUGUCCAGGAUAUAAUU | 1966 | |
| | | DMD | | | GCAACCAAAUUAUAUCC | 1967 | |
| | | DMD | | | GAAAUUAAACUACACAC | 1968 | |
| | | DMD | | | UUUACCUGCAUAUUCAA | 1969 | |
| | | DMD | | | UACACAUUUUUAGGCUUGAC | 1970 | |
| | | DMD | | | CAUUCCUGGGAGUCUGUCAU | 1971 | |
| | | DMD | | | UGUAUGAUGCUAUAAUACCA | 1972 | |
| | | DMD | | | GUGGAAAGUACAUAGGACCU | 1973 | |
| | | DMD | | | UCUUAUCAUAACUCUUACCA | 1974 | |
| | | DMD | | | ACAUUUUUAGGCUUGAC | 1975 | |
| | | DMD | | | UCCUGGGAGUCUGUCAU | 1976 | |
| | | DMD | | | AUGAUGCUAUAAUACCA | 1977 | |
| | | DMD | | | GAAAGUACAUAGGACCU | 1978 | |
| | | DMD | | | UAUCAUAACUCUUACCA | 1979 | |
| | | DMD | | | GAGTTCCTACTCAGACTGTTACTC | 1980 | |
| | | DMD | | | GTGAGTTCCTACTCAGACTGTTACTC | 1981 | |
| | | DMD | | | GTCTGAGTTCCTACTCAGACTGTTACT | 1982 | |
| | | | | | C | | |
| | | DMD | | | AAAGATATATAATGTCATGAAT | 1983 | |
| | | DMD | | | GCAGAATCAAATATAATAGTCT | 1984 | |
| | | DMD | | | AACAAATATCCCTTAGTATC | 1985 | |
| | | DMD | | | AATGTATTTCTTCTATTCAA | 1986 | |
| | | DMD | | | AACAATAAGTCAAATTTAATTG | 1987 | |
| | | DMD | | | GAACTGGTGGGAAATGGTCTAG | 1988 | |
| | | DMD | | | TCCTTTGGTAAATAAAAGTCCT | 1989 | |
| | | DMD | | | TAGGAATCAAATGGACTTGGAT | 1990 | |
| | | DMD | | | TAATTCTTTCTAGAAAGAGCCT | 1991 | |
| | | DMD | | | CTCTTGCATCTTGCACATGTCC | 1992 | |
| | | DMD | | | ACTTAGAGGTCTTCTACATACA | 1993 | |
| | | DMD | | | TCAGAGGTGAGTGGTGAGGGGA | 1994 | |
| | | DMD | | | ACACACAGCTGGGTTATCAGAG | 1995 | |
| | | DMD | | | CACAGCTGGGTTATCAGAG | 1996 | |
| | | DMD | | | ACACAGCTGGGTTATCAGAG | 1997 | |
| | | DMD | | | CACACAGCTGGGTTATCAGAG | 1998 | |
| | | DMD | | | AACACACAGCTGGGTTATCAGAG | 1999 | |
| | | DMD | | | CTGSTGGGARATGGTCTAG | 2000 | |
| | | DMD | | | ACTGGTGGGAAATGGTCTAG | 2001 | |
| | | DMD | | | AACTGGTGGGAAATGGTCTAG | 2002 | |
| | | DMD | | | AGAACTGGTGGGAAATGGTCTAG | 2003 | |
| | | DMD | | | ATATCTTCTTAAATACCCGA | 2004 | |
| | | DMD | | | AGTCTCACAAAACTGCAGAG | 2005 | |
| | | DMD | | | TACTTATGTATTTTAAAAAC | 2006 | |
| | | DMD | | | GAATAATTTCTATTATATTACA | 2007 | |
| | | DMD | | | TTCGAAAATTTCAGGTAAGCCG | 2008 | |
| | | DMD | | | TCATTTCTAAAAGTCTTTTGCC | 2009 | |
| | | DMD | | | TTTGAGACACAGTATAGGTTAT | 2010 | |
| | | DMD | | | ATATAATAGAAATTATTCAT | 2011 | |
| | | DMD | | | TAATATGCCCTGTAATATAA | 2012 | |
| | | DMD | | | TGATATCATCAATATCTTTG | 2013 | |
| | | DMD | | | GCAATTAATTGGAAAATGTG | 2014 | |
| | | DMD | | | CTTTAAGCTTAGGTAAAATCA | 2015 | |
| | | DMD | | | CAGTAATGTGTCATACCTTC | 2016 | |
| | | DMD | | | CAGGGCATATTATATTTAGA | 2017 | |
| | | DMD | | | CAAAAGCCAAATCTATTTCA | 2018 | |
| | | DMD | | | ATGCTTTGGTGGGAAGAAGTAGAGGA | 2019 | |
| | | DMD | | | ATGCTTTGGTGGGAAGAATAGAGGAC | 2020 | |
| | | DMD | | | TTGTGACAAGCTCACTAATTAGG | 2021 | |
| | | DMD | | | AAGTTTGAAGAACTTTTACCAGG | 2022 | |
| | | DMD | | | AGGCAGCGATAAAAAAAACCTGG | 2023 | |
| | | DMD | | | GCTTTGGTGGGAAGAAGTAGAGG | 2024 | |

### Scaffold

The scaffold sequence is the sequence within the gRNA that is responsible for nuclease (e.g., Cas9) binding. The scaffold sequence does not include the spacer/targeting sequence.

In some embodiments, the scaffold may be about 60 to about 70, about 70 to about 80, about 80 to about 90, about 90 to about 100, about 100 to about 110, about 110 to about 120, or about 120 to about 130 nucleotides in length. In some embodiments, the scaffold may be about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, about 99, about 100, about 101, about 102, about 103, about 104, about 105, about 106, about 107, about 108, about 109, about 110, about 111, about 112, about 113, about 114, about 115, about 116, about 117, about 118, about 119, about 120, about 121, about 122, about 123, about 124, or about 125 nucleotides in length. In some embodiments, the scaffold may be at least 60, at least 70, at least 80, at least 90, at least 100, at least 110, at least 120, or at least 125 nucleotides in length. In some embodiments, the scaffold may be 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, or 125 nucleotides in length.

In some embodiments, the scaffold may have a sequence of any one of SEQ ID NO: 2357-2362 or 2348 (shown in Table 16 below), or a sequence that is at least 90%, at least 95%, at least 96%, at least 97%, at least 98%, or at least 99% identical thereto.

TABLE 16
Exemplary Scaffold Sequences

| | SEQ ID |
|---|---|
| Sequence | NO: |
| GTTTAAGAGCTATGCTGGAAACAGCATAGCAAGTTTAAATAAGGCTAGTCCGTTATCA | 2348 |
| ACTTGAAAAAGTGGCACCGAGTCGGTGCTTTTTTT | |
| GTTTTAGAGCTAGAAATAGCAGTTAAAATAAGGCTAGTCCGTTATCAACTTGAAAAAG | 2357 |
| TGGCACCGAGTCGGTG | |
| GTTGGAACCATTCAAAACAGCATAGCAAGTTAAAATAAGGCTAGTCCGTTATCAACTT | 2358 |
| GAAAAAGTGGCACCGAGTCGGTGCTTTTTT | |
| GTTTAAGAGCTATGAAACAGCATAGCAAGTTTAAATAAGGCTAGTCCGTTATCAACTT | 2359 |
| GAAAAAGTGGCACCGAGTCGGTGCTTTTTTT | |
| GTTTAAGAGCTATGCGAAACAGCATAGCAAGTTTAAATAAGGCTAGTCCGTTATCAAC | 2360 |
| TTGAAAAAGTGGCACCGAGTCGGTGCTTTTTTT | |
| GTTTAAGAGCTATGCTGTTTGAAACAGCATAGCAAGTTTAAATAAGGCTAGTCCGTTA | 2361 |
| TCAACTTGAAAAAGTGGCACCGAGTCGGTGCTTTTTTT | |
| GTTTAAGAGCTATGCTGTTTTGGAAACAGCATAGCAAGTTTAAATAAGGCTAGTCCGT | 2362 |
| TATCAACTTGAAAAAGTGGCACCGAGTCGGTGCTTTTTTT | |

In some embodiments, the scaffold sequence is selected from any one of SEQ ID NO: 2357-2362 or 2348.

In some embodiments, a gRNA (spacer+scaffold) comprises a scaffold and a spacer as shown in Table 17, wherein “X” indicates that the particular combination is contemplated by the instant disclosure.

TABLE 17
Exemplary sgRNA (spacer + scaffold) sequences

| | Scaffold | Scaffold | Scaffold | Scaffold | Scaffold | Scaffold | Scaffold |
|---|---|---|---|---|---|---|---|
| | sequence | sequence | sequence | sequence | sequence | sequence | sequence |
| Spacer | (SEQ ID NO: | (SEQ ID NO: | (SEQ ID NO: | (SEQ ID NO: | (SEQ ID NO: | (SEQ ID NO: | (SEQ ID NO: |
| Sequence | 2348) | 2357) | 2358) | 2359) | 2360) | 2361) | 2362) |
| Any DMD | X | X | X | X | X | X | X |
| Spacer of | | | | | | | |
| the | | | | | | | |
| disclosure | | | | | | | |
| Any one of | X | X | X | X | X | X | X |
| DMD | | | | | | | |
| Spacers | | | | | | | |
| SEQ ID | | | | | | | |
| NOs: 13, 29- | | | | | | | |
| 2024 | | | | | | | |
| DMD | X | X | X | X | X | X | X |
| Spacer SEQ | | | | | | | |
| ID NO: 13 | | | | | | | |
| DMD | X | X | X | X | X | X | X |
| Spacer SEQ | | | | | | | |
| ID NO: 929 | | | | | | | |

### Expression Control Sequences

The AAV expression cassettes of the disclosure may comprise one or more expression control sequences. Typically, expression control sequences are nucleic acid sequence(s) that influence expression of an operably linked polynucleotide, such as a promoter, an enhancer, a splicing signal, etc. Such elements typically act in cis but may also act in trans. Expression control sequences may be constitutive or inducible.

Exemplary promoters which may be used in the AAV expression cassettes of the disclosure include naturally occurring and synthetic promoters. In some embodiments, the promoter is selected from any one of the following promoters, or derivatives thereof: Chicken beta actin (CBA) promoter, cytomegalovirus immediate early (CMV) promoter, ubiquitin C (UBC) promoter, elongation factor 1-alpha promoter (EF1-alpha) promoter, a phosphoglycerate kinase-1 promoter (PGK) promoter, a chimeric liver-specific promoter (LSP), a cytomegalovirus enhancer/chicken beta-actin promoter (CAG), a tetracycline responsive promoter (TRE), a transthyretin promoter (TTR), a simian virus 40 promoter (SV40) and a CK6 promoter.

In some embodiments, the promoter is the U6 promoter, which has the sequence shown below (SEQ ID NO: 15), or a sequence at least 95% or at least 99% identical thereto:

| | |
|---|---|
| 1 | CGAGTCCAAC ACCCGTGGGA ATCCCATGGG CACCATGGCC |
| | CCTCGCTCCA AAAATGCTTT |
| 61 | CGCGTCGCGC AGACACTGCT CGGTAGTTTC GGGGATCAGC |
| | GTTTGAGTAA GAGCCCGCGT |
| 121 | CTGAACCCTC CGCGCCGCCC CGGCCCCAGT GGAAAGACGC |
| | GCAGGCAAAA CGCACCACGT |
| 181 | GACGGAGCGT GACCGCGCGC CGAGCGCGCG CCAAGGTCGG |
| | GCAGGAAGAG GGCCTATTTC |
| 241 | CCATGATTCC TTCATATTTG CATATACGAT ACAAGGCTGT |
| | TAGAGAGATA ATTAGAATTA |
| 301 | ATTTGACTGT AAACACAAAG ATATTAGTAC AAAATACGTG |
| | ACGTAGAAAG TAATAATTTC |
| 361 | TTGGGTAGTT TGCAGTTTTA AAATTATGTT TTAAAATGGA |
| | CTATCATATG CTTACCGTAA |
| 421 | CTTGAAAGTA TTTCGATTTC TTGGCTTTAT ATATCTTGTG |
| | GAAAGGACGA AA |

In some embodiments, the promoter is the H1 promoter, which has the sequence shown below (SEQ ID NO: 16), or a sequence at least 95% or at least 99% identical thereto:

| | |
|---|---|
| 1 | CTTCGGCGCG CCCATATTTG CATGTCGCTA TGTGTTCTGG |
| | GAAATCACCA TAAACGTGAA |
| 61 | ATGTCTTTGG ATTTGGGAAT CTTATAAGTT CTGTATGAGA |
| | CCACGGTA |

In some embodiments, the promoter is the 7SK promoter, which has the sequence shown below (SEQ ID NO: 17), or a sequence at least 95% or at least 99% identical thereto:

| | |
|---|---|
| 1 | TGACGGCGCG CCCTGCAGTA TTTAGCATGC CCCACCCATC |
| | TGCAAGGCAT TCTGGATAGT |
| 61 | GTCAAAACAG CCGGAAATCA AGTCCGTTTA TCTCAAACTT |
| | TAGCATTTTG GGAATAAATG |
| 121 | ATATTTGCTA TGCTGGTTAA ATTAGATTTT AGTTAAATTT |
| | CCTGCTGAAG CTCTAGTACG |
| 181 | ATAAGTAACT TGACCTAAGT GTAAAGTTGA GATTTCCTTC |
| | AGGTTTATAT AGCTTGTGCG |
| 241 | CCGCCTGGGT A |

In some embodiments, the naturally-occurring or synthetic promoter may be a tissue-specific promoter. The tissue-specific promoter may be specific, for example, to the liver, brain, central nervous system, spinal cord, eye, retina, bone, muscle (including cardiac, skeletal, and and/or smooth muscle), lung, pancreas, heart, kidney, etc.

In some embodiments, the promoter is muscle-specific. The muscle-specific promoter may be for example, a myosin light chain (MLC) promoter, for example MLC2 (Gene ID No. 4633; representative GenBank Accession No. NG_007554.1); a myosin heavy chain (MHC) promoter, for example alpha-MHC (Gene ID No. 4624; representative GenBank Accession No. NG_023444.1); a desmin promoter (Gene ID No. 1674; representative GenBank Accession No. NG_008043.1); a cardiac troponin C promoter (Gene ID No. 7134; representative GenBank Accession No. NG_008963.1); a troponin I promoter (Gene ID Nos. 7135, 7136, and 7137: representative GenBank Accession Nos. NG_016649.1, NG_011621.1, and NG_007866.2); a myoD gene family promoter (Gene ID No. 4654; representative GenBank Accession No. NM_002478); an actin alpha promoter (Gene ID Nos. 58, 59, and 70; representative GenBank Accession Nos. NG_006672.1, NG_011541.1, and NG_007553.1); an actin beta promoter (Gene ID No. 60; representative GenBank Accession No. NG_007992.1); an actin gamma promoter (Gene ID No. 71 and 72; representative GenBank Accession No. NG_011433.1 and NM_001199893); a muscle-specific promoter residing within intron 1 of the ocular form of Pitx3 (Gene ID No. 5309) (the muscle-specific promoter corresponds to residues 11219-11527 of representative GenBank Accession No. NG_008147). In some embodiments, the promoter may be a muscle-specific creatine kinase 8 promoter (e.g., CK8 or CK8e).

In some embodiments, the muscle-specific promoter is the CK8 promoter. The CK8 promoter has the following sequence (SEQ ID NO: 18):

| | |
|---|---|
| 1 | CTAGACTAGC ATGCTGCCCA TGTAAGGAGG CAAGGCCTGG |
| | GGACACCCGA GATGCCTGGT |
| 61 | TATAATTAAC CCAGACATGT GGCTGCCCCC CCCCCCCCAA |
| | CACCTGCTGC CTCTAAAAAT |
| 121 | AACCCTGCAT GCCATGTTCC CGGCGAAGGG CCAGCTGTCC |
| | CCCGCCAGCT AGACTCAGCA |
| 181 | CTTAGTTTAG GAACCAGTGA GCAAGTCAGC CCTTGGGGCA |
| | GCCCATACAA GGCCATGGGG |
| 241 | CTGGGCAAGC TGCACGCCTG GGTCCGGGGT GGGCACGGTG |
| | CCCGGGCAAC GAGCTGAAAG |
| 301 | CTCATCTGCT CTCAGGGGCC CCTCCCTGGG GACAGCCCCT |
| | CCTGGCTAGT CACACCCTGT |
| 361 | AGGCTCCTCT ATATAACCCA GGGGCACAGG GGCTGCCCTC |
| | ATTCTACCAC CACCTCCACA |
| 421 | GCACAGACAG ACACTCAGGA GCCAGCCAGC. |

In some embodiments, the muscle-specific specific promoter is a variant of the CK8 promoter, called CK8e. The CK8e promoter has the following sequence (SEQ ID NO. 19):

| | |
|---|---|
| 1 | TGCCCATGTA AGGAGGCAAG GCCTGGGGAC ACCCGAGATG |
| | CCTGGTTATA ATTAACCCAG |
| 61 | ACATGTGGCT GCCCCCCCCC CCCCAACACC TGCTGCCTCT |
| | AAAAATAACC CTGCATGCCA |
| 121 | TGTTCCCGGC GAAGGGCCAG CTGTCCCCCG CCAGCTAGAC |
| | TCAGCACTTA GTTTAGGAAC |
| 181 | CAGTGAGCAA GTCAGCCCTT GGGGCAGCCC ATACAAGGCC |
| | ATGGGGCTGG GCAAGCTGCA |
| 241 | CGCCTGGGTC CGGGGTGGGC ACGGTGCCCG GGCAACGAGC |
| | TGAAAGCTCA TCTGCTCTCA |
| 301 | GGGGCCCCTC CCTGGGGACA GCCCCTCCTG GCTAGTCACA |
| | CCCTGTAGGC TCCTCTATAT |
| 361 | AACCCAGGGG CACAGGGGCT GCCCTCATTC TACCACCACC |
| | TCCACAGCAC AGACAGACAC |
| 421 | TCAGGAGCCA GCCAGC. |

In some embodiments, the AAV expression cassettes of the disclosure may comprise multiple expression control sequences, such as two, three, four, five, or more. In some embodiments, the AAV expression cassettes of the disclosure comprise two, three, four, five, or more promoters, enhancers, or combinations thereof. In some embodiments, the promoters/enhancers are the same. In other embodiments, the promoters/enhancers are different.

In some embodiments, the AAV expression cassettes comprise two promoters, wherein each promoter is independently selected from the U6 promoter, the H1 promoter, and the 7SK promoter. In some embodiments, the AAV expression cassettes comprise three promoters, wherein each promoter is independently selected from the U6 promoter, the H1 promoter, and the 7SK promoter. In some embodiments, the AAV expression cassettes comprise four or more promoters, wherein each promoter is independently selected from the U6 promoter, the H1 promoter, and the 7SK promoter.

### Filler or Stuffer Sequence

In some embodiments, the AAV expression cassette of the disclosure may comprise a filler or stuffer sequence. As used herein, a “filler” or “stuffer” sequence refers to a sequence that is inert or innocuous and has no function or activity. In some embodiments, a filler or stuffer polynucleotide sequence is not a bacterial polynucleotide sequence. In some embodiments, a filler or stuffer polynucleotide sequence is not a sequence that encodes a protein or peptide, a filler or stuffer polynucleotide sequence is a sequence distinct from any of: a gRNA, an AAV inverted terminal repeat (ITR) sequence, an expression control element (e.g., a promoter), an origin of replication, a selectable marker or a poly-Adenine (poly-A) sequence.

In some embodiments, a filler or stuffer sequence is an intron sequence that is related to or unrelated to the transgene sequence. In particular aspects, the intron sequence is positioned within the transgene sequence.

In some embodiments, the filler or stuffer sequence does not contain any open reading frames (ORFs). In some embodiments, the filler or stuffer sequence is isolated or derived from a 5′ or 3′ UTR sequence. In some embodiments, the filler or stuffer sequence is a 5′ or a 3′ UTR sequence which doesn't contain any ORFs. In some embodiments, the filler or stuffer sequence is isolated or derived from a gene expressed in muscle, such as desmin. In some embodiments, the filler or stuffer sequence is derived from a gene expressed in muscle, such as desmin, wherein all ORFs have been removed.

In some embodiments, the filler or stuffer sequence comprises a 3′ UTR sequence isolated or derived from a gene expressed in muscle. In some embodiments, a filler or stuffer sequence comprises or consists of the sequence of SEQ ID NO: 20, or a sequence at least 95%, at least 96%, at least 97%, at least 98%, or at least 99% identical thereto.

### AAV Nuclease Vector

In some embodiments, and AAV expression cassette may be used to produce an AAV vector comprising a nuclease. In some embodiments, the AAV expression cassette comprises sequences encoding a first ITR, a nuclease, and a second ITR. In some embodiments, the AAV expression cassette comprises sequences encoding a first ITR, a first promoter, a nuclease, and a second ITR. In some embodiments, the AAV expression cassette comprises sequences encoding a first ITR (e.g., a sequence with at least 95% or 100% identity to SEQ ID NO: 1), a first promoter, a nuclease, and a second ITR (e.g., a sequence with at least 95% or 100% identity to SEQ ID NO: 2). In some embodiments, the AAV expression cassette comprises sequences encoding a first ITR (e.g., a sequence with at least 95% or 100% identity to SEQ ID NO: 3), a first promoter, a nuclease, and a second ITR (e.g., a sequence with at least 95% or 100% identity to SEQ ID NO: 2). In some embodiments, the nuclease sequence is optimized for expression in mammalian cells. In some embodiments, the promoter drives expression of the nuclease in mammalian cells.

In some embodiments, the nuclease is a Type II, Type V-A, Type V-B, Type V-C, Type V-U, Type VI-B nuclease. In some embodiments, the nuclease is a Cas9, Cas12a, Cas12b, Cas12c, Tnp-B like, Cas13a (C2c2), or Cas13b nuclease. In some embodiments, the nuclease is a TAL nuclease, a meganuclease, or a zinc-finger nuclease. In some embodiments, the nuclease is a Cas9 nuclease. In some embodiments, the nuclease is a Cpf1 nuclease.

In embodiments, the nuclease is a Cas9 nuclease derived from S. pyogenes (SpCas9). An exemplary SpCas9 sequence is provided below (SEQ ID NO: 21):

| | |
|---|---|
| 1 | MDKKYSIGLD IGTNSVGWAV ITDEYKVPSK KFKVLGNTDR |
| | HSIKKNLIGA LLFDSGETAE |
| 61 | ATRLKRTARR RYTRRKNRIC YLQEIFSNEM AKVDDSFFHR |
| | LEESFLVEED KKHERHPIFG |
| 121 | NIVDEVAYHE KYPTIYHLRK KLVDSTDKAD LRLIYLALAH |
| | MIKFRGHFLI EGDLNPDNSD |
| 181 | VDKLFIQLVQ TYNQLFEENP INASGVDAKA ILSARLSKSR |
| | RLENLIAQLP GEKKNGLFGN |
| 241 | LIALSLGLTP NFKSNFDLAE DAKLQLSKDT YDDDLDNLLA |
| | QIGDQYADLF LAAKNLSDAI |
| 301 | LLSDILRVNT EITKAPLSAS MIKRYDEHHQ DLTLLKALVR |
| | QQLPEKYKEI FFDQSKNGYA |
| 361 | GYIDGGASQE EFYKFIKPIL EKMDGTEELL VKLNREDLLR |
| | KQRTFDNGSI PHQIHLGELH |
| 421 | AILRRQEDFY PFLKDNREKI EKILTFRIPY YVGPLARGNS |
| | RFAWMTRKSE ETITPWNFEE |
| 481 | VVDKGASAQS FIERMINFDK NLPNEKVLPK HSLLYEYFTV |
| | YNELTKVKYV TEGMRKPAFL |
| 541 | SGEQKKAIVD LLFKTNRKVT VKQLKEDYFK KIECFDSVEI |
| | SGVEDRFNAS LGTYHDLLKI |
| 601 | IKDKDFLDNE ENEDILEDIV LTLTLFEDRE MIEERLKTYA |
| | HLFDDKVMKQ LKRRRYTGWG |
| 661 | RLSRKLINGI RDKQSGKTIL DFLKSDGFAN RNFMQLIHDD |
| | SLTFKEDIQK AQVSGQGDSL |
| 721 | HEHIANLAGS PAIKKGILQT VKVVDELVKV MGRHKPENIV |
| | IEMARENQTT QKGQKNSRER |
| 781 | MKRIEEGIKE LGSQILKEHP VENTQLQNEK LYLYYLQNGR |
| | DMYVDQELDI NRLSDYDVDH |
| 841 | IVPQSFLKDD SIDNKVLTRS DKNRGKSDNV PSEEVVKKMK |
| | NYWRQLLNAK LITQRKFDNL |
| 901 | TKAERGGLSE LDKAGFIKRQ LVETRQITKH VAQILDSRMN |
| | TKYDENDKLI REVKVITLKS |
| 961 | KLVSDFRKDF QFYKVREINN YHHAHDAYLN AVVGTALIKK |
| | YPKLESEFVY GDYKVYDVRK |
| 1021 | MIAKSEQEIG KATAKYFFYS NIMNFFKTEI TLANGEIRKR |
| | PLIETNGETG EIVWDKGRDF |
| 1081 | ATVRKVLSMP QVNIVKKTEV QTGGFSKESI LPKRNSDKLI |
| | ARKKDWDPKK YGGFDSPTVA |
| 1141 | YSVLVVAKVE KGKSKKLKSV KELLGITIME RSSFEKNPID |
| | FLEAKGYKEV KKDLIIKLPK |
| 1201 | YSLFELENGR KRMLASAGEL QKGNELALPS KYVNFLYLAS |
| | HYEKLKGSPE DNEQKQLFVE |
| 1261 | QHKHYLDEII EQISEFSKRV ILADANLDKV LSAYNKHRDK |
| | PIREQAENII HLFTLTNLGA |
| 1321 | PAAFKYFDTT IDRKRYTSTK EVLDATLIHQ SITGLYETRI |
| | DLSQLGGD |

In embodiments, the nuclease is a Cas9 derived from S. aureus (SaCas9). An exemplary SaCas9 sequence is provided below (SEQ ID NO: 22):

| | |
|---|---|
| 1 | MKRNYILGLD IGITSVGYGI IDYETRDVID AGVRLFKEAN |
| | VENNEGRRSK RGARRLKRRR |
| 61 | RHRIQRVKKL LFDYNLLTDH SELSGINPYE ARVKGLSQKL |
| | SEEEFSAALL HLAKRRGVHN |
| 121 | VNEVEEDTGN ELSTKEQISR NSKALEEKYV AELQLERLKK |
| | DGEVRGSINR FKTSDYVKEA |
| 181 | KQLLKVQKAY HQLDQSFIDT YIDLLETRRT YYEGPGEGSP |
| | FGWKDIKEWY EMLMGHCTYF |
| 241 | PEELRSVKYA YNADLYNALN DLNNLVITRD ENEKLEYYEK |
| | FQIIENVFKQ KKKPTLKQIA |
| 301 | KEILVNEEDI KGYRVTSTGK PEFTNLKVYH DIKDITARKE |
| | IIENAELLDQ IAKILTIYQS |
| 361 | SEDIQEELTN LNSELTQEEI EQISNLKGYT GTHNLSLKAI |
| | NLILDELWHT NDNQIAIFNR |
| 421 | LKLVPKKVDL SQQKEIPTTL VDDFILSPVV KRSFIQSIKV |
| | INAIIKKYGL PNDIIIELAR |
| 481 | EKNSKDAQKM INEMQKRNRQ TNERIEEIIR TTGKENAKYL |
| | IEKIKLHDMQ EGKCLYSLEA |
| 541 | IPLEDLLNNP FNYEVDHIIP RSVSFDNSFN NKVLVKQEEN |
| | SKKGNRTPFQ YLSSSDSKIS |
| 601 | YETFKKHILN LAKGKGRISK TKKEYLLEER DINRFSVQKD |
| | FINRNLVDTR YATRGLMNLL |
| 661 | RSYFRVNNLD VKVKSINGGF TSFLRRKWKF KKERNKGYKH |
| | HAEDALIIAN ADFIFKEWKK |
| 721 | LDKAKKVMEN QMFEEKQAES MPEIETEQEY KEIFITPHQI |
| | KHIKDFKDYK YSHRVDKKPN |
| 781 | RELINDTLYS TRKDDKGNTL IVNNLNGLYD KDNDKLKKLI |
| | NKSPEKLLMY HHDPQTYQKL |
| 841 | KLIMEQYGDE KNPLYKYYEE TGNYLTKYSK KDNGPVIKKI |
| | KYYGNKLNAH LDITDDYPNS |
| 901 | RNKVVKLSLK PYRFDVYLDN GVYKFVTVKN LDVIKKENYY |
| | EVNSKCYEEA KKLKKISNQA |
| 961 | EFIASFYNND LIKINGELYR VIGVNNDLLN RIEVNMIDIT |
| | YREYLENMND KRPPRIIKTI |
| 1021 | ASKTQSIKKY STDILGNLYE VKSKKHPQII |

In embodiments, the Cpf1 is a Cpf1 enzyme from Acidaminococcus (species BV3L6, UniProt Accession No. U2UMQ6; SEQ ID NO: 23), having the sequence set forth below:

| | |
|---|---|
| 1 | MTQFEGFTNL YQVSKTLRFE LIPQGKTLKH IQEQGFIEED |
| | KARNDHYKEL KPIIDRIYKT |
| 61 | YADQCLQLVQ LDWENLSAAI DSYRKEKTEE TRNALIEEQA |
| | TYRNAIHDYF IGRIDNLIDA |
| 121 | INKRHAEIYK GLFKAELENG KVLKQLGTVT TTEHENALLR |
| | SFDKFTTYFS GFYENRKNVF |
| 181 | SAEDISTAIP HRIVQDNFPK FKENCHIFTR LITAVPSLRE |
| | HFENVKKAIG IFVSTSIEEV |
| 241 | FSFPFYNQLL TQTQIDLYNQ LLGGISREAG TEKIKGLNEV |
| | LNLAIQKNDE TAHIIASLPH |
| 301 | RFIPLFKQIL SDRNTLSFIL EEFKSDEEVI QSFCKYKTLL |
| | RNENVLETAE ALFNELNSID |
| 361 | LTHIFISHKK LETISSALCD HWDTLRNALY ERRISELTGK |
| | ITKSAKEKVQ RSLKHEDINL |
| 421 | QEIISAAGKE LSEAFKQKTS EILSHAHAAL DQPLPTTLKK |
| | QEEKEILKSQ LDSLLGLYHL |
| 481 | LDWFAVDESN EVDPEFSARL TGIKLEMEPS LSFYNKARNY |
| | ATKKPYSVEK FKLNFQMPTL |
| 541 | ASGWDVNKEK NNGAILFVKN GLYYLGIMPK QKGRYKALSF |
| | EPTEKTSEGF DKMYYDYFPD |
| 601 | AAKMIPKCST QLKAVTAHFQ THTTPILLSN NFIEPLEITK |
| | EIYDLNNPEK EPKKFQTAYA |
| 661 | KKTGDQKGYR EALCKWIDFT RDFLSKYTKT TSIDLSSLRP |
| | SSQYKDLGEY YAELNPLLYH |
| 721 | ISFQRIAEKE IMDAVETGKL YLFQIYNKDF AKGHHGKPNL |
| | HTLYWTGLFS PENLAKTSIK |
| 781 | LNGQAELFYR PKSRMKRMAH RLGEKMLNKK LKDQKTPIPD |
| | TLYQELYDYV NHRLSHDLSD |
| 841 | EARALLPNVI TKEVSHEIIK DRRFTSDKFF FHVPITLNYQ |
| | AANSPSKFNQ RVNAYLKEHP |
| 901 | ETPIIGIDRG ERNLIYITVI DSTGKILEQR SLNTIQQFDY |
| | QKKLDNREKE RVAARQAWSV |
| 961 | VGTIKDLKQG YLSQVIHEIV DLMIHYQAVV VLENLNFGFK |
| | SKRTGIAEKA VYQQFEKMLI |
| 1021 | DKLNCLVLKD YPAEKVGGVL NPYQLTDQFT SFAKMGTQSG |
| | FLFYVPAPYT SKIDPLTGFV |
| 1081 | DPFVWKTIKN HESRKHFLEG FDFLHYDVKT GDFILHFKMN |
| | RNLSFQRGLP GFMPAWDIVF |
| 1141 | EKNETQFDAK GTPFIAGKRI VPVIENHRFT GRYRDLYPAN |
| | ELIALLEEKG IVFRDGSNIL |
| 1201 | PKLLENDDSH AIDTMVALIR SVLQMRNSNA ATGEDYINSP |
| | VRDLNGVCFD SRFQNPEWPM |
| 1261 | DADANGAYHI ALKGQLLLNH LKESKDLKLQ NGISNQDWLA |
| | YIQELRN. |

In some embodiments, the Cpf1 is a Cpf1 enzyme from Lachnospiraceae (species ND2006, UniProt Accession No. A0A182DWE3; SEQ ID NO: 24), having the sequence set forth below:

| | |
|---|---|
| 1 | AASKLEKFTN CYSLSKTLRF KAIPVGKTQE NIDNKRLLVE |
| | DEKRAEDYKG VKKLLDRYYL |
| 61 | SFINDVLHSI KLKNLNNYIS LFRKKTRTEK ENKELENLEI |
| | NLRKEIAKAF KGAAGYKSLF |
| 121 | KKDIIETILP EAADDKDEIA LVNSENGETT AFTGFFDNRE |
| | NMFSEEAKST SIAFRCINEN |
| 181 | LTRYISNMDI FEKVDAIFDK HEVQEIKEKI LNSDYDVEDF |
| | FEGEFFNFVL TQEGIDVYNA |
| 241 | IIGGFVTESG EKIKGLNEYI NLYNAKTKQA LPKFKPLYKQ |
| | VLSDRESLSF YGEGYTSDEE |
| 301 | VLEVFRNTLN KNSEIFSSIK KLEKLFKNFD EYSSAGIFVK |
| | NGPAISTISK DIFGEWNLIR |
| 361 | DKWNAEYDDI HLKKKAVVTE KYEDDRRKSF KKIGSFSLEQ |
| | LQEYADADLS VVEKLKEIII |
| 421 | QKVDEIYKVY GSSEKLFDAD FVLEKSLKKN DAVVAIMKDL |
| | LDSVKSFENY IKAFFGEGKE |
| 481 | TNRDESFYGD FVLAYDILLK VDHIYDAIRN YVTQKPYSKD |
| | KFKLYFQNPQ FMGGWDKDKE |
| 541 | TDYRATILRY GSKYYLAIMD KKYAKCLQKI DKDDVNGNYE |
| | KINYKLLPGP NKMLPKVFFS |
| 601 | KKWMAYYNPS EDIQKIYKNG TFKKGDMFNL NDCHKLIDFF |
| | KDSISRYPKW SNAYDFNFSE |
| 661 | TEKYKDIAGF YREVEEQGYK VSFESASKKE VDKLVEEGKL |
| | YMFQIYNKDF SDKSHGTPNL |
| 721 | HTMYFKLLFD ENNHGQIRLS GGAELFMRRA SLKKEELVVH |
| | PANSPIANKN PDNPKKTTTL |
| 781 | SYDVYKDKRF SEDQYELHIP IAINKCPKNI FKINTEVRVL |
| | LKHDDNPYVI GIDRGERNLL |
| 841 | YIVVVDGKGN IVEQYSLNEI INNFNGIRIK TDYHSLLDKK |
| | EKERFEARQN WTSIENIKEL |
| 901 | KAGYISQVVH KICELVEKYD AVIALEDLNS GFKNSRVKVE |
| | KQVYQKFEKM LIDKLNYMVD |
| 961 | KKSNPCATGG ALKGYQITNK FESFKSMSTQ NGFIFYIPAW |
| | LTSKIDPSTG FVNLLKTKYT |
| 1021 | SIADSKKFIS SFDRIMYVPE EDLFEFALDY KNFSRTDADY |
| | IKKWKLYSYG NRIRIFAAAK |
| 1081 | KNNVFAWEEV CLTSAYKELF NKYGINYQQG DIRALLCEQS |
| | DKAFYSSFMA LMSLMLQMRN |
| 1141 | SITGRTDVDF LISPVKNSDG IFYDSRNYEA QENAILPKNA |
| | DANGAYNIAR KVLWAIGQFK |
| 1201 | KAEDEKLDKV KIAISNKEWL EYAQTSVK |

### Vectors

The AAV expression cassettes disclosed herein may be packaged into a vector, and used to deliver DNA sequences to a cell or tissue of interest. The vector may be, for example, a non-viral vector (such as a plasmid or a nanoparticle), or a viral vector (such as an AAV or a baculovirus).

Thus, provided herein is an AAV vector comprising an AAV expression cassette of the disclosure. The AAV vector may be of any serotype. For example, the AAV vector may be an AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, or an ovine AAV vector. In some embodiments, the AAV vector is non-naturally occurring. For example, the AAV vector may be chimeric or an AAV vector whose capsid protein has one or more mutations compared to wildtype. In some embodiments, the AAV vector may be replication-defective or conditionally replication defective. In some embodiments, the AAV vector may be self-complimentary (scAAV).

In some embodiments, the AAV vector is selected from one of the serotypes listed in Table 18.

TABLE 18
AAV Serotypes and Capsid Sequences

| | Seq ID |
|---|---|
| Serotype | No. |
| VOY1O1 | 2379 |
| VOY201 | 2380 |
| PHP.N/PHP.B- | 2381 |
| DGT | |
| AAVPHP.B or | 2382 |
| G2B-26 | |
| AAVPHP.B | 2383 |
| AAVG2B-13 | 2384 |
| AAVTH1.1-32 | 2385 |
| AAVTH1.1-35 | 2386 |
| PHP.S/G2A12 | 2387 |
| AAV9/hu.14 | 2388 |
| K449R | |
| AAV1 | 2389 |
| AAV1 | 2390 |
| AAV1 | 2391 |
| AAV1.3 | 2392 |
| AAV1O | 2393 |
| AAV1O | 2394 |
| AAV1O | 2395 |
| AAV11 | 2396 |
| AAV12 | 2397 |
| AAV2 | 2398 |
| AAV2 | 2399 |
| AAV2 | 2400 |
| AAV2 | 2401 |
| AAV2 | 2402 |
| AAV2.5T | 2403 |
| AAV223.10 | 2404 |
| AAV223.2 | 2405 |
| AAV223.2 | 2406 |
| AAV223.4 | 2407 |
| AAV223.4 | 2408 |
| AAV223.5 | 2409 |
| AAV223.5 | 2410 |
| AAV223.6 | 2411 |
| AAV223.6 | 2412 |
| AAV223.7 | 2413 |
| AAV223.7 | 2414 |
| AAV29.3 | 2415 |
| AAV29.4 | 2416 |
| AAV29.5 | 2417 |
| AAV29.5 | 2418 |
| (AAVbb .2) | |
| AAV3 | 2419 |
| AAV3 | 2420 |
| AAV3 | 2421 |
| AAV3.3b | 2422 |
| AAV3-3 | 2423 |
| AAV3-3 | 2424 |
| AAV3a | 2425 |
| AAV3a | 2426 |
| AAV3b | 2427 |
| AAV3b | 2428 |
| AAV3b | 2429 |
| AAV4 | 2430 |
| AAV4 | 2431 |
| AAV4 | 2432 |
| AAV4 | 2433 |
| AAV4 | 2434 |
| AAV4 | 2435 |
| AAV4 | 2436 |
| AAV4 | 2437 |
| AAV4 | 2438 |
| AAV4 | 2439 |
| AAV4 | 2440 |
| AAV4 | 2441 |
| AAV4 | 2442 |
| AAV4 | 2443 |
| AAV4 | 2444 |
| AAV4 | 2445 |
| AAV4 | 2446 |
| AAV4 | 2447 |
| AAV4 | 2448 |
| AAV4 | 2449 |
| AAV42.2 | 2450 |
| AAV42.2 | 2451 |
| AAV42.3b | 2452 |
| AAV42.3B | 2453 |
| AAV42.4 | 2454 |
| AAV42.4 | 2455 |
| AAV42.8 | 2456 |
| AAV42.8 | 2457 |
| AAV43.1 | 2458 |
| AAV43.1 | 2459 |
| AAV43.12 | 2460 |
| AAV43.12 | 2461 |
| AAV43.20 | 2462 |
| AAV43.20 | 2463 |
| AAV43.21 | 2464 |
| AAV43.21 | 2465 |
| AAV43.23 | 2466 |
| AAV43.23 | 2467 |
| AAV43.25 | 2468 |
| AAV43.25 | 2469 |
| AAV43.5 | 2470 |
| AAV43.5 | 2471 |
| AAV4-4 | 2472 |
| AAV4-4 | 2473 |
| AAV44.1 | 2474 |
| AAV44.1 | 2475 |
| AAV44.5 | 2476 |
| AAV44.5 | 2477 |
| AAV4407 | 2478 |
| AAV5 | 2479 |
| AAV5 | 2480 |
| AAV5 | 2481 |
| AAV5 | 2482 |
| AAV6 | 2483 |
| AAV6 | 2484 |
| AAV6 | 2485 |
| AAV6 | 2486 |
| AAV6 | 2487 |
| AAV6 | 2488 |
| AAV6.1 | 2489 |
| AAV6.12 | 2490 |
| AAV6.2 | 2491 |
| AAV7 | 2492 |
| AAV7 | 2493 |
| AAV7 | 2494 |
| AAV7 | 2495 |
| AAV7 | 2496 |
| AAV7 | 2497 |
| AAV7 | 2498 |
| AAV8 | 2499 |
| AAV8 | 2500 |
| AAV8 | 2501 |
| AAV8 | 2502 |
| AAV8 | 2503 |
| AAV8 | 2504 |
| AAV-8b | 2505 |
| AAV-8b | 2506 |
| AAV-8h | 2507 |
| AAV-8h | 2508 |
| AAV9 | 2509 |
| AAV9 | 2510 |
| AAV9 | 2511 |
| AAV9 | 2512 |
| AAV9 | 2513 |
| AAV9 | 2514 |
| (AAVhu.14) | |
| AAV9 | 2515 |
| (AAVhu.14) | |
| AAVA3.1 | 2516 |
| AAVA3.3 | 2517 |
| AAVA3.3 | 2518 |
| AAVA3.4 | 2519 |
| AAVA3.4 | 2520 |
| AAVA3.5 | 2521 |
| AAVA3.5 | 2522 |
| AAVA3.7 | 2523 |
| AAVA3.7 | 2524 |
| AAV29.3 | 2525 |
| (AAVbb.1) | |
| AAVC2 | 2526 |
| AAVCh.5 | 2527 |
| AAVcy.2 | 2528 |
| (AAV13.3) | |
| AAV24.1 | 2529 |
| AAVcy.3 | 2530 |
| (AAV24.1) | |
| AAV27.3 | 2531 |
| AAVcy.4 | 2532 |
| (AAV27.3) | |
| AAVcy.5 | 2533 |
| AAV7.2 | 2534 |
| AAVcy.5 | 2535 |
| (AAV7.2) | |
| AAV16.3 | 2536 |
| AAVcy.6 | 2537 |
| (AAV16.3) | |
| AAVcy.5 | 2538 |
| AAVcy.5 | 2539 |
| AAVCy.5R1 | 2540 |
| AAVCy.5R2 | 2541 |
| AAVCy.5R3 | 2542 |
| AAVCy.5R4 | 2543 |
| AAVDJ | 2544 |
| AAVDJ | 2545 |
| AAVDJ-8 | 2546 |
| AAVDJ-8 | 2547 |
| AAVF5 | 2548 |
| AAVH2 | 2549 |
| AAVH6 | 2550 |
| AAVhE1.1 | 2551 |
| AAVhEr1.14 | 2552 |
| AAVhEr1.16 | 2553 |
| AAVhEr1.18 | 2554 |
| AAVhEr1.23 | 2555 |
| (AAVhEr2.29) | |
| AAVhEr1.35 | 2556 |
| AAVhEr1.36 | 2557 |
| AAVhEr1.5 | 2558 |
| AAVhEr1.7 | 2559 |
| AAVhEr1.8 | 2560 |
| AAVhEr2.16 | 2561 |
| AAVhEr2.30 | 2562 |
| AAVhEr2.31 | 2563 |
| AAVhEr2.36 | 2564 |
| AAVhEr2.4 | 2565 |
| AAVhEr3.1 | 2566 |
| AAVhu.1 | 2567 |
| AAVhu.1 | 2568 |
| AAVhu. 10 | 2569 |
| (AAV16. 8) | |
| AAVhu. 10 | 2570 |
| (AAV16. 8) | |
| AAVhu.11 | 2571 |
| (AAV16.12) | |
| AAVhu.11 | 2572 |
| (AAV16.12) | |
| AAVhu.12 | 2573 |
| AAVhu.12 | 2574 |
| AAVhu.13 | 2575 |
| AAVhu.13 | 2576 |
| AAVhu.136.1 | 2577 |
| AAVhu.140.1 | 2578 |
| AAVhu.140.2 | 2579 |
| AAVhu.145.6 | 2580 |
| AAVhu.15 | 2581 |
| AAVhu.15 | 2582 |
| (AAV33 .4) | |
| AAVhu.156.1 | 2583 |
| AAVhu.16 | 2584 |
| AAVhu.16 | 2585 |
| (AAV33 .8) | |
| AAVhu.17 | 2586 |
| AAVhu.17 | 2587 |
| (AAV33.12) | |
| AAVhu.172.1 | 2588 |
| AAVhu.172.2 | 2589 |
| AAVhu.173.4 | 2590 |
| AAVhu.173.8 | 2591 |
| AAVhu.18 | 2592 |
| AAVhu.18 | 2593 |
| AAVhu.19 | 2594 |
| AAVhu.19 | 2595 |
| AAVhu.2 | 2596 |
| AAVhu.2 | 2597 |
| AAVhu.20 | 2598 |
| AAVhu.20 | 2599 |
| AAVhu.21 | 2600 |
| AAVhu.21 | 2601 |
| AAVhu.22 | 2602 |
| AAVhu.22 | 2603 |
| AAVhu.23 | 2604 |
| AAVhu.23.2 | 2605 |
| AAVhu.24 | 2606 |
| AAVhu.24 | 2607 |
| AAVhu.25 | 2608 |
| AAVhu.25 | 2609 |
| AAVhu.26 | 2610 |
| AAVhu.26 | 2611 |
| AAVhu.27 | 2612 |
| AAVhu.27 | 2613 |
| AAVhu.28 | 2614 |
| AAVhu.28 | 2615 |
| AAVhu.29 | 2616 |
| AAVhu.29 | 2617 |
| AAVhu.29 | 2618 |
| AAVhu.29R | 2619 |
| AAVhu.3 | 2620 |
| AAVhu.3 | 2621 |
| AAVhu.30 | 2622 |
| AAVhu.30 | 2623 |
| AAVhu.31 | 2624 |
| AAVhu.31 | 2625 |
| AAVhu.32 | 2626 |
| AAVhu.32 | 2627 |
| AAVhu.33 | 2628 |
| AAVhu.33 | 2629 |
| AAVhu.34 | 2630 |
| AAVhu.34 | 2631 |
| AAVhu.35 | 2632 |
| AAVhu.35 | 2633 |
| AAVhu.36 | 2634 |
| AAVhu.36 | 2635 |
| AAVhu.37 | 2636 |
| AAVhu.37 | 2637 |
| (AAV106.1) | |
| AAVhu.38 | 2638 |
| AAVhu.39 | 2639 |
| AAVhu.39 | 2640 |
| (AAVLG-9) | |
| AAVhu.4 | 2641 |
| AAVhu.4 | 2642 |
| AAVhu.40 | 2643 |
| AAVhu.40 | 2644 |
| (AAV1 14.3) | |
| AAVhu.41 | 2645 |
| AAVhu.41 | 2646 |
| (AAV127.2) | |
| AAVhu.42 | 2647 |
| AAVhu.42 | 2648 |
| (AAV127.5) | |
| AAVhu.43 | 2649 |
| AAVhu.43 | 2650 |
| AAVhu.43 | 2651 |
| (AAV128. 1) | |
| AAVhu.44 | 2652 |
| AAVhu.44 | 2653 |
| (AAV128.3) | |
| AAVhu.44R1 | 2654 |
| AAVhu.44R2 | 2655 |
| AAVhu.44R3 | 2656 |
| AAVhu.45 | 2657 |
| AAVhu.45 | 2658 |
| AAVhu.46 | 2659 |
| AAVhu.46 | 2660 |
| AAVhu.46 | 2661 |
| AAVhu.47 | 2662 |
| AAVhu.47 | 2663 |
| AAVhu.48 | 2664 |
| AAVhu.48 | 2665 |
| AAVhu.48 | 2666 |
| (AAV130.4) | |
| AAVhu.48R1 | 2667 |
| AAVhu.48R2 | 2668 |
| AAVhu.48R3 | 2669 |
| AAVhu.49 | 2670 |
| AAVhu.49 | 2671 |
| AAVhu.5 | 2672 |
| AAVhu.5 | 2673 |
| AAVhu.51 | 2674 |
| AAVhu.51 | 2675 |
| AAVhu.52 | 2676 |
| AAVhu.52 | 2677 |
| AAVhu.53 | 2678 |
| AAVhu.53 | 2679 |
| AAVhu.53 | 2680 |
| (AAV145.1) | |
| AAVhu.54 | 2681 |
| AAVhu.54 | 2682 |
| (AAV145.5) | |
| AAVhu.55 | 2683 |
| AAVhu.56 | 2684 |
| AAVhu.56 | 2685 |
| (AAV145.6) | |
| AAVhu.56 | 2686 |
| (AAV145.6) | |
| AAVhu.57 | 2687 |
| AAVhu.57 | 2688 |
| AAVhu.57 | 2689 |
| AAVhu.58 | 2690 |
| AAVhu.58 | 2691 |
| AAVhu.6 | 2692 |
| (AAV3.1) | |
| AAVhu.6 | 2693 |
| (AAV3.1) | |
| AAVhu.60 | 2694 |
| AAVhu.60 | 2695 |
| (AAV161.10) | |
| AAVhu.61 | 2696 |
| AAVhu.61 | 2697 |
| (AAV161.6) | |
| AAVhu.63 | 2698 |
| AAVhu.63 | 2699 |
| AAVhu.64 | 2700 |
| AAVhu.64 | 2701 |
| AAVhu.66 | 2702 |
| AAVhu.67 | 2703 |
| AAVhu.67 | 2704 |
| AAVhu.7 | 2705 |
| AAVhu.7 | 2706 |
| AAVhu.7 | 2707 |
| (AAV7.3) | |
| AAVhu.71 | 2708 |
| AAVhu.8 | 2709 |
| AAVhu.8 | 2710 |
| AAVhu.8 | 2711 |
| AAVhu.9 | 2712 |
| (AAV3.1) | |
| AAVhu.9 | 2713 |
| (AAV3.1) | |
| AAV-LKO1 | 2714 |
| AAV-LKO1 | 2715 |
| AAV-LK02 | 2716 |
| AAV-LK02 | 2717 |
| AAV-LK03 | 2718 |
| AAV-LK03 | 2719 |
| AAV-LK04 | 2720 |
| AAV-LK04 | 2721 |
| AAV-LK05 | 2722 |
| AAV-LK05 | 2723 |
| AAV-LK06 | 2724 |
| AAV-LK06 | 2725 |
| AAV-LK07 | 2726 |
| AAV-LK07 | 2727 |
| AAV-LK08 | 2728 |
| AAV-LK08 | 2729 |
| AAV-LK09 | 2730 |
| AAV-LK09 | 2731 |
| AAV-LK1O | 2732 |
| AAV-LK1O | 2733 |
| AAV-LK11 | 2734 |
| AAV-LK11 | 2735 |
| AAV-LK12 | 2736 |
| AAV-LK12 | 2737 |
| AAV-LK13 | 2738 |
| AAV-LK13 | 2739 |
| AAV-LK14 | 2740 |
| AAV-LK14 | 2741 |
| AAV-LK15 | 2742 |
| AAV-LK15 | 2743 |
| AAV-LK16 | 2744 |
| AAV-LK16 | 2745 |
| AAV-LK17 | 2746 |
| AAV-LK17 | 2747 |
| AAV-LK18 | 2748 |
| AAV-LK18 | 2749 |
| AAV-LK19 | 2750 |
| AAV-LK19 | 2751 |
| AAV-PAEC | 2752 |
| AAV-PAEC | 2753 |
| AAV-PAEC11 | 2754 |
| AAV-PAEC11 | 2755 |
| AAV-PAEC12 | 2756 |
| AAV-PAEC12 | 2757 |
| AAV-PAEC13 | 2758 |
| AAV-PAEC13 | 2759 |
| AAV-PAEC2 | 2760 |
| AAV-PAEC2 | 2761 |
| AAV-PAEC4 | 2762 |
| AAV-PAEC4 | 2763 |
| AAV-PAEC6 | 2764 |
| AAV-PAEC6 | 2765 |
| AAV-PAEC7 | 2766 |
| AAV-PAEC7 | 2767 |
| AAV-PAEC8 | 2768 |
| AAV-PAEC8 | 2769 |
| AAVpi.1 | 2770 |
| AAVpi.1 | 2771 |
| AAVpi.2 | 2772 |
| AAVpi.2 | 2773 |
| AAVpi.3 | 2774 |
| AAVpi.3 | 2775 |
| AAVrh.10 | 2776 |
| AAVrh.10 | 2777 |
| AAV44.2 | 2778 |
| AAVrh.10 | 2779 |
| (AAV44.2) | |
| AAV42.1B | 2780 |
| AAVrh.12 | 2781 |
| (AAV42.1b) | |
| AAVrh.13 | 2782 |
| AAVrh.13 | 2783 |
| AAVrh.13 | 2784 |
| AAVrh.13R | 2785 |
| AAV42.3A | 2786 |
| AAVrh.14 | 2787 |
| (AAV42.3a) | |
| AAV42.5A | 2788 |
| AAVrh.17 | 2789 |
| (AAV42.5a) | |
| AAV42.5B | 2790 |
| AAVrh.18 | 2791 |
| (AAV42.5b) | |
| AAV42.6B | 2792 |
| AAVrh.19 | 2793 |
| (AAV42.6b) | |
| AAVrh.2 | 2794 |
| AAVrh.2 | 2795 |
| AAVrh.20 | 2796 |
| AAV42.10 | 2797 |
| AAVrh.21 | 2798 |
| (AAV42.10) | |
| AAV42.11 | 2799 |
| AAVrh.22 | 2800 |
| (AAV42.11) | |
| AAV42.12 | 2801 |
| AAVrh.23 | 2802 |
| (AAV42.12) | |
| AAV42.13 | 2803 |
| AAVrh.24 | 2804 |
| (AAV42.13) | |
| AAV42.15 | 2805 |
| AAVrh.25 | 2806 |
| (AAV42.15) | |
| AAVrh.2R | 2807 |
| AAVrh.31 | 2808 |
| (AAV223.1) | |
| AAVC1 | 2809 |
| AAVrh.32 | 2810 |
| (AAVC1) | |
| AAVrh.32/33 | 2811 |
| AAVrh.33 | 2812 |
| (AAVC3) | |
| AAVC5 | 2813 |
| AAVrh.34 | 2814 |
| (AAVC5) | |
| AAVF1 | 2815 |
| AAVrh.35 | 2816 |
| (AAVF1) | |
| AAVF3 | 2817 |
| AAVrh.36 | 2818 |
| (AAVF3) | |
| AAVrh.37 | 2819 |
| AAVrh.37 | 2820 |
| AAVrh.37 | 2821 |
| AAVrh.37R2 | 2822 |
| AAVrh.38 | 2823 |
| (AAVLG-4) | |
| AAVrh.38 | 2824 |
| (AAVLG-4) | |
| AAVrh.39 | 2825 |
| AAVrh.39 | 2826 |
| AAVrh.40 | 2827 |
| AAVrh.40 | 2828 |
| (AAVLG-10) | |
| AAVrh.43 | 2829 |
| (AAVN721-8) | |
| AAVrh.43 | 2830 |
| (AAVN721-8) | |
| AAVrh.44 | 2831 |
| AAVrh.44 | 2832 |
| AAVrh.45 | 2833 |
| AAVrh.45 | 2834 |
| AAVrh.46 | 2835 |
| AAVrh.46 | 2836 |
| AAVrh.47 | 2837 |
| AAVrh.47 | 2838 |
| AAVrh.48 | 2839 |
| AAVrh.48.1 | 2840 |
| AAVrh.48.1.2 | 2841 |
| AAVrh.48.2 | 2842 |
| AAVrh.48 | 2843 |
| (AAV1-7) | |
| AAVrh.49 | 2844 |
| (AAV1-8) | |
| AAVrh.49 | 2845 |
| (AAV1-8) | |
| AAVrh.50 | 2846 |
| (AAV2-4) | |
| AAVrh.50 | 2847 |
| (AAV2-4) | |
| AAVrh.51 | 2848 |
| (AAV2-5) | |
| AAVrh.51 | 2849 |
| (AAV2-5) | |
| AAVrh.52 | 2850 |
| (AAV3-9) | |
| AAVrh.52 | 2851 |
| (AAV3-9) | |
| AAVrh.53 | 2852 |
| AAVrh.53 | 2853 |
| (AAV3-11) | |
| AAVrh.53 | 2854 |
| (AAV3-11) | |
| AAVrh.54 | 2855 |
| AAVrh.54 | 2856 |
| AAVrh.55 | 2857 |
| AAVrh.55 | 2858 |
| (AAV4-19) | |
| AAVrh.56 | 2859 |
| AAVrh.56 | 2860 |
| AAVrh.57 | 2861 |
| AAVrh.57 | 2862 |
| AAVrh.58 | 2863 |
| AAVrh.58 | 2864 |
| AAVrh.58 | 2865 |
| AAVrh.59 | 2866 |
| AAVrh.59 | 2867 |
| AAVrh.60 | 2868 |
| AAVrh.60 | 2869 |
| AAVrh.61 | 2870 |
| AAVrh.61 | 2871 |
| (AAV2-3) | |
| AAVrh.62 | 2872 |
| (AAV2-15) | |
| AAVrh.62 | 2873 |
| (AAV2-15) | |
| AAVrh.64 | 2874 |
| AAVrh.64 | 2875 |
| AAVrh.64 | 2876 |
| AAVRh.64R1 | 2877 |
| AAVRh.64R2 | 2878 |
| AAVrh.65 | 2879 |
| AAVrh.65 | 2880 |
| AAVrh.67 | 2881 |
| AAVrh.67 | 2882 |
| AAVrh.67 | 2883 |
| AAVrh.68 | 2884 |
| AAVrh.68 | 2885 |
| AAVrh.69 | 2886 |
| AAVrh.69 | 2887 |
| AAVrh.70 | 2888 |
| AAVrh.70 | 2889 |
| AAVrh.71 | 2890 |
| AAVrh.72 | 2891 |
| AAVrh.73 | 2892 |
| AAVrh.74 | 2893 |
| AAVrh.8 | 2894 |
| AAVrh.8 | 2895 |
| AAVrh.8R | 2896 |
| AAVrh.8R | 2897 |
| A586R mutant | |
| AAVrh.8R | 2898 |
| R533A mutant | |
| BAAV (bovine | 2899 |
| AAV) | |
| BAAV (bovine | 2900 |
| AAV) | |
| BAAV (bovine | 2901 |
| AAV) | |
| BAAV (bovine | 2902 |
| AAV) | |
| BAAV (bovine | 2903 |
| AAV) | |
| BAAV (bovine | 2904 |
| AAV) | |
| BAAV (bovine | 2905 |
| AAV) | |
| BAAV (bovine | 2906 |
| AAV) | |
| BAAV (bovine | 2907 |
| AAV) | |
| BAAV (bovine | 2908 |
| AAV) | |
| BAAV (bovine | 2909 |
| AAV) | |
| BAAV (bovine | 2910 |
| AAV) | |
| BAAV (bovine | 2911 |
| AAV) | |
| BNP61 AAV | 2912 |
| BNP61 AAV | 2913 |
| BNP62AAV | 2914 |
| BNP63 AAV | 2915 |
| caprine AAV | 2916 |
| caprine AAV | 2917 |
| true type AAV | 2918 |
| (ttAAV) | |
| AAAV (Avian | 2919 |
| AAV) | |
| AAAV (Avian | 2920 |
| AAV) | |
| AAAV (Avian | 2921 |
| AAV) | |
| AAAV (Avian | 2922 |
| AAV) | |
| AAAV (Avian | 2923 |
| AAV) | |
| AAAV (Avian | 2924 |
| AAV) | |
| AAAV (Avian | 2925 |
| AAV) | |
| AAAV (Avian | 2926 |
| AAV) | |
| AAAV (Avian | 2927 |
| AAV) | |
| AAAV (Avian | 2928 |
| AAV) | |
| AAAV (Avian | 2929 |
| AAV) | |
| AAAV (Avian | 2930 |
| AAV) | |
| AAAV (Avian | 2931 |
| AAV) | |
| AAAV (Avian | 2932 |
| AAV) | |
| AAAV (Avian | 2933 |
| AAV) | |
| AAV Shuffle | 2934 |
| 100-1 | |
| AAV Shuffle | 2935 |
| 100-1 | |
| AAV Shuffle | 2936 |
| 100-2 | |
| AAV Shuffle | 2937 |
| 100-2 | |
| AAV Shuffle | 2938 |
| 100-3 | |
| AAV Shuffle | 2939 |
| 100-3 | |
| AAV Shuffle | 2940 |
| 100-7 | |
| AAV Shuffle | 2941 |
| 100-7 | |
| AAV Shuffle 10- | 2942 |
| 2 | |
| AAV Shuffle 10- | 2943 |
| 2 | |
| AAV Shuffle 10- | 2944 |
| 6 | |
| AAV Shuffle 10- | 2945 |
| 6 | |
| AAV Shuffle 10- | 2946 |
| 8 | |
| AAV Shuffle 10- | 2947 |
| 8 | |
| AAVSM 100-10 | 2948 |
| AAVSM 100-10 | 2949 |
| AAV SM 100-3 | 2950 |
| AAV SM 100-3 | 2951 |
| AAV SM 10-1 | 2952 |
| AAV SM 10-1 | 2953 |
| AAVSM 10-2 | 2954 |
| AAVSM 10-2 | 2955 |
| AAV SM 10-8 | 2956 |
| AAV SM 10-8 | 2957 |
| AAVF1/HSC1 | 2958 |
| AAVF2/HSC2 | 2959 |
| AAVF3/HSC3 | 2960 |
| AAVF4/HSC4 | 2961 |
| AAVF5/HSC5 | 2962 |
| AAVF6/HSC6 | 2963 |
| AAVF7/HSC7 | 2964 |
| AAVF8/HSC8 | 2965 |
| AAVF9/HSC9 | 2966 |
| AAVF1 1/HSC1 | 2967 |
| 1 | |
| AAVF12/HSC12 | 2968 |
| AAVF13/HSC13 | 2969 |
| AAVF14/HSC14 | 2970 |
| AAVF15/HSC15 | 2971 |
| AAVF16/HSC16 | 2972 |
| AAVF17/HSC17 | 2973 |
| AAVF1/HSC1 | 2974 |
| AAVF2/HSC2 | 2975 |
| AAVF3/HSC3 | 2976 |
| AAVF4/HSC4 | 2977 |
| AAVF5/HSC5 | 2978 |
| AAVF6/HSC6 | 2979 |
| AAVF7/HSC7 | 2980 |
| AAVF8/HSC8 | 2981 |
| AAVF9/HSC9 | 2982 |
| AAVF1 1/HSC1 | 2983 |
| 1 | |
| AAVF12/HSC12 | 2984 |
| AAVF13/HSC13 | 2985 |
| AAVF14/HSC14 | 2986 |
| AAVF15/HSC15 | 2987 |
| AAVF16/HSC16 | 2988 |
| AAVF17/HSC17 | 2989 |
| AAVCBr-E1 | 2990 |
| AAVCBr-E2 | 2991 |
| AAVCBr-E3 | 2992 |
| AAVCBr-E4 | 2993 |
| AAVCBr-E5 | 2994 |
| AAVCBr-e5 | 2995 |
| AAVCBr-E6 | 2996 |
| AAVCBr-E7 | 2997 |
| AAVCBr-E8 | 2998 |
| AAVCLv-D1 | 2999 |
| AAVCLv-D2 | 3000 |
| AAVCLv-D3 | 3001 |
| AAVCLv-D4 | 3002 |
| AAVCLv-D5 | 3003 |
| AAVCLv-D6 | 3004 |
| AAVCLv-D7 | 3005 |
| AAVCLv-D8 | 3006 |
| AAVCLv-E1 | 3007 |
| AAVCLv-R1 | 3008 |
| AAVCLv-R2 | 3009 |
| AAVCLv-R3 | 3010 |
| AAVCLv-R4 | 3011 |
| AAVCLv-R5 | 3012 |
| AAVCLv-R6 | 3013 |
| AAVCLv-R7 | 3014 |
| AAVCLv-R8 | 3015 |
| AAVCLv-R9 | 3016 |
| AAVCLg-F1 | 3017 |
| AAVCLg-F2 | 3018 |
| AAVCLg-F3 | 3019 |
| AAVCLg-F4 | 3020 |
| AAVCLg-F5 | 3021 |
| AAVCLg-F6 | 3022 |
| AAVCLg-F7 | 3023 |
| AAVCLg-F8 | 3024 |
| AAVCSp-1 | 3025 |
| AAVCSp-10 | 3026 |
| AAVCSp-11 | 3027 |
| AAVCSp-2 | 3028 |
| AAVCSp-3 | 3029 |
| AAVCSp-4 | 3030 |
| AAVCSp-6 | 3031 |
| AAVCSp-7 | 3032 |
| AAVCSp-8 | 3033 |
| AAVCSp-9 | 3034 |
| AAVCHt-2 | 3035 |
| AAVCHt-3 | 3036 |
| AAVCKd-1 | 3037 |
| AAVCKd-10 | 3038 |
| AAVCKd-2 | 3039 |
| AAVCKd-3 | 3040 |
| AAVCKd-4 | 3041 |
| AAVCKd-6 | 3042 |
| AAVCKd-7 | 3043 |
| AAVCKd-8 | 3044 |
| AAVCLv-1 | 3045 |
| AAVCLv-12 | 3046 |
| AAVCLv-13 | 3047 |
| AAVCLv-2 | 3048 |
| AAVCLv-3 | 3049 |
| AAVCLv-4 | 3050 |
| AAVCLv-6 | 3051 |
| AAVCLv-8 | 3052 |
| AAVCKd-B1 | 3053 |
| AAVCKd-B2 | 3054 |
| AAVCKd-B3 | 3055 |
| AAVCKd-B4 | 3056 |
| AAVCKd-B5 | 3057 |
| AAVCKd-B6 | 3058 |
| AAVCKd-B7 | 3059 |
| AAVCKd-B8 | 3060 |
| AAVCKd-H1 | 3061 |
| AAVCKd-H2 | 3062 |
| AAVCKd-H3 | 3063 |
| AAVCKd-H4 | 3064 |
| AAVCKd-H5 | 3065 |
| AAVCKd-H6 | 3066 |
| AAV CHt-1 | 3067 |
| AAVCLvl-1 | 3068 |
| AAVCLv1-2 | 3069 |
| AAVCLv1-3 | 3070 |
| AAVCLv1-4 | 3071 |
| AAVC1v1-7 | 3072 |
| AAVC1v1-8 | 3073 |
| AAVC1v1-9 | 3074 |
| AAVC1v1-10 | 3075 |
| AAV.VR-355 | 3076 |
| AAV.hu.48R3 | 3077 |
| AAVCBr-E1 | 3078 |
| AAVCBr-E2 | 3079 |
| AAVCBr-E3 | 3080 |
| AAVCBr-E4 | 3081 |
| AAVCBr-E5 | 3082 |
| AAVCBr-e5 | 3083 |
| AAVCBr-E6 | 3084 |
| AAVCBr-E7 | 3085 |
| AAVCBr-E8 | 3086 |
| AAVCLv-D1 | 3087 |
| AAVCLv-D2 | 3088 |
| AAVCLv-D3 | 3089 |
| AAVCLv-D4 | 3090 |
| AAVCLv-D5 | 3091 |
| AAVCLv-D6 | 3092 |
| AAVCLv-D7 | 3093 |
| AAVCLv-D8 | 3094 |
| AAVCLv-E1 | 3095 |
| AAVCLv-R1 | 3096 |
| AAVCLv-R2 | 3097 |
| AAVCLv-R3 | 3098 |
| AAVCLv-R4 | 3099 |
| AAVCLv-R5 | 3100 |
| AAVCLv-R6 | 3101 |
| AAVCLv-R7 | 3102 |
| AAVCLv-R8 | 3103 |
| AAVCLv-R9 | 3104 |
| AAVCLg-F1 | 3105 |
| AAVCLg-F2 | 3106 |
| AAVCLg-F3 | 3107 |
| AAVCLg-F4 | 3108 |
| AAVCLg-F5 | 3109 |
| AAVCLg-F6 | 3110 |
| AAVCLg-F7 | 3111 |
| AAVCLg-F8 | 3112 |
| AAVCSp-1 | 3113 |
| AAVCSp-10 | 3114 |
| AAVCSp-11 | 3115 |
| AAVCSp-2 | 3116 |
| AAVCSp-3 | 3117 |
| AAVCSp-4 | 3118 |
| AAVCSp-6 | 3119 |
| AAVCSp-7 | 3120 |
| AAVCSp-8 | 3121 |
| AAVCSp-9 | 3122 |
| AAVCHt-2 | 3123 |
| AAVCHt-3 | 3124 |
| AAVCKd-1 | 3125 |
| AAVCKd-10 | 3126 |
| AAVCKd-2 | 3127 |
| AAVCKd-3 | 3128 |
| AAVCKd-4 | 3129 |
| AAVCKd-6 | 3130 |
| AAVCKd-7 | 3131 |
| AAVCKd-8 | 3132 |
| AAVCLv-1 | 3133 |
| AAVCLv-12 | 3134 |
| AAVCLv-13 | 3135 |
| AAVCLv-2 | 3136 |
| AAVCLv-3 | 3137 |
| AAVCLv-4 | 3138 |
| AAVCLv-6 | 3139 |
| AAVCLv-8 | 3140 |
| AAVCKd-B1 | 3141 |
| AAVCKd-B2 | 3142 |
| AAVCKd-B3 | 3143 |
| AAVCKd-B4 | 3144 |
| AAVCKd-B5 | 3145 |
| AAVCKd-B6 | 3146 |
| AAVCKd-B7 | 3147 |
| AAVCKd-B8 | 3148 |
| AAVCKd-H1 | 3149 |
| AAVCKd-H2 | 3150 |
| AAVCKd-H3 | 3151 |
| AAVCKd-H4 | 3152 |
| AAVCKd-H5 | 3153 |
| AAVCKd-H6 | 3154 |
| AAV CHt-1 | 3155 |
| AAVCHt-P2 | 3156 |
| AAVCHt-P5 | 3157 |
| AAVCHt-P9 | 3158 |
| AAVCBr-7.1 | 3159 |
| AAVCBr-7.2 | 3160 |
| AAVCBr-7.3 | 3161 |
| AAVCBr-7.4 | 3162 |
| AAVCBr-7.5 | 3163 |
| AAVCBr-7.7 | 3164 |
| AAVCBr-7.8 | 3165 |
| AAV CBr-7.10 | 3166 |
| AAVCKd-N3 | 3167 |
| AAVCKd-N4 | 3168 |
| AAVCKd-N9 | 3169 |
| AAVCLv-L4 | 3170 |
| AAVCLv-L5 | 3171 |
| AAVCLv-L6 | 3172 |
| AAVCLv-K1 | 3173 |
| AAVCLv-K3 | 3174 |
| AAVCLv-K6 | 3175 |
| AAVCLv-M1 | 3176 |
| AAVCLv-M11 | 3177 |
| AAVCLv-M2 | 3178 |
| AAVCLv-M5 | 3179 |
| AAVCLv-M6 | 3180 |
| AAVCLv-M7 | 3181 |
| AAVCLv-M8 | 3182 |
| AAVCLv-M9 | 3183 |
| AAVCHt-P1 | 3184 |
| AAVCHt-P6 | 3185 |
| AAVCHt-P8 | 3186 |
| AAVCHt-6.1 | 3187 |
| AAV CHt-6.10 | 3188 |
| AAVCHt-6.5 | 3189 |
| AAVCHt-6.6 | 3190 |
| AAVCHt-6.7 | 3191 |
| AAVCHt-6.8 | 3192 |
| AAVCSp-8.10 | 3193 |
| AAVCSp-8.2 | 3194 |
| AAVCSp-8.4 | 3195 |
| AAVCSp-8.5 | 3196 |
| AAVCSp-8.6 | 3197 |
| AAVCSp-8.7 | 3198 |
| AAVCSp-8.8 | 3199 |
| AAVCSp-8.9 | 3200 |
| AAV CBr-B7.3 | 3201 |
| AAV CBr-B7.4 | 3202 |
| AAV3B | 3203 |
| AAV4 | 3204 |
| AAV5 | 3205 |
| AAVCHt-P2 | 3206 |
| AAVCHt-P5 | 3207 |
| AAVCHt-P9 | 3208 |
| AAVCBr-7.1 | 3209 |
| AAVCBr-7.2 | 3210 |
| AAVCBr-7.3 | 3211 |
| AAVCBr-7.4 | 3212 |
| AAVCBr-7.5 | 3213 |
| AAVCBr-7.7 | 3214 |
| AAVCBr-7.8 | 3215 |
| AAV CBr-7.10 | 3216 |
| AAVCKd-N3 | 3217 |
| AAVCKd-N4 | 3218 |
| AAVCKd-N9 | 3219 |
| AAVCLv-L4 | 3220 |
| AAVCLv-L5 | 3221 |
| AAVCLv-L6 | 3222 |
| AAVCLv-K1 | 3223 |
| AAVCLv-K3 | 3224 |
| AAVCLv-K6 | 3225 |
| AAVCLv-M1 | 3226 |
| AAVCLv-M11 | 3227 |
| AAVCLv-M2 | 3228 |
| AAVCLv-M5 | 3229 |
| AAVCLv-M6 | 3230 |
| AAVCLv-M7 | 3231 |
| AAVCLv-M8 | 3232 |
| AAVCLv-M9 | 3233 |
| AAVCHt-P1 | 3234 |
| AAVCHt-P6 | 3235 |
| AAVCHt-P8 | 3236 |
| AAVCHt-6.1 | 3237 |
| AAV CHt-6.10 | 3238 |
| AAVCHt-6.5 | 3239 |
| AAVCHt-6.6 | 3240 |
| AAVCHt-6.7 | 3241 |
| AAVCHt-6.8 | 3242 |
| AAVCSp-8.10 | 3243 |
| AAVCSp-8.2 | 3244 |
| AAVCSp-8.4 | 3245 |
| AAVCSp-8.5 | 3246 |
| AAVCSp-8.6 | 3247 |
| AAV CSp-8.7 | 3248 |
| AAVCSp-8.8 | 3249 |
| AAVCSp-8.9 | 3250 |
| AAV CBr-B7.3 | 3251 |
| AAV CBr-B7.4 | 3252 |
| AAV3B | 3253 |
| AAV4 | 3254 |
| AAV5 | 3255 |
| GPV | 3256 |
| B19 | 3257 |
| MVM | 3258 |
| FPV | 3259 |
| CPV | 3260 |
| AAV6 | 3261 |
| AAV6 | 3262 |
| AAV2 | 3263 |
| ShH1O | 3264 |
| ShH13 | 3265 |
| ShH1O | 3266 |
| ShH1O | 3267 |
| ShH1O | 3268 |
| ShH1O | 3269 |
| ShH1O | 3270 |
| rh74 | 3271 |
| rh74 | 3272 |
| AAV8 | 3273 |
| rh74 | 3274 |
| rh74 (RHM4-1) | 3275 |
| rh74 (RHM15-1) | 3276 |
| rh74 (RHM15-2) | 3277 |
| rh74 (RHM15- | 3278 |
| 3/RHM15-5) | |
| rh74 (RHM15-4) | 3279 |
| rh74 (RHM15-6) | 3280 |
| rh74 (RHM4-1) | 3281 |
| rh74 (RHM15-1) | 3282 |
| rh74 (RHM15-2) | 3283 |
| rh74 (RHM15- | 3284 |
| 3/RHM15-5) | |
| rh74 (RHM15-4) | 3285 |
| rh74 (RHM15-6) | 3286 |
| AAV2 (comprising lung | 3287 |
| specific polypeptide) | |
| AAV2 (comprising lung | 3288 |
| specific polypeptide) | |
| Anc80 | 3289 |
| Anc80 | 3290 |
| Anc81 | 3291 |
| Anc80 | 3292 |
| Anc82 | 3293 |
| Anc82 | 3294 |
| Anc83 | 3295 |
| Anc83 | 3296 |
| Anc84 | 3297 |
| Anc84 | 3298 |
| Anc94 | 3299 |
| Anc94 | 3300 |
| Anc113 | 3301 |
| Anc113 | 3302 |
| Anc126 | 3303 |
| Anc126 | 3304 |
| Anc127 | 3305 |
| Anc127 | 3306 |
| Anc80L27 | 3307 |
| Anc80L59 | 3308 |
| Anc80L60 | 3309 |
| Anc80L62 | 3310 |
| Anc80L65 | 3311 |
| Anc80L33 | 3312 |
| Anc80L36 | 3313 |
| Anc80L44 | 3314 |
| Anc80L1 | 3315 |
| Anc80L1 | 3316 |
| AAV-X1 | 3317 |
| AAV-X1b | 3318 |
| AAV-X5 | 3319 |
| AAV-X19 | 3320 |
| AAV-X21 | 3321 |
| AAV-X22 | 3322 |
| AAV-X23 | 3323 |
| AAV-X24 | 3324 |
| AAV-X25 | 3325 |
| AAV-X26 | 3326 |
| AAV-X1 | 3327 |
| AAV-X1b | 3328 |
| AAV-X5 | 3329 |
| AAV-X19 | 3330 |
| AAV-X21 | 3331 |
| AAV-X22 | 3332 |
| AAV-X23 | 3333 |
| AAV-X24 | 3334 |
| AAV-X25 | 3335 |
| AAV-X26 | 3336 |
| AAVrh8 | 3337 |
| AAVrh8VP2FC5 | 3338 |
| AAVrh8VP2FC44 | 3339 |
| AAVrh8VP2ApoB100 | 3340 |
| AAVrh8VP2RVG | 3341 |
| AAVrh8VP2Angiopep-2 | 3342 |
| VP2 | |
| AAV9.47VP1.3 | 3343 |
| AAV9.47VP2ICAMg3 | 3344 |
| AAV9.47VP2RVG | 3345 |
| AAV9.47VP2Angiopep- | 3346 |
| 2 | |
| AAV9.47VP2A-string | 3347 |
| AAVrh8VP2FC5 VP2 | 3348 |
| AAVrh8VP2FC44 VP2 | 3349 |
| AAVrh8VP2ApoB100 | 3350 |
| VP2 | |
| AAVrh8VP2RVG VP2 | 3351 |
| AAVrh8VP2Angiopep-2 | 3352 |
| VP2 | |
| AAV9.47VP2ICAMg3 | 3353 |
| VP2 | |
| AAV9.47VP2RVG VP2 | 3354 |
| AAV9.47VP2Angiopep- | 3355 |
| 2 VP2 | |
| AAV9.47VP2A- | 3356 |
| string VP2 | |
| rAAV-B1 | 3357 |
| rAAV-B2 | 3358 |
| rAAV-B3 | 3359 |
| rAAV-B4 | 3360 |
| rAAV-B1 | 3361 |
| rAAV-B2 | 3362 |
| rAAV-B3 | 3363 |
| rAAV-B4 | 3364 |
| rAAV-L1 | 3365 |
| rAAV-L2 | 3366 |
| rAAV-L3 | 3367 |
| rAAV-L4 | 3368 |
| rAAV-L1 | 3369 |
| rAAV-L2 | 3370 |
| rAAV-L3 | 3371 |
| rAAV-L4 | 3372 |
| AAV9 | 3373 |
| rAAV | 3374 |
| rAAV | 3375 |
| rAAV | 3376 |
| rAAV | 3377 |
| rAAV | 3378 |
| rAAV | 3379 |
| rAAV | 3380 |
| rAAV | 3381 |
| rAAV | 3382 |
| rAAV | 3383 |
| rAAV | 3384 |
| rAAV | 3385 |
| rAAV | 3386 |
| rAAV | 3387 |
| rAAV | 3388 |
| rAAV | 3389 |
| rAAV | 3390 |
| rAAV | 3391 |
| rAAV | 3392 |
| rAAV | 3393 |
| rAAV | 3394 |
| rAAV | 3395 |
| rAAV | 3396 |
| rAAV | 3397 |
| rAAV | 3398 |
| rAAV | 3399 |
| rAAV | 3400 |
| rAAV | 3401 |
| rAAV | 3402 |
| rAAV | 3403 |
| rAAV | 3404 |
| rAAV | 3405 |
| rAAV | 3406 |
| rAAV | 3407 |
| rAAV | 3408 |
| rAAV | 3409 |
| rAAV | 3410 |
| rAAV | 3411 |
| rAAV | 3412 |
| rAAV | 3413 |
| rAAV | 3414 |
| rAAV | 3415 |
| rAAV | 3416 |
| rAAV | 3417 |
| rAAV | 3418 |
| rAAV | 3419 |
| rAAV | 3420 |
| rAAV | 3421 |
| rAAV | 3422 |
| rAAV | 3423 |
| rAAV | 3424 |
| rAAV | 3425 |
| rAAV | 3426 |
| rAAV | 3427 |
| rAAV | 3428 |
| rAAV | 3429 |
| rAAV | 3430 |
| rAAV | 3431 |
| rAAV | 3432 |
| rAAV | 3433 |
| rAAV | 3434 |
| rAAV | 3435 |
| rAAV | 3436 |
| rAAV | 3437 |
| rAAV | 3438 |
| rAAV | 3439 |
| rAAV | 3440 |
| rAAV | 3441 |
| rAAV | 3442 |
| rAAV | 3443 |
| rAAV | 3444 |
| rAAV | 3445 |
| rAAV | 3446 |
| rAAV | 3447 |
| rAAV | 3448 |
| rAAV | 3449 |
| rAAV | 3450 |
| rAAV | 3451 |
| rAAV | 3452 |
| rAAV | 3453 |
| rAAV | 3454 |
| rAAV | 3455 |
| rAAV | 3456 |
| rAAV | 3457 |
| rAAV | 3458 |
| rAAV | 3459 |
| rAAV | 3460 |
| rAAV | 3461 |
| rAAV | 3462 |
| rAAV | 3463 |
| rAAV | 3464 |
| rAAV | 3465 |
| rAAV | 3466 |
| rAAV | 3467 |
| rAAV | 3468 |
| rAAV | 3469 |
| rAAV | 3470 |
| rAAV | 3471 |
| rAAV | 3472 |
| rAAV | 3473 |
| rAAV | 3474 |
| rAAV | 3475 |
| rAAV | 3476 |
| rAAV | 3477 |
| rAAV | 3478 |
| rAAV | 3479 |
| rAAV | 3480 |
| rAAV | 3481 |
| rAAV | 3482 |
| rAAV | 3483 |
| rAAV | 3484 |
| rAAV | 3485 |
| rAAV | 3486 |
| rAAV | 3487 |
| rAAV | 3488 |
| rAAV | 3489 |
| rAAV | 3490 |
| rAAV | 3491 |
| rAAV | 3492 |
| rAAV | 3493 |
| rAAV | 3494 |
| rAAV | 3495 |
| rAAV | 3496 |
| rAAV | 3497 |
| rAAV | 3498 |
| rAAV | 3499 |
| rAAV | 3500 |
| rAAV | 3501 |
| AAV8E532K | 3502 |
| AAV8E532K | 3503 |
| rAAV4 | 3504 |
| rAAV4 | 3505 |
| rAAV4 | 3506 |
| rAAV4 | 3507 |
| rAAV4 | 3508 |
| rAAV4 | 3509 |
| rAAV4 | 3510 |
| rAAV4 | 3511 |
| rAAV4 | 3512 |
| rAAV4 | 3513 |
| rAAV4 | 3514 |
| rAAV4 | 3515 |
| rAAV4 | 3516 |
| rAAV4 | 3517 |
| rAAV4 | 3518 |
| rAAV4 | 3519 |
| rAAV4 | 3520 |
| rAAV4 | 3521 |
| rAAV4 | 3522 |
| rAAV4 | 3523 |
| AAV11 | 3524 |
| AAV12 | 3525 |
| rh32 | 3526 |
| rh33 | 3527 |
| rh34 | 3528 |
| rAAV4 | 3529 |
| rAAV4 | 3530 |
| rAAV4 | 3531 |
| rAAV4 | 3532 |
| rAAV4 | 3533 |
| rAAV4 | 3534 |
| AAV2/8 | 3535 |
| AAV2/8 | 3536 |
| ancestral AAV | 3537 |
| ancestral AAV variant | 3538 |
| C4 | |
| ancestral AAV variant | 3539 |
| C7 | |
| ancestral AAV variant | 3540 |
| G4 | |
| consensus amino acid | 3541 |
| sequence of ancestral | |
| AAV variants, C4, C7 | |
| and G4 | |
| consensus amino acid | 3542 |
| sequence of ancestral | |
| AAV variants, C4 and | |
| C7 | |
| AAVS (with an AAV2 | 3543 |
| phospholipase | |
| domain) | |
| AAVVR-942n | 3544 |
| AAVS-A (M569V) | 3545 |
| AAVS-A (M569V) | 3546 |
| AAVS-A (Y585V) | 3547 |
| AAVS-A (Y585V) | 3548 |
| AAVS-A (L587T) | 3549 |
| AAVS-A (L587T) | 3550 |
| AAVS-A | 3551 |
| (Y585V/L587T) | |
| AAVS-A | 3552 |
| (Y585V/L587T) | |
| AAV5-B (D652A) | 3553 |
| AAV5-B (D652A) | 3554 |
| AAV5-B (T362M) | 3555 |
| AAV5-B (T362M) | 3556 |
| AAV5-B (Q359D) | 3557 |
| AAV5-B (Q359D) | 3558 |
| AAV5-B (E350Q) | 3559 |
| AAV5-B (E350Q) | 3560 |
| AAV5-B (P533S) | 3561 |
| AAV5-B (P533S) | 3562 |
| AAV5-B (P533G) | 3563 |
| AAV5-B (P533G) | 3564 |
| AAVS-mutation in loop | 3565 |
| V11 | |
| AAVS-mutation in loop | 3566 |
| V11 | |
| AAVS | 3567 |
| Mut A (LK03/AAVS) | 3568 |
| Mut B (LK03/AAVS) | 3569 |
| Mut C (AAV8/AAV3B) | 3570 |
| MutD (AAV5/AAV3B) | 3571 |
| Mut E (AAV8/AAV3B) | 3572 |
| Mut F (AAV3B/AAV8) | 3573 |
| AAV44.9 | 3574 |
| AAV44.9 | 3575 |
| AAVrh8 | 3576 |
| AAV44.9 (S470N) | 3577 |
| rh74 VP1 | 3578 |
| AAV-LK03 (L125I) | 3579 |
| AAV3B (S663V + T492V) | 3580 |
| Anc80 | 3581 |
| Anc80 | 3582 |
| Anc81 | 3583 |
| Anc81 | 3584 |
| Anc82 | 3585 |
| Anc82 | 3586 |
| Anc83 | 3587 |
| Anc83 | 3588 |
| Anc84 | 3589 |
| Anc84 | 3590 |
| Anc94 | 3591 |
| Anc94 | 3592 |
| Anc113 | 3593 |
| Anc113 | 3594 |
| Anc126 | 3595 |
| Anc126 | 3596 |
| Anc127 | 3597 |
| Anc127 | 3598 |
| Anc80L27 | 3599 |
| Anc80L59 | 3600 |
| Anc80L60 | 3601 |
| Anc80L62 | 3602 |
| Anc80L65 | 3603 |
| Anc80L33 | 3604 |
| Anc80L36 | 3605 |
| Anc80L44 | 3606 |
| Anc80L1 | 3607 |
| Anc80L1 | 3608 |
| AAVrh1O | 3609 |
| Anc11O | 3610 |
| Anc11O | 3611 |
| AAVrh32.33 | 3612 |
| AAVrh74 | 3613 |
| AAV2 | 3614 |
| AAV2 | 3615 |
| AAV2 | 3616 |
| PaNo-like vims | 3617 |
| PaNo-like vims | 3618 |
| PaNo-like vims | 3619 |
| PaNo-like vims | 3620 |
| PaNo-like vims | 3621 |
| PaNo-like vims | 3622 |
| AAVrh.10 | 3623 |
| AAVrh.10 | 3624 |
| AAV2tYF | 3625 |
| AAV-SPK | 3626 |
| AAV2.5 | 3627 |
| AAV1.1 | 3628 |
| AAV6.1 | 3629 |
| AAV6.3.1 | 3630 |
| AAV2i8 | 3631 |
| AAV2i8 | 3632 |
| ttAAV | 3633 |
| ttAAV-S312N | 3634 |
| ttAAV-S312N | 3635 |
| AAV6 (Y705, Y731, and | 3636 |
| T492) | |
| AAV2 | 3637 |
| AAV2 | 3638 |

### AAV Production

The AAV vectors described herein may be produced using an AAV production cell line, such as a mammalian cell line (e.g., HEK293) or an insect cell line (e.g., Sf9). The AAV vectors may be produced using known methods for AAV production, including the helper-free transfection method and the baculovirus production method.

In some embodiments, an AAV viral vector of the disclosure is produced using a helper-free transfection method. The AAV expression cassette may be introduced into an AAV production cell line, along with Rep and Cap sequences and required “helper” sequences comprising genes from adenovirus. These helper sequences (E4, E2a and VA genes) mediate AAV replication. According to some embodiments, a transfer plasmid (comprising the AAV expression cassette), a Rep/Cap plasmid, and a helper plasmid (comprising E4, E2a, and VA) are transfected into viral production cells (e.g., HEK293 cells), to produce infectious AAV particles. Rep/Cap and the adenovirus helper genes may also be combined into a single plasmid.

In some embodiments, an AAV viral vector is produced using a baculovirus production method. In some embodiments, AAV production cells (e.g., Sf9 cells or derivatives thereof) are infected with one, two, or three baculovirus particles. In some embodiments, a first baculovirus particle comprises a sequence encoding the AAV expression cassette. In some embodiments, a second baculovirus particle comprises a sequence encoding AAV Rep and Cap genes.

AAV particles may be collected from AAV producer cell lysate, or from the tissue culture media without lysing the cells. The AAV particles may then be further purified, formulated for clinical use, and/or sterile filtered.

### AAV9-CK8e-spCas9

The disclosure provides an AAV vector of serotype 9 (AAV9) having 5′ and 3′ ITR sequences derived from an AAV of serotype 2 (AAV2), flanking a human codon optimized sequence encoding Streptococcus pyogenes (S. pyogenes) Cas9 (SpCas9) under the control of a truncated M-creatine kinase regulatory cassette (CK8e). Optionally, the human codon optimized sequence encoding SpCas9 is further flanked by two nuclear localization sequences (NLS), at the 5′ end, an SV40 NLS sequence and at the 3′ end, a nucleoplasm in NLS. Optionally, a sequence encoding a polyadenylation signal (polyA) or a miniature polyA is positioned 3′ of the nucleoplasmin NLS.

In some embodiments, the AAV9 vector comprises from 5′ to 3′, a sequence encoding an AAV2 5′ ITR, a sequence encoding a truncated M-creatine kinase regulatory cassette (CK8e), a human codon optimized sequence encoding SpCas9, and a sequence encoding an AAV2 3′ ITR.

In some embodiments, the AAV9 vector comprises from 5′ to 3′, a sequence encoding an AAV2 5′ ITR, a sequence encoding a truncated M-creatine kinase regulatory cassette (CK8e), a human codon optimized sequence encoding SpCas9, a sequence encoding a mini polyA and a sequence encoding an AAV2 3′ ITR.

In some embodiments, the AAV9 vector comprises from 5′ to 3′, a sequence encoding an AAV2 5′ ITR, an SV40 NLS, a sequence encoding a truncated M-creatine kinase regulatory cassette (CK8e), a human codon optimized sequence encoding SpCas9, a nucleoplasmin NLS, a sequence encoding a mini polyA and a sequence encoding an AAV2 3′ ITR.

In some embodiments, the AAV2 5′ ITR comprises the sequence of

(SEQ ID NO: 27)

| | |
|---|---|
| 1 | TTGGCCACTC CCTCTCTGCG CGCTCGCTCG CTCACTGAGG |
| | CCGGGCGACC AAAGGTCGCC |
| 61 | CGACGCCCGG GCTTTGCCCG GGCGGCCTCA GTGAGCGAGC |
| | GAGCGCGCAG AGAGGGAGTG |
| 121 | GCCAACTCCA TCACTAGGGG TTCCT. |

In some embodiments, the AAV2 3′ ITR comprises the sequence of

(SEQ ID NO: 28)

| | |
|---|---|
| 1 | AGGAACCCCT AGTGATGGAG TTGGCCACTC CCTCTCTGCG |
| | CGCTCGCTCG CTCACTGAGG |
| 61 | CCGCCCGGGC AAAGGCCGGG CGTCGGGCGA CCTTTGGTCG |
| | CCCGGCCTCA GTGAGCGAGC |
| 121 | GAGCGCGGAG AGAGGGAGTG GCCAA. |

In some embodiments, the SV40 NLS comprises the sequence of

(SEQ ID NO: 2363)
ccaaagaagaagcggaaggtc.

In some embodiments, the nucleoplasmin NLS comprises the sequence of

(SEQ ID NO: 2364)
aaaaggccggcggccacgaaaaaggccggccaggcaaaaaagaaaaag.

In some embodiments, the sequence encoding the truncated M-creatine kinase regulatory cassette (CK8e) comprises the sequence of

(SEQ ID NO: 19)

| | |
|---|---|
| 1 | TGCCCATGTA AGGAGGCAAG GCCTGGGGAC ACCCGAGATG |
| | CCTGGTTATA ATTAACCCAG |
| 61 | ACATGTGGCT GCCCCCCCCC CCCCAACACC TGCTGCCTCT |
| | AAAAATAACC CTGCATGCCA |
| 121 | TGTTCCCGGC GAAGGGCCAG CTGTCCCCCG CCAGCTAGAC |
| | TCAGCACTTA GTTTAGGAAC |
| 181 | CAGTGAGCAA GTCAGCCCTT GGGGCAGCCC ATACAAGGCC |
| | ATGGGGCTGG GCAAGCTGCA |
| 241 | CGCCTGGGTC CGGGGTGGGC ACGGTGCCCG GGCAACGAGC |
| | TGAAAGCTCA TCTGCTCTCA |
| 301 | GGGGCCCCTC CCTGGGGACA GCCCCTCCTG GCTAGTCACA |
| | CCCTGTAGGC TCCTCTATAT |
| 361 | AACCCAGGGG CACAGGGGCT GCCCTCATTC TACCACCACC |
| | TCCACAGCAC AGACAGACAC |
| 421 | TCAGGAGCCA GCCAGC. |

In some embodiments, the human codon optimized sequence encoding SpCas9 comprises the sequence of

(SEQ ID NO: 2365)

| | |
|---|---|
| 1 | gacaagaagt acagcatcgg cctggacatc ggcaccaact |
| | ctgtgggctg ggccgtgatc |
| 61 | accgacgagt acaaggtgcc cagcaagaaa ttcaaggtgc |
| | tgggcaacac cgaccggcac |
| 121 | agcatcaaga agaacctgat cggagccctg ctgttcgaca |
| | gcggcgaaac agccgaggcc |
| 181 | acccggctga agagaaccgc cagaagaaga tacaccagac |
| | ggaagaaccg gatctgctat |
| 241 | ctgcaagaga tcttcagcaa cgagatggcc aaggtggacg |
| | acagcttctt ccacagactg |
| 301 | gaagagtcct tcctggtgga agaggataag aagcacgagc |
| | ggcaccccat cttcggcaac |
| 361 | atcgtggacg aggtggccta ccacgagaag taccccacca |
| | tctaccacct gagaaagaaa |
| 421 | ctggtggaca gcaccgacaa ggccgacctg cggctgatct |
| | atctggccct ggcccacatg |
| 481 | atcaagttcc ggggccactt cctgatcgag ggcgacctga |
| | accccgacaa cagcgacgtg |
| 541 | gacaagctgt tcatccagct ggtgcagacc tacaaccagc |
| | tgttcgagga aaaccccatc |
| 601 | aacgccagcg gcgtggacgc caaggccatc ctgtctgcca |
| | gactgagcaa gagcagacgg |
| 661 | ctggaaaatc tgatcgccca gctgcccggc gagaagaaga |
| | atggcctgtt cggaaacctg |
| 721 | attgccctga gcctgggcct gacccccaac ttcaagagca |
| | acttcgacct ggccgaggat |
| 781 | gccaaactgc agctgagcaa ggacacctac gacgacgacc |
| | tggacaacct gctggcccag |
| 841 | atcggcgacc agtacgccga cctgtttctg gccgccaaga |
| | acctgtccga cgccatcctg |
| 901 | ctgagcgaca tcctgagagt gaacaccgag atcaccaagg |
| | cccccctgag cgcctctatg |
| 961 | atcaagagat acgacgagca ccaccaggac ctgaccctgc |
| | tgaaagctct cgtgcggcag |
| 1021 | cagctgcctg agaagtacaa agagattttc ttcgaccaga |
| | gcaagaacgg ctacgccggc |
| 1081 | tacattgacg gcggagccag ccaggaagag ttctacaagt |
| | tcatcaagcc catcctggaa |
| 1141 | aagatggacg gcaccgagga actgctcgtg aagctgaaca |
| | gagaggacct gctgcggaag |
| 1201 | cagcggacct tcgacaacgg cagcatcccc caccagatcc |
| | acctgggaga gctgcacgcc |
| 1261 | attctgcggc ggcaggaaga tttttaccca ttcctgaagg |
| | acaaccggga aaagatcgag |
| 1321 | aagatcctga ccttccgcat cccctactac gtgggccctc |
| | tggccagggg aaacagcaga |
| 1381 | ttcgcctgga tgaccagaaa gagcgaggaa accatcaccc |
| | cctggaactt cgaggaagtg |
| 1441 | gtggacaagg gcgcttccgc ccagagcttc atcgagcgga |
| | tgaccaactt cgataagaac |
| 1501 | ctgcccaacg agaaggtgct gcccaagcac agcctgctgt |
| | acgagtactt caccgtgtat |
| 1561 | aacgagctga ccaaagtgaa atacgtgacc gagggaatga |
| | gaaagcccgc cttcctgagc |
| 1621 | ggcgagcaga aaaaggccat cgtggacctg ctgttcaaga |
| | ccaaccggaa agtgaccgtg |
| 1681 | aagcagctga aagaggacta cttcaagaaa atcgagtgct |
| | tcgactccgt ggaaatctcc |
| 1741 | ggcgtggaag atcggttcaa cgcctccctg ggcacatacc |
| | acgatctgct gaaaattatc |
| 1801 | aaggacaagg acttcctgga caatgaggaa aacgaggaca |
| | ttctggaaga tatcgtgctg |
| 1861 | accctgacac tgtttgagga cagagagatg atcgaggaac |
| | ggctgaaaac ctatgcccac |
| 1921 | ctgttcgacg acaaagtgat gaagcagctg aagcggcgga |
| | gatacaccgg ctggggcagg |
| 1981 | ctgagccgga agctgatcaa cggcatccgg gacaagcagt |
| | ccggcaagac aatcctggat |
| 2041 | ttcctgaagt ccgacggctt cgccaacaga aacttcatgc |
| | agctgatcca cgacgacagc |
| 2101 | ctgaccttta aagaggacat ccagaaagcc caggtgtccg |
| | gccagggcga tagcctgcac |
| 2161 | gagcacattg ccaatctggc cggcagcccc gccattaaga |
| | agggcatcct gcagacagtg |
| 2221 | aaggtggtgg acgagctcgt gaaagtgatg ggccggcaca |
| | agcccgagaa catcgtgatc |
| 2281 | gaaatggcca gagagaacca gaccacccag aagggacaga |
| | agaacagccg cgagagaatg |
| 2341 | aagcggatcg aagagggcat caaagagctg ggcagccaga |
| | tcctgaaaga acaccccgtg |
| 2401 | gaaaacaccc agctgcagaa cgagaagctg tacctgtact |
| | acctgcagaa tgggcgggat |
| 2461 | atgtacgtgg accaggaact ggacatcaac cggctgtccg |
| | actacgatgt ggaccatatc |
| 2521 | gtgcctcaga gctttctgaa ggacgactcc atcgacaaca |
| | aggtgctgac cagaagcgac |
| 2581 | aagaaccggg gcaagagcga caacgtgccc tccgaagagg |
| | tcgtgaagaa gatgaagaac |
| 2641 | tactggcggc agctgctgaa cgccaagctg attacccaga |
| | gaaagttcga caatctgacc |
| 2701 | aaggccgaga gaggcggcct gagcgaactg gataaggccg |
| | gcttcatcaa gagacagctg |
| 2761 | gtggaaaccc ggcagatcac aaagcacgtg gcacagatcc |
| | tggactcccg gatgaacact |
| 2821 | aagtacgacg agaatgacaa gctgatccgg gaagtgaaag |
| | tgatcaccct gaagtccaag |
| 2881 | ctggtgtccg atttccggaa ggatttccag ttttacaaag |
| | tgcgcgagat caacaactac |
| 2941 | caccacgccc acgacgccta cctgaacgcc gtcgtgggaa |
| | ccgccctgat caaaaagtac |
| 3001 | cctaagctgg aaagcgagtt cgtgtacggc gactacaagg |
| | tgtacgacgt gcggaagatg |
| 3061 | atcgccaaga gcgagcagga aatcggcaag gctaccgcca |
| | agtacttctt ctacagcaac |
| 3121 | atcatgaact ttttcaagac cgagattacc ctggccaacg |
| | gcgagatccg gaagcggcct |
| 3181 | ctgatcgaga caaacggcga aaccggggag atcgtgtggg |
| | ataagggccg ggattttgcc |
| 3241 | accgtgcgga aagtgctgag catgccccaa gtgaatatcg |
| | tgaaaaagac cgaggtgcag |
| 3301 | acaggcggct tcagcaaaga gtctatcctg cccaagagga |
| | acagcgataa gctgatcgcc |
| 3361 | agaaagaagg actgggaccc taagaagtac ggcggcttcg |
| | acagccccac cgtggcctat |
| 3421 | tctgtgctgg tggtggccaa agtggaadag ggcaagtcca |
| | agaaactgaa gagtgtgaaa |
| 3481 | gagctgctgg ggatcaccat catggaaaga agcagcttcg |
| | agaagaatcc catcgacttt |
| 3541 | ctggaagcca agggctacaa agaagtgaaa aaggacctga |
| | tcatcaagct gcctaagtac |
| 3601 | tccctgttcg agctggaaaa cggccggaag agaatgctgg |
| | cctctgccgg cgaactgcag |
| 3661 | aagggaaacg aactggccct gccctccaaa tatgtgaact |
| | tcctgtacct ggccagccac |
| 3721 | tatgagaagc tgaagggctc ccccgaggat aatgagcaga |
| | aacagctgtt tgtggaacag |
| 3781 | cacaagcact acctggacga gatcatcgag cagatcagcg |
| | agttctccaa gagagtgatc |
| 3841 | ctggccgacg ctaatctgga caaagtgctg tccgcctaca |
| | acaagcaccg ggataagccc |
| 3901 | atcagagagc aggccgagaa tatcatccac ctgtttaccc |
| | tgaccaatct gggagcccct |
| 3961 | gccgccttca agtactttga caccaccatc gaccggaaga |
| | ggtacaccag caccaaagag |
| 4021 | gtgctggacg ccaccctgat ccaccagagc atcaccggcc |
| | tgtacgagac acggatcgac |
| 4081 | ctgtctcagc tgggaggcga c. |

### AAV9-H-sgRNA

The disclosure provides an AAV vector of serotype 9 (AAV9) having 5′ and 3′ ITR sequences derived from an AAV of serotype 2 (AAV2), flanking triple gRNA construct. In some embodiments, the triple guide construct comprises three single guide RNAs, each under the control of a unique promoter. In some embodiments, the triple guide construct comprises three single guide RNAs, each under the control of a unique RNA polymerase (pol) III promoter. In some embodiments, the triple guide construct comprises three single guide RNAs, each under the control of one of a U6 promoter, an H1 promoter or a 7SK promoter. In some embodiments, the triple guide construct comprises three single guide RNAs, each under the control of, from 5′ to 3′, a U6 promoter, an H1 promoter and a 7SK promoter. In some embodiments, the triple guide construct comprises, from 5′ to 3′, a sequence encoding a U6 promoter region, a sequence encoding a U6 promoter, a sequence encoding a first single guide RNA (sgRNA), a sequence encoding an H1 promoter, a sequence encoding a second sgRNA, a sequence encoding a 7SK promoter, a sequence encoding a third sgRNA. In some embodiments, the triple guide construct comprises, from 5′ to 3′, a sequence encoding a U6 promoter region, a sequence encoding a U6 promoter, a sequence encoding a first spacer sequence, a sequence encoding a first scaffold sequence, a sequence encoding an H1 promoter, a sequence encoding a second spacer sequence, a sequence encoding a second scaffold sequence, a sequence encoding a 7SK promoter, a sequence encoding a third spacer sequence, and a sequence encoding a third scaffold sequence.

In some embodiments, the sequence encoding the AAV2 5′ ITR lacks a terminal resolution sequence, resulting in the generation of a self-complementary vector.

In some embodiments, the sequence encoding the first sgRNA, the sequence encoding the second sgRNA, and the sequence encoding the third sgRNA, are identical. In some embodiments, the sequence encoding the first sgRNA, the sequence encoding the second sgRNA, and the sequence encoding the third sgRNA, are not identical.

In some embodiments, the sequence encoding the first spacer sequence, the sequence encoding the second spacer sequence, and the sequence encoding the third spacer sequence, are identical. In some embodiments, the sequence encoding the first spacer sequence, the sequence encoding the second spacer sequence, and the sequence encoding the third spacer sequence, are not identical.

In some embodiments, the sequence encoding the first scaffold sequence, the sequence encoding the second scaffold sequence, and the sequence encoding the third scaffold sequence, are identical. In some embodiments, the sequence encoding the first scaffold sequence, the sequence encoding the second scaffold sequence, and the sequence encoding the third scaffold sequence, are not identical. In some embodiments, the scaffold sequence specifically binds Cas9 or SpCas9. In some embodiments, the scaffold sequence comprises an extension of a first tetra loop to increase SpCas9 or Cas9 binding. In some embodiments, the scaffold sequence comprises an extension of at least 5 base pairs to the first tetra loop to increase SpCas9 or Cas9 binding. Alternatively, or in addition, in some embodiments, the scaffold sequence comprises a mutation that removes a transcription stop signal of “TTTT” to disrupt Pol III terminator binding and/or to promote sgRNA transcription. In some embodiments, the scaffold sequence comprises substitution of an adenosine (A) for a thymine (T) within a transcription stop signal of “TTTT” to disrupt Pol III terminator binding and/or to promote sgRNA transcription. In some embodiments, the scaffold sequence comprises the sequence of any one of SEQ ID NOs: 2348 or 2357-2362.

In some embodiments, the disclosure provides an AAV9 vector comprising, from 5′ to 3′, a sequence encoding an AAV2 5′ ITR sequence, a sequence encoding a U6 promoter region, a sequence encoding a U6 promoter, a sequence encoding a first spacer sequence, a sequence encoding a first scaffold sequence, a sequence encoding an H1 promoter, a sequence encoding a second spacer sequence, a sequence encoding a second scaffold sequence, a sequence encoding a 7SK promoter, a sequence encoding a third spacer sequence, a sequence encoding a third scaffold sequence and a sequence encoding an AAV2 3′ ITR sequence. In some embodiments, the spacer sequence may be referred to as a human sgRNA (H-sgRNA) and the scaffold sequence may be referred to as a sgRNA-constant.

In some embodiments, the disclosure provides a self-complementary AAV9 (scAAV9) vector comprising, from 5′ to 3′, a sequence encoding an AAV2 5′ ITR sequence lacking a terminal resolution sequence, a sequence encoding a U6 promoter region, a sequence encoding a U6 promoter, a sequence encoding a first spacer sequence, a sequence encoding a first scaffold sequence, a sequence encoding an H1 promoter, a sequence encoding a second spacer sequence, a sequence encoding a second scaffold sequence, a sequence encoding a 7SK promoter, a sequence encoding a third spacer sequence, a sequence encoding a third scaffold sequence and a sequence encoding an AAV2 3′ ITR sequence. In some embodiments, the spacer sequence may be referred to as a human sgRNA (H-sgRNA) and the scaffold sequence may be referred to as a sgRNA-constant.

In some embodiments, the sequence encoding the AAV2 5′ ITR comprises the sequence of

(SEQ ID NO: 2)

| | |
|---|---|
| 1 | CTGCGCGCTG GCTCGCTCAC TGAGGCCGCC CGGGCAAAGC |
| | CCGGGCGTCG GGCGACCTTT |
| 61 | GGTCGCCCGG CCTCAGTGAG CGAGCGAGCG CGCAGAGAGG |
| | GAGTGGCCAA CTCCATCACT |
| 121 | AGGGGTTCCT. |

In some embodiments, the sequence encoding the AAV2 5′ ITR lacking a terminal resolution sequence comprises the sequence of

(SEQ ID NO: 1)

| | |
|---|---|
| 1 | CCTGCAGGCA GCTGCGCGCT CGCTCGCTCA CTGAGGCCGC |
| | CCGGGCAAAG CCCGGGCGTC |
| 61 | GGGCGACCTT TGGTCGCCCG GCCTCAGTGA GCGAGCGAGC |
| | GCGCAGAGAG GGAGTGG. |

In some embodiments, the sequence encoding the AAV2 3′ ITR comprises the sequence of

(SEQ ID NO: 3)

| | |
|---|---|
| 1 | AGGAACCCCT AGTGATGGAG TTGGCCACTC CCTCTCTGCG |
| | CGCTCGCTCG CTCACTGAGG |
| 61 | CCGGGCGACC AAAGGTCGCC CGACGCCCGG GCTTTGCCCG |
| | GGCGGCCTCA GTGAGCGAGC |
| 121 | GAGCGCGCAG. |

In some embodiments, the sequence encoding the U6 promoter comprises the sequence of

(SEQ ID NO: 15)

| | |
|---|---|
| 1 | CGAGTCCAAC ACCCGTGGGA ATCCCATGGG CACCATGGCC |
| | CCTCGCTCCA AAAATGCTTT |
| 61 | CGCGTCGCGC AGACACTGCT CGGTAGTTTC GGCGATCAGC |
| | GTTTGAGTAA GAGCCCGCGT |
| 121 | CTGAACCCTC CGCGCCGCCC CGGCCCCAGT GGAAAGACGC |
| | GCAGGCAAAA CGCACCACGT |
| 181 | GACGGAGCGT GACCGCGCGC CGAGCGCGCG CCAAGGTCGG |
| | GCAGGAAGAG GGCCTATTTC |
| 241 | CCATGATTCC TTCATATTTG CATATACGAT ACAAGGCTGT |
| | TAGAGAGATA ATTAGAATTA |
| 301 | ATTTGACTGT AAACACAAAG ATATTAGTAC AAAATACGTG |
| | ACGTAGAAAG TAATAATTTC |
| 361 | TTGGGTAGTT TGCAGTTTTA AAATTATGTT TTAAAATGGA |
| | CTATCATATG CTTACCGTAA |
| 421 | CTTGAAAGTA TTTCGATTTC TTGGCTTTAT ATATCTTGTG |
| | GAAAGGACGA AA. |

In some embodiments, the sequence encoding the H1 promoter comprises the sequence of

(SEQ ID NO: 16)

| | |
|---|---|
| 1 | CTTCGGCGCG CCCATATTTG CATGTCGCTA TGTGTTCTGG |
| | GAAATCACCA TAAACGTGAA |
| 61 | ATGTCTTTGG ATTTGGGAAT CTTATAAGTT CTGTATGAGA |
| | CCACGGTA. |

In some embodiments, the sequence encoding the 7SK promoter comprises the sequence of

(SEQ ID NO: 17)

| | |
|---|---|
| 1 | TGACGGCGCG CCCTGCAGTA TTTAGCATGC CCCACCCATC |
| | TGCAAGGCAT TCTGGATAGT |
| 61 | GTCAAAACAG CCGGAAATCA AGTCCGTTTA TCTCAAACTT |
| | TAGCATTTTG GGAATAAATG |
| 121 | ATATTTGCTA TGCTGGTTAA ATTAGATTTT AGTTAAATTT |
| | CCTGCTGAAG CTCTAGTACG |
| 161 | ATAAGTAACT TGACCTAAGT GTAAAGTTGA GATTTCCTTC |
| | AGGTTTATAT AGCTTGTGCG |
| 241 | CCGCCTGGGT A. |

### AAV9-H-sqRNA-Exon 51

The disclosure provides a self-complementary AAV9 (scAAV9) vector comprising, from 5′ to 3′, a sequence encoding an AAV2 5′ ITR sequence lacking a terminal resolution sequence, a sequence encoding a U6 promoter region, a sequence encoding a U6 promoter, a sequence encoding a first spacer sequence, a sequence encoding a first scaffold sequence, a sequence encoding an H1 promoter, a sequence encoding a second spacer sequence, a sequence encoding a second scaffold sequence, a sequence encoding a 7SK promoter, a sequence encoding a third spacer sequence, a sequence encoding a third scaffold sequence and a sequence encoding an AAV2 3′ ITR sequence. In some embodiments, the spacer sequence may be referred to as a human sgRNA (H-sgRNA) and the scaffold sequence may be referred to as a sgRNA-constant.

In some embodiments, the self-complementary AAV9 (scAAV9) vector comprising, from 5′ to 3′, a sequence encoding an AAV2 5′ ITR sequence lacking a terminal resolution sequence, a sequence encoding a U6 promoter region, a sequence encoding a first single guide RNA (sgRNA), a sequence encoding an H1 promoter, a sequence encoding a second sgRNA, a sequence encoding a 7SK promoter, a sequence encoding a third sgRNA and a sequence encoding an AAV2 3′ ITR sequence.

In some embodiments, the sequence encoding a first spacer sequence, the sequence encoding a second spacer sequence and the sequence encoding a third spacer sequence comprise the sequence of CACCAGAGTAACAGTCTGAG (SEQ ID NO: 13), or a sequence at least 95% or at least 99% similar thereto. In some embodiments, the sequence encoding a first spacer sequence, the sequence encoding a second spacer sequence and the sequence encoding a third spacer sequence comprise the sequence of CACCAGAGTAACAGTCTGAG (SEQ ID NO: 13). In some embodiments, the sequence encoding a first scaffold sequence, the sequence encoding a second scaffold sequence and the sequence encoding a third scaffold sequence comprise the sequence of GTTTAAGAGCTATGCTGGAAACAGCATAGCAAGTTTAAATAAGGCTAGTCCGTTAT CAACTTGAAAAAGTGGCACCGAGTCGGTGCTTTTTTT (SEQ ID NO: 2348), or a sequence at least 95% or at least 99% similar thereto. In some embodiments, the sequence encoding a first scaffold sequence, the sequence encoding a second scaffold sequence and the sequence encoding a third scaffold sequence comprise the sequence of GTTTAAGAGCTATGCTGGAAACAGCATAGCAAGTTTAAATAAGGCTAGTCCGTTAT CAACTTGAAAAAGTGGCACCGAGTCGGTGCTTTTTTT (SEQ ID NO: 2348). In some embodiments, the sequence encoding a first scaffold sequence, the sequence encoding a second scaffold sequence and the sequence encoding a third scaffold sequence comprise the sequence of GTTTTAGAGCTAGAAATAGCAGTTAAAATAAGGCTAGTCCGTTATCAACTTGAAAAA GTGGCACCGAGTCGGTG (SEQ ID NO: 2357), or a sequence at least 95% or at least 99% similar thereto. In some embodiments, the sequence encoding a first scaffold sequence, the sequence encoding a second scaffold sequence and the sequence encoding a third scaffold sequence comprise the sequence of GTTTTAGAGCTAGAAATAGCAGTTAAAATAAGGCTAGTCCGTTATCAACTTGAAAAA GTGGCACCGAGTCGGTG (SEQ ID NO: 2357). In some embodiments, the sequence encoding a first scaffold sequence, the sequence encoding a second scaffold sequence and the sequence encoding a third scaffold sequence comprise the sequence of GTTGGAACCATTCAAAACAGCATAGCAAGTTAAAATAAGGCTAGTCCGTTATCAACT TGAAAAAGTGGCACCGAGTCGGTGCTTTTTT (SEQ ID NO: 2358), or a sequence at least 95% or at least 99% similar thereto. In some embodiments, the sequence encoding a first scaffold sequence, the sequence encoding a second scaffold sequence and the sequence encoding a third scaffold sequence comprise the sequence of GTTGGAACCATTCAAAACAGCATAGCAAGTTAAAATAAGGCTAGTCCGTTATCAACT TGAAAAAGTGGCACCGAGTCGGTGCTTTTTT (SEQ ID NO: 2358). In some embodiments, the sequence encoding a first scaffold sequence, the sequence encoding a second scaffold sequence and the sequence encoding a third scaffold sequence comprise the sequence of GTTTAAGAGCTATGAAACAGCATAGCAAGTTTAAATAAGGCTAGTCCGTTATCAACT TGAAAAAGTGGCACCGAGTCGGTGCTTTTTTT (SEQ ID NO: 2359), or a sequence at least 95% or at least 99% similar thereto. In some embodiments, the sequence encoding a first scaffold sequence, the sequence encoding a second scaffold sequence and the sequence encoding a third scaffold sequence comprise the sequence of GTTTAAGAGCTATGAAACAGCATAGCAAGTTTAAATAAGGCTAGTCCGTTATCAACT TGAAAAAGTGGCACCGAGTCGGTGCTTTTTTT (SEQ ID NO: 2359). In some embodiments, the sequence encoding a first scaffold sequence, the sequence encoding a second scaffold sequence and the sequence encoding a third scaffold sequence comprise the sequence of GTTTAAGAGCTATGCGAAACAGCATAGCAAGTTTAAATAAGGCTAGTCCGTTATCA ACTTGAAAAAGTGGCACCGAGTCGGTGCTTTTTTT (SEQ ID NO: 2360), or a sequence at least 95% or at least 99% similar thereto. In some embodiments, the sequence encoding a first scaffold sequence, the sequence encoding a second scaffold sequence and the sequence encoding a third scaffold sequence comprise the sequence of GTTTAAGAGCTATGCGAAACAGCATAGCAAGTTTAAATAAGGCTAGTCCGTTATCA ACTTGAAAAAGTGGCACCGAGTCGGTGCTTTTTTT (SEQ ID NO: 2360). In some embodiments, the sequence encoding a first scaffold sequence, the sequence encoding a second scaffold sequence and the sequence encoding a third scaffold sequence comprise the sequence of GTTTAAGAGCTATGCTGTTTGAAACAGCATAGCAAGTTTAAATAAGGCTAGTCCGTT ATCAACTTGAAAAAGTGGCACCGAGTCGGTGCTTTTTTT (SEQ ID NO: 2361), or a sequence at least 95% or at least 99% similar thereto. In some embodiments, the sequence encoding a first scaffold sequence, the sequence encoding a second scaffold sequence and the sequence encoding a third scaffold sequence comprise the sequence of GTTTAAGAGCTATGCTGTTTGAAACAGCATAGCAAGTTTAAATAAGGCTAGTCCGTT ATCAACTTGAAAAAGTGGCACCGAGTCGGTGCTTTTTTT (SEQ ID NO: 2361). In some embodiments, the sequence encoding a first scaffold sequence, the sequence encoding a second scaffold sequence and the sequence encoding a third scaffold sequence comprise the sequence of GTTTAAGAGCTATGCTGTTTTGGAAACAGCATAGCAAGTTTAAATAAGGCTAGTCC GTTATCAACTTGAAAAAGTGGCACCGAGTCGGTGCTTTTTTT (SEQ ID NO: 2362), or a sequence at least 95% or at least 99% similar thereto. In some embodiments, the sequence encoding a first scaffold sequence, the sequence encoding a second scaffold sequence and the sequence encoding a third scaffold sequence comprise the sequence of

(SEQ ID NO: 2362)
GTTTAAGAGCTATGCTGTTTTGGAAACAGCATAGCAAGTTTAAATAAGGCT
AGTCCGTTATCAACTTGAAAAAGTGGCACCGAGTCGGTGCTTTTTTT.

In some embodiments, the first sgRNA, the second sgRNA and/or the third sgRNA comprises a spacer sequence comprising SEQ ID NO: 13 and a scaffold sequence comprising SEQ ID NO: 2348, the combination of which comprises the sequence of

(SEQ ID NO: 2365)
CACCAGAGTAACAGTCTGAGGTTTAAGAGCTATGCTGGAAACAGCATAGCA
AGTTTAAATAAGGCTAGTCCGTTATCAACTTGAAAAAGTGGCACCGAGTCG
GTGCTTTTTTT.

In some embodiments, the first sgRNA, the second sgRNA and/or the third sgRNA comprises a spacer sequence comprising SEQ ID NO: 13 and a scaffold sequence comprising SEQ ID NO: 2357, the combination of which comprises the sequence of

(SEQ ID NO: 2366)
CACCAGAGTAACAGTCTGAGGTTTTAGAGCTAGAAATAGCAGTTAAAATAA
GGCTAGTCCGTTATCAACTTGAAAAAGTGGCACCGAGTCGGTG.

In some embodiments, the first sgRNA, the second sgRNA and/or the third sgRNA comprises a spacer sequence comprising SEQ ID NO: 13 and a scaffold sequence comprising SEQ ID NO: 2358, the combination of which comprises the sequence of

(SEQ ID NO: 2367)
CACCAGAGTAACAGTCTGAGGTTGGAACCATTCAAAACAGCATAGCAAGTT
AAAATAAGGCTAGTCCGTTATCAACTTGAAAAAGTGGCACCGAGTCGGTGC
TTTTTT.

In some embodiments, the first sgRNA, the second sgRNA and/or the third sgRNA comprises a spacer sequence comprising SEQ ID NO: 13 and a scaffold sequence comprising SEQ ID NO: 2359, the combination of which comprises the sequence of

(SEQ ID NO: 2368)
CACCAGAGTAACAGTCTGAGGTTTAAGAGCTATGAAACAGCATAGCAAGTT
TAAATAAGGCTAGTCCGTTATCAACTTGAAAAAGTGGCACCGAGTCGGTGC
TTTTTTT.

In some embodiments, the first sgRNA, the second sgRNA and/or the third sgRNA comprises a spacer sequence comprising SEQ ID NO: 13 and a scaffold sequence comprising SEQ ID NO: 2360, the combination of which comprises the sequence of

(SEQ ID NO: 2369)
CACCAGAGTAACAGTCTGAGGTTTAAGAGCTATGCGAAACAGCATAGCAAG
TTTAAATAAGGCTAGTCCGTTATCAACTTGAAAAAGTGGCACCGAGTCGGT
GCTTTTTTT.

In some embodiments, the first sgRNA, the second sgRNA and/or the third sgRNA comprises a spacer sequence comprising SEQ ID NO: 13 and a scaffold sequence comprising SEQ ID NO: 2361, the combination of which comprises the sequence of

(SEQ ID NO: 2370)
CACCAGAGTAACAGTCTGAGGTTTAAGAGCTATGCTGTTTGAAACAGCATA
GCAAGTTTAAATAAGGCTAGTCCGTTATCAACTTGAAAAAGTGGCACCGAG
TCGGTGCTTTTTTT.

In some embodiments, the first sgRNA, the second sgRNA and/or the third sgRNA comprises a spacer sequence comprising SEQ ID NO: 13 and a scaffold sequence comprising SEQ ID NO: 2362, the combination of which comprises the sequence of

(SEQ ID NO: 2371)
CACCAGAGTAACAGTCTGAGGTTTAAGAGCTATGCTGTTTTGGAAACAGCA
TAGCAAGTTTAAATAAGGCTAGTCCGTTATCAACTTGAAAAAGTGGCACCG
AGTCGGTGCTTTTTTT.

### AAV9-H-sqRNA-Exon 45

The disclosure provides a self-complementary AAV9 (scAAV9) vector comprising, from 5′ to 3′, a sequence encoding an AAV2 5′ ITR sequence lacking a terminal resolution sequence, a sequence encoding a U6 promoter region, a sequence encoding a U6 promoter, a sequence encoding a first spacer sequence, a sequence encoding a first scaffold sequence, a sequence encoding an H1 promoter, a sequence encoding a second spacer sequence, a sequence encoding a second scaffold sequence, a sequence encoding a 7SK promoter, a sequence encoding a third spacer sequence, a sequence encoding a third scaffold sequence and a sequence encoding an AAV2 3′ ITR sequence. In some embodiments, the spacer sequence may be referred to as a human sgRNA (H-sgRNA) and the scaffold sequence may be referred to as a sgRNA-constant.

In some embodiments, the self-complementary AAV9 (scAAV9) vector comprising, from 5′ to 3′, a sequence encoding an AAV2 5′ ITR sequence lacking a terminal resolution sequence, a sequence encoding a U6 promoter region, a sequence encoding a first single guide RNA (sgRNA), a sequence encoding an H1 promoter, a sequence encoding a second sgRNA, a sequence encoding a 7SK promoter, a sequence encoding a third sgRNA and a sequence encoding an AAV2 3′ ITR sequence.

In some embodiments, the sequence encoding a first spacer sequence, the sequence encoding a second spacer sequence and the sequence encoding a third spacer sequence comprise the sequence of ATCTTACAGGAACTCCAGGA (SEQ ID NO: 929), or a sequence at least 95% or at least 99% similar thereto. In some embodiments, the sequence encoding a first spacer sequence, the sequence encoding a second spacer sequence and the sequence encoding a third spacer sequence comprise the sequence of ATCTTACAGGAACTCCAGGA (SEQ ID NO: 929). In some embodiments, the inclusion of the 5′ nucleotides “AT” of the sequence of ATCTTACAGGAACTCCAGGA (SEQ ID NO: 929) reduces predicted off-target editing compared to a sequence that does not comprise one or both of these 5′ nucleotides. the sequence encoding a first spacer sequence, the sequence encoding a second spacer sequence and the sequence encoding a third spacer sequence comprise the sequence of ATCTTACAGGAACTCCAGGA (SEQ ID NO: 929), or a sequence at least 95% or at least 99% similar thereto, which maintains one or both of the 5′ nucleotides “AT”.

In some embodiments, the sequence encoding a first spacer sequence, the sequence encoding a second spacer sequence and the sequence encoding a third spacer sequence comprise the sequence of ATCTTACAGGAACTCCAGGA (SEQ ID NO: 929), or a sequence at least 95% or at least 99% similar thereto. In some embodiments, the sequence encoding a first spacer sequence, the sequence encoding a second spacer sequence and the sequence encoding a third spacer sequence comprise the sequence of ATCTTACAGGAACTCCAGGA (SEQ ID NO: 929). In some embodiments, the sequence encoding a first scaffold sequence, the sequence encoding a second scaffold sequence and the sequence encoding a third scaffold sequence comprise the sequence of GTTTAAGAGCTATGCTGGAAACAGCATAGCAAGTTTAAATAAGGCTAGTCCGTTAT CAACTTGAAAAAGTGGCACCGAGTCGGTGCTTTTTTT (SEQ ID NO: 2348), or a sequence at least 95% or at least 99% similar thereto. In some embodiments, the sequence encoding a first scaffold sequence, the sequence encoding a second scaffold sequence and the sequence encoding a third scaffold sequence comprise the sequence of GTTTAAGAGCTATGCTGGAAACAGCATAGCAAGTTTAAATAAGGCTAGTCCGTTAT CAACTTGAAAAAGTGGCACCGAGTCGGTGCTTTTTTT (SEQ ID NO: 2348). In some embodiments, the sequence encoding a first scaffold sequence, the sequence encoding a second scaffold sequence and the sequence encoding a third scaffold sequence comprise the sequence of GTTTTAGAGCTAGAAATAGCAGTTAAAATAAGGCTAGTCCGTTATCAACTTGAAAAA GTGGCACCGAGTCGGTG (SEQ ID NO: 2357), or a sequence at least 95% or at least 99% similar thereto. In some embodiments, the sequence encoding a first scaffold sequence, the sequence encoding a second scaffold sequence and the sequence encoding a third scaffold sequence comprise the sequence of GTTTTAGAGCTAGAAATAGCAGTTAAAATAAGGCTAGTCCGTTATCAACTTGAAAAA GTGGCACCGAGTCGGTG (SEQ ID NO: 2357). In some embodiments, the sequence encoding a first scaffold sequence, the sequence encoding a second scaffold sequence and the sequence encoding a third scaffold sequence comprise the sequence of GTTGGAACCATTCAAAACAGCATAGCAAGTTAAAATAAGGCTAGTCCGTTATCAACT TGAAAAAGTGGCACCGAGTCGGTGCTTTTTT (SEQ ID NO: 2358), or a sequence at least 95% or at least 99% similar thereto. In some embodiments, the sequence encoding a first scaffold sequence, the sequence encoding a second scaffold sequence and the sequence encoding a third scaffold sequence comprise the sequence of GTTGGAACCATTCAAAACAGCATAGCAAGTTAAAATAAGGCTAGTCCGTTATCAACT TGAAAAAGTGGCACCGAGTCGGTGCTTTTTT (SEQ ID NO: 2358). In some embodiments, the sequence encoding a first scaffold sequence, the sequence encoding a second scaffold sequence and the sequence encoding a third scaffold sequence comprise the sequence of GTTTAAGAGCTATGAAACAGCATAGCAAGTTTAAATAAGGCTAGTCCGTTATCAACT TGAAAAAGTGGCACCGAGTCGGTGCTTTTTTT (SEQ ID NO: 2359), or a sequence at least 95% or at least 99% similar thereto. In some embodiments, the sequence encoding a first scaffold sequence, the sequence encoding a second scaffold sequence and the sequence encoding a third scaffold sequence comprise the sequence of GTTTAAGAGCTATGAAACAGCATAGCAAGTTTAAATAAGGCTAGTCCGTTATCAACT TGAAAAAGTGGCACCGAGTCGGTGCTTTTTTT (SEQ ID NO: 2359). In some embodiments, the sequence encoding a first scaffold sequence, the sequence encoding a second scaffold sequence and the sequence encoding a third scaffold sequence comprise the sequence of GTTTAAGAGCTATGCGAAACAGCATAGCAAGTTTAAATAAGGCTAGTCCGTTATCA ACTTGAAAAAGTGGCACCGAGTCGGTGCTTTTTTT (SEQ ID NO: 2360), or a sequence at least 95% or at least 99% similar thereto. In some embodiments, the sequence encoding a first scaffold sequence, the sequence encoding a second scaffold sequence and the sequence encoding a third scaffold sequence comprise the sequence of GTTTAAGAGCTATGCGAAACAGCATAGCAAGTTTAAATAAGGCTAGTCCGTTATCA ACTTGAAAAAGTGGCACCGAGTCGGTGCTTTTTTT (SEQ ID NO: 2360). In some embodiments, the sequence encoding a first scaffold sequence, the sequence encoding a second scaffold sequence and the sequence encoding a third scaffold sequence comprise the sequence of GTTTAAGAGCTATGCTGTTTGAAACAGCATAGCAAGTTTAAATAAGGCTAGTCCGTT ATCAACTTGAAAAAGTGGCACCGAGTCGGTGCTTTTTTT (SEQ ID NO: 2361), or a sequence at least 95% or at least 99% similar thereto. In some embodiments, the sequence encoding a first scaffold sequence, the sequence encoding a second scaffold sequence and the sequence encoding a third scaffold sequence comprise the sequence of GTTTAAGAGCTATGCTGTTTGAAACAGCATAGCAAGTTTAAATAAGGCTAGTCCGTT ATCAACTTGAAAAAGTGGCACCGAGTCGGTGCTTTTTTT (SEQ ID NO: 2361). In some embodiments, the sequence encoding a first scaffold sequence, the sequence encoding a second scaffold sequence and the sequence encoding a third scaffold sequence comprise the sequence of GTTTAAGAGCTATGCTGTTTTGGAAACAGCATAGCAAGTTTAAATAAGGCTAGTCC GTTATCAACTTGAAAAAGTGGCACCGAGTCGGTGCTTTTTTT (SEQ ID NO: 2362), or a sequence at least 95% or at least 99% similar thereto. In some embodiments, the sequence encoding a first scaffold sequence, the sequence encoding a second scaffold sequence and the sequence encoding a third scaffold sequence comprise the sequence of

(SEQ ID NO: 2362)
GTTTAAGAGCTATGCTGTTTTGGAAACAGCATAGCAAGTTTAAATAAGGCT
AGTCCGTTATCAACTTGAAAAAGTGGCACCGAGTCGGTGCTTTTTTT.

In some embodiments, the first sgRNA, the second sgRNA and/or the third sgRNA comprises a spacer sequence comprising SEQ ID NO: 929 and a scaffold sequence comprising SEQ ID NO: 2348, the combination of which comprises the sequence of

(SEQ ID NO: 2372)
ATCTTACAGGAACTCCAGGAGTTTAAGAGCTATGCTGGAAACAGCATAGCA
AGTTTAAATAAGGCTAGTCCGTTATCAACTTGAAAAAGTGGCACCGAGTCG
GTGCTTTTTTT.

In some embodiments, the first sgRNA, the second sgRNA and/or the third sgRNA comprises a spacer sequence comprising SEQ ID NO: 929 and a scaffold sequence comprising SEQ ID NO: 2357, the combination of which comprises the sequence of

(SEQ ID NO: 2373)
ATCTTACAGGAACTCCAGGAGTTTTAGAGCTAGAAATAGCAGTTAAAATAA
GGCTAGTCCGTTATCAACTTGAAAAAGTGGCACCGAGTCGGTG.

In some embodiments, the first sgRNA, the second sgRNA and/or the third sgRNA comprises a spacer sequence comprising SEQ ID NO: 929 and a scaffold sequence comprising SEQ ID NO: 2358, the combination of which comprises the sequence of

(SEQ ID NO: 2374)
ATCTTACAGGAACTCCAGGAGTTGGAACCATTCAAAACAGCATAGCAAGTT
AAAATAAGGCTAGTCCGTTATCAACTTGAAAAAGTGGCACCGAGTCGGTGC
TTTTTT.

In some embodiments, the first sgRNA, the second sgRNA and/or the third sgRNA comprises a spacer sequence comprising SEQ ID NO: 929 and a scaffold sequence comprising SEQ ID NO: 2359, the combination of which comprises the sequence of

(SEQ ID NO: 2375)
ATCTTACAGGAACTCCAGGAGTTTAAGAGCTATGAAACAGCATAGCAAGTT
TAAATAAGGCTAGTCCGTTATCAACTTGAAAAAGTGGCACCGAGTCGGTGC
TTTTTTT.

In some embodiments, the first sgRNA, the second sgRNA and/or the third sgRNA comprises a spacer sequence comprising SEQ ID NO: 929 and a scaffold sequence comprising SEQ ID NO: 2360, the combination of which comprises the sequence of

(SEQ ID NO: 2376)
ATCTTACAGGAACTCCAGGAGTTTAAGAGCTATGCGAAACAGCATAGCAAG
TTTAAATAAGGCTAGTCCGTTATCAACTTGAAAAAGTGGCACCGAGTCGGT
GCTTTTTTT.

In some embodiments, the first sgRNA, the second sgRNA and/or the third sgRNA comprises a spacer sequence comprising SEQ ID NO: 929 and a scaffold sequence comprising SEQ ID NO: 2361, the combination of which comprises the sequence of

(SEQ ID NO: 2377)
ATCTTACAGGAACTCCAGGAGTTTAAGAGCTATGCTGTTTGAAACAGCATA
GCAAGTTTAAATAAGGCTAGTCCGTTATCAACTTGAAAAAGTGGCACCGAG
TCGGTGCTTTTTTT.

In some embodiments, the first sgRNA, the second sgRNA and/or the third sgRNA comprises a spacer sequence comprising SEQ ID NO: 929 and a scaffold sequence comprising SEQ ID NO: 2362, the combination of which comprises the sequence of

(SEQ ID NO: 2378)
ATCTTACAGGAACTCCAGGAGTTTAAGAGCTATGCTGTTTTGGAAACAGCA
TAGCAAGTTTAAATAAGGCTAGTCCGTTATCAACTTGAAAAAGTGGCACCG
AGTCGGTGCTTTTTTT.

AAV9-H-sqRNA-Exon 45 (18-mer)

The disclosure provides a self-complementary AAV9 (scAAV9) vector comprising, from 5′ to 3′, a sequence encoding an AAV2 5′ ITR sequence lacking a terminal resolution sequence, a sequence encoding a U6 promoter region, a sequence encoding a U6 promoter, a sequence encoding a first spacer sequence, a sequence encoding a first scaffold sequence, a sequence encoding an H1 promoter, a sequence encoding a second spacer sequence, a sequence encoding a second scaffold sequence, a sequence encoding a 7SK promoter, a sequence encoding a third spacer sequence, a sequence encoding a third scaffold sequence and a sequence encoding an AAV2 3′ ITR sequence. In some embodiments, the spacer sequence may be referred to as a human sgRNA (H-sgRNA) and the scaffold sequence may be referred to as a sgRNA-constant.

In some embodiments, the self-complementary AAV9 (scAAV9) vector comprising, from 5′ to 3′, a sequence encoding an AAV2 5′ ITR sequence lacking a terminal resolution sequence, a sequence encoding a U6 promoter region, a sequence encoding a first single guide RNA (sgRNA), a sequence encoding an H1 promoter, a sequence encoding a second sgRNA, a sequence encoding a 7SK promoter, a sequence encoding a third sgRNA and a sequence encoding an AAV2 3′ ITR sequence.

In some embodiments, the sequence encoding a first spacer sequence, the sequence encoding a second spacer sequence and the sequence encoding a third spacer sequence comprise the sequence of CTTACAGGAACTCCAGGA (SEQ ID NO: 2356), or a sequence at least 95% or at least 99% similar thereto. In some embodiments, the sequence encoding a first spacer sequence, the sequence encoding a second spacer sequence and the sequence encoding a third spacer sequence comprise the sequence of CTTACAGGAACTCCAGGA (SEQ ID NO: 2356).

In some embodiments, the sequence encoding a first spacer sequence, the sequence encoding a second spacer sequence and the sequence encoding a third spacer sequence comprise the sequence of CTTACAGGAACTCCAGGA (SEQ ID NO: 2356), or a sequence at least 95% or at least 99% similar thereto. In some embodiments, the sequence encoding a first spacer sequence, the sequence encoding a second spacer sequence and the sequence encoding a third spacer sequence comprise the sequence of CTTACAGGAACTCCAGGA (SEQ ID NO: 2356). In some embodiments, the sequence encoding a first scaffold sequence, the sequence encoding a second scaffold sequence and the sequence encoding a third scaffold sequence comprise the sequence of GTTTAAGAGCTATGCTGGAAACAGCATAGCAAGTTTAAATAAGGCTAGTCCGTTAT CAACTTGAAAAAGTGGCACCGAGTCGGTGCTTTTTTT (SEQ ID NO: 2348), or a sequence at least 95% or at least 99% similar thereto. In some embodiments, the sequence encoding a first scaffold sequence, the sequence encoding a second scaffold sequence and the sequence encoding a third scaffold sequence comprise the sequence of GTTTAAGAGCTATGCTGGAAACAGCATAGCAAGTTTAAATAAGGCTAGTCCGTTAT CAACTTGAAAAAGTGGCACCGAGTCGGTGCTTTTTTT (SEQ ID NO: 2348). In some embodiments, the sequence encoding a first scaffold sequence, the sequence encoding a second scaffold sequence and the sequence encoding a third scaffold sequence comprise the sequence of GTTTTAGAGCTAGAAATAGCAGTTAAAATAAGGCTAGTCCGTTATCAACTTGAAAAA GTGGCACCGAGTCGGTG (SEQ ID NO: 2357), or a sequence at least 95% or at least 99% similar thereto. In some embodiments, the sequence encoding a first scaffold sequence, the sequence encoding a second scaffold sequence and the sequence encoding a third scaffold sequence comprise the sequence of GTTTTAGAGCTAGAAATAGCAGTTAAAATAAGGCTAGTCCGTTATCAACTTGAAAAA GTGGCACCGAGTCGGTG (SEQ ID NO: 2357). In some embodiments, the sequence encoding a first scaffold sequence, the sequence encoding a second scaffold sequence and the sequence encoding a third scaffold sequence comprise the sequence of GTTGGAACCATTCAAAACAGCATAGCAAGTTAAAATAAGGCTAGTCCGTTATCAACT TGAAAAAGTGGCACCGAGTCGGTGCTTTTTT (SEQ ID NO: 2358), or a sequence at least 95% or at least 99% similar thereto. In some embodiments, the sequence encoding a first scaffold sequence, the sequence encoding a second scaffold sequence and the sequence encoding a third scaffold sequence comprise the sequence of GTTGGAACCATTCAAAACAGCATAGCAAGTTAAAATAAGGCTAGTCCGTTATCAACT TGAAAAAGTGGCACCGAGTCGGTGCTTTTTT (SEQ ID NO: 2358). In some embodiments, the sequence encoding a first scaffold sequence, the sequence encoding a second scaffold sequence and the sequence encoding a third scaffold sequence comprise the sequence of GTTTAAGAGCTATGAAACAGCATAGCAAGTTTAAATAAGGCTAGTCCGTTATCAACT TGAAAAAGTGGCACCGAGTCGGTGCTTTTTTT (SEQ ID NO: 2359), or a sequence at least 95% or at least 99% similar thereto. In some embodiments, the sequence encoding a first scaffold sequence, the sequence encoding a second scaffold sequence and the sequence encoding a third scaffold sequence comprise the sequence of GTTTAAGAGCTATGAAACAGCATAGCAAGTTTAAATAAGGCTAGTCCGTTATCAACT TGAAAAAGTGGCACCGAGTCGGTGCTTTTTTT (SEQ ID NO: 2359). In some embodiments, the sequence encoding a first scaffold sequence, the sequence encoding a second scaffold sequence and the sequence encoding a third scaffold sequence comprise the sequence of GTTTAAGAGCTATGCGAAACAGCATAGCAAGTTTAAATAAGGCTAGTCCGTTATCA ACTTGAAAAAGTGGCACCGAGTCGGTGCTTTTTTT (SEQ ID NO: 2360), or a sequence at least 95% or at least 99% similar thereto. In some embodiments, the sequence encoding a first scaffold sequence, the sequence encoding a second scaffold sequence and the sequence encoding a third scaffold sequence comprise the sequence of GTTTAAGAGCTATGCGAAACAGCATAGCAAGTTTAAATAAG GCTAGTCCGTTATCA ACTTGAAAAAGTGGCACCGAGTCGGTGCTTTTTTT (SEQ ID NO: 2360). In some embodiments, the sequence encoding a first scaffold sequence, the sequence encoding a second scaffold sequence and the sequence encoding a third scaffold sequence comprise the sequence of GTTTAAGAGCTATGCTGTTTGAAACAGCATAGCAAGTTTAAATAAGGCTAGTCCGTT ATCAACTTGAAAAAGTGGCACCGAGTCGGTGCTTTTTTT (SEQ ID NO: 2361), or a sequence at least 95% or at least 99% similar thereto. In some embodiments, the sequence encoding a first scaffold sequence, the sequence encoding a second scaffold sequence and the sequence encoding a third scaffold sequence comprise the sequence of GTTTAAGAGCTATGCTGTTTGAAACAGCATAGCAAGTTTAAATAAGGCTAGTCCGTT ATCAACTTGAAAAAGTGGCACCGAGTCGGTGCTTTTTTT (SEQ ID NO: 2361). In some embodiments, the sequence encoding a first scaffold sequence, the sequence encoding a second scaffold sequence and the sequence encoding a third scaffold sequence comprise the sequence of GTTTAAGAGCTATGCTGTTTTGGAAACAGCATAGCAAGTTTAAATAAGGCTAGTCC GTTATCAACTTGAAAAAGTGGCACCGAGTCGGTGCTTTTTTT (SEQ ID NO: 2362), or a sequence at least 95% or at least 99% similar thereto. In some embodiments, the sequence encoding a first scaffold sequence, the sequence encoding a second scaffold sequence and the sequence encoding a third scaffold sequence comprise the sequence of

(SEQ ID NO: 2362)
GTTTAAGAGCTATGCTGTTTTGGAAACAGCATAGCAAGTTTAAATAAGGCT
AGTCCGTTATCAACTTGAAAAAGTGGCACCGAGTCGGTGCTTTTTTT.

In some embodiments, the first sgRNA, the second sgRNA and/or the third sgRNA comprises a spacer sequence comprising SEQ ID NO: 2356 and a scaffold sequence comprising SEQ ID NO: 2348, the combination of which comprises the sequence of

(SEQ ID NO: 2349)
CTTACAGGAACTCCAGGAGTTTAAGAGCTATGCTGGAAACAGCATAGCAAG
TTTAAATAAGGCTAGTCCGTTATCAACTTGAAAAAGTGGCACCGAGTCGGT
GCTTTTTTT.

In some embodiments, the first sgRNA, the second sgRNA and/or the third sgRNA comprises a spacer sequence comprising SEQ ID NO: 2356 and a scaffold sequence comprising SEQ ID NO: 2357, the combination of which comprises the sequence of

(SEQ ID NO: 2350)
CTTACAGGAACTCCAGGAGTTTTAGAGCTAGAAATAGCAGTTAAAATAAGG
CTAGTCCGTTATCAACTTGAAAAAGTGGCACCGAGTCGGTG.

In some embodiments, the first sgRNA, the second sgRNA and/or the third sgRNA comprises a spacer sequence comprising SEQ ID NO: 2356 and a scaffold sequence comprising SEQ ID NO: 2358, the combination of which comprises the sequence of

(SEQ ID NO: 2351)
CTTACAGGAACTCCAGGAGTTGGAACCATTCAAAACAGCATAGCAAGTTAA
AATAAGGCTAGTCCGTTATCAACTTGAAAAAGTGGCACCGAGTCGGTGCTT
TTTT.

In some embodiments, the first sgRNA, the second sgRNA and/or the third sgRNA comprises a spacer sequence comprising SEQ ID NO: 2356 and a scaffold sequence comprising SEQ ID NO: 2359, the combination of which comprises the sequence of

(SEQ ID NO: 2352)
CTTACAGGAACTCCAGGAGTTTAAGAGCTATGAAACAGCATAGCAAGTTTA
AATAAGGCTAGTCCGTTATCAACTTGAAAAAGTGGCACCGAGTCGGTGCTT
TTTTT.

In some embodiments, the first sgRNA, the second sgRNA and/or the third sgRNA comprises a spacer sequence comprising SEQ ID NO: 2356 and a scaffold sequence comprising SEQ ID NO: 2360, the combination of which comprises the sequence of

(SEQ ID NO: 2353)
CTTACAGGAACTCCAGGAGTTTAAGAGCTATGCGAAACAGCATAGCAAGTT
TAAATAAGGCTAGTCCGTTATCAACTTGAAAAAGTGGCACCGAGTCGGTGC
TTTTTTT.

In some embodiments, the first sgRNA, the second sgRNA and/or the third sgRNA comprises a spacer sequence comprising SEQ ID NO: 2356 and a scaffold sequence comprising SEQ ID NO: 2361, the combination of which comprises the sequence of

(SEQ ID NO: 2354)
ATCTTACAGGAACTCCAGGAGTTTAAGAGCTATGCTGTTTGAAACAGCATA
GCAAGTTTAAATAAGGCTAGTCCGTTATCAACTTGAAAAAGTGGCACCGAG
TCGGTGCTTTTTTT.

In some embodiments, the first sgRNA, the second sgRNA and/or the third sgRNA comprises a spacer sequence comprising SEQ ID NO: 2356 and a scaffold sequence comprising SEQ ID NO: 2362, the combination of which comprises the sequence of

(SEQ ID NO: 2355)
ATCTTACAGGAACTCCAGGAGTTTAAGAGCTATGCTGTTTTGGAAACAGCA
TAGCAAGTTTAAATAAGGCTAGTCCGTTATCAACTTGAAAAAGTGGCACCG
AGTCGGTGCTTTTTTT.

### Pharmaceutical Composition

Also provided herein is a pharmaceutical composition comprising an AAV vector of the disclosure. In some embodiments, the composition may further comprise a pharmaceutically-acceptable carrier and/or other medicinal agents, pharmaceutical agents, carriers, adjuvants, diluents, etc. In general, a “pharmaceutically acceptable carrier” is one that is non-toxic or unduly detrimental to cells. Exemplary physiologically acceptable carriers include sterile, pyrogen-free water and sterile, pyrogen-free phosphate buffered saline.

### Methods of Treatment

The AAV vectors disclosed herein may be used to treat or prevent a disease or disorder, such as a genetic disease or disorder, in a subject in need thereof. In some embodiments, the genetic disease or disorder is a muscle disease or disorder. The muscle disease or disorder may be selected from, for example, Duchenne Muscular Dystrophy (DMD), Becker muscular dystrophy (BMD), Emery-Dreifuss dystrophy, myotonic dystrophy, limb-girdle muscular dystrophy, oculopharyngeal muscular dystrophy, congenital dystrophy, familial periodic paralysis. In some embodiments, the muscle disease or disorder may be mitochondrial oxidative phosphorylation disorder, or a glycogen storage disease (e.g., von Gierke's disease, Pompe's disease, Forbes-Cori disease, Andersen's disease, McArdle's disease, Hers' disease, Tarui's disease, or Fanconi-Bickel syndrome.) In some embodiments, the AAV vectors disclosed herein are used to treat or prevent DMD.

The subject may be a mammal, such as a primate, ungulate (e.g., cow, pig, horse), cat, dog, domestic pet or domesticated mammal. In some cases, the mammal may be a rabbit, pig, horse, sheep, cow, cat or dog, or a human. In some embodiments, the subject is a human. In some embodiments, the subject is an adult human. In some embodiments, the subject is a juvenile human. In some embodiments, the subject is greater than about 18 years old, greater than about 25 years old, or greater than about 35 years old. In some embodiments, the subject is less than about 18 years old, less than about 16 years old, less than about 14 years old, less than about 12 years old, less than about 10 years old, less than about 8 years old, less than about 6 years old, less than about 5 years old, less than about 4 years old, less than about 3 years old, less than about 2 years old, less than about 1 year old, or less than about 6 months old.

Exemplary doses for achieving therapeutic effects are virus titers of at least about 10⁵, at least about 10⁶, at least about 10⁷, at least about 10⁸, at least about 10⁹, at least about 10¹⁰, at least about 10¹¹, at least about 10¹², at least about 10¹³, at least about 10¹⁴, at least about 10¹⁵ transducing units or more, for example about 10⁸-10¹³ transducing units.

Exemplary modes of administration of the AAV vectors include oral, rectal, transmucosal, topical, transdermal, inhalation, parenteral (e.g., intravenous, subcutaneous, intradermal, intramuscular, and intra-articular, as well as direct tissue or organ injection, alternatively, intrathecal, direct intramuscular, intraventricular, intravenous, intraperitoneal, intranasal, or intraocular injections. Injectables can be prepared in conventional forms, either as liquid solutions or suspensions, solid forms suitable for solution or suspension in liquid prior to injection, or as emulsions. Alternatively the virus may be administered locally, for example in a depot or sustained-release formulation.

The AAV vectors of the disclosure may optionally be administered simultaneously or sequentially with a second vector comprising an expression cassette for a nuclease. The second vector may be a viral vector (e.g., an AAV) or a non-viral vector (e.g., a plasmid or nanoparticle). The nuclease may be, for example, a Cas9 or a Cpf1 nuclease.

In some embodiments, the nuclease is codon optimized for expression in mammalian cells. In some embodiments, the nuclease is codon optimized for expression in human cells or mouse cells.

In some embodiments, a first AAV vector comprising an AAV expression cassette of the disclosure is administered simultaneously or sequentially with a second AAV vector comprising an expression cassette for a nuclease (e.g., a Cas9 nuclease).

In some embodiments a first AAV vector and a second AAV vector are administered to a subject, wherein the first vector comprises an expression cassette comprising sequences encoding a first inverted terminal repeat (ITR); a first promoter; a first gRNA comprising a first gRNA targeting region; a second promoter; a second gRNA comprising a second gRNA targeting region; a third promoter; a third gRNA comprising a third gRNA targeting region; and a second ITR, and wherein the second vector comprises an expression cassette for a Cas9 nuclease. Optionally, one or both of the first vector and the second-vector are self-complimentary. In some embodiments, one or both of the first vector and the second vector further comprise a stuffer sequence.

In some embodiments a first AAV vector and a second AAV vector are administered to a subject, wherein the first vector comprises an expression cassette comprising sequences encoding a first inverted terminal repeat (ITR); a first promoter; a first gRNA comprising a first gRNA targeting region (SEQ ID NO: 13) and a scaffold region; a second promoter; a second gRNA comprising a second gRNA targeting region (SEQ ID NO: 13) and a scaffold region; a third promoter; a third gRNA comprising a third gRNA targeting region (SEQ ID NO: 13) and a scaffold region; and a second ITR, and wherein the second vector comprises an expression cassette for a Cas9 nuclease. Optionally, one or both of the first vector and the second-vector are self-complimentary. In some embodiments, one or both of the first vector and the second vector further comprise a stuffer sequence.

In some embodiments a first AAV vector and a second AAV vector are administered to a subject, wherein the first vector comprises an expression cassette comprising sequences encoding a first inverted terminal repeat (ITR); the U6 promoter; a first gRNA comprising a first gRNA targeting region (SEQ ID NO: 13) and a scaffold region; the H1 promoter; a second gRNA comprising a second gRNA targeting region (SEQ ID NO: 13) and a scaffold region; the 7SK promoter; a third gRNA comprising a third gRNA targeting region (SEQ ID NO: 13) and a scaffold region; and a second ITR, and wherein the second vector comprises an expression cassette for a Cas9 nuclease. Optionally, one or both of the first vector and the second-vector are self-complimentary. In some embodiments, one or both of the first vector and the second vector further comprise a stuffer sequence.

In some embodiments a first AAV vector and a second AAV vector are administered to a subject, wherein the first vector comprises an expression cassette comprising sequences encoding a first inverted terminal repeat (ITR) (SEQ ID NO: 1); the U6 promoter (SEQ ID NO: 15); a first gRNA comprising a first gRNA targeting region (SEQ ID NO: 13) and a scaffold region; the H1 promoter (SEQ ID NO: 16); a second gRNA comprising a second gRNA targeting region (SEQ ID NO: 13) and a scaffold region; the 7SK promoter (SEQ ID NO: 17); a third gRNA comprising a third gRNA targeting region (SEQ ID NO: 13) and a scaffold region; and a second ITR (SEQ ID NO: 2), and wherein the second vector comprises an expression cassette for a Cas9 nuclease. Optionally, one or both of the first vector and the second-vector are self-complimentary. In some embodiments, one or both of the first vector and the second vector further comprise a stuffer sequence.

In some embodiments a first AAV vector and a second AAV vector are administered to a subject, wherein the first vector comprises an expression cassette comprising sequence at least 95% identical to or 100% identical to the sequence of SEQ ID NO: 25 or 26, and wherein the second vector comprises an expression cassette for a Cas9 nuclease. Optionally, one or both of the first vector and the second-vector are self-complimentary. In some embodiments, one or both of the first vector and the second vector further comprise a stuffer sequence.

### EXAMPLES

The following examples, which are included herein for illustration purposes only, are not intended to be limiting.

### Example 1

An AAV expression cassette having the sequence of SEQ ID NO: 25 is prepared using standard cloning techniques.

The AAV expression cassette comprises from 5′ to 3′, a first ITR, the U6 promoter, a first gRNA, the H1 promoter, a second gRNA, the 7SK promoter, a third gRNA, and a second ITR. The first, second, and third gRNA sequences are the same (SEQ ID NO: 13), and target the human dystrophin gene. The AAV expression cassette is self-complimentary.

### Example 2

An AAV expression cassette having the sequence of SEQ ID NO: 26 is prepared using standard cloning techniques.

The AAV expression cassette comprises from 5′ to 3′, a first ITR, the U6 promoter, a first gRNA, the H1 promoter, a second gRNA, the 7SK promoter, a third gRNA, a stuffer sequence, and a second ITR. The first, second, and third gRNA sequences are the same (SEQ ID NO: 13), and target the human dystrophin gene. The AAV expression cassette is single-stranded (i.e., not self-complimentary).

### Example 3

An AAV vector is prepared by transfecting an AAV production cell (e.g., HEK293) with a first plasmid comprising an AAV expression cassette of the disclosure (e.g., SEQ ID NO: 25 or 26), a second plasmid comprising the AAV rev and cap genes, and a third plasmid comprising adenoviral E4, E2a and VA genes. After incubation of the cells for a predetermined period of time, the cells are lysed. AAV vectors comprising the expression cassette are purified and quantified. A pharmaceutical composition is prepared by combining the purified AAV vector with a pharmaceutically acceptable carrier. The pharmaceutical composition is frozen until use.

### Example 4

An AAV vector is prepared by infecting an AAV production cell (e.g., Sf9) with a first baculovirus comprising an AAV expression cassette of the disclosure (e.g., SEQ ID NO: 25 or 26), and a second baculovirus comprising the AAV rev and cap genes. After incubation of the cells fora predetermined period of time, the cells are lysed. AAV vectors comprising the expression cassette are purified and quantified. A pharmaceutical composition is prepared by combining the purified AAV vector with a pharmaceutically acceptable carrier. The pharmaceutical composition is frozen until use.

### Example 5

A human subject suffering from DMD is treated by administering to the subject either the AAV vector of Example 3 or the AAV vector of Example 4, in combination with an AAV vector comprising an expression vector for a Cas9 nuclease. The vectors are administered either serially or sequentially, and are administered locally (e.g., intramuscularly) or systemically (e.g., intravenously). The dose of each vector administered to the subject ranges from about 10⁸ to about 10¹³ transducing units.

### Example 6

A dose ratio study was performed to determine optimal doses and ratios of AAV-Cas9 and self-complementary AAV-sgRNA for use in vivo. The study design is outlined in Table 19. Briefly, 4-week old mice (P4) were injected intraperitoneally (IP) with AAV9-Cas9 and AAV9-sgRNA. Various ratios (vector genomes per kilogram) were tested. At 4-weeks post-dose, various tissues were collected for analysis of tissue dystrophin protein, on-target editing efficiency, and Cas9 protein expression.

TABLE 19
Dose ratio study design

| Age at | AAV9- | AAV9- | | Total | | |
|---|---|---|---|---|---|---|
| dosing/ | Cas9 | sgRNA | | Dose | N | |
| ROA | (vg/kg) | (vg/kg) | Ratio | (vg/kg) | per group | Study Endpoints |
| P4/IP | 5 × 10¹³ | 2 × 10¹³ | 1:0.4 | 7 × 10¹³ | 12 | Tissue collection 4-weeks |
| | | 5 × 10¹³ | 1:1 | 1 × 10¹⁴ | 16 | post-dose for analysis of |
| | | 1 × 10¹⁴ | 1:2 | 1.5 × 10¹⁴ | 16 | tissue dystrophin protein, |
| | | 2 × 10¹⁴ | 1:4 | 2.5 × 10¹⁴ | 16 | on-target editing efficiency, |
| | 1 × 10¹⁴ | 5 × 10¹³ | 1:0.5 | 1.5 × 10¹⁴ | 12 | Cas9 protein, vector |
| | | 1 × 10¹⁴ | 1:1 | 2 × 10¹⁴ | 11 | genomes and transgene |
| | | 2 × 10¹⁴ | 1:2 | 3 × 10¹⁴ | 10 | expression |

ROA = Route of Administration; IP = Intraperitoneal

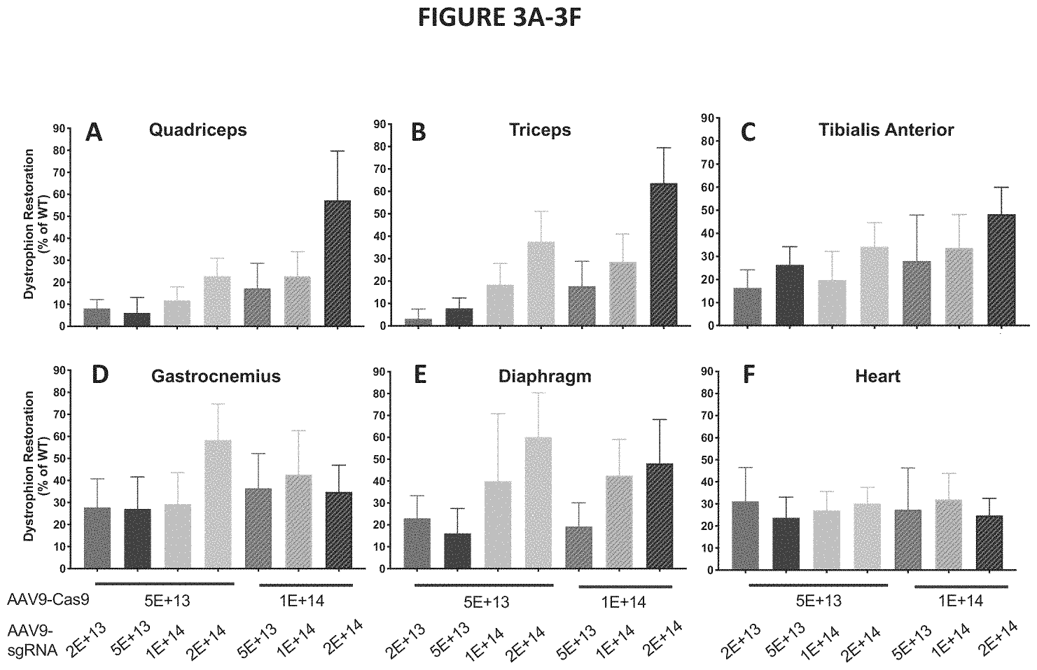

Tissue dystrophin quantification was performed on tissue samples from the quadriceps (FIG. 3A), triceps (FIG. 3B), tibialis anterior (FIG. 3C), gastrocnemius (FIG. 3D), diaphragm (FIG. 3E), and heart (FIG. 3F). Samples from these tissues were visualized using a capillary electrophoresis Western blot. Quantification was performed by comparing samples with a standard curve, and dystrophin restoration was expressed as percent of wildtype. Results are shown in FIGS. 3A-3F. In general, greater dystrophin restoration was observed with increased AAV9-sgRNA dose in the majority of skeletal muscles in and in the diaphragm.

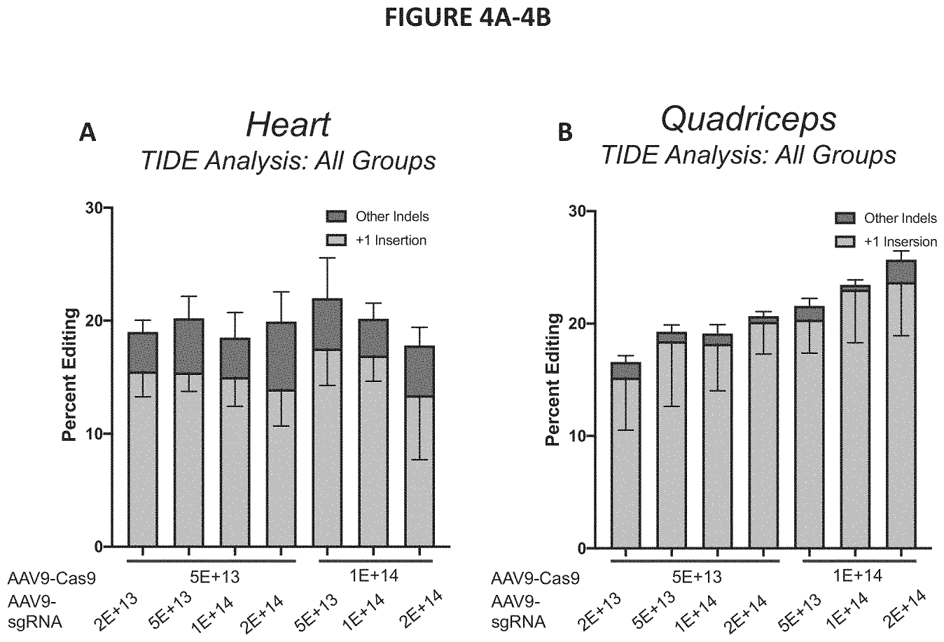

Editing efficiency was evaluated using TIDE (Tracking Indels by Decomposition) analysis in the heart and quadriceps (FIG. 4A-4B). A dose-response was observed in the quadriceps (FIG. 4B), but none was observed in the heart (FIG. 4A).

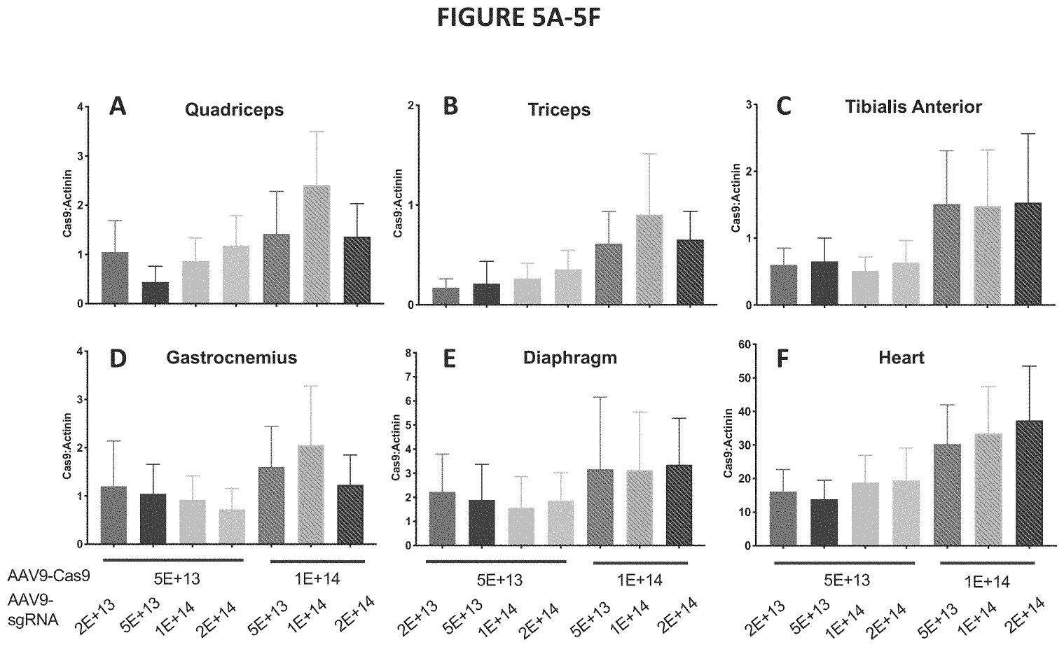

Cas9 levels were also measured in various tissues, including quadriceps (FIG. 5A), triceps (FIG. 5B), tibialis anterior (FIG. 5C), gastrocnemius (FIG. 5D), diaphragm (FIG. 5E) and heart (FIG. 5F). Samples from these tissues were visualized using a capillary electrophoresis Western blot. Quantification was performed by comparing samples with a standard curve. In each of these tissues, higher vector doses led to higher Cas9 expression levels.

The foregoing is illustrative of the present invention, and is not to be construed as limiting thereof. The invention is defined by the following claims, with equivalents of the claims to be included therein.

What is claimed is:

1. An AAV expression cassette comprising sequences encoding:
a first inverted terminal repeat (ITR);
a first promoter;
a first gRNA comprising a first gRNA targeting region and a scaffold region;
a second promoter;
a second gRNA comprising a second gRNA targeting region and a scaffold region;
a third promoter;
a third gRNA comprising a third gRNA targeting region and a scaffold region; and
a second ITR;
wherein the AAV expression cassette is self-complimentary.

2. The AAV expression cassette of claim 1, wherein the expression cassette further comprises a fourth promoter and a fourth gRNA comprising a fourth gRNA targeting region and a scaffold region.

3. The AAV expression cassette of claim 2, wherein the expression cassette further comprises a fifth promoter and a fifth gRNA comprising a fifth gRNA targeting sequence and a scaffold region.

4. The AAV expression cassette of any one of claims 1-3, wherein one or both of the first ITR and the second ITR are isolated or derived from any one of AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV.

5. The AAV expression cassette of any one of claims 1-4, wherein the one or both of the first ITR and the second ITR are isolated or derived from AAV2.

6. The AAV expression cassette of any one of claims 1-5, wherein the first ITR has a sequence that is at least 95% identical or 100% identical to SEQ ID NO: 1.

7. The AAV expression cassette of any one of claims 1-6, wherein the second ITR has a sequence that is at least 95% identical or 100% identical to the sequence of SEQ ID NO: 2.

8. The AAV expression cassette of any one of claims 1-7, wherein the at least one of the first, second, and third gRNA targeting sequences targets the dystrophin gene.

9. The AAV expression cassette of any one of claims 1-8, wherein the at least two of the first, second, and third gRNA targeting sequences are different.

10. The AAV expression cassette of any one of claims 1-9, wherein the first, second, and third gRNA targeting sequences are the same.

11. The AAV expression cassette of any one of claims 1-10, wherein the at least one of the first, second, and third promoters is the U6 promoter.

12. The AAV expression cassette of claim 11, wherein at least one of the first, second, and third promoters has a sequence at least 95% identical or 100% identical to the sequence of SEQ ID NO: 15.

13. The AAV expression cassette of any one of claims 1-12, wherein the at least one of the first, second, and third promoters is the H1 promoter.

14. The AAV expression cassette of claim 13, wherein at least one of the first, second, and third promoters has a sequence at least 95% identical or 100% identical to the sequence of SEQ ID NO: 16.

15. The AAV expression cassette of any one of claims 1-14, wherein the at least one of the first, second, and third promoters is the 7SK promoter.

16. The AAV expression cassette of claim 15, wherein at least one of the first, second, and third promoters has a sequence at least 95% identical or 100% identical to the sequence of SEQ ID NO: 17.

17. The AAV expression cassette of any one of claims 1-16, wherein the first promoter is the U6 promoter, the second promoter is the H1 promoter, and the third promoter is the 7SK promoter.

18. The AAV expression cassette of claim 17, wherein the first promoter has a sequence at least 95% identical or 100% identical to the sequence of SEQ ID NO: 15; the second promoter has a sequence at least 95% identical or 100% identical to the sequence of SEQ ID NO: 16; and the third promoter has a sequence at least 95% identical or 100% identical to the sequence of SEQ ID NO: 17.

19. The AAV expression cassette of claim 17, wherein the first ITR has a sequence at least 95% identical or 100% identical to the sequence of SEQ ID NO: 1; the first promoter has a sequence at least 95% identical or 100% identical to the sequence of SEQ ID NO: 15; the second promoter has a sequence at least 95% identical or 100% identical to the sequence of SEQ ID NO: 16; the third promoter has a sequence at least 95% identical or 100% identical to the sequence of SEQ ID NO: 17; and the second ITR has a sequence a sequence at least 95% identical or 100% identical to the sequence of SEQ ID NO: 2.

20. An AAV expression cassette comprising sequences encoding:
a first ITR;
a first promoter;
a first gRNA comprising a first gRNA targeting region and a scaffold region;
a second promoter;
a second gRNA comprising a second gRNA targeting sequence and a scaffold region;
a third promoter;
a third gRNA comprising a third gRNA targeting sequence and a scaffold region;
a first stuffer sequence; and
a second ITR;
wherein the stuffer sequence is a 3′ UTR sequence isolated or derived from a gene expressed in muscle.

21. The AAV expression cassette of claim 20, wherein the expression cassette further comprises a fourth promoter and a fourth gRNA comprising a fourth gRNA targeting region and a scaffold region.

22. The AAV expression cassette of claim 21, wherein the expression cassette further comprises a fifth promoter and a fifth gRNA comprising a fifth gRNA targeting sequence and a scaffold region.

23. The AAV expression cassette of any one of claims 20-22, wherein one or both of the first ITR and the second ITR are isolated or derived from any one of AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV.

24. The AAV expression cassette of claim 23, wherein the one or both of the first ITR and the second ITR are isolated or derived from AAV2.

25. The AAV expression cassette of any one of claims 20-24, wherein the first ITR has a sequence that is at least 95% identical or 100% identical to SEQ ID NO: 3.

26. The AAV expression cassette of any one of claims 20-25, wherein the second ITR has a sequence that is at least 95% identical or 100% identical to the sequence of SEQ ID NO: 2.

27. The AAV expression cassette of any one of claims 20-26, wherein the at least one of the first, second, and third gRNA targeting sequences targets the dystrophin gene.

28. The AAV expression cassette of any one of claims 20-27, wherein the at least two of the first, second, and third gRNA targeting sequences are different.

29. The AAV expression cassette of any one of claims 20-27, wherein the first, second, and third gRNA targeting sequences are the same.

30. The AAV expression cassette of any one of claims 20-29, wherein the at least one of the first, second, and third promoters is the U6 promoter.

31. The AAV expression cassette of claim 30, wherein at least one of the first, second, and third promoters has a sequence at least 95% identical or 100% identical to the sequence of SEQ ID NO: 15.

32. The AAV expression cassette of any one of claims 20-31, wherein the at least one of the first, second, and third promoters is the H1 promoter.

33. The AAV expression cassette of claim 32, wherein at least one of the first, second, and third promoters has a sequence at least 95% identical or 100% identical to the sequence of SEQ ID NO: 16.

34. The AAV expression cassette of any one of claims 20-33, wherein the at least one of the first, second, and third promoters is the 7SK promoter.

35. The AAV expression cassette of claim 34, wherein at least one of the first, second, and third promoters has a sequence at least 95% identical or 100% identical to the sequence of SEQ ID NO: 17.

36. The AAV expression cassette of any one of claims 20-35, wherein the first promoter is the U6 promoter, the second promoter is the H1 promoter, and the third promoter is the 7SK promoter.

37. The AAV expression cassette of claim 36, wherein the first promoter has a sequence at least 95% identical or 100% identical to the sequence of SEQ ID NO: 15; the second promoter has a sequence at least 95% identical or 100% identical to the sequence of SEQ ID NO: 16; and the third promoter has a sequence at least 95% identical or 100% identical to the sequence of SEQ ID NO: 17.

38. The AAV expression cassette of claim 36, wherein the first ITR has a sequence at least 95% identical or 100% identical to the sequence of SEQ ID NO: 3; the first promoter has a sequence at least 95% identical or 100% identical to the sequence of SEQ ID NO: 15; the second promoter has a sequence at least 95% identical or 100% identical to the sequence of SEQ ID NO: 16; the third promoter has a sequence at least 95% identical or 100% identical to the sequence of SEQ ID NO: 17; and the second ITR has a sequence a sequence at least 95% identical or 100% identical to the sequence of SEQ ID NO: 2.

39. A vector comprising the AAV expression cassette of any one of claims 1-38.

40. The vector of claim 39, wherein the vector is a non-viral vector.

41. The vector of claim 40, wherein the vector is a plasmid.

42. The vector of claim 39, wherein the vector is an AAV vector.

43. The vector of claim 42, wherein the AAV vector is a self-complimentary AAV (scAAV).

44. The vector of claim 42 or 43, wherein the AAV vector is selected from one of the following serotypes: AAV1, AAV2, AAV3, AAV4, AAV5, AAV6, AAV7, AAV8, AAV9, AAV10, AAV11, AAV12, AAVRh74, AAV2i8, AAVRh10, AAV39, AAV43, AAVRh8, avian AAV, bovine AAV, canine AAV, equine AAV, and ovine AAV.

45. An AAV particle comprising:
a capsid protein, and
the AAV expression cassette of any one of claims 1-38 encapsidated by the capsid protein.

46. The AAV particle of claim 45, wherein the capsid protein is isolated or derived from a wildtype AAV capsid of the following serotypes: AAV1, AAV2, AAV3, AAV4, AAV5, AAV6, AAV7, AAV8, AAV9, AAV10, AAV11, AAV12, AAVRh.74, AAV2i8, AAVRh.10, AAV39, AAV43, AAVRh.8, avian AAV, bovine AAV, canine AAV, equine AAV, and ovine AAV.

47. A baculovirus vector comprising the AAV expression cassette of any one of claims 1-38.

48. A method of producing an AAV vector comprising contacting a vector comprising the AAV expression cassette of any one of claims 1-38 with an AAV producer cell.

49. The method of claim 48, wherein the AAV producer cell is a mammalian cell.

50. The method of claim 49, wherein the mammalian cell is a HEK293 cell.

51. The method of claim 48, wherein the AAV producer cell is an insect cell.

52. The method of claim 51, wherein the insect cell is a Sf9 cell.

53. A method of correcting a gene defect in a cell, the method comprising contacting an AAV vector comprising the AAV expression cassette of any one of claims 1-38 with the cell.

54. The method of claim 53, wherein the cell is a human cell.

55. The method of claim 53 or 54, wherein the gene defect is a gene defect in the dystrophin gene.

56. The method of any one of claims 53-55, wherein the method also comprises contacting the cell with an AAV vector comprising an expression cassette for a CRISPR nuclease, e.g., a Cas9 nuclease.

57. A method of treating a subject in need thereof comprising administering to the subject an AAV vector comprising the AAV expression cassette of any one of claims 1-38.

58. The method of claim 57, wherein the subject is a human.

59. The method of claim 57 or 58, wherein the subject suffers from Duchenne Muscular Dystrophy (DMD).

60. The method of any one of claims 57-59, wherein the method also comprises administering to the subject an AAV vector comprising an expression cassette for a Cas9 nuclease.
